(19) United States
(12) Patent Application Publication
(10) Pub. No.: US 2025/0289793 A1
(43) Pub. Date: Sep. 18, 2025

(54) MACROCYCLIC INHIBITORS OF ATP CITRATE LYASE

(71) Applicant: Esperion Therapeutics, Inc., Ann Arbor, MI (US)

(72) Inventors: Stephen Lawrence Pinkosky, Plymouth, MI (US); Adam James Davenport, Abingdon, Oxfordshire (GB); Laura Jane Gleave, Abingdon, Oxfordshire (GB); Michelle Wing Yan Southey, Abingdon, Oxfordshire (GB); Nina Connelly Ursinyova, Abingdon, Oxfordshire (GB); Patrick Ross Walker, Abingdon, Oxfordshire (GB); Christopher John Yarnold, Abingdon, Oxfordshire (GB)

(21) Appl. No.: 18/862,295

(22) PCT Filed: May 1, 2023
(86) PCT No.: PCT/US2023/020546

§ 371 Date: Nov. 1, 2024

Related U.S. Application Data
(60) Provisional application No. 63/337,344, filed on May 2, 2022.
(60) Provisional application No. 63/481,045, filed on Jan. 23, 2023.

(51) Int. Cl. C07D 291/08; A61K 31/395; A61K 31/407; A61K 31/439; C07D 498/08

(52) U.S. Cl. CPC C07D 291/08; A61K 31/395; A61K 31/407; A61K 31/439; C07D 498/08

(57) ABSTRACT

The present disclosure provides, in part, compounds of formula (I), or a stereoisomer and/or a pharmaceutically acceptable salt thereof, wherein the variables are as defined herein; pharmaceutical compositions comprising the compounds; and methods of using the compounds to treat conditions, diseases, and disorders associated with aberrant levels of lipids.

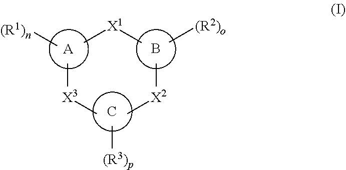

### CROSS-REFERENCE TO RELATED APPLICATIONS

This application claims priority to and the benefit of U.S. Provisional Patent Application No. 63/337,344, filed May 2, 2022, and U.S. Provisional Patent Application No. 63/481,045, filed Jan. 23, 2023, the content of each of which is incorporated by reference herein in its entirety.

### BACKGROUND

A combination of human genetic factors, overnutrition, and a sedentary lifestyle promote derangements in cholesterol and triglyceride metabolism. These derangements can manifest as one or more risk factors associated with increased probability of developing a number of life-threatening metabolic and/or cardiovascular diseases. The importance of maintaining cholesterol homeostasis in humans is strongly supported by both epidemiologic cohort studies and meta-analyses of multiple Mendelian and statin randomized trials that clearly demonstrate an association between elevated plasma levels of low-density lipoprotein cholesterol (LDL-C) (hypercholesterolemia) and atherosclerotic cardiovascular disease (ASCVD) risk. (Ference et al. (2017) Low-density lipoproteins cause atherosclerotic cardiovascular disease. 1. Evidence from genetic, epidemiologic, and clinical studies. A consensus statement from the European Atherosclerosis Society Consensus Panel., Eur. Heart J., 38, 2459-2472; Silverman et al., (2016) Association between lowering LDL-C and cardiovascular risk reduction among different therapeutic interventions: a systematic review and meta-analysis., JAMA, 316, 1289-1297).

While an association between ASCVD and circulating triglyceride levels is less clear (Helgadottir et al., (2016), Variants with large effects on blood lipids and the role of cholesterol and triglycerides in coronary disease., Nat. Genet., 48, 634-639; Miller et al., (2011), Triglycerides and cardiovascular disease: a scientific statement from the American Heart Association., Circulation, 123, 2292-2333), aberrations in triglyceride metabolism also manifest as other metabolic ASCVD risk factors including insulin resistance, type 2 diabetes, and nonalcoholic fatty liver disease (NAFLD). (Cohen et al., (2011), Human fatty liver disease: old questions and new insights., Science, 332, 1519-1523; Armstrong et al., (2014), Extrahepatic complications of nonalcoholic fatty liver disease., Hepatology 59, 1174-1197). Moreover, NAFLD poses an independent health challenge as one of the most common causes of chronic liver disease and hepatocellular carcinoma, leading causes of liver-related morbidity and mortality in the Western world. (Loomba and Sanyal, (2013), The global NAFLD epidemic., Nat. Rev. Gastroenterol. Hepatol., 10, 686-690).

Neither ASCVD nor NAFLD is adequately addressed by currently available treatment options. Many patients are not effectively treated for lipid disorders with the current standard of care, ASCVD remains the leading cause of death and disability in the Western world. (Mendis, (2010), The contribution of the Framingham Heart Study to the prevention of cardiovascular disease: a global perspective., Prog. Cardiovasc. Dis., 53, 10-14). As such, new therapeutic strategies that target cholesterol and triglyceride metabolism are required. ATP-citrate lyase (ACLY) is an enzyme uniquely positioned at the intersection of nutrient catabolism, and cholesterol and fatty acid biosynthesis, a metabolic nexus shown to be dysregulated in multiple disease states. Significant evidence supports that ACLY-derived acetyl-coenzyme A (CoA) serves not only as carbon precursor for cholesterol and fatty acid biosynthesis, but also as a key metabolic checkpoint used by multiple cell types to sense nutrient availability and coordinate metabolic adaptions with multiple effector functions. Thus, there is an unmet need to develop new therapeutic agents that modulate (e.g., inhibit) ACLY activity to treat metabolic and/or cardiovascular diseases.

### SUMMARY

Provided herein are compounds designed to function as modulators (e.g., inhibitors) of ATP citrate lyase (ACLY). Such compounds can be useful as therapeutic agents for treating conditions, diseases, and disorders associated with aberrant metabolism, such as NAFLD, nonalcoholic steatohepatitis (NASH), type-2 diabetes, chronic kidney disease, inflammation, autoimmunity, and cancer.

In one aspect, provided herein are compounds of formula (I)

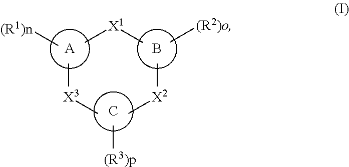

or a stereoisomer and/or a pharmaceutically acceptable salt thereof, wherein the variables are as defined herein.

In another aspect, provided herein are compounds of formula (Ia)

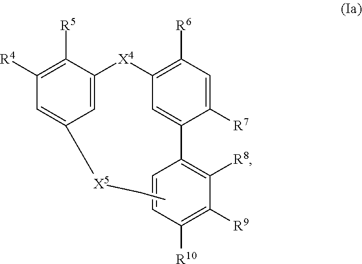

or a stereoisomer and/or a pharmaceutically acceptable salt thereof, wherein the variables are as defined herein.

In certain embodiments, the compounds of formula (I) or formula (Ia) are selected from the compounds of Table 1 or a stereoisomer and/or a pharmaceutically acceptable salt thereof.

In another aspect, provided herein are pharmaceutical compositions comprising a compound disclosed herein or a stereoisomer and/or a pharmaceutically acceptable salt thereof, and one or more pharmaceutically acceptable carriers.

A compound of formula (I) or a stereoisomer and/or a pharmaceutically acceptable salt thereof; or a pharmaceutical composition of the invention can be used in treating the various conditions, diseases, and disorders described herein. For example, the methods of treatment can include inhibiting ACLY, inhibiting cholesterol synthesis, and/or suppressing fatty acid biosynthesis. In some embodiments, the condition, disease, or disorder can be a liver condition, disease, or disorder such as NAFLD or NASH and the methods include treating the liver condition, disease, or disorder such as NAFLD or NASH. In some embodiments, the condition, disease, or disorder can be type-2 diabetes and the methods include treating type-2 diabetes. In some embodiments, the condition, disease, or disorder can be inflammation and the methods include treating inflammation. In some embodiments, the condition, disease, or disorder is chronic kidney disease and the methods include treating chronic kidney disease. In some embodiments, the condition, disease, or disorder is autoimmunity and the methods include treating autoimmunity. In some embodiments, the condition, disease, or disorder is cancer and the methods include treating cancer.

### DETAILED DESCRIPTION

As generally described herein, the disclosure provides compounds of formula (I), e.g., a compound of formula (Ia), formula (Ib) or formula (II), or a stereoisomer and/or a pharmaceutically acceptable salt thereof, and pharmaceutical compositions containing the same. The compounds and compositions described herein function as modulators (e.g., inhibitors) of ACLY. The disclosure also provides methods of using the compounds and compositions disclosed herein to treat a variety of conditions, diseases, and disorders associated with aberrant metabolism. Such conditions, diseases, and disorders include, but are not limited to NAFLD, NASH, type-2 diabetes, chronic kidney disease, inflammation, autoimmunity, and cancer.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element. By way of further example “an analogue” means one analogue or more than one analogue.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, +5%, +3%, +2%, or ±1% variation from the nominal value unless otherwise indicated or inferred from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃-6, C₃₋₅, C₃₋₄, C₄₋₆, C₄₋₅, and C₅₋₆ alkyl. By way of another example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The compounds of the disclosure can contain one or more chiral centers and/or double bonds and therefore, can exist as stereoisomers, such as geometric isomers, and enantiomers or diastereomers. The term “stereoisomers,” when used herein, consists of all geometric isomers, enantiomers and/or diastereomers of the compound. For example, when a compound is shown with specific chiral center(s), the compound depicted without such chirality at that and other chiral centers of the compound are within the scope of the present disclosure, i.e., the compound depicted in two-dimensions with “flat” or “straight” bonds rather than in three dimensions, for example, with solid or dashed wedge bonds.

More specifically, a compound described herein (e.g., a compound of formula (I), formula (Ia), formula (Ib), or formula (II))) can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Carreira and Kvaerno, Classics in Stereoselective Synthesis, (Wiley-VCH: Weinheim, 2009); Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

Geometric isomers, resulting from the arrangement of substituents around a carbon-carbon double bond or arrangement of substituents around a cycloalkyl or heterocycloalkyl, can also exist in the compounds of the present disclosure. The symbol [symbol] denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

A compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and F may be in any isotopic form, including ¹⁸F and ¹⁹F. Other examples of isotopes that can be incorporated into compounds described herein include isotopes of nitrogen, phosphorus, and chlorine, such as ¹⁵N, ³¹P, ³²P ³⁵S, and ³⁶Cl, respectively. As an example of an isotopic compound, a compound described herein can have one or more H atoms replaced with deuterium.

The terms described herein are intended to have the meanings presented and can be useful in understanding the description and intended scope of the present disclosure. When describing the disclosure, which may include a compound disclosed herein or a stereoisomer and/or a pharmaceutically acceptable salt thereof, pharmaceutical compositions containing such compounds and methods of using such compounds and compositions, the defined terms, if present, have their ascribed meanings unless otherwise indicated.

Unless otherwise stated, the term “substituted” is to be defined as set out herein. It should be further understood that the terms “groups” and “radicals” can be considered interchangeable when used herein.

As used herein, “alkyl” refers to a radical of a straight-chain or branched saturated hydrocarbon group, e.g., having 1 to 20 carbon atoms (“C₁₋₂₀ alkyl”) such as a straight-chain or branched group of 1-6, 1-4, or 1-3 carbon atoms, referred to herein as C₁-C₆ alkyl, C₁-C₄ alkyl, and C₁-C₃ alkyl, respectively. For example, “C₁-C₆ alkyl” refers to a straight-chain or branched saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl. In another example, “C₁-C₄ alkyl” refers to a straight-chain or branched saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”); 3 to 7 ring carbon atoms (“C₃₋₇ carbocyclyl”); 3 to 6 ring carbon atoms (“C₃₋₆ carbocyclyl”); or 5 to 10 ring carbon atoms (“C₅₋₁₀ carbocyclyl”). Exemplary C₃₋₈ carbocyclyl groups include, without limitation, cyclopropyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated.

As used herein, “cycloalkyl” refers to a monovalent saturated cyclic, bicyclic, or bridged cyclic (e.g., adamantyl) hydrocarbon group of 3-12, 3-8, 3-6, 4-8, or 4-6 carbons, referred to herein, e.g., as “C₃₋₆ cycloalkyl,” derived from a cycloalkane. Exemplary cycloalkyl groups include, but are not limited to, cyclohexanes such as cyclohexyl and cyclohexenyl, cyclopentanes such as cyclopentyl and cyclopentenyl, cyclobutanes such cyclobutyl, and cyclopropanes such as cyclopropyl.

As used herein, “heteroatom” refers to an atom of any element other than carbon or hydrogen and includes, for example, nitrogen (N), oxygen (O), silicon (Si), sulfur (S), phosphorus (P), and selenium (Se).

As used herein, “heterocyclyl” or “heterocyclic” refer to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In some embodiments, a heterocyclyl is 5- to 10-membered (“5-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

As used herein, “heteroaryl” refers to a radical of a 5-14 membered monocyclic or bicyclic 4n+2 aromatic ring system (e.g., having 6 or 10 electrons shared in a cyclic array) having ring carbon atoms and 1-4 ring heteroatoms provided in the aromatic ring system (“5-10 membered heteroaryl”). In certain embodiments, each heteroatom is independently selected from nitrogen, oxygen and sulfur. In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Other non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, and isoquinolinyl.

As used herein, “=hetero” can be used to describe a compound or a group present on a compound where one or more carbon atoms in the compound or group have been replaced by a heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

As used herein, “carbonyl” refers to the radical —C(O)— or C═O.

As used herein, “cyano” refers to —CN.

As used herein, “hydroxy” and “hydroxyl” refer to the radical —OH.

As used herein, “oxo” refers to the radical ═O (double bonded oxygen).

As used herein, “halo” and “halogen” refer to an atom selected from fluorine (fluoro, —F), chlorine (chloro, —Cl), bromine (bromo, —Br), and iodine (iodo, —I). In certain embodiments, the halo group is either fluoro or chloro.

As used herein, “alkoxy” refers to an alkyl group which is attached to another moiety via an oxygen atom (—O(alkyl)). Alkoxy groups can have 1-6 or 2-6 carbon atoms and are referred to herein as C₁-C₆ alkoxy and C₂-C₆ alkoxy, respectively. Exemplary alkoxy groups include, but are not limited to, methoxy, ethoxy, propyloxy, isopropoxy, and tert-butoxy.

As used herein, “haloalkyl” refers to mono, poly, and perhaloalkyl groups substituted with one or more halogen atoms where the halogens are independently selected from fluorine, chlorine, bromine, and iodine. In some embodiments, a haloalkyl has 1 to 6 carbon atoms (“C₁₋₆haloalkyl”).

As used herein, “haloalkoxy” refers to a haloalkyl group which is attached to another moiety via an oxygen atom such as, e.g., but are not limited to —OCHCF₂ or —OCF₃. In some embodiments, a haloalkoxy has 1 to 6 carbon atoms (“C₁₋₆haloalkoxy”).

As generally used herein, “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

As used herein, “compound” refers to the compound itself and its pharmaceutically acceptable salts, hydrates, esters and N-oxides including its various stereoisomers and its isotopically-labelled forms, unless otherwise understood from the context of the description or expressly limited to one particular form of the compound, i.e., the compound itself, a specific stereoisomer and/or isotopically-labelled compound, or a pharmaceutically acceptable salt, a hydrate, an ester, or an N-oxide thereof. It should be understood that a compound can refer to a pharmaceutically acceptable salt, or a hydrate, an ester or an N-oxide of a stereoisomer of the compound and/or an isotopically-labelled compound.

Further, if a variable is not accompanied by a definition, then the variable is defined as found elsewhere in the disclosure unless understood to be different from the context. In addition, the definition of each variable and/or substituent, for example, C₁-C₆ alkyl, R², R^{b}, w and the like, when it occurs more than once in any structure or compound, can be independent of its definition elsewhere in the same structure or compound.

Definitions of the variables and/or substituents in formulae and/or compounds herein encompass multiple chemical groups. The present disclosure includes embodiments where, for example, i) the definition of a variable and/or substituent is a single chemical group selected from those chemical groups set forth herein, ii) the definition is a collection of two or more of the chemical groups selected from those set forth herein, and iii) the compound is defined by a combination of variables and/or substituents in which the variables and/or substituents are defined by (i) or (ii).

### Other Definitions

As used herein, “pharmaceutically acceptable” and “pharmacologically acceptable,” refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, general safety and purity standards as required by FDA Office of Biologics standards.

As used herein, “pharmaceutically acceptable carrier” and “pharmaceutically acceptable excipient,” refer to any and all solvents, dispersion media, coatings, isotonic and absorption delaying agents, and the like, that are compatible with pharmaceutical administration. Pharmaceutical acceptable carriers can include phosphate buffered saline solution, water, emulsions (e.g., such as an oil/water or water/oil emulsions), and various types of wetting agents. The compositions also can include stabilizers and preservatives.

As used herein, “pharmaceutically acceptable salt” refers to any salt of an acidic or a basic group that may be present in a compound of the present disclosure, which salt is compatible with pharmaceutical administration. As is known to those of skill in the art, “salts” of the compounds of the present disclosure may be derived from inorganic or organic acids and bases. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound (e.g., a compound of the present invention) is an amount sufficient to provide a therapeutic benefit in the treatment of a disease, disorder or condition, or to delay or minimize one or more symptoms associated with the disease, disorder or condition. A therapeutically effective amount of a compound (e.g., a compound of the present invention) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, “disease,” “disorder,” “condition,” or “illness,” can be used interchangeably unless otherwise underacted or understood from the context, refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, or illness e.g., through administration of the compound of formula (I), or a stereoisomer and/or a pharmaceutically acceptable salt thereof.

As used herein, “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, “liver disorder” refers generally to a disease, a disorder, and/or a condition affecting the liver, and may have a wide range of severity encompassing, for example, simple accumulation of fat in the hepatocytes (steatosis), macrovesicular steatosis, periportal and lobular inflammation (steatohepatitis), cirrhosis, fibrosis, liver cancers, and liver failure.

As used herein, “fatty liver disease” (“FLD”), which is also called “fatty liver,” refers to a disease leading to liver injury caused by abnormal fat accumulation in liver cells. FLD may arise from a number of sources, including excessive alcohol consumption and metabolic disorders, such as those associated with insulin resistance, obesity, and hypertension.

As used herein, “non-alcoholic fatty liver disease” (“NAFLD”) refers to the spectrum of disorders resulting from an accumulation of fat in liver cells in individuals with no history of excessive alcohol consumption. In the mildest form, NAFLD refers to hepatic steatosis.

As used herein, “drug-induced liver disease” or “toxic liver injury” refers to a disease or a condition in which an active agent has caused injury to the liver.

As used herein, “alcoholic liver disease,” also called “alcoholic liver injury,” refers to a disease caused by fat accumulation in liver cells, caused at least in part by alcohol ingestion. Examples include, but are not limited to, diseases such as alcoholic simple fatty liver, alcoholic steatohepatitis (“ASH”), alcoholic hepatic fibrosis, alcoholic cirrhosis, alcoholic fatty liver disease, and the like. It should be noted that alcoholic steatohepatitis is also called alcoholic fatty hepatitis and includes alcoholic hepatic fibrosis.

As used herein, “fatty liver of pregnancy” refers to acute fatty liver conditions that can arise during pregnancy and can be life-threatening.

As used herein, “altering lipid metabolism” refers to an observable (measurable) change in at least one aspect of lipid metabolism, including, but not limited to, total blood lipid content, blood HDL cholesterol, blood LDL cholesterol, blood VLDL cholesterol, blood triglyceride, blood Lp(a), blood apo A-I, blood apo E and blood non-esterified fatty acids.

As used herein, “altering glucose metabolism” refers to an observable (measurable) change in at least one aspect of glucose metabolism, including, but not limited to, total blood glucose content, blood insulin, the blood insulin to blood glucose ratio, insulin sensitivity, and oxygen consumption.

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

### Compounds

Disclosed herein, in one aspect, are compounds of formula (I):

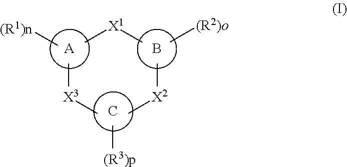

or a stereoisomer and/or a pharmaceutically acceptable salt thereof, wherein:
Ring A is phenyl or pyridonyl, wherein the nitrogen atom of the pyridonyl may optionally be substituted by C₁₋₆alkyl;
Ring B is phenyl or 5-10 membered heterocyclyl;
Ring C is phenyl, 5-10 membered heterocyclyl, or 5-10 membered heteroaryl; or
Ring C is absent;
R¹ is independently, for each occurrence, selected from the group consisting of halogen, hydroxyl, C₁₋₆alkyl, C₁₋₆alkoxy, C₁₋₆haloalkoxy, —O—C(O)C₁₋₆alkyl, and —O—C(O)C₃₋₆cycloalkyl;
R² is independently, for each occurrence, selected from the group consisting of halogen, hydroxyl, C₁₋₆haloalkyl, C₁₋₆alkoxy, and COOH;
R³ is cyano, halogen, or C₃₋₆cycloalkyl;
X¹ is selected from the group consisting of *—S(O)₂N(R^{A})—**, —C(O)—, *—C(O)N(R^{A})—**, *—CH₂N(R^{A})—**, and *—S(O)₂CH₂—**, wherein * denotes the point of attachment to Ring A and ** denotes the point of attachment to Ring B;
X² is a bond or —O—;
X³ is #-L¹-L²-L³-##, wherein # denotes the point of attachment to Ring A and ##denotes the point of attachment to Ring C or to Ring B when Ring C is absent;
L¹ is selected from the group consisting of —CH₂—, —O—, —C(O)—, —C(O)N(R^{B})—, —C(O)O—, C₁₋₆alkyl-O—, and 5-6 membered heteroaryl;
L² is C₁₋₆alkyl or 4-6 membered heterocyclyl, wherein the 4-6 membered heterocyclyl may be optionally substituted with oxo;
L³ is selected from the group consisting of a bond, —O—, —O—C₁₋₆alkyl, C₁₋₆alkyl-O—, and 4-6 membered heterocyclyl;
R^{A} is hydrogen or C₁₋₆alkyl;
R^{B} is hydrogen or C₁₋₆alkyl;
n is 0, 1, or 2;
o is 1 or 2; and
p is 0 or 1.

In another aspect, provided herein are compounds of formula (I):

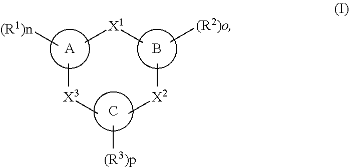

or a stereoisomer and/or a pharmaceutically acceptable salt thereof, wherein:
Ring A is phenyl, pyridinyl, or pyridonyl, wherein the nitrogen atom of the pyridonyl may optionally be substituted by C₁₋₆alkyl;
Ring B is phenyl or 5-10 membered heterocyclyl;
Ring C is phenyl, 5-10 membered heterocyclyl, or 5-10 membered heteroaryl; or
Ring C is absent;
R¹ is independently, for each occurrence, selected from the group consisting of halogen, hydroxyl, C₁₋₆alkyl, C₁₋₆haloalkyl, C₁₋₆alkoxy, C₁₋₆haloalkoxy, —O—C(O)C₁₋₆alkyl, —O—C(O)C₃₋₆cycloalkyl, and N(R^{E})₂;
R² is independently, for each occurrence, selected from the group consisting of halogen, hydroxyl, C₁₋₆alkyl, C₁₋₆haloalkyl, C₁₋₆alkoxy, C₁₋₆haloalkoxy, C₃₋₆cycloalkyl, and COOH;
R³ is selected from the group consisting of cyano, halogen, C₁₋₆alkyl, and C₃₋₆cycloalkyl;
X¹ is selected from the group consisting of *—S(O)₂N(R^{A})—**, —C(O)—, *—C(O)N(R^{A})—**, *—CH₂N(R^{A})—**, and *—S(O)₂CH₂—**, wherein * denotes the point of attachment to Ring A and ** denotes the point of attachment to Ring B;
X² is a bond or —O—;
X³ is #-L¹-L²-L³-##, wherein # denotes the point of attachment to Ring A and ##denotes the point of attachment to Ring C or to Ring B when Ring C is absent;
L¹ is selected from the group consisting of —CH₂—, —O—, —C(O)—, —C(O)N(R^{B})—, —C(O)O—, —CH₂—C(O)O—, —CH₂—N(R^{B})C(O)—, C₁₋₆alkyl-O—, and 5-6 membered heteroaryl;
L² is C₁₋₆alkyl or 4-6 membered heterocyclyl, wherein the 4-6 membered heterocyclyl may be optionally substituted with oxo; or
L² is absent;
L³ is selected from the group consisting of a bond, —O—, —O—C₁₋₆alkyl, C₁₋₆alkyl-O—, and 4-6 membered heterocyclyl;
R^{A} is hydrogen or C₁₋₆alkyl;
R^{B} is hydrogen or C₁₋₆alkyl;
R^{E} is independently, for each occurrence, hydrogen or C₁₋₆alkyl;
n is 0, 1, or 2;
o is 1 or 2; and
p is 0 or 1.

In another aspect, provided herein are compounds of formula (I):

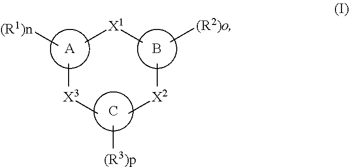

or a stereoisomer and/or a pharmaceutically acceptable salt thereof, wherein:
Ring A is phenyl, pyridinyl, or pyridonyl, wherein the nitrogen atom of the pyridonyl may optionally be substituted by C₁₋₆alkyl;
Ring B is phenyl or 5-10 membered heterocyclyl;
Ring C is phenyl, 5-10 membered heterocyclyl, or 5-10 membered heteroaryl; or
Ring C is absent;
R¹ is independently, for each occurrence, selected from the group consisting of halogen, hydroxyl, C₁₋₆alkyl, C₁₋₆haloalkyl, C₁₋₆alkoxy, C₁₋₆haloalkoxy, —O—C(O)C₁₋₆alkyl, —O—C(O)C₃₋₆cycloalkyl, and N(R^{E})₂;
R² is independently, for each occurrence, selected from the group consisting of halogen, hydroxyl, C₁₋₆alkyl, C₁₋₆haloalkyl, C₁₋₆alkoxy, C₁₋₆haloalkoxy, C₃₋₆cycloalkyl, and COOH;
R³ is selected from the group consisting of cyano, halogen, C₁₋₆alkyl, and C₃₋₆cycloalkyl;
X¹ is selected from the group consisting of *—S(O)₂N(R^{A})—**, —C(O)—, *—C(O)N(R^{A})—**, *—CH₂N(R^{A})—**, and *—S(O)₂CH₂—**, wherein * denotes the point of attachment to Ring A and ** denotes the point of attachment to Ring B;
X² is a bond or —O—;
X³ is #-L¹-L²-L³-##, wherein # denotes the point of attachment to Ring A and ##denotes the point of attachment to Ring C or to Ring B when Ring C is absent;
L¹ is selected from the group consisting of —CH₂—, —C(O)—, —C(O)O—, —CH₂—C(O)O—, —CH₂—N(R^{B})C(O)—, C₁₋₆alkyl-O—, and 5-6 membered heteroaryl;
L² is C₁₋₆alkyl or 4-6 membered heterocyclyl, wherein the 4-6 membered heterocyclyl may be optionally substituted with oxo; or
L² is absent;
L³ is selected from the group consisting of a bond, —O—, —O—C₁₋₆alkyl, C₁₋₆alkyl-O—, and 4-6 membered heterocyclyl;
R^{A} is hydrogen or C₁₋₆alkyl;
R^{B} is hydrogen or C₁₋₆alkyl;
R^{E} is independently, for each occurrence, hydrogen or C₁₋₆alkyl;
n is 0, 1, or 2;
o is 1 or 2; and
p is 0 or 1.

In some embodiments, Ring A is phenyl or 2-pyridonyl, wherein the nitrogen atom of the 2-pyridonyl may optionally be substituted by C₁₋₆alkyl. In some embodiments, Ring A is phenyl, pyridinyl, or 2-pyridonyl, wherein the nitrogen atom of the 2-pyridonyl may optionally be substituted by C₁₋₆alkyl.

In some embodiments, Ring A is phenyl or 2-pyridonyl, wherein the nitrogen atom of the 2-pyridonyl may optionally be substituted by CH₃. In some embodiments, Ring A is phenyl, pyridinyl, or 2-pyridonyl, wherein the nitrogen atom of the 2-pyridonyl may optionally be substituted by CH₃.

In some embodiments, Ring A is

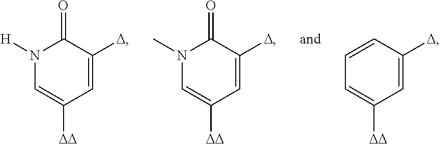

wherein Δ denotes the point of attachment to X and ΔΔ denotes the point of attachment to X³. In some embodiments, Ring A is selected from the group consisting of

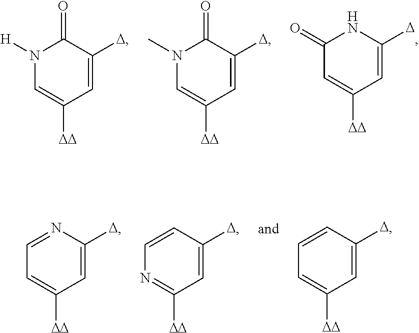

wherein Δ denotes the point of attachment to X¹ and ΔΔ denotes the point of attachment to X³.

In some embodiments, n is 0. In some embodiments, n is 1. In some embodiments, n is 2.

In some embodiments, R¹ is chloro or hydroxyl. In some embodiments, R¹ is selected from the group consisting of chloro, hydroxyl, CH₃, CHF₂, and NH₂.

In some embodiments, R¹ is independently, for each occurrence, selected from the group consisting of chloro, hydroxyl, CH₃, —O—CH₃, —O—CHF₂, —O—C(O)CH₃, and

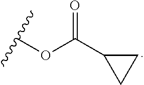 [SMILES: *OC(=O)C1CC1]

In some embodiments, R¹ is independently, for each occurrence, selected from the group consisting of chloro, fluoro, hydroxyl, CH₃, CHF₂, —O—CH₃, —O—CHF₂, —O—C(O)CH₃, and

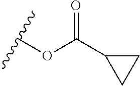 [SMILES: *OC(=O)C1CC1]

In some embodiments, Ring B is phenyl or 9-membered heterocyclyl.

In some embodiments, wherein Ring B is phenyl or.

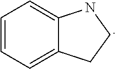

In some embodiments, Ring B is

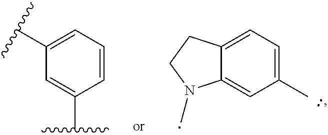

wherein ● denotes the point of attachment to X¹ and ●● denotes the point of attachment to X².

In some embodiments, o is 1. In some embodiments, o is 2.

In some embodiments, R² is selected from the group consisting of fluoro, hydroxyl, —O—CH₃, and C(O)OH. In some embodiments, R² is selected from the group consisting of fluoro, hydroxyl, cyclopropyl, CF₃, —O—CH₃, —O—CHF₂, —O—CF₃, and C(O)OH.

In some embodiments, R² is independently, for each occurrence, selected from the group consisting of chloro, fluoro, CF₃, and —O—CH₃. In some embodiments, R² is independently, for each occurrence, selected from the group consisting of chloro, fluoro, CH₃, CF₃, and —O—CH₃.

In some embodiments, Ring C is absent.

In some embodiments, Ring C is selected from the group consisting of phenyl, pyrrolidinyl, piperidinyl, pyridinyl,

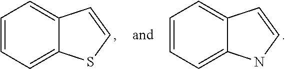 [SMILES: c1ccc2sccc2c1 ; c1ccc2[nH]ccc2c1]

In some embodiments, Ring C is selected from the group consisting of phenyl, pyrrolidinyl, piperidinyl, pyridinyl,

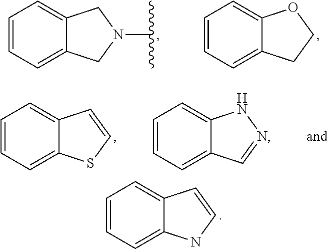

In some embodiments, Ring C is selected from the group consisting of

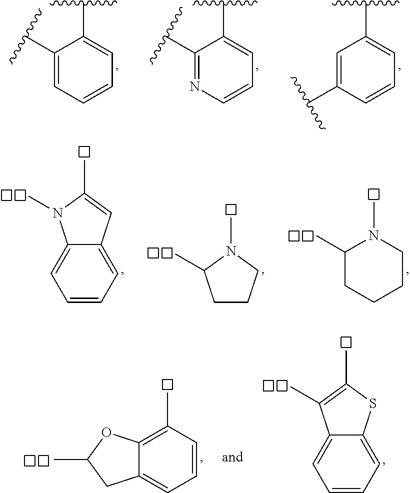

wherein □ denotes the point of attachment to X² and □□ denotes the point of attachment to X³.

In some embodiments, Ring C is selected from the group consisting of

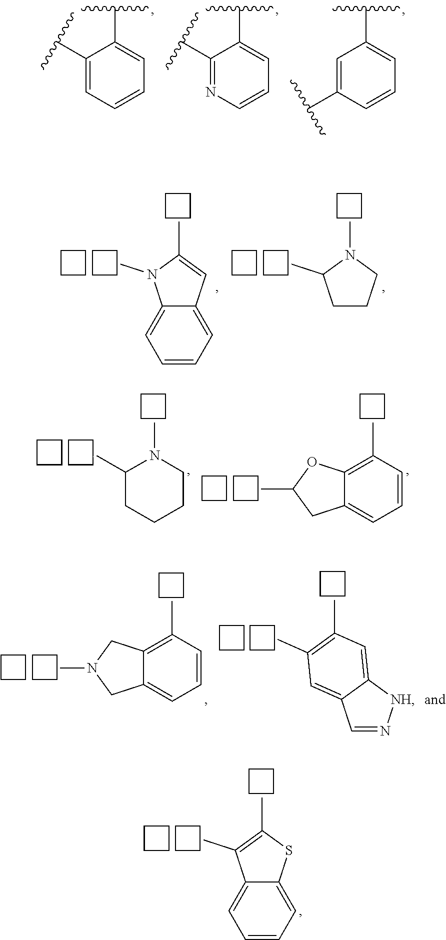

wherein □ denotes the point of attachment to X² and □□ denotes the point of attachment to X³.

In some embodiments, p is 1. In some embodiments, p is 0.

In some embodiments, R³ is selected from the group consisting of bromo, chloro, fluoro, cyano, and cyclopropyl. In some embodiments, R³ is selected from the group consisting of bromo, chloro, fluoro, cyano, CH₃, and cyclopropyl.

In some embodiments, X¹ is selected from the group consisting of *—S(O)₂N(H)—**, *—S(O)₂N(CH₃)—**, —C(O)—, *—C(O)N(H)—**, *—CH₂N(H)—**, and *—S(O)₂CH₂—**, wherein * denotes the point of attachment to Ring A and ** denotes the point of attachment to Ring B.

In some embodiments, X² is a bond. In some embodiments, X² is —O—.

In some embodiments, L¹ is selected from the group consisting of —C(O)N(H)—, —C(O)N(CH₃)—, —C(O)O—, —CH₂—, —CH₂—O—, —C(O)—, —O—, and

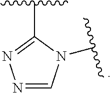 [SMILES: *c1nncn1*]

In some embodiments, L¹ is selected from the group consisting of —C(O)N(H)—, —C(O)N(CH₃)—, —C(O)O—, —CH₂—, —CH₂—O—, —C(O)—, —CH₂—C(O)O—, —CH₂—N(CH₃)C(O)—, —O—, and

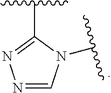 [SMILES: *c1nncn1*]

In some embodiments, L¹ is selected from the group consisting of —C(O)O—, —CH₂—, —CH₂—O—, —C(O)—, —CH₂—C(O)O—, —CH₂—N(CH₃)C(O)—, and

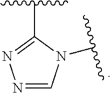 [SMILES: *c1nncn1*]

In some embodiments, L² is selected from the group consisting of —CH₂CH₂—, —(CH₂)₃—, —CH₂—, —(CH₂)₄—, —CH(CH₃)CH₂—,

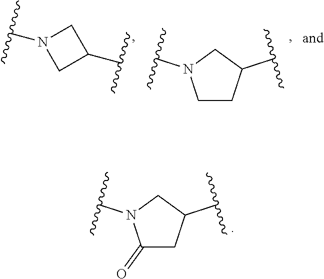

In some embodiments, L² is selected from the group consisting of —CH₂CH₂—, —(CH₂)₃—, —CH₂—, —(CH₂)₄—, —CH(CH₃)CH₂—, —CH₂CH₂C(H)(CH₃)—,

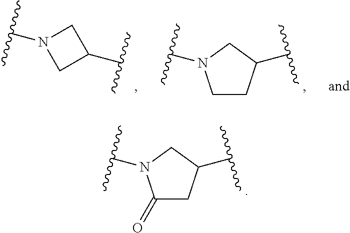 [SMILES: *C1CN(*)C1 ; *C1CCN(*)C1 ; *C1CC(=O)N(*)C1]

In some embodiments, L² is absent.

In some embodiments, L³ is a bond.

In some embodiments, L³ is —O— or

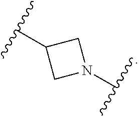 [SMILES: *C1CN(*)C1]

In some embodiments, L³ is —O—, —CH₂—O—, or

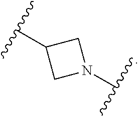 [SMILES: *C1CN(*)C1]

In some embodiments, X³ is selected from the group consisting of

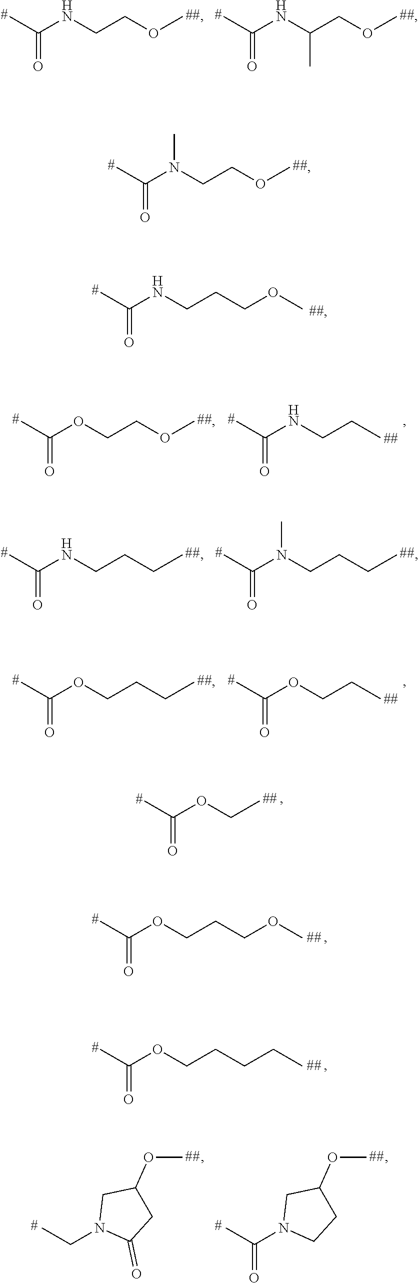

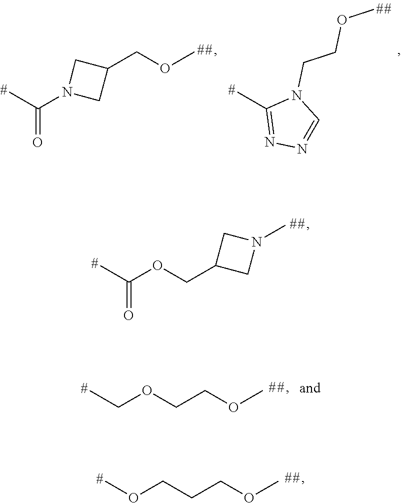

wherein #denotes the point of attachment to Ring A and ## denotes the point of attachment to Ring C.

In some embodiments, X³ is selected from the group consisting of

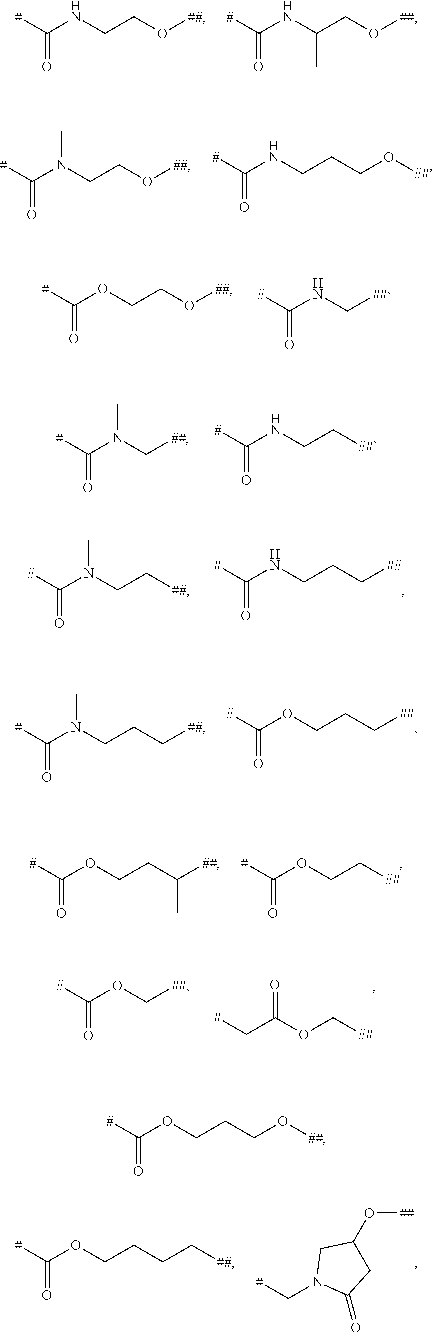

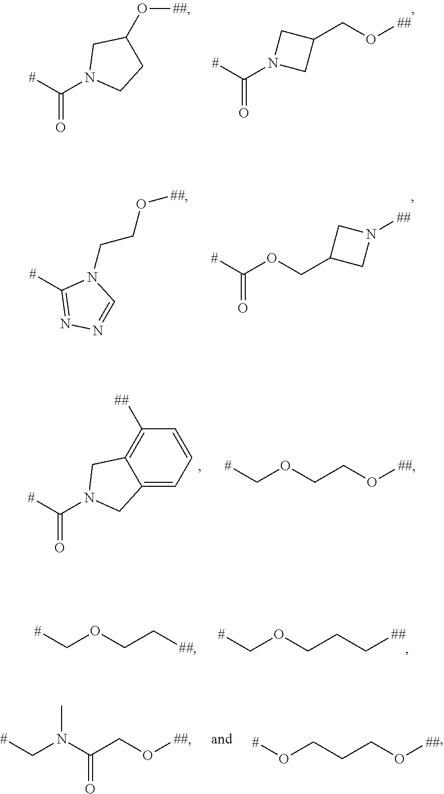

wherein # denotes the point of attachment to Ring A and ## denotes the point of attachment to Ring C.

In some embodiments, X³ is selected from the group consisting of

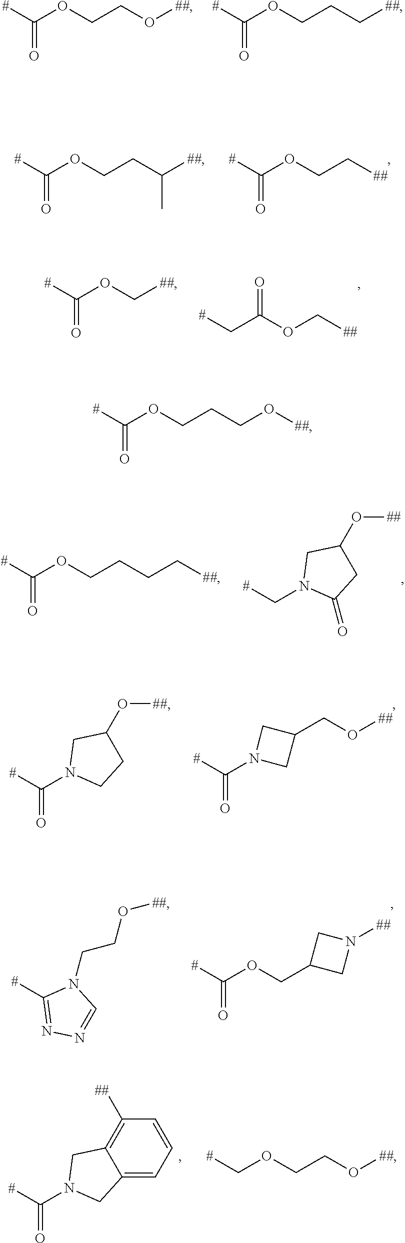

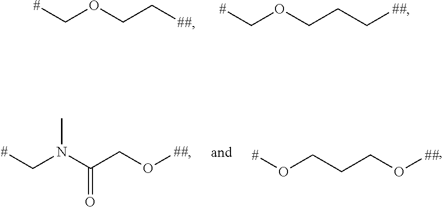

wherein # denotes the point of attachment to Ring A and ## denotes the point of attachment to Ring C.

Also disclosed herein, in various embodiments, are compounds of formula (Ia):

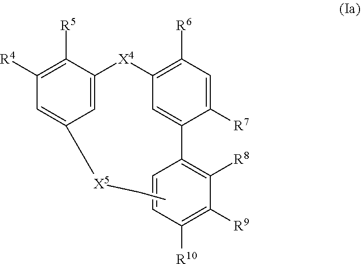

or a stereoisomer and/or a pharmaceutically acceptable salt thereof, wherein:
R⁴ is hydrogen, halogen, or C₁₋₆alkyl;
R⁵ is selected from the group consisting of hydrogen, hydroxy, C₁₋₆alkoxy, C₁₋₆haloalkoxy, —C(O)OC₁₋₆alkyl, and —C(O)OC₁₋₆cycloalkyl;
R⁶ is selected from the group consisting of hydrogen, halogen, hydroxy, C₁₋₆alkoxy, C₁₋₆alkyl, and C(O)OH;
R⁷ is selected from the group consisting of hydrogen, halogen, C₁₋₆haloalkyl, and C₁₋₆alkoxy;
R⁸ is hydrogen or halogen;
R⁹ is selected from the group consisting of hydrogen, cyano, halogen, and C₃₋₆cycloalkyl;
R¹⁰ is hydrogen or halogen;
X⁴ is selected from the group consisting of *—S(O)₂N(R^{C})—**, *—C(O)N(R^{C})—**, *—CH₂N(R^{C})—**, and *—S(O)₂CH₂—**, wherein * denotes the point of attachment to

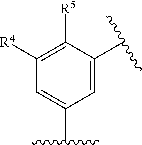

and ** denotes the point of attachment to

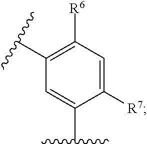

X⁵ is #-L⁴-L⁵-L⁶-##, wherein # denotes the point of attachment to

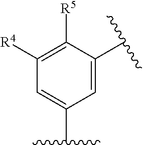

and ## denotes the point of attachment to

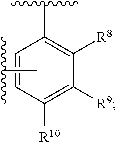

L⁴ is selected from the group consisting of CH₂, C₁₋₆alkyl-O—, —O—, —C(O)—, —C(O)N(R^{D})—, —C(O)O—, and 5-6 membered heteroaryl;
L⁵ is 4-6 membered heterocyclyl, wherein the 4-6 membered heterocyclyl may be optionally substituted with oxo;
L⁶ is selected from the group consisting of a bond, —O—, C₁₋₆alkyl-O—, and 4-6 membered heterocyclyl;
R^{C} is hydrogen or C₁₋₆alkyl; and
R^{D} is hydrogen or C₁₋₆alkyl.

In various embodiments, provided herein are compounds of formula (Ia):

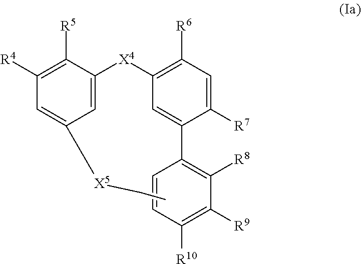

or a stereoisomer and/or a pharmaceutically acceptable salt thereof, wherein:
R⁴ is selected from the group consisting of hydrogen, hydroxyl, halogen, and C₁₋₆alkyl;
R⁵ is selected from the group consisting of hydrogen, halogen, hydroxyl, C₁₋₆alkoxy, C₁₋₆haloalkyl, C₁₋₆haloalkoxy, —C(O)OC₁₋₆alkyl, and —C(O)OC₁₋₆cycloalkyl;
R⁶ is selected from the group consisting of hydrogen, halogen, hydroxyl, C₁₋₆alkoxy, C₁₋₆haloalkoxy, C₁₋₆alkyl, C₁₋₆haloalkyl, C₃₋₆cycloalkyl, and C(O)OH;
R⁷ is selected from the group consisting of hydrogen, halogen, C₁₋₆alkyl, C₁₋₆haloalkyl, and C₁₋₆alkoxy;
R⁸ is hydrogen, halogen, or C₁₋₆alkyl;
R⁹ is selected from the group consisting of hydrogen, cyano, halogen, and C₃₋₆cycloalkyl;
R¹⁰ is hydrogen, cyano, or halogen;
X⁴ is selected from the group consisting of *—S(O)₂N(R^{C})—**, *—C(O)N(R^{C})—**, *—CH₂N(R^{C})—**, and *—S(O)₂CH₂—**, wherein * denotes the point of attachment to

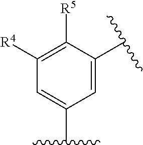

and ** denotes the point of attachment to

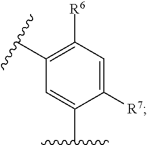

X⁵ is #-L⁴-L⁵-L⁶-##, wherein # denotes the point of attachment to

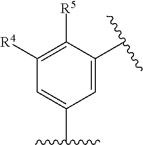

and ## denotes the point of attachment to

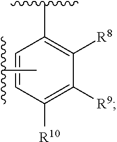

L⁴ is selected from the group consisting of CH₂, C₁₋₆alkyl-O—, —O—, —C(O)—, —C(O)N(R^{D})—, —C(O)O—, —CH₂—C(O)O—, —CH₂—N(R^{D})C(O)—, and 5-6 membered heteroaryl;
L⁵ is C₁₋₆alkyl or 4-6 membered heterocyclyl, wherein the 4-6 membered heterocyclyl may be optionally substituted with oxo;
L⁶ is selected from the group consisting of a bond, —O—, C₁₋₆alkyl-O—, and 4-6 membered heterocyclyl;
R^{C} is hydrogen or C₁₋₆alkyl; and
R^{D} is hydrogen or C₁₋₆alkyl.

In various embodiments, provided herein are compounds of formula (Ia):

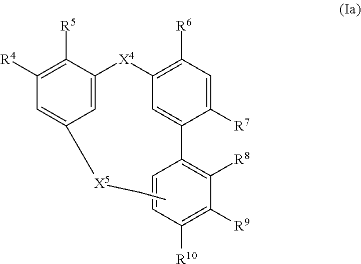

or a stereoisomer and/or a pharmaceutically acceptable salt thereof, wherein:
R⁴ is selected from the group consisting of hydrogen, hydroxyl, halogen, and C₁₋₆alkyl;
R⁵ is selected from the group consisting of hydrogen, halogen, hydroxyl, C₁₋₆alkoxy, C₁₋₆haloalkyl, and C₁₋₆haloalkoxy;
R⁶ is selected from the group consisting of halogen, hydroxyl, C₁₋₆alkoxy, C₁₋₆haloalkoxy, C₁₋₆haloalkyl, and C₃₋₆cycloalkyl;
R⁷ is selected from the group consisting of hydrogen, halogen, C₁₋₆alkyl, and C₁₋₆alkoxy;
R⁸ is hydrogen or C₁₋₆alkyl;
R⁹ is selected from the group consisting of hydrogen, cyano, halogen, and C₃₋₆cycloalkyl;
R¹⁰ is hydrogen, cyano, or halogen;
X⁴ is selected from the group consisting of *—S(O)₂N(R^{C})—**, *—C(O)N(R^{C})—**, *—CH₂N(R^{C})—**, and *—S(O)₂CH₂—**, wherein * denotes the point of attachment to

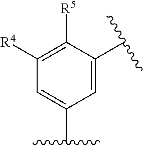

and ** denotes the point of attachment to

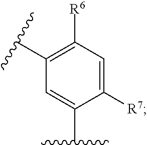

X⁵ is #-L⁴-L⁵-L⁶-##, wherein # denotes the point of attachment to

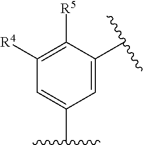

and ## denotes the point of attachment to

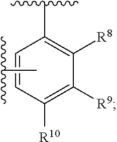

L⁴ is selected from the group consisting of C₁₋₆alkyl-O—, —C(O)—, —C(O)N(R^{D})—, —C(O)O—, —CH₂—C(O)O—, —CH₂—N(R^{D})C(O)—, and 5-6 membered heteroaryl;
L⁵ is C₁₋₆alkyl or 4-6 membered heterocyclyl;
L⁶ is selected from the group consisting of a bond, —O—, C₁₋₆alkyl-O—, and 4-6 membered heterocyclyl;
R^{C} is hydrogen or C₁₋₆alkyl; and
R^{D} is hydrogen or C₁₋₆alkyl.

In various embodiments, provided herein are compounds of formula (Ia):

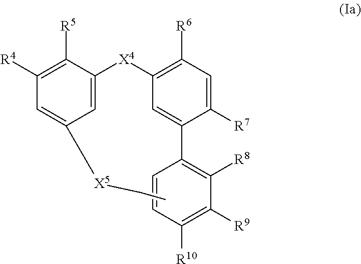

or a stereoisomer and/or a pharmaceutically acceptable salt thereof, wherein:
R⁴ is selected from the group consisting of hydrogen, hydroxyl, halogen, and C₁₋₆alkyl;
R⁵ is selected from the group consisting of hydrogen, halogen, hydroxyl, C₁₋₆alkoxy, C₁₋₆haloalkyl, and C₁₋₆haloalkoxy;
R⁶ is selected from the group consisting of halogen, hydroxyl, C₁₋₆alkoxy, C₁₋₆haloalkoxy, C₁₋₆haloalkyl, and C₃₋₆cycloalkyl;
R⁷ is selected from the group consisting of hydrogen, halogen, C₁₋₆alkyl, and C₁₋₆alkoxy;
R⁸ is hydrogen or C₁₋₆alkyl;
R⁹ is selected from the group consisting of hydrogen, cyano, halogen, and C₃₋₆cycloalkyl;
R¹⁰ is hydrogen, cyano, or halogen; X⁴ is selected from the group consisting of *—S(O)₂N(R^{C})—**, *—C(O)N(R^{C})—**, *—CH₂N(R^{C})—**, and *—S(O)₂CH₂—**, wherein * denotes the point of attachment to

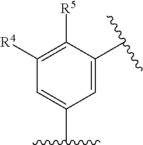

and ** denotes the point of attachment to

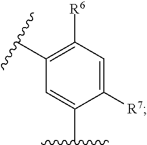

X⁵ is #-L⁴-L⁵-L⁶-##, wherein # denotes the point of attachment to

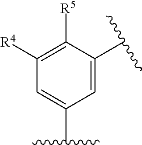

and ## denotes the point of attachment td

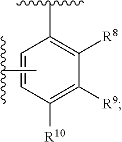

L⁴ is selected from the group consisting of C₁₋₆alkyl-O—, —C(O)—, —C(O)O—, —CH₂—C(O)O—, —CH₂—N(R^{D})C(O)—, and 5-6 membered heteroaryl;
L⁵ is C₁₋₆alkyl or 4-6 membered heterocyclyl;
L⁶ is selected from the group consisting of a bond, —O—, C₁₋₆alkyl-O—, and 4-6 membered heterocyclyl;
R^{C} is hydrogen or C₁₋₆alkyl; and
R^{D} is hydrogen or C₁₋₆alkyl.

In some embodiments, R⁴ is hydrogen, chloro, or CH₃. In certain embodiments, R⁴ is hydrogen, hydroxyl, chloro, or CH₃.

In some embodiments, R⁵ is selected from the group consisting of hydrogen, hydroxyl, —O—CH₃, —O—CHF₂, —O—C(O)CH₃, and

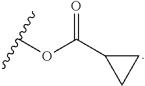 [SMILES: *OC(=O)C1CC1]

In some embodiments, R⁵ is selected from the group consisting of hydrogen, fluoro, hydroxyl, CHF₂, —O—CH₃, —O—CHF₂, —O—C(O)CH₃, and

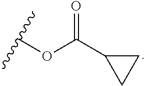 [SMILES: *OC(=O)C1CC1]

In some embodiments, R⁵ is selected from the group consisting of hydrogen, fluoro, hydroxyl, CHF₂, —O—CH₃, and —O—CHF₂.

In some embodiments, R⁶ is selected from the group consisting of hydrogen, hydroxyl, fluoro, chloro, —O—CH₃, and C(O)OH. In some embodiments, R⁶ is selected from the group consisting of hydrogen, hydroxyl, fluoro, chloro, cyclopropyl, CF₃, —O—CH₃, —O—CHF₂, —O—CF₃, and C(O)OH. In some embodiments, R⁶ is selected from the group consisting of hydroxyl, fluoro, cyclopropyl, CF₃, —O—CH₃, —O—CHF₂, and —O—CF₃.

In some embodiments, R⁷ is selected from the group consisting of hydrogen, chloro, fluoro, —O—CH₃, and CF₃. In some embodiments, R⁷ is selected from the group consisting of hydrogen, fluoro, —O—CH₃, and CH₃.

In some embodiments, R⁸ is hydrogen or chloro. In some embodiments, R⁸ is hydrogen, CH₃, or chloro. In some embodiments, R⁸ is hydrogen or CH₃.

In some embodiments, R⁹ is selected from the group consisting of hydrogen, cyano, chloro, bromo, fluoro, and cyclopropyl. In some embodiments, R⁹ is selected from the group consisting of hydrogen, cyano, chloro, bromo, fluoro, and cyclopropyl.

In some embodiments, R¹⁰ is hydrogen, chloro, or fluoro. In some embodiments, R¹⁰ is selected from the group consisting of hydrogen, cyano, chloro, and fluoro.

In some embodiments, X⁴ is selected from the group consisting of *—S(O)₂N(H)—**, *—S(O)₂N(CH₃)—**, *—C(O)N(H)—**, *—CH₂N(H)—**, and *—S(O)₂CH₂—**, wherein * denotes the point of attachment to

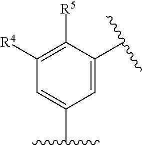

and ** denotes the point of attachment to

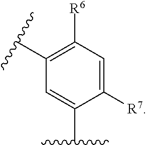

In some embodiments, L⁴ is selected from the group consisting of —CH₂—, —O—, —C(O)—, —C(O)N(H)—, —C(O)N(CH₃)—, —C(O)O—, —CH₂—O—, and

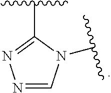 [SMILES: *c1nncn1*]

In some embodiments, L⁴ is selected from the group consisting of —CH₂—, —O—, —C(O)—, —C(O)N(H)—, —C(O)N(CH₃)—, —C(O)O—, —CH₂—C(O)O—, —CH₂—N(CH₃)C(O)—, —CH₂—O—, and

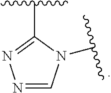 [SMILES: *c1nncn1*]

In some embodiments, L⁴ is selected from the group consisting of —CH₂—, —C(O)—, —C(O)N(H)—, —C(O)N(CH₃)—, —C(O)O—, —CH₂—C(O)O—, —CH₂—N(CH₃)C(O)—, —CH₂—O—, and

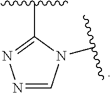 [SMILES: *c1nncn1*]

In some embodiments. L⁴ is selected from the group consisting of —CH₂—, —C(O)—, —C(O)O—, —CH₂—C(O)O—, —CH₂—N(CH₃)C(O)—, —CH₂—O—, and

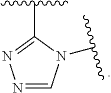 [SMILES: *c1nncn1*]

In some embodiments, L⁵ is selected from the group consisting of —CH₂—, —CH₂CH₂—, —(CH₂)₃—, —CH(CH₃)CH₂—, —(CH₂)₄—,

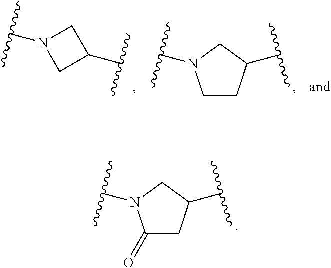

In some embodiments, L⁵ is selected from the group consisting of —CH₂—, —CH₂CH₂—, —(CH₂)₃—, —C(H)(CH₃)CH₂—, —CH₂CH₂C(H)(CH₃)—, —(CH₂)₄—,

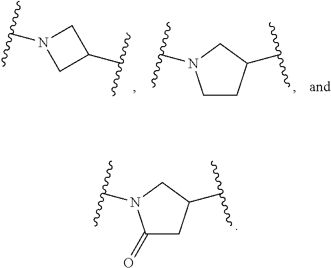

In some embodiments, L⁵ is selected from the group consisting of —CH₂—, —CH₂CH₂—, —(CH₂)₃—, —C(H)(CH₃)CH₂—, —CH₂CH₂C(H)(CH₃)—, and

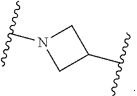 [SMILES: *C1CN(*)C1]

In some embodiments, L⁶ is a bond.

In some embodiments, L⁶ is —O— or

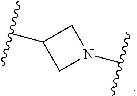 [SMILES: *C1CN(*)C1]

In some embodiments, L⁶ is —O—, —CH₂—O—, or

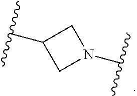 [SMILES: *C1CN(*)C1]

In some embodiments, X⁵ is selected from the group consisting of

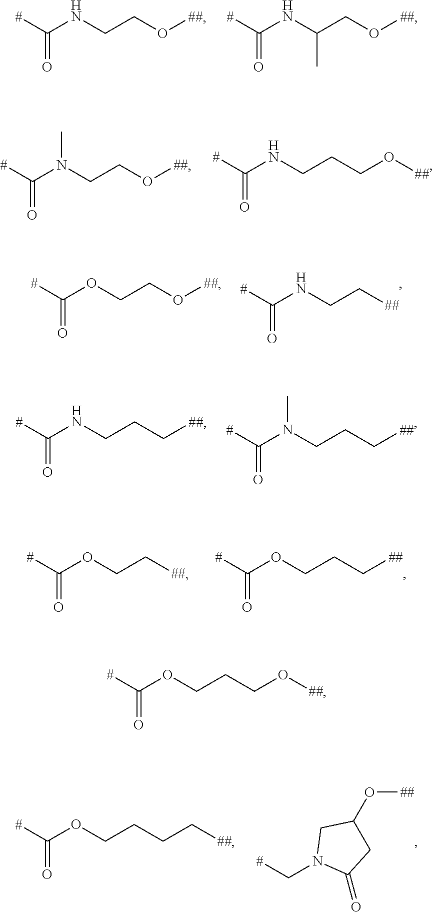

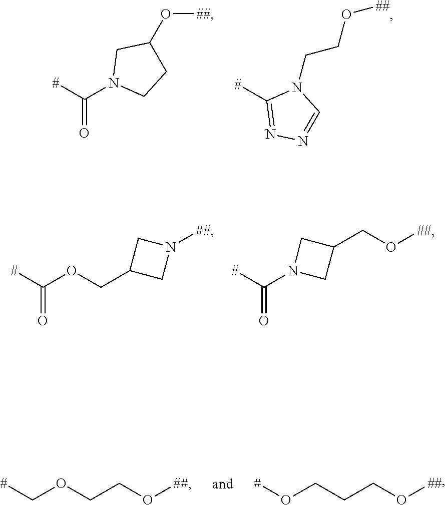

wherein # denotes the point of attachment to

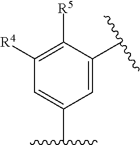

and ## denotes the point of attachment to

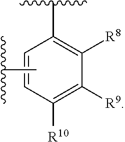

In some embodiments, X⁵ is selected from the group consisting of

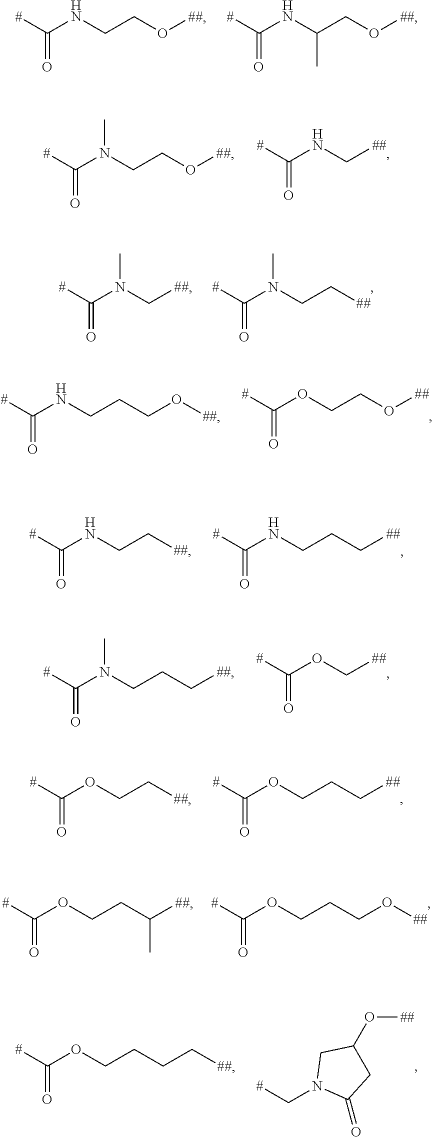

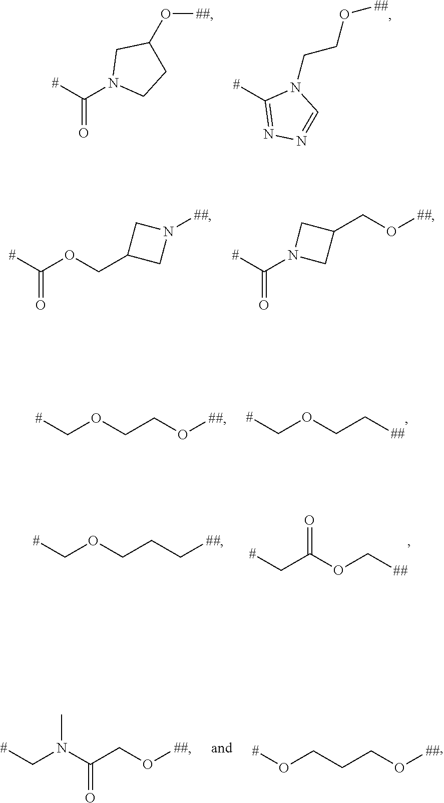

wherein # denotes the point of attachment to

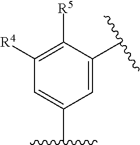

and ## denotes the point of attachment to

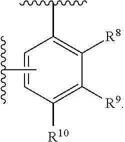

In some embodiments, X⁵ is selected from the group consisting of

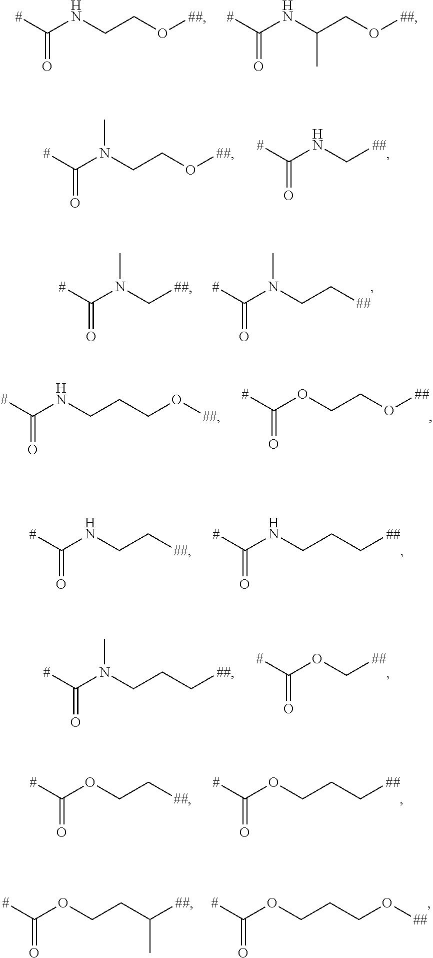

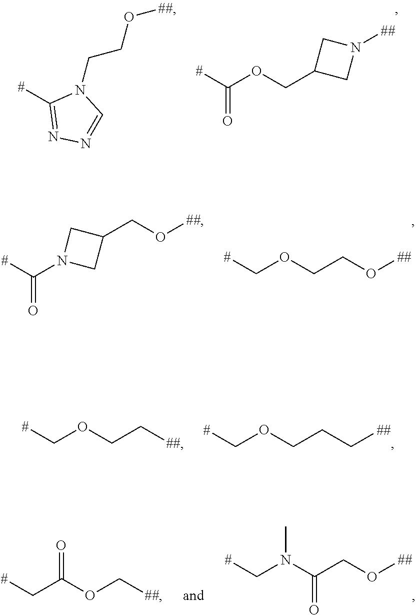

wherein # denotes the point of attachment to

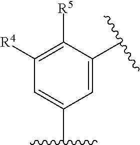

and ## denotes the point of attachment to

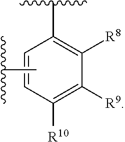

In some embodiments, X⁵ is selected from the group consisting of

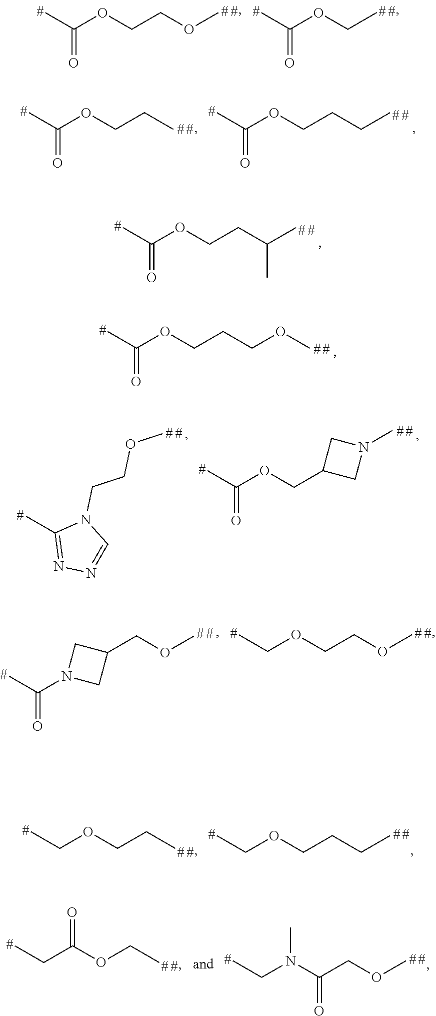

wherein # denotes the point of attachment to

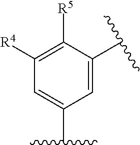

and ## denotes the point of attachment to

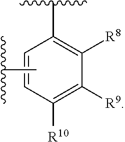

In various embodiments, provided herein are compounds of formula (Ib):

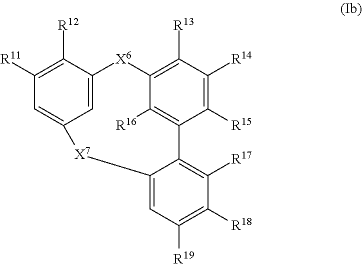

or a stereoisomer and/or a pharmaceutically acceptable salt thereof, wherein:
R¹¹ is selected from the group consisting of hydrogen, hydroxyl, halogen, and C₁₋₆alkyl;
R¹² is selected from the group consisting of hydrogen, halogen, hydroxyl, C₁₋₆alkoxy, C₁₋₆haloalkyl, and C₁₋₆haloalkoxy;
R¹³ is selected from the group consisting of halogen, hydroxyl, C₁₋₆alkoxy, C₁₋₆haloalkoxy, C₁₋₆haloalkyl, and C₃₋₆cycloalkyl;
R¹⁴ is hydrogen or halogen;
R¹¹⁵ is selected from the group consisting of hydrogen, halogen, C₁₋₆alkyl, and C₁₋₆alkoxy;
R¹⁶ is hydrogen or halogen;
R¹⁷ is hydrogen or C₁₋₆alkyl;
R¹⁸ is selected from the group consisting of hydrogen, cyano, halogen, and C₃₋₆cycloalkyl;
R¹⁹ is hydrogen, cyano, or halogen;
X⁶ is selected from the group consisting of *—S(O)₂N(R^{E})—**, *—C(O)N(R^{E})—**, *—CH₂N(R^{E})—**, and *—S(O)₂CH₂—**, wherein * denotes the point of attachment to

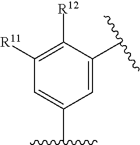

and ** denotes the point of attachment to

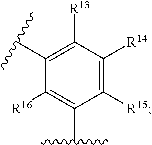

X⁷ is #-L⁷-L⁸-L⁹-##, wherein # denotes the point of attachment to

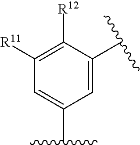

and ## denotes the point of attachment to

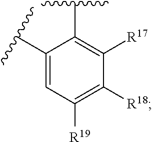

L⁷ is selected from the group consisting of C₁₋₆alkyl-O—, —C(O)—, —C(O)N(R^{F})—, —C(O)O—, —CH₂—C(O)O—, —CH₂—N(R^{F})C(O)—, and 5-6 membered heteroaryl;
L⁸ is Ci₆alkyl or 4-6 membered heterocyclyl;
L⁹ is selected from the group consisting of a bond, —O—, C₁₋₆alkyl-O—, and 4-6 membered heterocyclyl;
R^{E} is hydrogen or C₁₋₆alkyl; and
R^{F} is hydrogen or C₁₋₆alkyl.

In various embodiments, provided herein are compounds of formula (Ib):

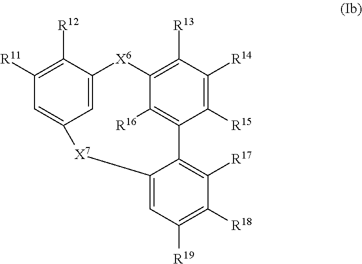

or a stereoisomer and/or a pharmaceutically acceptable salt thereof, wherein:
R¹¹ is selected from the group consisting of hydrogen, hydroxyl, halogen, and C₁₋₆alkyl;
R¹² is selected from the group consisting of hydrogen, halogen, hydroxyl, C₁₋₆alkoxy, C₁₋₆haloalkyl, and C₁₋₆haloalkoxy;
R¹³ is selected from the group consisting of halogen, hydroxyl, C₁₋₆alkoxy, C₁₋₆haloalkoxy, C₁₋₆haloalkyl, and C₃₋₆cycloalkyl;
R¹⁴ is hydrogen or halogen;
R¹⁵ is selected from the group consisting of hydrogen, halogen, C₁₋₆alkyl, and C₁₋₆alkoxy;
R¹⁶ is hydrogen or halogen;
R¹⁷ is hydrogen or C₁₋₆alkyl;
R¹⁸ is selected from the group consisting of hydrogen, cyano, halogen, and C₃₋₆cycloalkyl;
R¹⁹ is hydrogen, cyano, or halogen;
X⁶ is selected from the group consisting of *—S(O)₂N(R^{E})—**, *—C(O)N(R^{E})—**, *—CH₂N(R^{E})—**, and *—S(O)₂CH₂—**, wherein * denotes the point of attachment to

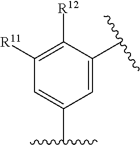

and ** denotes the point of attachment to

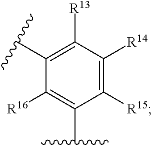

X⁷ is #-L⁷-L-L⁹-##, wherein # denotes the point of attachment to

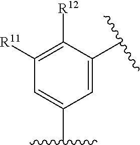

and ## denotes the point of attachment to

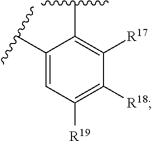

L⁷ is selected from the group consisting of C₁₋₆alkyl-O—, —C(O)—, —C(O)O—, —CH₂—C(O)O—, —CH₂—N(R^{F})C(O)—, and 5-6 membered heteroaryl;
L⁸ is C₁₋₆alkyl or 4-6 membered heterocyclyl;
L⁹ is selected from the group consisting of a bond, —O—, C₁₋₆alkyl-O—, and 4-6 membered heterocyclyl;
R^{E} is hydrogen or C₁₋₆alkyl; and
R^{F} is hydrogen or C₁₋₆alkyl.

In certain embodiments, R¹¹ is selected from the group consisting of hydrogen, hydroxyl, chloro, and CH₃.

In some embodiments, R¹² is selected from the group consisting of hydrogen, fluoro, hydroxyl, CHF₂, —O—CH₃, and —O—CHF₂.

In some embodiments, R¹³ is selected from the group consisting of hydroxyl, fluoro, cyclopropyl, CF₃, —O—CH₃, —O—CHF₂, and —O—CF₃.

In some embodiments, R¹⁴ is hydrogen or fluoro.

In some embodiments, R¹⁵ is selected from the group consisting of hydrogen, chloro, fluoro, —O—CH₃, CH₃, and CF₃.

In some embodiments, R¹⁶ is hydrogen or fluoro.

In some embodiments, R¹⁷ is hydrogen or CH₃.

In some embodiments, R¹⁸ is selected from the group consisting of hydrogen, cyano, chloro, bromo, fluoro, and cyclopropyl.

In some embodiments, R¹⁹ is selected from the group consisting of hydrogen, cyano, chloro, and fluoro.

In some embodiments, X⁶ is selected from the group consisting of *—S(O)₂N(H)—**, *—S(O)₂N(CH₃)—**, *—C(O)N(H)—**, *—CH₂N(H)—**, and *—S(O)₂CH₂—**, wherein * denotes the point of attachment to

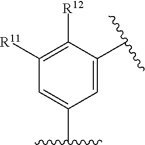

and ** denotes the point of attachment to

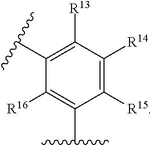

In some embodiments, L⁷ is selected from the group consisting of —CH₂—, —C(O)—, —C(O)N(H)—, —C(O)N(CH₃)—, —C(O)O—, —CH₂—C(O)O—, —CH₂—N(CH₃)C(O)—, —CH₂—O—, and

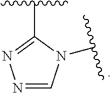 [SMILES: *c1nncn1*]

In some embodiments, L⁷ is selected from the group consisting of —CH₂—, —C(O)—, —C(O)O—, —CH₂—C(O)O—, —N(CH₃)C(O)—, —CH₂—O—, and

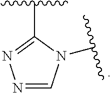 [SMILES: *c1nncn1*]

In some embodiments, L⁸ is selected from the group consisting of —CH₂—, —CH₂CH₂—(CH₂)₃—, —C(H)(CH₃)CH₂—, —CH₂CH₂C(H)(CH₃)—, and

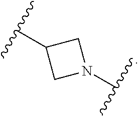 [SMILES: *C1CN(*)C1]

In some embodiments, L⁹ is a bond.

In some embodiments, L⁹ is —O—, —CH₂—O—, or

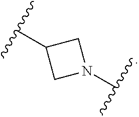 [SMILES: *C1CN(*)C1]

In some embodiments, X⁷ is selected from the group consisting of

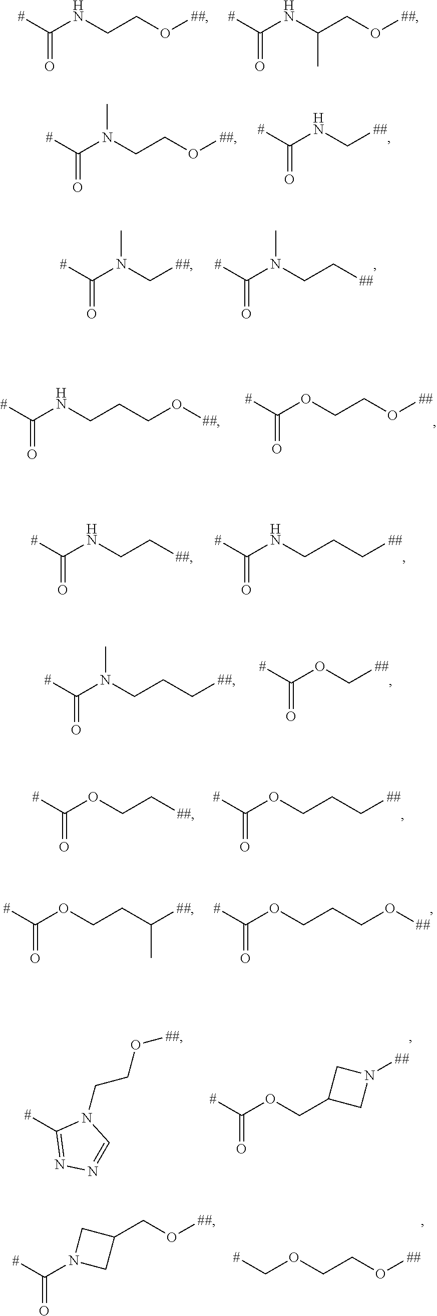

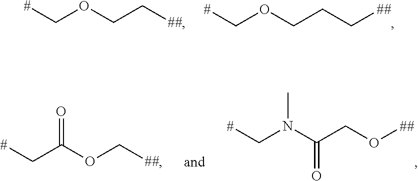

wherein # denotes the point of attachment to

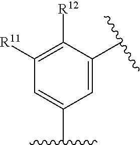

and ## denotes the point of attachment to

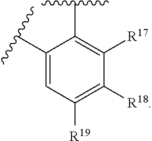

In some embodiments, X⁷ is selected from the group consisting of

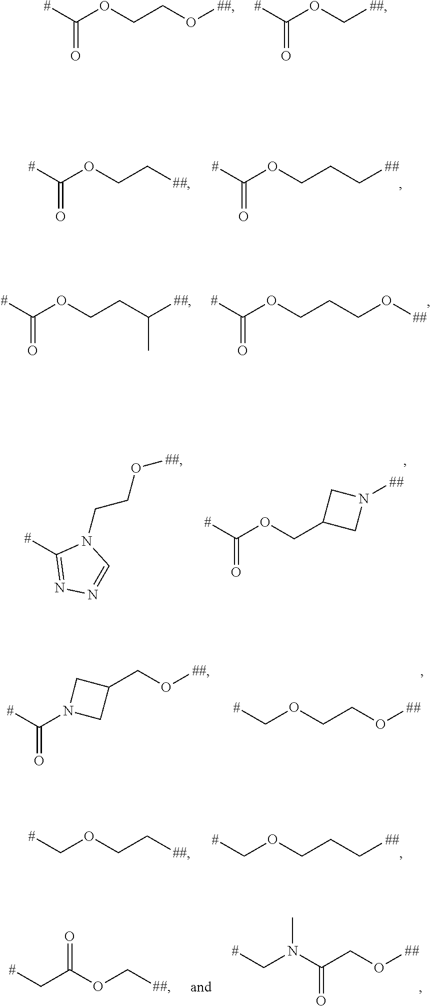

wherein # denotes the point of attachment to

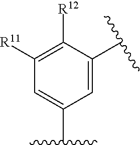

and ## denotes the point of attachment to

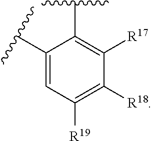

In various embodiments, provided herein are compounds of formula (II).

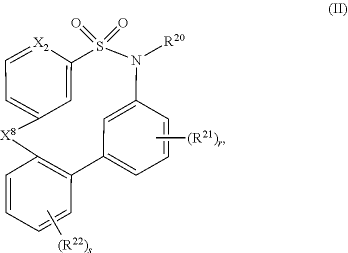

or a stereoisomer and/or a pharmaceutically acceptable salt thereof, wherein:
X₁ and X₂ are C(R^{G}), or
X₁ is C═O and X₂ is N(R^{H}), or
X₁ is N(R^{H}) and X₂ is C═O;
[symbol] is a single bond when X₁ is C═O and X₂ is N(R^{H}), or
[symbol] is a double bond when X₁ is C(R^{G});
R²⁰ is H or C₁₋₃alkyl;
R²¹ is independently, for each occurrence, selected from the group consisting of halogen, hydroxyl, C₁₋₆alkyl, C₁₋₆haloalkyl, C₁₋₆alkoxy, C₁₋₆haloalkoxy, C₃₋₆cycloalkyl, and COOH;
R²² is selected from the group consisting of cyano, halogen, C₁₋₆alkyl, and C₃₋₆cycloalkyl;
X⁸ is —C(O)C₁₋₆alkyl-, —C(O)—(C₁₋₆alkyl)-O—, or -(5-6 membered heteroaryl)-(C₁₋₆alkyl)-O—;
R^{G} is independently, for each occurrence, selected from the group consisting of halogen, hydroxyl, C₁₋₆alkyl, C₁₋₆alkoxy, C₁₋₆haloalkoxy, —O—C(O)C₁₋₆alkyl, and —O—C(O)C₃₋₆cycloalkyl;
R^{H} is H or C₁₋₃alkyl;
r is 0, 1, or 2; and
s is 0 or 1.

In some embodiments, X₁ and X₂ are C(R^{G}). In some embodiments, R^{G} is independently, for each occurrence, halogen or hydroxyl. In some embodiments, R^{G} is independently, for each occurrence, chloro or hydroxyl.

In some embodiments, X₁ is C═O and X₂ is N(R^{H}). In some embodiments, R^{H} is hydrogen. In some embodiments, R^{H} is C₁₋₃alkyl.

In some embodiments, X₁ is N(R^{H}) and X₂ is C═O. In some embodiments, R^{H} is hydrogen. In some embodiments, R^{H} is C₁₋₃alkyl.

In some embodiments, R²⁰ is hydrogen. In some embodiments, R²⁰ is C₁₋₃alkyl.

In some embodiments, r is 0.

In some embodiments, r is 1. In some embodiments, R²¹ is C₁₋₆haloalkyl. In some embodiments, R²¹ is CF₃.

In some embodiments, r is 2. In some embodiments, R²¹ is independently, for each occurrence, halogen. In some embodiments, R²¹ is independently, for each occurrence, fluoro.

In some embodiments, s is 0.

In some embodiments, s is 1. In some embodiments, R²² is halogen. In some embodiments, R²² is fluoro.

In some embodiments, X⁸ is —C(O)CH₂—, —C(O)(CH₂)₂O—, or

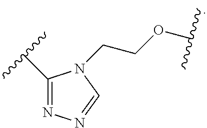 [SMILES: *OCCn1cnnc1*]

In some embodiments, X⁸ is #—C(O)CH₂—##, #—C(O)(CH₂)₂O-##, or

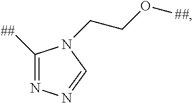

wherein # denotes the point of attachment to

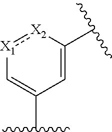

and ## denotes the point of attachment to

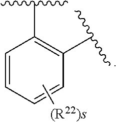

In some embodiments, the compound is selected from Table 1:

TABLE 1
List of compounds.

| Compound No. | Structure |
|---|---|
| 1 | 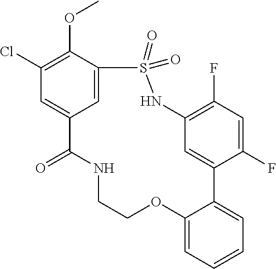 [SMILES: COc1c(Cl)cc2cc1S(=O)(=O)Nc1cc(c(F)cc1F)-c1ccccc1OCCNC2=O] |
| 2 | 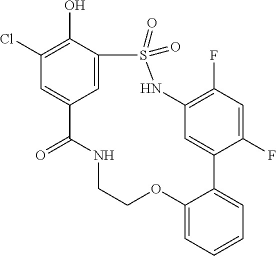 [SMILES: O=C1NCCOc2ccccc2-c2cc(c(F)cc2F)NS(=O)(=O)c2cc1cc(Cl)c2O] |
| 3 | 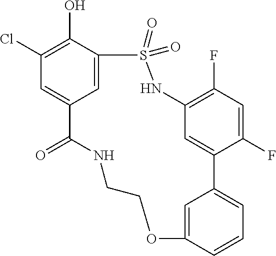 [SMILES: O=C1NCCOc2cccc(c2)-c2cc(c(F)cc2F)NS(=O)(=O)c2cc1cc(Cl)c2O] |
| 4 | 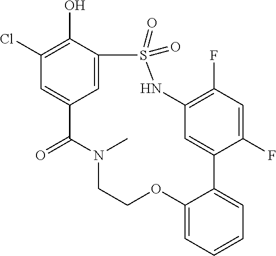 [SMILES: CN1CCOc2ccccc2-c2cc(c(F)cc2F)NS(=O)(=O)c2cc(cc(Cl)c2O)C1=O] |
| 5 | 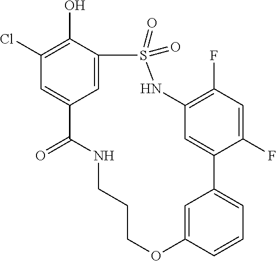 [SMILES: O=C1NCCCOc2cccc(c2)-c2cc(c(F)cc2F)NS(=O)(=O)c2cc1cc(Cl)c2O] |
| 6 | 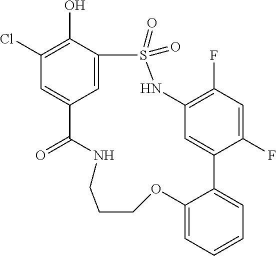 [SMILES: O=C1NCCCOc2ccccc2-c2cc(c(F)cc2F)NS(=O)(=O)c2cc1cc(Cl)c2O] |
| 7 | 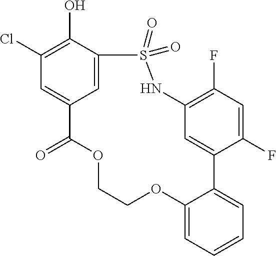 [SMILES: O=C1OCCOc2ccccc2-c2cc(c(F)cc2F)NS(=O)(=O)c2cc1cc(Cl)c2O] |
| 8 | 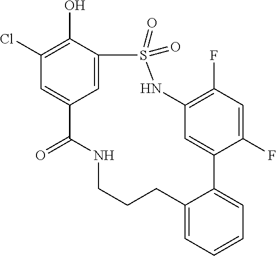 [SMILES: O=C1NCCCc2ccccc2-c2cc(c(F)cc2F)NS(=O)(=O)c2cc1cc(Cl)c2O] |
| 9 | 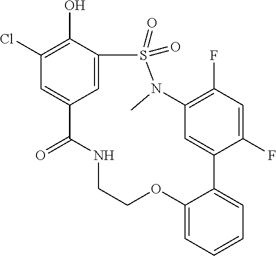 [SMILES: CN1c2cc(c(F)cc2F)-c2ccccc2OCCNC(=O)c2cc(Cl)c(O)c(c2)S1(=O)=O] |
| 10 | 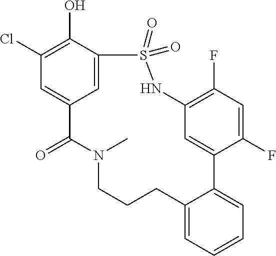 [SMILES: CN1CCCc2ccccc2-c2cc(c(F)cc2F)NS(=O)(=O)c2cc(cc(Cl)c2O)C1=O] |
| 11 | 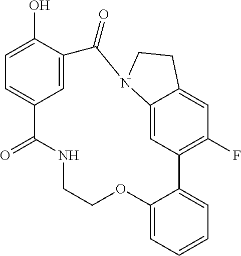 [SMILES: O=C1NCCOc2ccccc2-c2cc3c(cc2F)CCN3C(=O)c2cc1ccc2O] |
| 12 | 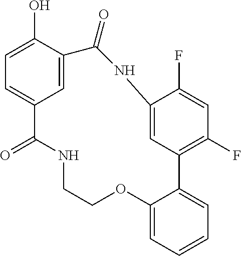 [SMILES: O=C1NCCOc2ccccc2-c2cc(c(F)cc2F)NC(=O)c2cc1ccc2O] |
| 13 | 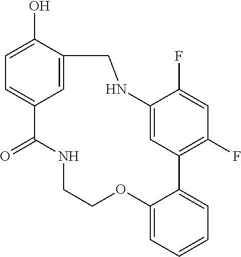 [SMILES: O=C1NCCOc2ccccc2-c2cc(c(F)cc2F)NCc2cc1ccc2O] |
| 14 | 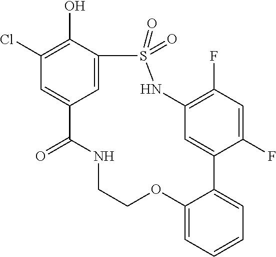 [SMILES: O=C1NCCOc2ccccc2-c2cc(c(F)cc2F)NS(=O)(=O)c2cc1cc(Cl)c2O] |
| 15 | 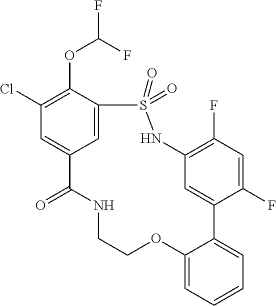 [SMILES: O=C1NCCOc2ccccc2-c2cc(c(F)cc2F)NS(=O)(=O)c2cc1cc(Cl)c2OC(F)F] |
| 16 | 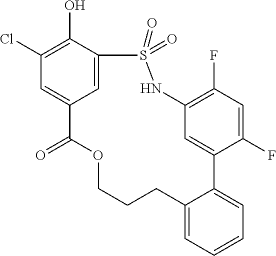 [SMILES: O=C1OCCCc2ccccc2-c2cc(c(F)cc2F)NS(=O)(=O)c2cc1cc(Cl)c2O] |
| 17 | 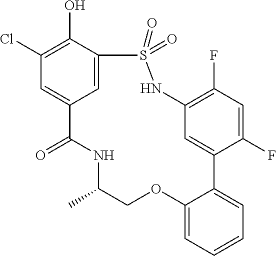 [SMILES: C[C@H]1COc2ccccc2-c2cc(c(F)cc2F)NS(=O)(=O)c2cc(cc(Cl)c2O)C(=O)N1] |
| 18 | 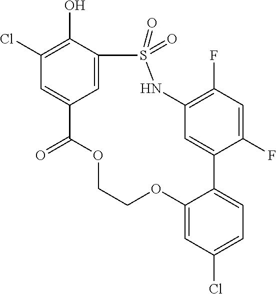 [SMILES: O=C1OCCOc2cc(Cl)ccc2-c2cc(c(F)cc2F)NS(=O)(=O)c2cc1cc(Cl)c2O] |
| 19 | 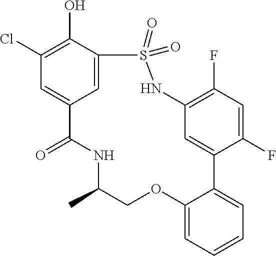 [SMILES: C[C@@H]1COc2ccccc2-c2cc(c(F)cc2F)NS(=O)(=O)c2cc(cc(Cl)c2O)C(=O)N1] |
| 20 | 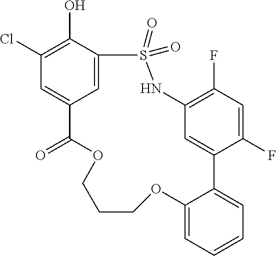 [SMILES: O=C1OCCCOc2ccccc2-c2cc(c(F)cc2F)NS(=O)(=O)c2cc1cc(Cl)c2O] |
| 21 | 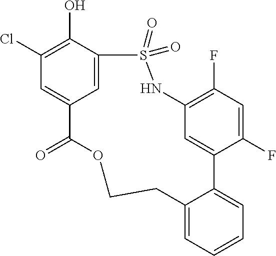 [SMILES: O=C1OCCc2ccccc2-c2cc(c(F)cc2F)NS(=O)(=O)c2cc1cc(Cl)c2O] |
| 22 | 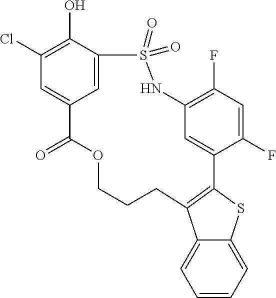 [SMILES: O=C1OCCCc2c(sc3ccccc23)-c2cc(c(F)cc2F)NS(=O)(=O)c2cc1cc(Cl)c2O] |
| 23 | 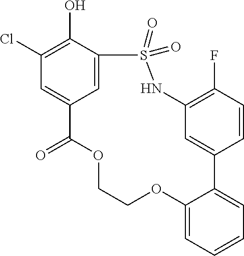 [SMILES: O=C1OCCOc2ccccc2-c2ccc(F)c(c2)NS(=O)(=O)c2cc1cc(Cl)c2O] |
| 24 | 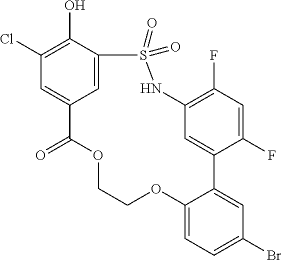 [SMILES: O=C1OCCOc2ccc(Br)cc2-c2cc(c(F)cc2F)NS(=O)(=O)c2cc1cc(Cl)c2O] |
| 25 | 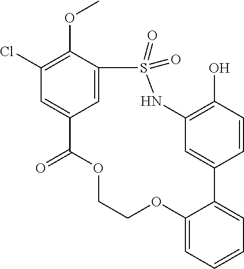 [SMILES: COc1c(Cl)cc2cc1S(=O)(=O)Nc1cc(ccc1O)-c1ccccc1OCCOC2=O] |
| 26 | 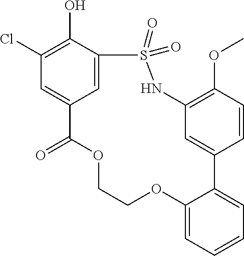 [SMILES: COc1ccc2cc1NS(=O)(=O)c1cc(cc(Cl)c1O)C(=O)OCCOc1ccccc1-2] |
| 27 | 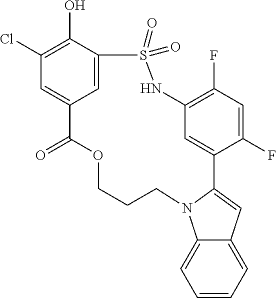 [SMILES: O=C1OCCCn2c(cc3ccccc32)-c2cc(c(F)cc2F)NS(=O)(=O)c2cc1cc(Cl)c2O] |
| 28 | 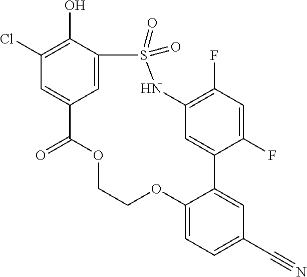 [SMILES: N#Cc1ccc2c(c1)-c1cc(c(F)cc1F)NS(=O)(=O)c1cc(cc(Cl)c1O)C(=O)OCCO2] |
| 29 | 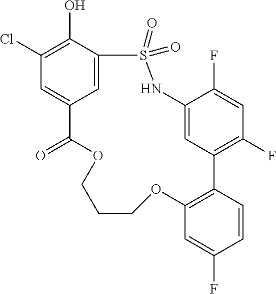 [SMILES: O=C1OCCCOc2cc(F)ccc2-c2cc(c(F)cc2F)NS(=O)(=O)c2cc1cc(Cl)c2O] |
| 30 | 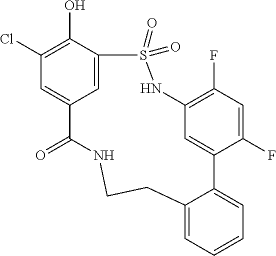 [SMILES: O=C1NCCc2ccccc2-c2cc(c(F)cc2F)NS(=O)(=O)c2cc1cc(Cl)c2O] |
| 31 | 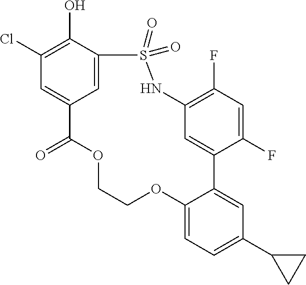 [SMILES: O=C1OCCOc2ccc(C3CC3)cc2-c2cc(c(F)cc2F)NS(=O)(=O)c2cc1cc(Cl)c2O] |
| 32 | 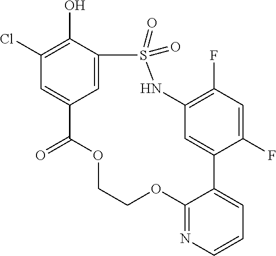 [SMILES: O=C1OCCOc2ncccc2-c2cc(c(F)cc2F)NS(=O)(=O)c2cc1cc(Cl)c2O] |
| 33 | 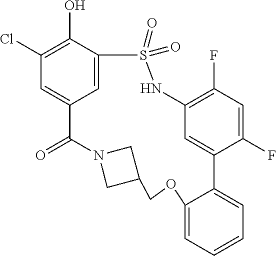 [SMILES: O=C1c2cc(Cl)c(O)c(c2)S(=O)(=O)Nc2cc(c(F)cc2F)-c2ccccc2OCC2CN1C2] |
| 34 | 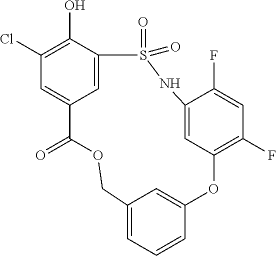 [SMILES: O=C1OCc2cccc(c2)Oc2cc(c(F)cc2F)NS(=O)(=O)c2cc1cc(Cl)c2O] |
| 35 | 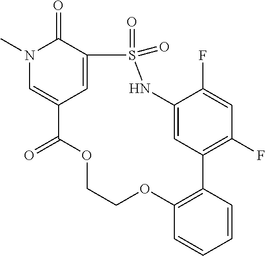 [SMILES: Cn1cc2cc(c1=O)S(=O)(=O)Nc1cc(c(F)cc1F)-c1ccccc1OCCOC2=O] |
| 36 | 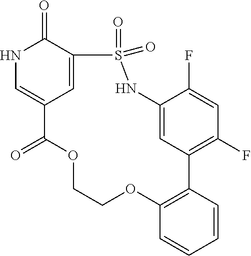 [SMILES: O=C1OCCOc2ccccc2-c2cc(c(F)cc2F)NS(=O)(=O)c2cc1c[nH]c2=O] |
| 37 | 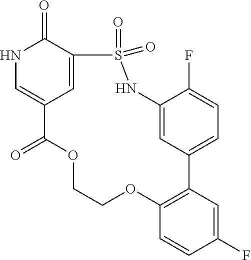 [SMILES: O=C1OCCOc2ccc(F)cc2-c2ccc(F)c(c2)NS(=O)(=O)c2cc1c[nH]c2=O] |
| 38 | 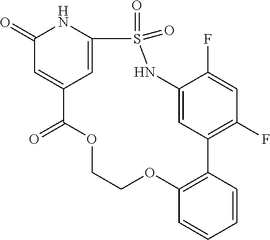 [SMILES: O=C1OCCOc2ccccc2-c2cc(c(F)cc2F)NS(=O)(=O)c2cc1cc(=O)[nH]2] |
| 39 | 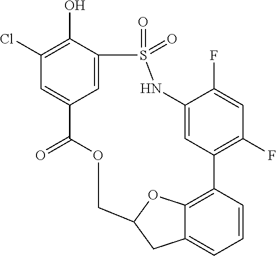 [SMILES: O=C1OCC2Cc3cccc(c3O2)-c2cc(c(F)cc2F)NS(=O)(=O)c2cc1cc(Cl)c2O] |
| 40 | 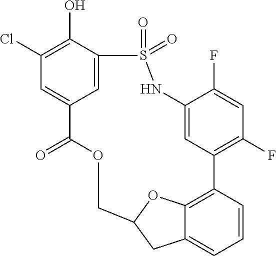 [SMILES: O=C1OCC2Cc3cccc(c3O2)-c2cc(c(F)cc2F)NS(=O)(=O)c2cc1cc(Cl)c2O] |
| | (enantiomer) |
| 41 | 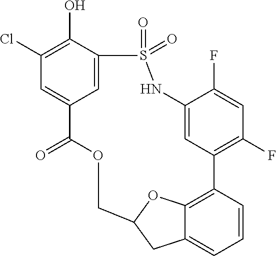 [SMILES: O=C1OCC2Cc3cccc(c3O2)-c2cc(c(F)cc2F)NS(=O)(=O)c2cc1cc(Cl)c2O] |
| | (enantiomer) |
| 42 | 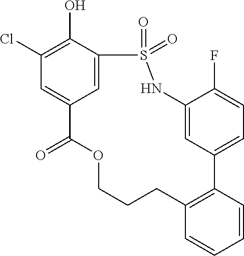 [SMILES: O=C1OCCCc2ccccc2-c2ccc(F)c(c2)NS(=O)(=O)c2cc1cc(Cl)c2O] |
| 43 | 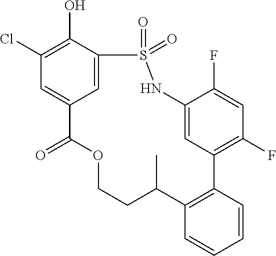 [SMILES: CC1CCOC(=O)c2cc(Cl)c(O)c(c2)S(=O)(=O)Nc2cc(c(F)cc2F)-c2ccccc21] |
| 44 | 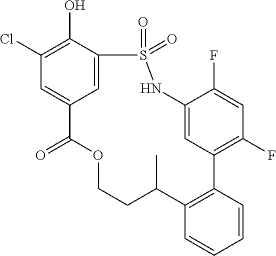 [SMILES: CC1CCOC(=O)c2cc(Cl)c(O)c(c2)S(=O)(=O)Nc2cc(c(F)cc2F)-c2ccccc21] |
| | (enantiomer) |
| 45 | 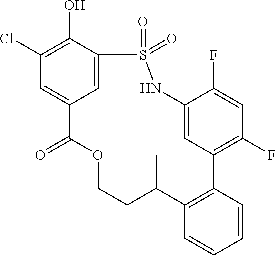 [SMILES: CC1CCOC(=O)c2cc(Cl)c(O)c(c2)S(=O)(=O)Nc2cc(c(F)cc2F)-c2ccccc21] |
| | (enantiomer) |
| 46 | 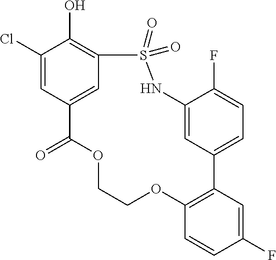 [SMILES: O=C1OCCOc2ccc(F)cc2-c2ccc(F)c(c2)NS(=O)(=O)c2cc1cc(Cl)c2O] |
| 47 | 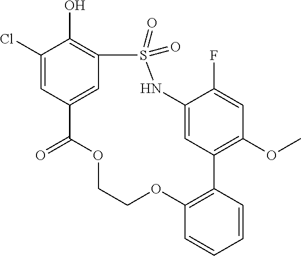 [SMILES: COc1cc(F)c2cc1-c1ccccc1OCCOC(=O)c1cc(Cl)c(O)c(c1)S(=O)(=O)N2] |
| 48 | 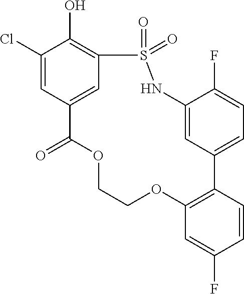 [SMILES: O=C1OCCOc2cc(F)ccc2-c2ccc(F)c(c2)NS(=O)(=O)c2cc1cc(Cl)c2O] |
| 49 | 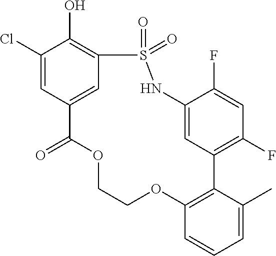 [SMILES: Cc1cccc2c1-c1cc(c(F)cc1F)NS(=O)(=O)c1cc(cc(Cl)c1O)C(=O)OCCO2] |
| 50 | 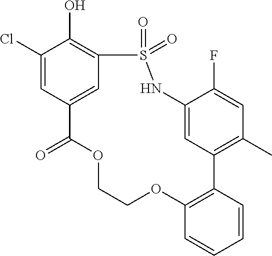 [SMILES: Cc1cc(F)c2cc1-c1ccccc1OCCOC(=O)c1cc(Cl)c(O)c(c1)S(=O)(=O)N2] |
| 51 | 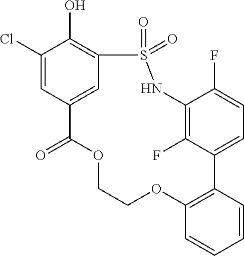 [SMILES: O=C1OCCOc2ccccc2-c2ccc(F)c(c2F)NS(=O)(=O)c2cc1cc(Cl)c2O] |
| 52 | 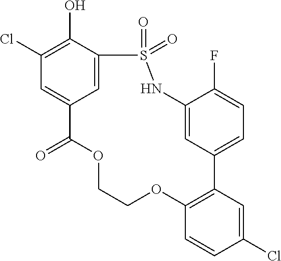 [SMILES: O=C1OCCOc2ccc(Cl)cc2-c2ccc(F)c(c2)NS(=O)(=O)c2cc1cc(Cl)c2O] |
| 53 | 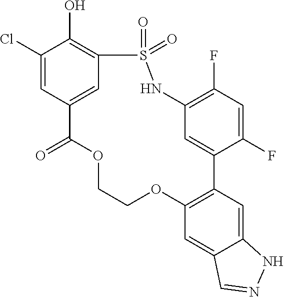 [SMILES: O=C1OCCOc2cc3cn[nH]c3cc2-c2cc(c(F)cc2F)NS(=O)(=O)c2cc1cc(Cl)c2O] |
| 54 | 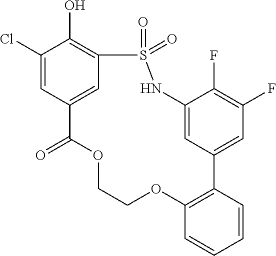 [SMILES: O=C1OCCOc2ccccc2-c2cc(F)c(F)c(c2)NS(=O)(=O)c2cc1cc(Cl)c2O] |
| 55 | 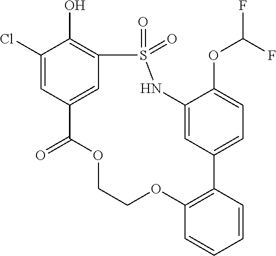 [SMILES: O=C1OCCOc2ccccc2-c2ccc(OC(F)F)c(c2)NS(=O)(=O)c2cc1cc(Cl)c2O] |
| 56 | 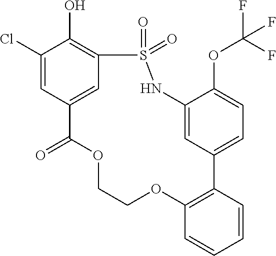 [SMILES: O=C1OCCOc2ccccc2-c2ccc(OC(F)(F)F)c(c2)NS(=O)(=O)c2cc1cc(Cl)c2O] |
| 57 | 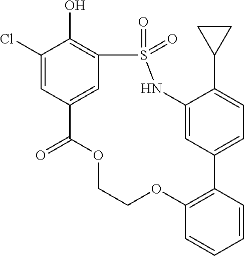 [SMILES: O=C1OCCOc2ccccc2-c2ccc(C3CC3)c(c2)NS(=O)(=O)c2cc1cc(Cl)c2O] |
| 58 | 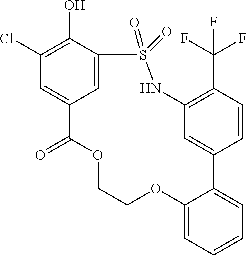 [SMILES: O=C1OCCOc2ccccc2-c2ccc(C(F)(F)F)c(c2)NS(=O)(=O)c2cc1cc(Cl)c2O] |
| 59 | 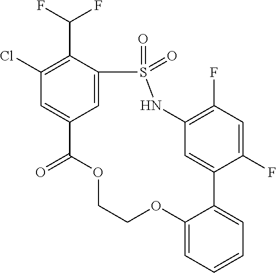 [SMILES: O=C1OCCOc2ccccc2-c2cc(c(F)cc2F)NS(=O)(=O)c2cc1cc(Cl)c2C(F)F] |
| 60 | 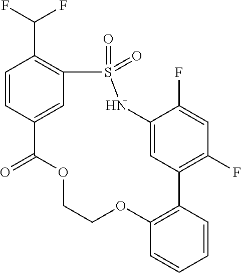 [SMILES: O=C1OCCOc2ccccc2-c2cc(c(F)cc2F)NS(=O)(=O)c2cc1ccc2C(F)F] |
| 61 | 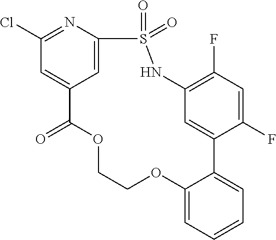 [SMILES: O=C1OCCOc2ccccc2-c2cc(c(F)cc2F)NS(=O)(=O)c2cc1cc(Cl)n2] |
| 62 | 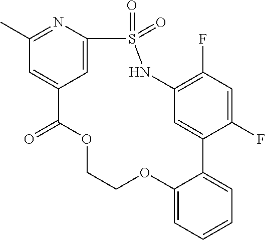 [SMILES: Cc1cc2cc(n1)S(=O)(=O)Nc1cc(c(F)cc1F)-c1ccccc1OCCOC2=O] |
| 63 | 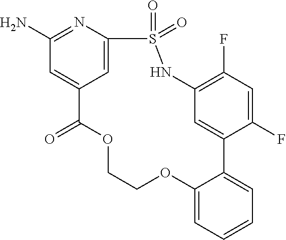 [SMILES: Nc1cc2cc(n1)S(=O)(=O)Nc1cc(c(F)cc1F)-c1ccccc1OCCOC2=O] |
| 64 | 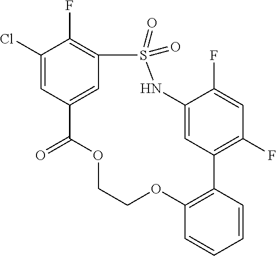 [SMILES: O=C1OCCOc2ccccc2-c2cc(c(F)cc2F)NS(=O)(=O)c2cc1cc(Cl)c2F] |
| 65 | 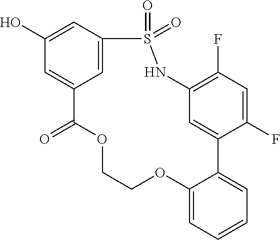 [SMILES: O=C1OCCOc2ccccc2-c2cc(c(F)cc2F)NS(=O)(=O)c2cc(O)cc1c2] |
| 66 | 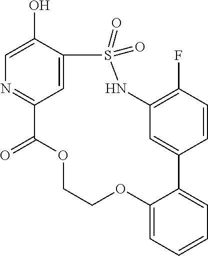 [SMILES: O=C1OCCOc2ccccc2-c2ccc(F)c(c2)NS(=O)(=O)c2cc1ncc2O] |
| 67 | 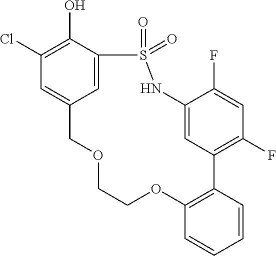 [SMILES: O=S1(=O)Nc2cc(c(F)cc2F)-c2ccccc2OCCOCc2cc(Cl)c(O)c1c2] |
| 68 | 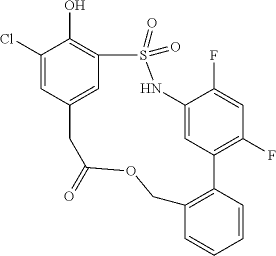 [SMILES: O=C1Cc2cc(Cl)c(O)c(c2)S(=O)(=O)Nc2cc(c(F)cc2F)-c2ccccc2CO1] |
| 69 | 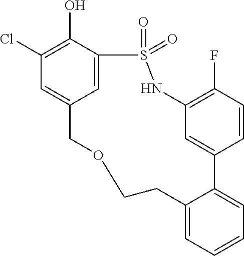 [SMILES: O=S1(=O)Nc2cc(ccc2F)-c2ccccc2CCOCc2cc(Cl)c(O)c1c2] |
| 70 | 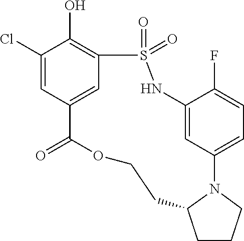 [SMILES: O=C1OCC[C@H]2CCCN2c2ccc(F)c(c2)NS(=O)(=O)c2cc1cc(Cl)c2O] |
| 71 | 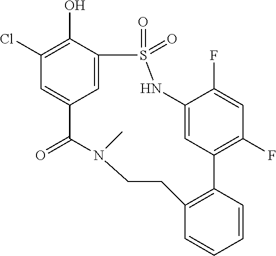 [SMILES: CN1CCc2ccccc2-c2cc(c(F)cc2F)NS(=O)(=O)c2cc(cc(Cl)c2O)C1=O] |
| 72 | 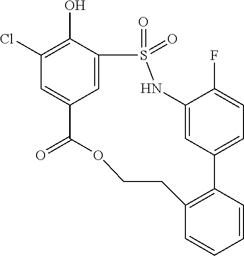 [SMILES: O=C1OCCc2ccccc2-c2ccc(F)c(c2)NS(=O)(=O)c2cc1cc(Cl)c2O] |
| 73 | 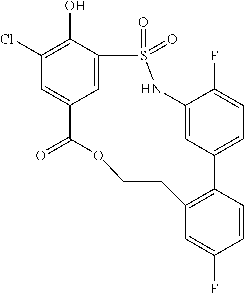 [SMILES: O=C1OCCc2cc(F)ccc2-c2ccc(F)c(c2)NS(=O)(=O)c2cc1cc(Cl)c2O] |
| 74 | 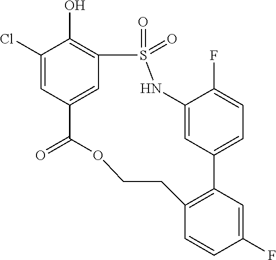 [SMILES: O=C1OCCc2ccc(F)cc2-c2ccc(F)c(c2)NS(=O)(=O)c2cc1cc(Cl)c2O] |
| 75 | 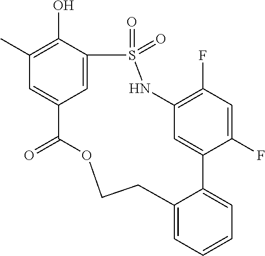 [SMILES: Cc1cc2cc(c1O)S(=O)(=O)Nc1cc(c(F)cc1F)-c1ccccc1CCOC2=O] |
| 76 | 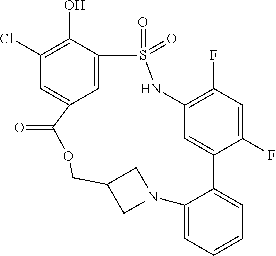 [SMILES: O=C1OCC2CN(C2)c2ccccc2-c2cc(c(F)cc2F)NS(=O)(=O)c2cc1cc(Cl)c2O] |
| 77 | 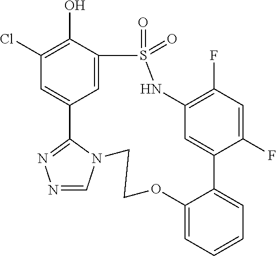 [SMILES: O=S1(=O)Nc2cc(c(F)cc2F)-c2ccccc2OCCn2cnnc2-c2cc(Cl)c(O)c1c2] |
| 78 | 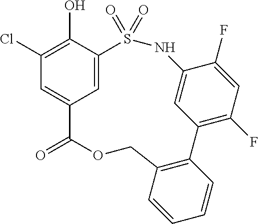 [SMILES: O=C1OCc2ccccc2-c2cc(c(F)cc2F)NS(=O)(=O)c2cc1cc(Cl)c2O] |
| 79 | 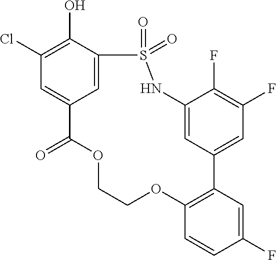 [SMILES: O=C1OCCOc2ccc(F)cc2-c2cc(F)c(F)c(c2)NS(=O)(=O)c2cc1cc(Cl)c2O] |
| 80 | 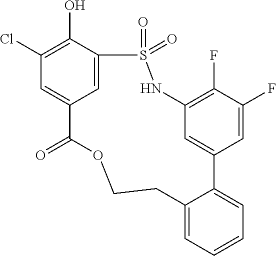 [SMILES: O=C1OCCc2ccccc2-c2cc(F)c(F)c(c2)NS(=O)(=O)c2cc1cc(Cl)c2O] |
| 81 | 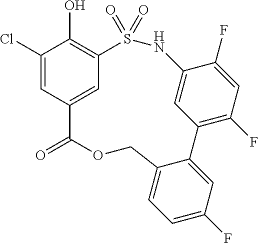 [SMILES: O=C1OCc2ccc(F)cc2-c2cc(c(F)cc2F)NS(=O)(=O)c2cc1cc(Cl)c2O] |
| 82 | 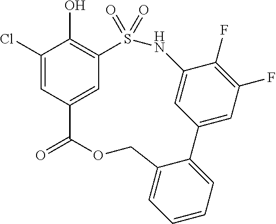 [SMILES: O=C1OCc2ccccc2-c2cc(F)c(F)c(c2)NS(=O)(=O)c2cc1cc(Cl)c2O] |
| 83 | 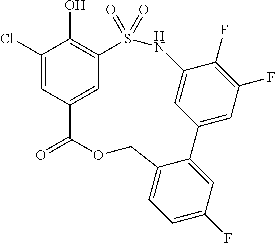 [SMILES: O=C1OCc2ccc(F)cc2-c2cc(F)c(F)c(c2)NS(=O)(=O)c2cc1cc(Cl)c2O] |
| 84 | 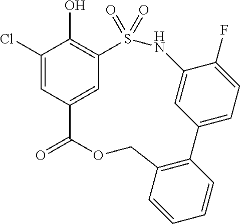 [SMILES: O=C1OCc2ccccc2-c2ccc(F)c(c2)NS(=O)(=O)c2cc1cc(Cl)c2O] |
| 85 | 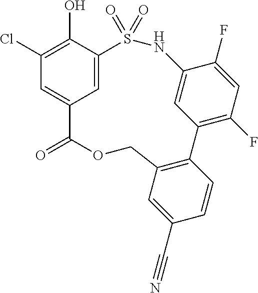 [SMILES: N#Cc1ccc2c(c1)COC(=O)c1cc(Cl)c(O)c(c1)S(=O)(=O)Nc1cc-2c(F)cc1F] |
| 86 | 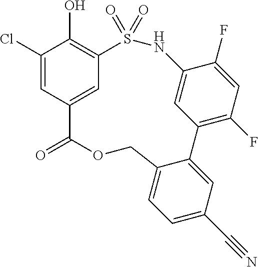 [SMILES: N#Cc1ccc2c(c1)-c1cc(c(F)cc1F)NS(=O)(=O)c1cc(cc(Cl)c1O)C(=O)OC2] |
| 87 | 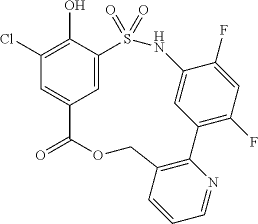 [SMILES: O=C1OCc2cccnc2-c2cc(c(F)cc2F)NS(=O)(=O)c2cc1cc(Cl)c2O] |
| 88 | 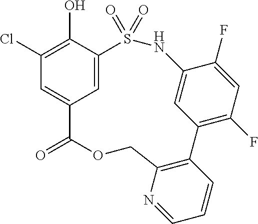 [SMILES: O=C1OCc2ncccc2-c2cc(c(F)cc2F)NS(=O)(=O)c2cc1cc(Cl)c2O] |
| 89 | 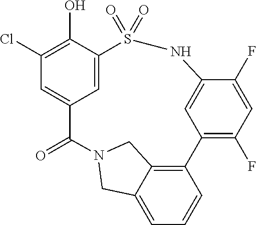 [SMILES: O=C1c2cc(Cl)c(O)c(c2)S(=O)(=O)Nc2cc(c(F)cc2F)-c2cccc3c2CN1C3] |
| 90 | 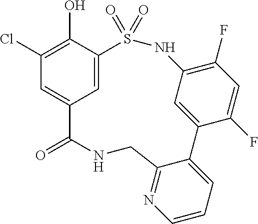 [SMILES: O=C1NCc2ncccc2-c2cc(c(F)cc2F)NS(=O)(=O)c2cc1cc(Cl)c2O] |
| 91 | 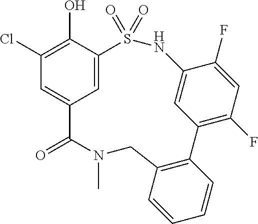 [SMILES: CN1Cc2ccccc2-c2cc(c(F)cc2F)NS(=O)(=O)c2cc(cc(Cl)c2O)C1=O] |
| 92 | 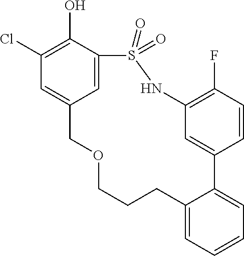 [SMILES: O=S1(=O)Nc2cc(ccc2F)-c2ccccc2CCCOCc2cc(Cl)c(O)c1c2] |
| 93 | 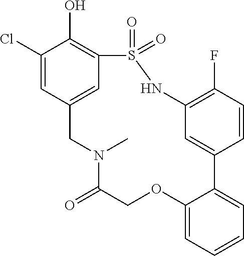 [SMILES: CN1Cc2cc(Cl)c(O)c(c2)S(=O)(=O)Nc2cc(ccc2F)-c2ccccc2OCC1=O] |

### Pharmaceutical Compositions and Routes of Administration

Compounds provided in accordance with the present disclosure are usually administered in the form of pharmaceutical compositions. This invention therefore provides pharmaceutical compositions that contain, as the active ingredient, one or more of the compounds described herein, or a stereoisomer and/or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions described herein may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions described herein may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the disclosure. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper, or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills, and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprises a disclosed compound (e.g., a compound of formula (I), formula (Ia), formula (Ib), or formula (II)), or a stereoisomer and/or a pharmaceutically acceptable salt thereof, and a pharmaceutically acceptable carrier.

### Methods of Treatment

In various embodiments, a compound disclosed herein (e.g., a compound of formula (I), formula (Ia), formula (Ib), or formula (II)), or a stereoisomer thereof and/or a pharmaceutically acceptable salt thereof, including a pharmaceutical composition, may be used for the treatment or prevention of a variety of conditions, diseases, and disorders. The methods of treating a condition, disease, or disorder described herein generally comprise administering to a patient in need thereof, a therapeutically effective amount of a compound described herein (e.g., a compound of formula (I), formula (Ia), formula (Ib), or formula (II)), or a stereoisomer and/or a pharmaceutically acceptable salt thereof, to treat the condition, disease, or disorder.

In typical embodiments, the present disclosure is intended to encompass a compound disclosed herein, or a stereoisomer thereof and/or a pharmaceutically acceptable salt thereof. In some embodiments, the present disclosure includes an enantiomer, a mixture of enantiomers, a stereoisomer, or mixture of stereoisomers (pure or as a racemic or non-racemic mixture) of a compound described herein (e.g., a compound of formula (I), formula (Ia), formula (Ib), or formula (II)).

Examples of conditions, diseases, and disorders include, but are not limited to, cardiovascular disease, atrial fibrillation, blood clotting, coronary heart disease, hypercoagulable states, ischemia, myocardial infarction, myopathy, myositis, pulmonary embolism, stroke, peripheral vascular disease, pulmonary hypertension, pulmonary arterial hypertension, dyslipidemia, dyslipoproteinemia, a disorder of glucose metabolism, Alzheimer's disease, Parkinson's disease, diabetic nephropathy, diabetic retinopathy, insulin resistance, metabolic syndrome disorders (e.g., Syndrome X), galactosemia, HIV infection, a peroxisome proliferator activated receptor-associated disorder, septicemia, a thrombotic disorder, obesity, pancreatitis, hypertension, renal disease, cancer, inflammation (e.g., liver inflammation), inflammatory muscle diseases (e.g., polymyalgia rheumatica, polymyositis, and fibrositis), impotence, gastrointestinal disease, irritable bowel syndrome, inflammatory bowel disease, inflammatory disorders (e.g., asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis), arthritis (e.g., rheumatoid arthritis, juvenile rheumatoid arthritis, and osteoarthritis), osteoporosis, soft tissue rheumatism (e.g., tendonitis), bursitis, autoimmune disease (e.g., systemic lupus and erythematosus), scleroderma, ankylosing spondylitis, gout, pseudogout, non-insulin dependent diabetes mellitus, diabetes (e.g., type 2), polycystic ovarian disease, hyperlipidemias (e.g., primary hyperlipidemia, familial hypercholesterolemia (FH), Hypercholesterolemia Frederickson Type IIa, Hypercholesterolemia Frederickson Type IIb, familial combined hyperlipidemia (FCH)), lipoprotein lipase deficiencies (e.g., hypertriglyceridemia, hypoalphalipoproteinemia, and hypercholesterolemia), lipoprotein abnormalities associated with diabetes, lipoprotein abnormalities associated with obesity, and lipoprotein abnormalities associated with Alzheimer's disease. In particular embodiments, the methods include treating and/or preventing hyperlipidemia such as primary hyperlipidemia. In some embodiments, the methods include treating and/or preventing cardiovascular disease.

In certain embodiments, a compound disclosed herein (e.g., a compound of formula (I), formula (Ia), formula (Ib), or formula (II)), or a stereoisomer and/or a pharmaceutically acceptable salt thereof, including a pharmaceutical composition, may be used for the treatment or prevention of one or more of high levels of low density lipoprotein cholesterol (LDL-C), high levels of apolipoprotein B (apoB), high levels of lipoprotein(a) (Lp(a)), high levels of very low density lipoprotein (VLDL), high levels of non-high density lipid cholesterol (non-HDL-C), high levels of total serum cholesterol (TC), high levels of high sensitivity c-reactive protein (hsCRP), high levels of fibrinogen, high levels of insulin, high levels of glucose, and low levels of high density lipoprotein cholesterol (HDL-C). In other words, methods of the disclosure can include lowering LDL-C, lowering apoB, lowering Lp(a), lowering VLDL, lowering non-HDL-C, lowering TC, and/or lowering hsCRP. Methods of the disclosure can include inhibiting ACLY, inhibiting cholesterol synthesis, and/or suppressing fatty acid biosynthesis. In some embodiments, an effective amount of a compound described herein (e.g., a compound of formula (I), formula (Ia), formula (Ib), or formula (II)), or a stereoisomer and/or a pharmaceutically acceptable salt thereof, or a pharmaceutical composition of the present disclosure may be used as an adjunct to diet and maximally tolerated statin therapy to lower LDL-C in adults with heterozygous familial hypercholesterolemia or established atherosclerotic cardiovascular disease. In some embodiments, an effective amount of a compound described herein (e.g., a compound of formula (I), formula (Ia), formula (Ib), or formula (II)), or a stereoisomer and/or a pharmaceutically acceptable salt thereof, or a pharmaceutical composition of the present disclosure may be used for the treatment of non-insulin dependent diabetes mellitus without increasing weight gain.

In certain embodiments, a compound disclosed herein (e.g., a compound of formula (I), formula (Ia), formula (Ib), or formula (II)), or a stereoisomer and/or a pharmaceutically acceptable salt thereof, including a pharmaceutical composition, may be used for the treatment or prevention of a variety of diseases and conditions, which include, but are not limited to aging, Alzheimer's disease, cancer, cardiovascular disease, diabetic nephropathy, diabetic retinopathy, a disorder of glucose metabolism, dyslipidemia, dyslipoproteinemia, enhancing bile production, hypertension, impotence, inflammation, insulin resistance, lipid elimination in bile, modulating C reactive protein, obesity, oxysterol elimination in bile, pancreatitis, pancreatitius, Parkinson's disease, a peroxisome proliferator activated receptor-associated disorder, phospholipid elimination in bile, renal disease, rhabdomyolysis, septicemia, sleep apnea, Syndrome X, and a thrombotic disorder.

In certain embodiments, provided herein is a method of treating a liver disorder selected from the group consisting of steatohepatitis, alcoholic liver disease, fatty liver, liver steatosis, liver cirrhosis, liver fibrosis, and acute fatty liver of pregnancy. In some embodiments, the disorder is steatohepatitis. In some embodiments, the steatohepatitis is NASH. In some embodiments, the steatohepatitis is NASH. In some embodiments, the disorder is alcoholic liver disease. In some embodiments, the disorder is fatty liver. In some embodiments, the disorder is liver steatosis, liver cirrhosis, or liver fibrosis. In some embodiments, the disorder is acute fatty liver of pregnancy. In some embodiments, the patient is an adult human.

In certain embodiments, the present disclosure provides a method for treating or preventing aging, Alzheimer's disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, pancreatitius, Parkinson's disease, a peroxisome proliferator activated receptor-associated disorder, phospholipid elimination in bile, renal disease, septicemia, metabolic syndrome disorders (e.g., Syndrome X), or a thrombotic disorder.

In certain embodiments, the disorder is selected from the group consisting of lipodystrophy, lysosomal acid lipase deficiency, and a glycogen storage disease. In some embodiments, the patient is an adult human.

In certain embodiments, the disorder is selected from the group consisting of hepatitis C, an infection by human immunodeficiency virus, an alpha 1-antitrypsin deficiency, Bassen-Kornzweig syndrome, hypobetalipoproteinemia, Celiac disease, Wilson's disease, and Weber-Christian syndrome. In some embodiments, the disorder is hepatitis B.

In some embodiments, the disorder is hepatitis C. In some embodiments, the disorder is an infection by human immunodeficiency virus. In some embodiments, the disorder is an alpha 1-antitrypsin deficiency. In some embodiments, the disorder is Bassen-Kornzweig syndrome.

In some embodiments, the disorder is hypobetalipoproteinemia. In some embodiments, the disorder is Celiac disease or Wilson's disease. In some embodiments, the disorder is Weber-Christian syndrome. In some embodiments, the patient is an adult human.

In certain embodiments, the condition is selected from the group consisting of toxic liver injury, total parenteral nutrition, severe surgical weight loss, environmental toxicity, malnutrition, and starvation. In some embodiments, the condition is toxic liver injury. In some embodiments, the condition is total parenteral nutrition or severe surgical weight loss.

In some embodiments, the condition is environmental toxicity. In some embodiments, the condition is malnutrition or starvation. In some embodiments, the patient is an adult human.

In various embodiments, provided herein are methods of treating NAFLD in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating NASH in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating type-2 diabetes in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating inflammation in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating chronic kidney disease in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating autoimmunity in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In various embodiments, provided herein are methods of treating cancer (e.g., a liver cancer) in a subject in need thereof, the methods generally comprise administering to the subject a therapeutically effect amount of a compound disclosed herein (e.g., a compound of formula (I), formula (Ia), formula (Ib), or formula (II)), or a stereoisomer thereof and/or a pharmaceutically acceptable salt thereof, or a pharmaceutical composition disclosed herein.

In certain embodiments, in order to prolong the effect of a drug, a compound described herein (e.g., a compound of formula (I), formula (Ia), formula (Ib), or formula (II)), or a stereoisomer and/or a pharmaceutically acceptable salt thereof, is administered by subcutaneous or intramuscular injection, or by dissolving or suspending the drug in an oil vehicle.

In certain embodiments, the actual dosage level of a compound described herein (e.g., a compound of formula (I), formula (Ia), formula (Ib), or formula (II)), or a stereoisomer and/or a pharmaceutically acceptable salt thereof, in the pharmaceutical compositions of the present disclosure may be varied so as to obtain an amount of the compound (e.g., a compound of formula (I), formula (Ia), formula (Ib), or formula (II)), or a stereoisomer and/or a pharmaceutically acceptable salt thereof, which is effective to achieve the desired therapeutic response for a particular patient, composition, and mode of administration, without being toxic to the patient.

In certain embodiments, the selected dosage level is dependent upon a variety of factors including the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In certain embodiments, a physician or veterinarian having ordinary skill in the art can readily determine and prescribe the effective amount of the pharmaceutical composition as required.

In certain embodiments, a suitable daily dose of a compound described herein (e.g., a compound of formula (I), formula (Ia), formula (Ib), or formula (II)), or a stereoisomer and/or a pharmaceutically acceptable salt thereof, will be an amount that corresponds to the lowest dose effective to produce a therapeutic effect. In certain embodiments, when a compound described herein (e.g., a compound of formula (I), formula (Ia), formula (Ib), or formula (II)), or a stereoisomer and/or a pharmaceutically acceptable salt thereof, is co-administered with another therapeutic agent, the effective amount may be less than when the compound (e.g., a compound of formula (I), formula (Ia), formula (Ib), or formula (II)), or a stereoisomer and/or a pharmaceutically acceptable salt thereof, is used in isolation.

In certain embodiments, the effective daily dose of a compound described herein (e.g., a compound of formula (I), formula (Ia), formula (Ib), or formula (II)), or a stereoisomer and/or a pharmaceutically acceptable salt thereof, may be administered as two, three, four, five, six or more sub-doses. In certain embodiments, the two, three, four, five, six or more sub-doses are administered separately at appropriate intervals throughout the day, optionally, in unit dosage forms. In some embodiments, dosing is one administration per day.

In some embodiments, a compound described herein (e.g., a compound of formula (I), formula (Ia), formula (Ib), or formula (II)), or a stereoisomer and/or a pharmaceutically acceptable salt thereof, is administered to a patient for 1 day, 5 days, 10 days, 20 days, 30 days, 1 week, 2 weeks, 3 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years. In some embodiments, a compound described herein (e.g., a compound of formula (I), formula (Ia), formula (Ib), or formula (II)), or a stereoisomer and/or a pharmaceutically acceptable salt thereof, is administered to a patient for the duration of the patient's life span.

### Combination Therapy

In various embodiments, a compound disclosed herein (e.g., a compound of formula (I), formula (Ia), formula (Ib), or formula (II)), or a stereoisomer and/or a pharmaceutically acceptable salt thereof, including pharmaceutical compositions of the present disclosure, can be part of a combination therapy. In certain embodiments, the combination therapy comprises a compound described herein (e.g., a compound of formula (I), formula (Ia), formula (Ib), or formula (II)), or a stereoisomer and/or a pharmaceutically acceptable salt thereof; and a second therapeutic agent. In certain embodiments, the combination therapy comprises a pharmaceutical composition comprising an effective amount of a compound described herein (e.g., a compound of formula (I), formula (Ia), formula (Ib), or formula (II)), or a stereoisomer and/or a pharmaceutically acceptable salt thereof; and a second therapeutic agent.

In some embodiments, the second therapeutic agent is selected from the group comprising a lovastatin, a thiazolidinedione or fibrate, a bile-acid-binding-resin, a niacin, an anti-obesity drug, a hormone, an antiviral agent (e.g., to treat an underlying hepatitis C infection causing liver disease in the patient), an anticancer agent (e.g., to treat hepatocellular carcinoma or other cancer causing liver disease or fatty liver), an antioxidant, a medication that decreases insulin resistance, or a medication that improves lipid metabolism (e.g., treatments for hyperlipidemia), a tyrophostine, a sulfonylurea-based drug, a biguanide, an α-glucosidase inhibitor, an apolipoprotein A-I agonist, apolipoprotein E, a cardiovascular drug, an HDL-raising drug, an HDL enhancer, or a regulator of the apolipoprotein A-I, apolipoprotein A-IV and/or apolipoprotein genes.

In various embodiments, the second therapeutic agent can be bempedoic acid, a statin and/or ezetimibe.

In certain embodiments, the second therapeutic agent is bempedoic acid. In certain embodiments, the second therapeutic agent is ezetimibe. In certain embodiments, the second therapeutic agent is a statin. Examples of statins include, but are not limited to, atorvastatin, fluvastatin, lovastatin, pitavastatin, pravastatin, rosuvastatin, and simvastatin.

In certain embodiments, administering a pharmaceutical composition of the present disclosure comprising a compound described herein (e.g., a compound of formula (I), formula (Ia), formula (Ib), or formula (II)), or a stereoisomer and/or a pharmaceutically acceptable salt thereof, and a second therapeutic agent is intended to provide a beneficial effect from the co-action of the compound (e.g., a compound of formula (I), formula (Ia), formula (Ib), or formula (II)), or a stereoisomer and/or a pharmaceutically acceptable salt thereof, and the second therapeutic agent. In some embodiments, the beneficial effect of the combination therapy may include pharmacokinetic or pharmacodynamic co-action resulting from the combination of the compound (e.g., a compound of formula (I), formula (Ia), formula (Ib), or formula (II)), or a stereoisomer and/or a pharmaceutically acceptable salt thereof, and the second therapeutic agent.

### Kits

In various embodiments, the disclosure provides kits for treating a condition, disease or disorder described herein. In some embodiments, a kit comprises: i) instructions for treating a condition, disease or disorder, for example, as described herein, and ii) a compound described herein (e.g., a compound of formula (I), formula (Ia), formula (Ib), or formula (II)), or a stereoisomer and/or a pharmaceutically acceptable salt thereof (e.g., a pharmaceutical composition comprising an effective amount of a compound described herein (e.g., a compound of formula (I), formula (Ia), formula (Ib), or formula (II)), or a stereoisomer and/or a pharmaceutically acceptable salt thereof). In some embodiments, the kit may comprise one or more unit dosage forms containing an amount of a compound described herein (e.g., a compound of formula (I), formula (Ia), formula (Ib), or formula (II)), or a stereoisomer and/or a pharmaceutically acceptable salt thereof, that is effective for treating the condition, disease, or disorder.

The description herein includes multiple aspects and embodiments of the present disclosure, including methods of making a compound described herein (e.g., a compound of formula (I), formula (Ia), formula (Ib), or formula (II)), or a stereoisomer and/or a pharmaceutically acceptable salt thereof; methods of using a compound described herein (e.g., a compound of formula (I), formula (Ia), formula (Ib), or formula (II)), or a stereoisomer and/or a pharmaceutically acceptable salt thereof; compositions comprising an effective amount of a compound described herein (e.g., a compound of formula (I), formula (Ia), formula (Ib), or formula (II)), or a stereoisomer and/or a pharmaceutically acceptable salt thereof; and kits. The disclosure specifically includes all combinations and permutations of the aspects and embodiments as described herein.

### EXAMPLES

The representative examples that follow are intended to help illustrate the disclosure, and are not intended to, nor should they be construed to, limit the scope of the disclosure.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### Analytical Methods

Method A: Ultra-high performance liquid chromatography-mass spectrometry (UHPLC-MS) was performed in reverse phase using a Phenomenex Kinetex-XB C18 column (2.1 mm×100 mm, 1.7 m; temperature: 40° C.), with an injection volume of 1 μL at a flow rate of 0.6 mL/min and a gradient of 5-100% B over 5.30 min, then 100% B for 0.50 min, where A=0.1% formic acid in water, and B=0.1% formic acid in acetonitrile. A second gradient of 100-5% B was then applied over 0.02 min and held for 1.18 min. UV spectra were recorded at 215 nm; spectrum range: 200-400 nm. ELS data was collected on a Waters ELS detector when reported. Mass spectra were obtained using a Waters SQD, SQD2 or a QDA detector; ionization mode: electrospray positive or negative. Data were integrated and reported using Waters MassLynx and OpenLynx software.

Method B: UHPLC-MS was performed in reverse phase system using a Waters UPLC™ BEH™ C18 column (2.1 mm×50 mm, 1.7 m; temperature: 40° C.), with an injection volume of 1 μL at a flow rate of 0.9 mL/min and a gradient of 5-100% B over 1.10 min, then 100% B for 0.25 min, where A=0.1% formic acid in water, and B=0.1% formic acid in acetonitrile. A second gradient of 100-5% B was then applied over 0.05 min and held for 0.10 min. UV spectra were recorded at 215 nm; spectrum range: 200-400 nm. Mass spectra were obtained using a Waters SQD, SQD2 or a QDA detector; ionization mode: electrospray positive or negative. Data were integrated and reported using Waters MassLynx and OpenLynx software.

Method C: UHPLC-MS was performed in reverse phase using a Waters UPLC™ CORTECS™ C8 column (2.1 mm×100 mm, 1.6 m; temperature: 40° C.), with an injection volume of 1 μL at a flow rate of 0.6 mL/min and a gradient of 5-100% B over 5.30 min, then 100% B for 0.50 min, where A=0.1% formic acid in water, and B=0.1% formic acid in acetonitrile. A second gradient of 100-5% B was then applied over 0.02 min and held for 1.18 min. UV spectra were recorded at 215 nm; spectrum range: 200-400 nm. ELS data was collected using a Waters ELS detector when reported. Mass spectra were obtained using a Waters SQD, QD2 or a QDA; ionization mode: electrospray positive or negative. Data were integrated and reported using Waters MassLynx and OpenLynx software.

### Preparative HPLC Methods

Method P1: Liquid chromatography (LC) was performed in reverse phase using a Waters Sunfire™ C18 column (30 mm×100 mm, 5 m; temperature: room temperature), with an injection volume of 1500 μL at a flow rate of 40 mL/min at 30% B for 1.90 min then a gradient of 30-95% B over 9.60 min and held for 1.97 min, where A=0.1% formic acid in water and B=0.1% formic acid in acetonitrile. A second gradient of 95-30% B was then applied over 0.33 min and held for 1.65 min. UV spectra were recorded at 215 nm.

Method P2: LC were performed in reverse phase using a Waters Sunfire™ C18 column (30 mm×100 mm, 5 m; temperature: room temperature), with an injection volume of 1500 μL at a flow rate of 40 mL/min at 10% B for 1.90 min then a gradient of 10-95% B over 14.10 min and held for 2.0 min, where A=0.1% formic acid in water and B=0.1% formic acid in acetonitrile. A second gradient of 95-10% B was then applied over 0.20 min and held for a further 1.25 min. UV spectra were recorded at 215 nm.

Method P3: LC were performed in reverse phase using a Waters XBridge™ C18 column (30 mm×100 mm, 5 m; temperature: room temperature), with an injection volume of 1500 μL at a flow rate of 40 mL/min at 10% B for 2.00 min then a gradient of 10-95% B over 14.00 min and held for 2.00 min, where A=0.2% ammonium hydroxide in water and B=0.2% ammonium hydroxide in acetonitrile. A second gradient of 95-10% B was then applied over 0.20 min and held for 1.25 min. UV spectra were recorded at 215 nm.

### Abbreviations

Aq. aqueous
Boc tert-butyl carbamoyl
Boc₂O di-tert-butyl dicarbonate
CDCl₃ deuterated chloroform
CHCl₃ chloroform
CO₂ carbon dioxide
CV column volumes
DAST diethylaminosulfur trifluoride
DCC N,N-dicyclohexylcarbodiimide
DCE dichloroethane
DCM dichloromethane
DIAD diisopropyl azodicarboxylate
DIBAL diisobutyl aluminum hydride
DIPEA N,N-diisopropylethylamine
DMAP 4-(dimethylamino)pyridine
DMF N,N-dimethylformamide
DMSO dimethyl sulfoxide
dppf 1,1′-bis(diphenylphosphino)ferrocene
ESI electrospray ionization
Et₂O diethyl ether
EtOH ethanol
EtOAc ethyl acetate
FCC flash column chromatography
HCl hydrogen chloride
H₂O water
HPLC high-performance liquid chromatography
IPA isopropanol
K₂CO₃ potassium carbonate
LCMS liquid chromatography-mass spectrometry
LiAlH₄ lithium aluminum hydride
LiHMDS lithium bis(trimethylsilyl)amide
M molar
mCPBA metachloroperbenzoic acid
MeCN acetonitrile
MeOH methanol
MgSO₄ magnesium sulfate
MnO₂ manganese (IV) oxide
NaHCO₃ sodium bicarbonate
Na₂SO₄ sodium sulfate
Na₂S₂O₃ sodium thiosulfate
NH₃ ammonia
NH₄Cl ammonium hydrochloride
NMR nuclear magnetic resonance
Pd₂(dba)₃ tris(dibenzylideneacetone)dipalladium(O)
Pd(dppf)Cl₂ 1,1′-bis(diphenylphosphino)ferrocene]dichloropalladium(II)
RT retention time
r.t. room temperature
sat. saturated
SCX strong cation exchange cartridge (benzenesulfonic acid-functionalized silica)
SFC supercritical fluid chromatography
SiO₂ silicon dioxide (silica gel for FCC)
T3P propylphosphonic anhydride
TBAF tetrabutylammonium fluoride
TBME tert-butyl methyl ether
TBS tert-butyl dimethylsilyl
THE tetrahydrofuran
UV ultraviolet
wt % weight percent

In some embodiments, compounds of the disclosure may be synthesized using the procedure outlined in General Scheme 1:

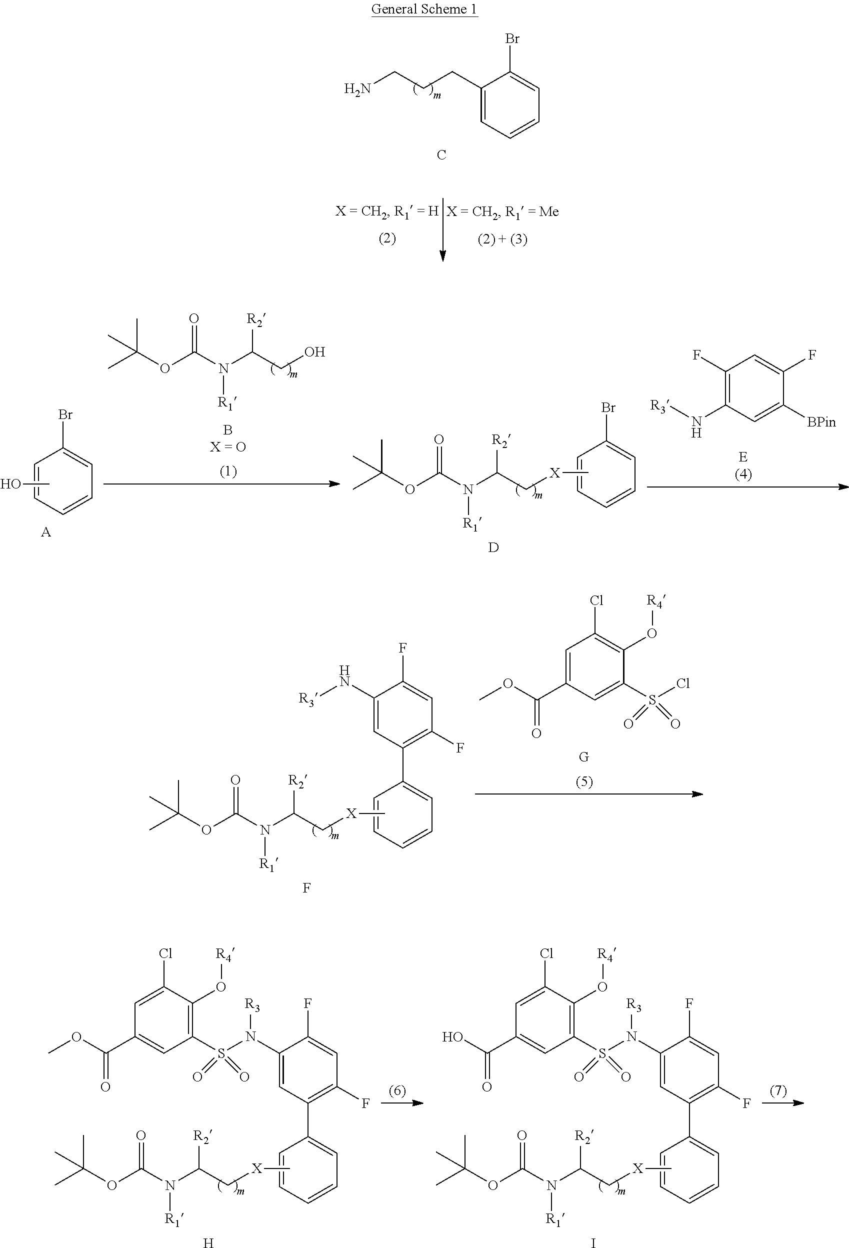

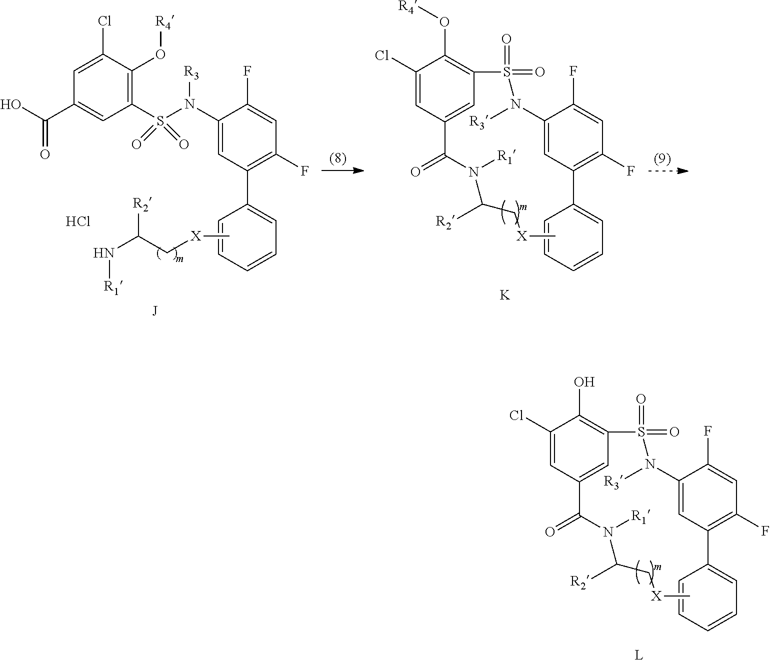

In General Scheme 1, starting material A is reacted with R₁′- and R₂′-bearing alcohol starting material B, for example, using the Mitsunobu reaction conditions (e.g., PPh₃, DIAD, THF, 30-40° C., 1-3 hours) to yield intermediate D, wherein X is oxygen. In some embodiments, R₁′ and R₂′ are selected from hydrogen and lower alkyl, e.g., methyl. In some embodiments, m is 1-3.

Alternatively, amine starting material C undergoes amine protection using Boc protecting conditions (e.g., Boc₂O, NaHCO₃ (aq.), THF, room temperature, 1 hour) to yield intermediate D, wherein R₁′ and R₂′ are both hydrogen, X is CH₂, and m is 0-1. Alternatively, starting material C undergoes amine protection using Boc protecting conditions (e.g., Boc₂O, NaHCO₃ (aq.), THF, room temperature, 1 hour) followed by methylation of the carbamate nitrogen using alkylation conditions (e.g., NaH, Mel, DMF, 1 hour) to yield intermediate D, wherein R₁′ is methyl, R₂′ is hydrogen, X is CH₂, and m is 0-1.

Intermediate D is reacted with R₃′-bearing pinacol boronic ester intermediate E using palladium-catalyzed cross-coupling conditions (e.g., Pd(dppf)Cl₂, K₂CO₃, dioxane/water, 100° C., about 18 hours) to yield intermediate F. In some embodiments, R₃′ is selected from H or lower alkyl, e.g., methyl. Intermediate F is reacted with R₄′-bearing sulfonyl chloride intermediate G under basic conditions (e.g., pyridine, 50° C., 1-2.5 hours) to yield intermediate H. In some embodiments, R₄′ is selected from lower alkyl (e.g., methyl) or haloalkyl (e.g., —CHF₂). Intermediate H is subjected to ester hydrolysis under basic conditions (e.g., NaOH (aq), THE or THF/MeOH, room temperature, 2-18 hours) to yield intermediate I. Intermediate I is subjected to Boc deprotection under acidic conditions (4 M HCl in dioxane, r.t., 1-2.5 hours) to yield intermediate J. Intermediate J then undergoes cyclization using amide coupling conditions (e.g., T3P, DIPEA, DMF, r.t., approximately 0.5-1 hours) to yield product K. Optionally, when R₄′ is methyl, intermediate K undergoes ether cleavage using, e.g., 1 M BBr₃ in DCM, DMF, 80° C., typically 18-24 hours or 1 M BBr₃ in DCM, room temperature, typically for 18 hours, to yield final product L.

In some embodiments, compounds of the disclosure may be synthesized using the procedure outlined in General Scheme 2:

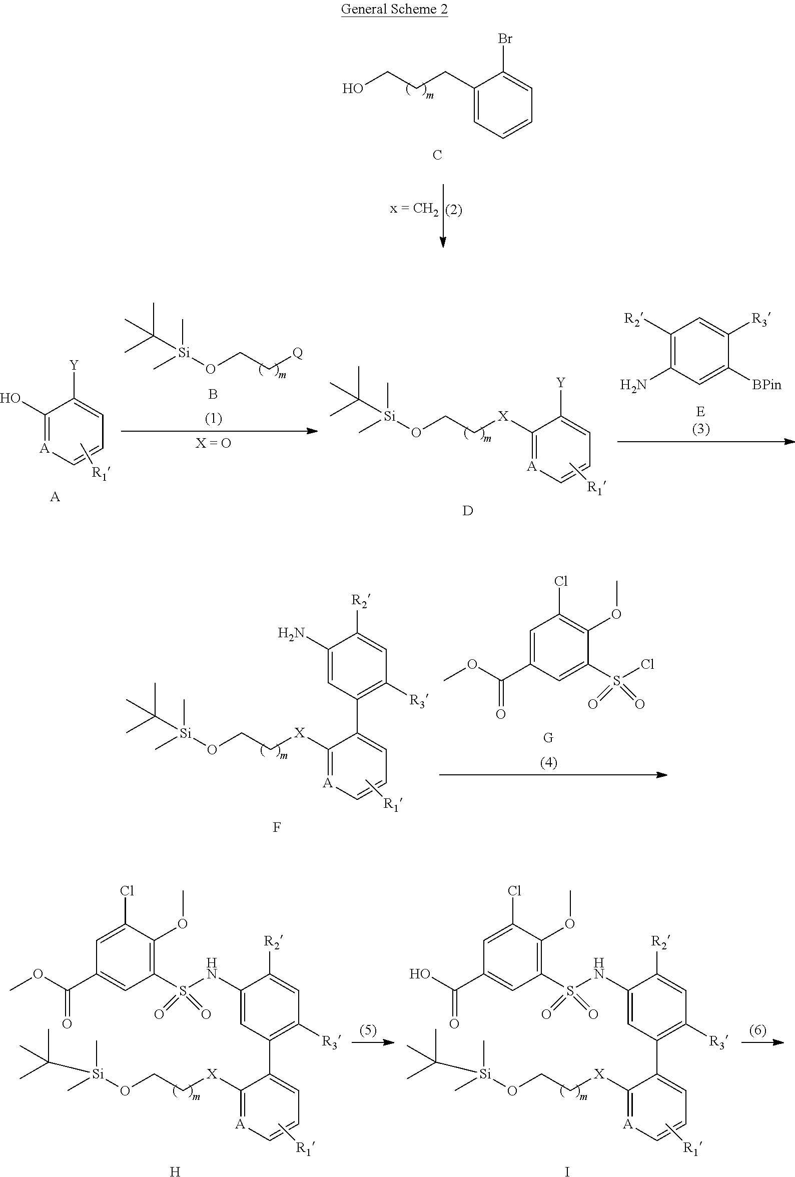

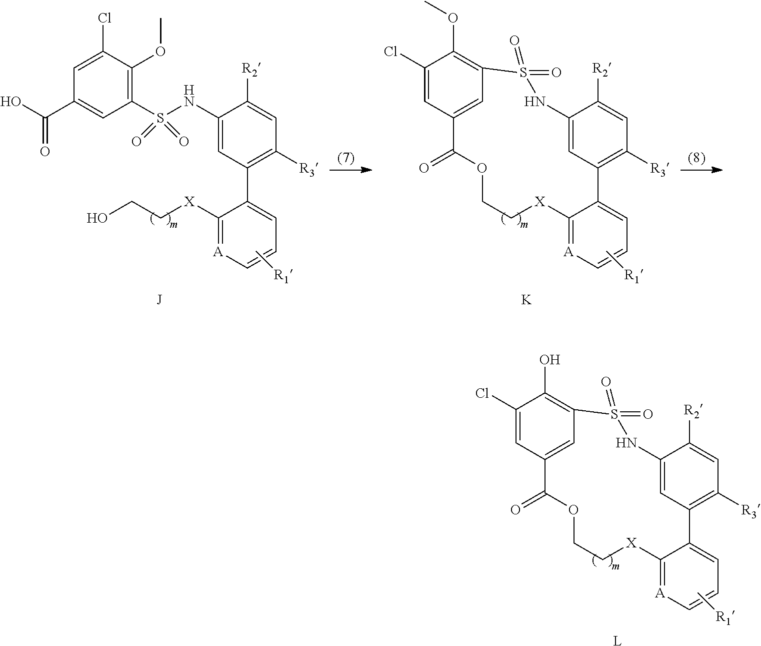

In General Scheme 2, starting material A is reacted with starting material B to yield intermediate D, wherein X is oxygen. In some embodiments, A is selected from CH or N; Y is selected from Br or I; and R₁′ is selected from halogen (e.g., F, Cl, Br), alkyl (e.g., methyl), cycloalkyl (e.g., cyclopropyl), or —CN. In some embodiments, Q is selected from hydroxyl or Br; and m is 1-2. When Q is hydroxyl, starting material A is reacted with starting material B using the Mitsunobu reaction conditions (e.g., PPh₃, DIAD, THF, 40° C., 1-3 hours) to yield intermediate D. When Q is Br, starting material A is reacted with starting material B using alkylation conditions (e.g., base (K₂CO₃), acetonitrile, ˜70° C., ˜18 hours) to yield intermediate D. Alternatively, alcohol starting material C, wherein m is 0-1, undergoes alcohol protection, using silyl ether protecting conditions (e.g., TBS-C₁, imidazole, DMF, RT, 4 hours) to yield intermediate D, wherein X is CH₂.

Intermediate D is reacted with R₂′- and R₃′-bearing pinacol boronic ester intermediate E using palladium-catalyzed cross-coupling conditions (e.g., Pd(dppf)Cl₂, base (K₂CO₃), dioxane, 100° C., 18 hours) to yield intermediate F. In some embodiments, R₂′ is selected from F or methoxy and R₃′ is selected from H, F, methyl, or methoxy. Intermediate F, is reacted with sulfonyl chloride intermediate G under basic conditions (e.g., pyridine, 50° C., 1-2.5 hours) to yield intermediate H. Intermediate H undergoes ester hydrolysis under basic conditions (e.g., NaOH (aq), THF, RT, 4 hours) to yield intermediate I. Intermediate I undergoes silyl ether deprotection using a fluoride source (e.g., TBAF, THF, RT, 18-72 hours) to yield intermediate J. Intermediate J then undergoes cyclization using ester coupling conditions, for example, using the Yamaguchi esterification conditions (e.g., 2,4,6-trichlorobenzoyl chloride, DIPEA, DMAP, room temperature 1 hour then 65° C. 3-18 hours. Purified by acidic reverse phase preparative HPLC.) to yield intermediate K. Finally, intermediate K undergoes ether cleavage using, e.g., 1 M BBr₃ in DCM, DMF, 80° C., 20 hours, to yield final product L.

In some embodiments, compounds of the disclosure may be synthesized using the procedure outlined in General Scheme 3:

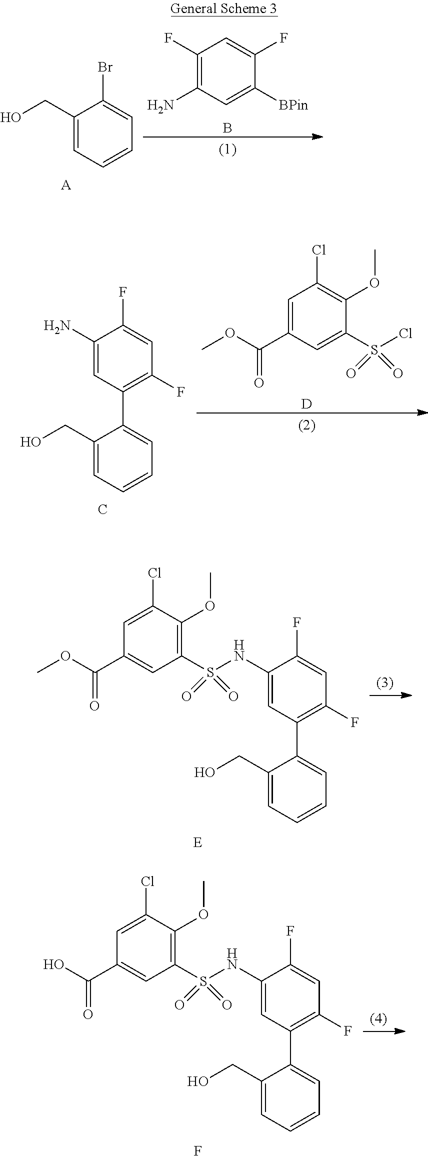

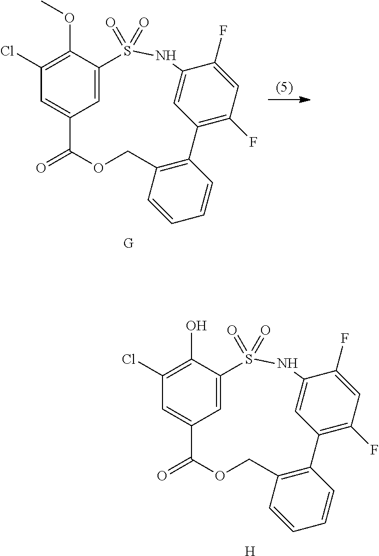

In General Scheme 3, starting material A is reacted with pinacol boronic ester intermediate B using palladium-catalyzed cross-coupling conditions (e.g., Pd(dppf)Cl₂, base (K₂CO₃), dioxane, 100° C., 18 hours) to yield intermediate C. Intermediate C is reacted with sulfonyl chloride intermediate D under basic conditions (e.g., pyridine, 50° C., 1 hour) to yield intermediate E. Intermediate E undergoes ester hydrolysis under basic conditions (e.g., NaOH (aq.), THF, r.t., 3 hours) to yield intermediate F. Intermediate F undergoes cyclization using ester coupling conditions (e.g., DCC, DMAP, DCM, r.t., 24 hours) to yield intermediate G. Finally, intermediate G undergoes ether cleavage (using e.g., iodocyclohexane, anhydrous DMF, 120° C., 1-3 hours; or lithium iodide, anhydrous pyridine, 80° C., 6-18 hours) to yield final product H.

Intermediate A may have additional substitution or may incorporate heteroatoms into aryl ring.

Intermediate B may have different or additional substitution and may incorporate heteroatoms into aryl ring.

### Example 1—Synthesis of 15-chloro-21,23-difluoro-16-methoxy-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 1)

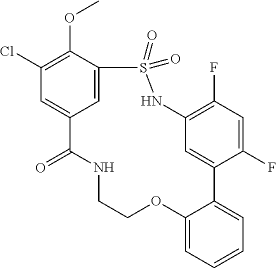 [SMILES: COc1c(Cl)cc2cc1S(=O)(=O)Nc1cc(c(F)cc1F)-c1ccccc1OCCNC2=O]

To a solution of DIPEA (0.13 mL, 0.740 mmol) and T3P in EtOAc (50 wt %, 0.44 mL, 0.740 mmol) in EtOAc (50 mL) was added a solution of Intermediate 6 (253 mg, 0.493 mmol) in DMF-anhydrous (50 mL) over 1.5 hours. The colorless solution was stirred at r.t. for 23 hours. To the reaction mixture was added T3P in EtOAc (50 wt %, 0.44 mL, 0.740 mmol) and DIPEA (0.13 mL, 0.740 mmol) and the reaction mixture was stirred at r.t. for 2.5 hours. The reaction mixture was diluted with H₂O (200 mL) and extracted with EtOAc (4×200 mL). The combined organic extracts were washed with brine (4×200 mL), dried over MgSO₄, filtered and concentrated in vacuo which gave a yellow residue (530 mg). The residue was triturated and sonicated with DCM/MeOH and the resultant white suspension was collected by suction filtration. The filter cake was washed with DCM and then dried in vacuo which gave the title compound as a white solid (180 mg, 72% yield, 98% purity).

¹H NMR (400 MHz, DMSO) δ 10.39 (s, 1H), 8.16-8.07 (m, 1H), 8.01 (d, J=1.7 Hz, 1H), 7.52 (d, J=2.1 Hz, 1H), 7.48-7.41 (m, 1H), 7.37-7.31 (m, 1H), 7.28-7.24 (m, 1H), 7.17-7.13 (m, 1H), 7.11-7.03 (m, 2H), 4.21 (t, J=5.0 Hz, 2H), 3.99 (s, 3H), 3.53-3.44 (m, 2H).

¹⁹F NMR (376 MHz, DMSO-d6) δ −109.21, −120.11.

LCMS: m/z=494.9/496.9 [M+H]+, (ESI+), RT=3.44, Method A

### Example 2—Synthesis of 15-chloro-21,23-difluoro-16-hydroxy-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 2)

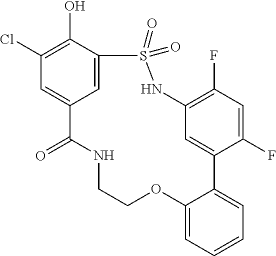 [SMILES: O=C1NCCOc2ccccc2-c2cc(c(F)cc2F)NS(=O)(=O)c2cc1cc(Cl)c2O]

To a suspension of Compound 1 (140 mg, 0.283 mmol) in DCM-anhydrous (2.0 mL) and toluene-anhydrous (2.0 mL) at 0° C. was added dropwise 1 M BBr₃ in DCM (0.85 mL, 0.849 mmol). The resulting white suspension was heated at 60° C. for 22 hours. The reaction mixture was cooled to r.t., transferred to a larger vial and diluted with anhydrous DCM (6 mL) and anhydrous toluene (6 mL). To the reaction mixture was added dropwise 1 M BBr₃ in DCM (0.85 mL, 0.849 mmol) and the reaction mixture was heated at 70° C. for 0.5 hours, then at 80° C. for 6 hours. The reaction mixture was allowed to cool to r.t, and was then slowly added to a saturated aqueous solution of NaHCO₃ at 0° C. The layers were separated and the organics were washed with brine, dried over MgSO₄, filtered and concentrated in vacuo. Purification by FCC (10 g SiO₂ column, 0-20% MeOH in DCM) gave the title compound as an off-white solid (76 mg, 54% yield, 96% purity).

¹H NMR (400 MHz, DMSO) δ 10.39 (broad s, 1H), 8.00-7.92 (m, 1H), 7.90 (d, J=1.9 Hz, 1H), 7.46-7.40 (m, 2H), 7.30-7.24 (m, 1H), 7.24-7.20 (m, 1H), 7.18-7.11 (m, 2H), 7.07-7.01 (m, 1H), 4.20 (t, J=4.7 Hz, 2H), 3.49-3.41 (m, 2H). 1H not observed.

¹⁹F NMR (376 MHz, DMSO-d6) δ −109.64, −120.02.

LCMS: m/z=480.9/482.9 [M+H]+, (ESI+), RT=3.22, Method A

### Example 3—Synthesis of 11-chloro-3,5-difluoro-10-hydroxy-18-oxa-8lambda6-thia-7,15-diazatetracyclo[17.3.1.1^{2,6}.1^{9,13}]pentacosa-1(22),2(25),3,5,9(24),10,12,19(23),20-nonaene-8,8,14-trione (Compound 3)

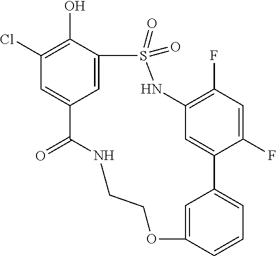 [SMILES: O=C1NCCOc2cccc(c2)-c2cc(c(F)cc2F)NS(=O)(=O)c2cc1cc(Cl)c2O]

To a solution of 11-chloro-3,5-difluoro-10-methoxy-8,8-dioxo-18-oxa-8λ6-thia-7,15-diazatetracyclo[17.3.1.12,6.19,13]pentacosa-1(22),2(25),3,5,9(24),10,12,19(23),20-nonaen-14-one (synthesized according to General Scheme 1, 94%, 100 mg, 0.190 mmol) in anhydrous DCE (2.5 mL) and anhydrous DMF (1.5 mL) at r.t. was added dropwise 1 M BBr₃ in DCM (0.19 mL, 0.190 mmol). The resulting solution was heated at 80° C. for 18 hours and was then allowed to cool to r.t. To the reaction mixture was added 1 M BBr₃ in DCM (0.76 mL, 0.760 mmol) and the reaction mixture was heated at 80° C. for 5 hours and was then allowed to cool to r.t. The reaction mixture was added slowly to a saturated aqueous solution of NaHCO₃. The mixture was diluted with water and 1:3 IPA/CHCl₃. The layers were separated and the aqueous was extracted with a 1:3 IPA/CHCl₃. The organics were combined and passed through a hydrophobic frit. Purification by FCC (10 g SiO₂, 0-30% MeOH in DCM) followed by trituration with MeCN afforded the title compound as an off-white solid (47 mg, 98% purity).

¹H NMR (500 MHz, DMSO) δ 8.72 (broad s, 1H), 8.36 (apparent s, 1H), 7.80 (apparent s, 1H), 7.34-7.27 (m, 1H), 7.25-7.12 (m, 2H), 7.11-7.05 (m, 1H), 7.03-6.97 (m, 1H), 6.73 (apparent s, 1H), 4.19-4.01 (m, 2H), 3.62-3.46 (m, 2H). 2H not observed.

LCMS: m/z=481.2/483.2 [M+H]+, (ESI+), RT=2.98, Method A

### Example 4—Synthesis of 15-chloro-21,23-difluoro-16-hydroxy-11-methyl-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 4)

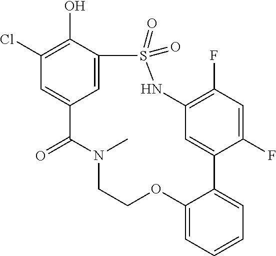 [SMILES: CN1CCOc2ccccc2-c2cc(c(F)cc2F)NS(=O)(=O)c2cc(cc(Cl)c2O)C1=O]

To a suspension of 15-chloro-21,23-difluoro-16-methoxy-11-methyl-18,18-dioxo-8-oxa-18λ6-thia-11,19-diazatetracyclo[18.3.1.113,17.02,7]pentacosa-1(24),2,4,6,13,15,17(25),20,22-nonaen-12-one (synthesized according to General Scheme 1, 92%, 250 mg, 0.452 mmol) in DCE-anhydrous (6.0 mL) and DMF-anhydrous (3.0 mL) at 0° C. was added dropwise 1 M BBr₃ in DCM (2.0 mL, 2.0 mmol). The resulting pale-yellow suspension was heated at 80° C. for 5 hours. The reaction mixture was cooled to 0° C., and additional 1 M BBr₃ in DCM (2.0 mL, 2.0 mmol) was added. The reaction mixture was heated at 80° C. for 17 hours and was then allowed to cool to r.t. The reaction mixture was slowly added to a saturated aqueous solution of NaHCO₃ (30 mL) at 0° C. The aqueous was extracted with DCM (2×20 mL) and the combined organics were washed with brine (50 mL), dried over Na₂SO₄, filtered and concentrated in vacuo. Purification by FCC (10 g SiO₂, 0-40% MeOH in DCM) gave product that was purified further by FCC (10 g SiO₂, 100% DCM then 0-30% MeOH in DCM) which gave a yellow-brown solid (110 mg). The solid was purified by preparative HPLC (Method P1) to afford the title compound as a white solid (34 mg, 15% Yield, 99% purity).

¹H NMR (400 MHz, DMSO) δ 11.21 (broad s, 1H), 10.29 (broad s, 1H), 7.67 (d, J=2.0 Hz, 1H), 7.48-7.35 (m, 1H), 7.35-7.16 (m, 5H), 7.12-7.05 (m, 1H), 4.12-3.93 (m, 2H), 2.90 (broad s, 3H). 2H not observed

LCMS: m/z=495.2/497.2 [M+H]+, (ESI+), RT=3.11, Method A

### Example 5—Synthesis of 11-chloro-3,5-difluoro-10-hydroxy-19-oxa-8lambda6-thia-7,15-diazatetracyclo[18.3.1.1^{2,6}.1^{9,13}]hexacosa-1(23),2(26),3,5,9(25),10,12,20(24),21-nonaene-8,8,14-trione (Compound 5)

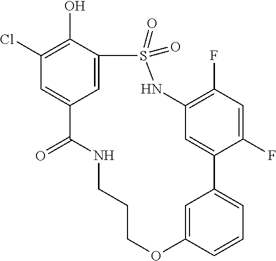 [SMILES: O=C1NCCCOc2cccc(c2)-c2cc(c(F)cc2F)NS(=O)(=O)c2cc1cc(Cl)c2O]

To a solution of 11-chloro-3,5-difluoro-10-methoxy-8,8-dioxo-19-oxa-8λ6-thia-7,15-diazatetracyclo[18.3.1.12,6.19,13]hexacosa-1(23),2(26),3,5,9(25),10,12,20(24),21-nonaen-14-one (synthesized according to General Scheme 1, 90%, 200 mg, 0.354 mmol) in anhydrous DMF (4.0 mL) at r.t. was added 1 M BBr₃ in DCM (0.71 mL, 0.710 mmol). The reaction mixture was heated at 80° C. for 6 hours and was then allowed to cool to r.t. To the reaction mixture was added 1 M BBr₃ in DCM (1.42 mL, 1.42 mmol) was added and the reaction mixture was heated at 80° C. for 16 hours. The reaction mixture was allowed to cool to r.t, and was then slowly added to a saturated aqueous solution of NaHCO₃. The mixture was extracted with DCM (3×) and the combined organics were passed through a hydrophobic frit. The residue was purified by preparative HPLC (Method P2) to afford the title compound as an off-white solid (93 mg, 53% yield, 100% purity).

¹H NMR (500 MHz, DMSO) δ 8.78 (t, J=6.2 Hz, 1H), 8.50 (d, J=2.2 Hz, 1H), 8.22 (d, J=2.2 Hz, 1H), 7.41-7.36 (m, 1H), 7.34-7.30 (m, 1H), 7.10-7.02 (m, 2H), 6.95 (dd, J=8.1, 2.5 Hz, 1H), 6.19-6.18 (m, 1H), 4.13 (t, J=5.8 Hz, 2H), 3.51-3.46 (m, 2H), 1.99-1.92 (m, 2H). 2H not observed LCMS: m/z=495.0/496.9 [M+H]+, (ESI+), RT=3.24, Method A

### Example 6—Synthesis of 16-chloro-22,24-difluoro-17-hydroxy-8-oxa-19lambda6-thia-12,20-diazatetracyclo[19.3.1.1^{14,18}.0^{2,7}]hexacosa-1(25),2,4,6,14,16,18(26),21,23-nonaene-13,19,19-trione (Compound 6)

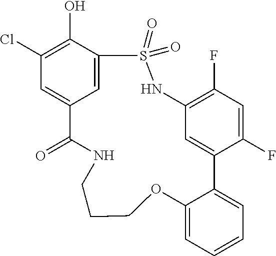 [SMILES: O=C1NCCCOc2ccccc2-c2cc(c(F)cc2F)NS(=O)(=O)c2cc1cc(Cl)c2O]

To a solution of 16-chloro-22,24-difluoro-17-methoxy-19,19-dioxo-8-oxa-19λ6-thia-12,20-diazatetracyclo[19.3.1.114,18.02,7]hexacosa-1(25),2,4,6,14,16,18(26),21,23-nonaen-13-one (synthesized according to General Scheme 1, 93%, 140 mg, 0.256 mmol) in anhydrous DMF (3.8 mL) at r.t. was added 1 M BBr₃ in DCM (1.00 mL, 1.00 mmol). The resulting solution was heated at 80° C. for 18 hours and was then allowed to cool to r.t. The reaction mixture was slowly added to a saturated aqueous solution of NaHCO₃ and the mixture was extracted with DCM (3×). The combined organics were passed through a phase separator and concentrated in vacuo. The residue was purified by preparative HPLC (Method P2) to afford the title compound as an off-white solid (14 mg, 11% yield, 99% purity).

¹H NMR (500 MHz, DMSO) δ 7.98-7.85 (m, 1H), 7.43-7.38 (m, 1H), 7.36-7.32 (m, 1H), 7.19-7.05 (m, 4H), 7.03-6.98 (m, 1H), 4.10 (t, J=4.8 Hz, 2H), 1.87-1.80 (m, 2H). 5H not observed

LCMS: m/z=495.2/497.2 [M+H]+, (ESI+), RT=3.36, Method A

### Example 7—Synthesis of 15-chloro-21,23-difluoro-16-hydroxy-8,11-dioxa-18lambda6-thia-19-azatetracyclo[18.3.1.1^{13,17}0^{2,7}]pentacosa-1(24),2,4,6,13,15,17(25),20,22-nonaene-12,18,18-trione (Compound 7)

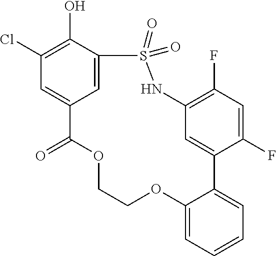 [SMILES: O=C1OCCOc2ccccc2-c2cc(c(F)cc2F)NS(=O)(=O)c2cc1cc(Cl)c2O]

To a suspension of Intermediate 12 (98%, 60 mg, 0.119 mmol) in DMF-anhydrous (3.0 mL) at 0° C. was added dropwise 1 M BBr₃ in DCM (2.0 mL, 2.0 mmol). The resulting pale-yellow solution was heated at 80° C. for 20 hours and was then allowed to cool to r.t.

The reaction mixture was added to a saturated aqueous solution of NaHCO₃ (30 mL) and the aqueous was extracted with DCM (2×20 mL). The organics were combined, washed with brine (50 mL), dried over Na₂SO₄, filtered and concentrated in vacuo. Purification was performed using preparative HPLC (Method P1) to afford the title compound as a white solid (42 mg, 74% yield, 100% purity).

¹H NMR (500 MHz, DMSO) δ 11.53-9.83 (m, 1H), 8.04 (d, J=2.1 Hz, 1H), 7.47 (d, J=2.2 Hz, 1H), 7.47-7.43 (m, 1H), 7.33-7.25 (m, 2H), 7.25-7.16 (m, 2H), 7.12-7.04 (m, 1H), 4.36-4.32 (m, 2H), 4.31-4.27 (m, 2H). 1H not observed.

LCMS: m/z=480.2/482.2 [M−H]−, (ESI−), RT=3.79, Method A

### Example 8—Synthesis of 15-chloro-21,23-difluoro-16-hydroxy-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 8)

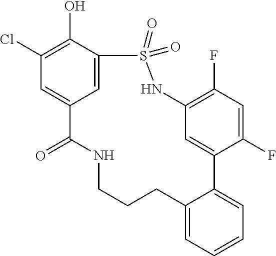 [SMILES: O=C1NCCCc2ccccc2-c2cc(c(F)cc2F)NS(=O)(=O)c2cc1cc(Cl)c2O]

To a solution of 15-chloro-21,23-difluoro-16-methoxy-18,18-dioxo-18λ6-thia-11,19-diazatetracyclo[18.3.1.113,17.02,7]pentacosa-1(24),2,4,6,13,15,17(25),20,22-nonaen-12-one (synthesized according to General Scheme 1, 80%, 170 mg, 0.276 mmol) in anhydrous DMF (4 mL) at r.t. was added 1 M BBr₃ in DCM (1.1 mL, 1.10 mmol). The reaction mixture was heated at 80° C. for 4 hours and was then allowed to cool to r.t. To the reaction mixture was added 1 M BBr₃ in DCM (1.1 mL, 1.10 mmol) and the reaction mixture was heated at 80° C. for 16 hours and was then allowed to cool to r.t. The reaction mixture was added to H₂O (40 mL). The aqueous was extracted with DCM (3×30 mL) and the combined organics were passed through a hydrophobic frit and concentrated in vacuo.

Purification was performed by FCC (10 g SiO₂, 0-30% MeOH in DCM). The product-containing fractions were combined and concentrated in vacuo and the resultant solid was purified by preparative HPLC (Method P1) to afford the title compound as a white solid (55 mg, 41% yield, 98% purity).

¹H NMR (400 MHz, DMSO) δ 11.07-9.88 (m, 2H), 8.30-8.18 (m, 1H), 7.88 (d, J=2.1 Hz, 1H), 7.60 (d, J=2.3 Hz, 1H), 7.45-7.35 (m, 2H), 7.33-7.20 (m, 3H), 7.20-7.14 (m, 1H), 3.50-3.38 (m, 1H), 3.08-2.94 (m, 1H), 2.47-2.41 (m, 1H), 2.26-2.06 (m, 2H), 1.73-1.54 (m, 1H).

LCMS: m/z=479.0/480.8 [M+H]+, (ESI+), RT=3.34, Method A

### Example 9—Synthesis of 15-chloro-21,23-difluoro-16-hydroxy-19-methyl-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 9)

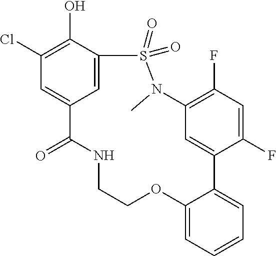 [SMILES: CN1c2cc(c(F)cc2F)-c2ccccc2OCCNC(=O)c2cc(Cl)c(O)c(c2)S1(=O)=O]

To a solution of 15-chloro-21,23-difluoro-16-methoxy-19-methyl-18,18-dioxo-8-oxa-18λ6-thia-11,19-diazatetracyclo[18.3.1.113,17.02,7]pentacosa-1(24),2,4,6,13,15,17(25),20,22-nonaen-12-one (synthesized according to General Scheme 1, 80%, 200 mg, 0.314 mmol) in anhydrous DMF (5.0 mL) at 0° C. was added dropwise 1 M BBr₃ in DCM (3.0 mL, 3.00 mmol). The resulting solution was heated at 80° C. for 20 hours and was then allowed to cool to r.t. The reaction mixture was added to a saturated aqueous solution of NaHCO₃ (30 mL) at 0° C. The organics were extracted with DCM (2×20 mL). The organics were combined, washed with brine (50 mL), dried over Na₂SO₄, filtered and concentrated in vacuo. Purification was performed by preparative HPLC (Method P1) to afford the title compound as a white solid (66 mg, 40% yield, 95% purity).

¹H NMR (400 MHz, DMSO) δ 10.59 (broad s, 1H), 8.24 (t, J=4.7 Hz, 1H), 7.95 (d, J=2.2 Hz, 1H), 7.46 (d, J=2.2 Hz, 1H), 7.41-7.34 (m, 2H), 7.14-7.03 (m, 3H), 7.00-6.93 (m, 1H), 4.21 (t, J=5.1 Hz, 2H), 3.47-3.43 (m, 2H), 3.19 (s, 3H).

LCMS: m/z=495.2/497.2 [M+H]+, (ESI+), RT=3.60, Method A

### Example 10—Synthesis of 15-chloro-21,23-difluoro-16-hydroxy-11-methyl-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 10)

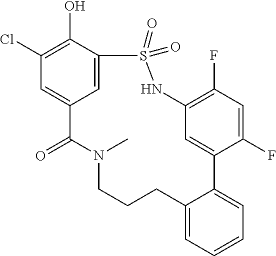 [SMILES: CN1CCCc2ccccc2-c2cc(c(F)cc2F)NS(=O)(=O)c2cc(cc(Cl)c2O)C1=O]

To a solution of 15-chloro-21,23-difluoro-16-methoxy-11-methyl-18,18-dioxo-18λ6-thia-11,19-diazatetracyclo[18.3.1.113,17.02,7]pentacosa-1(24),2,4,6,13,15,17(25),20,22-nonaen-12-one (synthesized according to General Scheme 1, 91%, 153 mg, 0.275 mmol) in anhydrous DMF (4.0 mL) at r.t. was added 1 M BBr₃ in DCM (1.1 mL, 1.10 mmol). The reaction mixture was heated at 80° C. for 18 hours and was then allowed to cool to r.t. The reaction mixture was added to H₂O (40 mL) and the organics were extracted with DCM (3×30 mL), combined and passed through a hydrophobic frit. Purification was performed by preparative HPLC (Method P1) to afford the title compound as a white solid (78 mg, 56% yield, 97% purity).

¹H NMR (400 MHz, DMSO) δ 11.46 (broad s, 1H), 10.28 (broad s, 1H), 7.61 (d, J=2.1 Hz, 1H), 7.41-7.23 (m, 6H), 7.18-7.15 (m, 1H), 2.89 (s, 3H), 2.85-2.61 (m, 2H), 2.42-2.24 (m, 1H), 1.97-1.79 (m, 1H), 1.72-1.54 (m, 1H), 1.38-1.16 (m, 1H).

LCMS: m/z=493.2/495.3 [M+H]+, (ESI+), RT=3.17, Method A

### Example 11—Synthesis of 24-fluoro-16-hydroxy-8-oxa-11,19-diazapentacyclo[17.5.2.1^{13,17}.0^{2,7}.0^{22,26}]heptacosa-1(24),2,4,6,13,15,17(27),22,25-nonaene-12,18-dione (Compound 11)

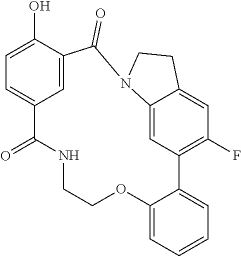 [SMILES: O=C1NCCOc2ccccc2-c2cc3c(cc2F)CCN3C(=O)c2cc1ccc2O]

### Step 1

A mixture of Intermediate 2 (92%, 500 mg, 1.45 mmol), 4,4,4′,4′,5,5,5′,5′-octamethyl-2,2′-bi-1,3,2-dioxaborolane (406 mg, 1.60 mmol) and potassium acetate (428 mg, 4.36 mmol) in anhydrous 1,4-dioxane (4.6 mL) was sparged with nitrogen for 20 minutes, before Pd(dppf)Cl₂ (107 mg, 0.145 mmol) was added and the solution sparged for a further 5 minutes. The reaction mixture was heated at 100° C. for a total of approximately 20 minutes. The mixture was then filtered through Celite, washing with EtOAc. To the filtrate was added water, and the layers separated. The aqueous phase was extracted with EtOAc. The combined organics were washed with water then brine, dried (Na₂SO₄), filtered and concentrated. The residue was purified by FCC (50 g SiO₂ column, 0-100% EtOAc in heptane) to afford tert-butyl N-[2-[2-(4,4,5,5-tetramethyl-1,3,2-dioxaborolan-2-yl)phenoxy]ethyl]carbamate (75.0%) (458 mg, 65% yield, 75% purity) as a pale-yellow oil.

¹H NMR (500 MHz, DMSO) δ 7.52 (dd, J=7.3, 1.8 Hz, 1H), 7.43 (ddd, J=8.2, 7.3, 1.8 Hz, 1H), 6.99-6.91 (m, 2H), 6.49 (t, J=5.6 Hz, 1H), 3.98 (t, J=5.6 Hz, 2H), 3.39-3.34 (m, 2H), 1.40 (s, 9H), 1.31 (s, 12H).

### Step 2

To a solution of tert-butyl N-[2-[2-(4,4,5,5-tetramethyl-1,3,2-dioxaborolan-2-yl)phenoxy]ethyl]carbamate (75%, 444 mg, 0.916 mmol) and 6-bromo-5-fluoro-indoline (90%, 200 mg, 0.833 mmol) in 1,4-dioxane (4.5 mL) was added potassium carbonate (230 mg, 1.67 mmol) and the mixture was sparged with nitrogen for 5 minutes. Pd(dppf)Cl₂ (61 mg, 0.0833 mmol) was then added and the reaction mixture sparged for another 5 minutes. The vessel was sealed and the reaction mixture heated at 100° C. overnight. The mixture was filtered through Celite, washing with EtOAc and the filtrate was concentrated under reduced pressure. The residue was purified by FCC (25 g SiO₂ column, 0-100% EtOAc in heptane) to afford tert-butyl N-[2-[2-(5-fluoroindolin-6-yl)phenoxy]ethyl]carbamate (283 mg, 67% yield, 74% purity) as a pale brown oil.

¹H NMR (400 MHz, DMSO) δ 7.32 (ddd, J=8.3, 7.3, 1.8 Hz, 1H), 7.20-7.15 (m, 1H), 7.08 (dd, J=8.4, 1.1 Hz, 1H), 6.99 (td, J=7.4, 1.0 Hz, 1H), 6.89 (d, J=9.7 Hz, 1H), 6.71 (t, J=5.5 Hz, 1H), 6.38 (d, J=6.1 Hz, 1H), 5.31 (d, J=2.3 Hz, 1H), 3.97-3.89 (m, 2H), 3.43 (td, J=8.5, 2.2 Hz, 2H), 3.17 (q, J=6.3 Hz, 2H), 2.93 (t, J=8.4 Hz, 2H), 1.37 (s, 9H).

LCMS: m/z=373.1 [M+H]+, (ESI+), RT=0.74, Method B

### Step 3

To a solution of 2-methoxy-5-methoxycarbonyl-benzoic acid (96%, 94 mg, 0.429 mmol), tert-butyl N-[2-[2-(5-fluoroindolin-6-yl)phenoxy]ethyl]carbamate (74%, 180 mg, 0.358 mmol) and DIPEA (249 μL, 1.43 mmol) in anhydrous DMF (6.3 mL) was added dropwise T3P (50% in EtOAc) (0.64 mL, 1.07 mmol). The mixture was stirred at r.t. for 1 hour. The mixture was diluted with water (40 ml) and extracted with EtOAc (2×40 mL). The combined organic was washed with water (30 mL) and brine (30 mL) then dried over MgSO₄, filtered and concentrated in vacuo. The residue was purified by FCC (10 g SiO₂ column, 0-100% EtOAc in heptane) to afford methyl 3-[6-[2-[2-(tert-butoxycarbonylamino)ethoxy]phenyl]-5-fluoro-indoline-1-carbonyl]-4-methoxybenzoate (95.0%) (155 mg, 73% yield, 95% purity) as a white solid.

¹H NMR (400 MHz, DMSO) δ 8.09 (d, J=6.7 Hz, 1H), 8.06 (dd, J=8.8, 2.2 Hz, 1H), 7.89 (d, J=2.3 Hz, 1H), 7.43-7.34 (m, 1H), 7.29 (d, J=8.9 Hz, 1H), 7.25 (dd, J=7.5, 1.8 Hz, 1H), 7.15 (dd, J=11.7, 8.8 Hz, 2H), 7.05 (td, J=7.5, 1.0 Hz, 1H), 6.73 (t, J=5.7 Hz, 1H), 3.99 (t, J=6.3 Hz, 2H), 3.92 (s, 3H), 3.87-3.76 (m, 5H), 3.20 (q, J=6.3 Hz, 2H), 3.12 (t, J=8.4 Hz, 2H), 1.36 (s, 9H).

LCMS: m/z=587.3 [M+Na]+, (ESI+), RT=1.05, Method B

### Step 4

Methyl 3-[6-[2-[2-(tert-butoxycarbonylamino)ethoxy]phenyl]-5-fluoro-indoline-1-carbonyl]-4-methoxybenzoate (95%, 155 mg, 0.261 mmol) was dissolved in THE (4 mL) and 2 M aq. sodium hydroxide (1.0 mL, 2.00 mmol) was added. The reaction mixture was stirred at r.t. for 45 minutes. 6 M aq. sodium hydroxide (1.0 mL, 6.00 mmol) was then added and the mixture stirred at r.t. for 2.5 hours. MeOH (1.5 mL) was then added and the mixture stirred at r.t. for 45 minutes. The mixture was then warmed to 40° C. for 3 hours. The mixture was concentrated under vacuum, the remaining aqueous diluted with water (20 mL) and acidified with 1 M aq. HCl. The mixture was extracted with DCM (3×20 mL), the combined organic passed through a phase separator and then concentrated to afford 3-[6-[2-[2-(tert-butoxycarbonylamino)ethoxy]phenyl]-5-fluoro-indoline-1-carbonyl]-4-methoxybenzoic acid (144 mg, 92% yield, 92% purity) as a pale-yellow solid.

LCMS: m/z=549.2 [M−H]−, (ESI−), RT=1.05, Method B

### Step 5

To 3-[6-[2-[2-(tert-butoxycarbonylamino)ethoxy]phenyl]-5-fluoro-indoline-1-carbonyl]-4-methoxybenzoic acid (92%, 140 mg, 0.234 mmol) was added 4 M HCl in dioxane (1.8 mL, 7.23 mmol). The mixture was stirred at r.t. for 1 hour. The reaction mixture was concentrated under reduced pressure. The residue was taken up in DCM and concentrated (3×) to afford 3-[6-[2-(2-aminoethoxy)phenyl]-5-fluoro-indoline-1-carbonyl]-4-methoxybenzoic acid·HCl (120 mg, 95% yield, 90% purity) as a pale-yellow solid.

LCMS: m/z=451.1 [M+H]+, (ESI+), RT=0.66, Method B

### Step 6

To a solution of T3P (50% in EtOAc) (330 μL, 0.555 mmol) and DIPEA (128 μL, 0.739 mmol) in anhydrous DMF (8 mL) was added dropwise a solution of 3-[6-[2-(2-aminoethoxy)phenyl]-5-fluoro-indoline-1-carbonyl]-4-methoxybenzoic acid·HCl (90%, 100 mg, 0.185 mmol) in anhydrous DMF (8 mL) over 5 minutes. The mixture was stirred at r.t. for 30 minutes. The mixture was concentrated under vacuum. The residue was taken up in EtOAc and water and the layers separated. The aqueous phase was extracted with further EtOAc. The combined organic was washed with water and brine then dried over MgSO₄, filtered and concentrated in vacuo. The residue was purified by reverse phase FCC (12 g C18 column, 10-100% MeCN (0.1% formic acid) in water (0.1% formic acid)) to afford 24-fluoro-16-methoxy-8-oxa-11,19-diazapentacyclo[17.5.2.113,17.02,7.022,26]heptacosa-1(24),2,4,6,13,15,17(27),22,25-nonaene-12,18-dione (17 mg, 24% yield, 100% purity) as a white solid.

¹H NMR (400 MHz, DMSO) δ 8.06 (s, 1H), 7.91 (d, J=8.7 Hz, 1H), 7.65-7.56 (m, 1H), 7.32-7.24 (m, 1H), 7.23-7.15 (m, 2H), 6.98 (d, J=8.3 Hz, 1H), 6.90-6.83 (m, 2H), 5.66 (d, J=6.2 Hz, 1H), 4.57-4.39 (m, 1H), 4.14-3.96 (m, 3H), 3.87-3.70 (m, 4H), 3.27-3.02 (m, 3H).

LCMS: m/z=433.0 [M+H]+, (ESI+), RT=0.78, Method B

### Step 7

24-fluoro-16-methoxy-8-oxa-11,19-diazapentacyclo[17.5.2.113,17.02,7.022,26]heptacosa-1(24),2,4,6,13,15,17(27),22,25-nonaene-12,18-dione (18 mg, 95% purity) was dissolved in 2,4,6-trimethylpyridine (1.3 mL, 9.71 mmol). To the reaction mixture was added lithium iodide (26 mg, 0.198 mmol) and the reaction mixture was heated at 80° C. for 18 hours and was then allowed to cool to r.t. The reaction was acidified by addition of 1 M aq. HCl. The organics were extracted with DCM (3×), combined, passed through a hydrophobic frit and concentrated in vacuo. Purification was performed by acidic reverse phase FCC (6 g C18 SiO₂, 10-100% Acetonitrile (0.1% formic acid) in water (0.1% formic acid)) to afford the title compound as an off-white solid (8 mg, 48% yield, 99% purity).

¹H NMR (500 MHz, DMSO) δ 11.07 (broad s, 1H), 8.23 (s, 1H), 7.83-7.76 (m, 2H), 7.29 (ddd, J=8.4, 7.1, 2.1 Hz, 1H), 7.25 (d, J=8.7 Hz, 1H), 6.99 (d, J=8.3 Hz, 1H), 6.96-6.78 (m, 3H), 6.20 (d, J=6.0 Hz, 1H), 4.47-4.39 (m, 1H), 4.13-3.95 (m, 3H), 3.80-3.69 (m, 1H), 3.26-3.02 (m, 3H).

LCMS: m/z=419.1 [M+H]+, (ESI+), RT=3.20, Method A

### Example 12—Synthesis of 21,23-difluoro-16-hydroxy-8-oxa-11,19-diazatetracyclo[18.3.1.1^{13,17}.0^{2,7}]pentacosa-1(23),2,4,6,13,15,17(25),20(24),21-nonaene-12,18-dione (Compound 12)

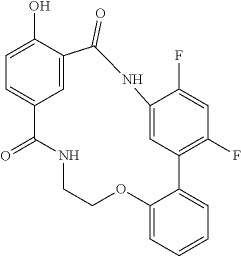 [SMILES: O=C1NCCOc2ccccc2-c2cc(c(F)cc2F)NC(=O)c2cc1ccc2O]

### Step 1

To a solution of 4-methoxy-3-methoxycarbonyl-benzoic acid (387 mg, 1.84 mmol), 2-(2-bromophenoxy)ethanamine hydrochloride (96%, 440 mg, 1.67 mmol) and DIPEA (1165 μL, 6.69 mmol) in anhydrous DMF (10 mL) was added T3P (50% in EtOAc) (2.5 mL, 4.18 mmol). The mixture was stirred at r.t. for 1.5 hours. Further T3P (50% in EtOAc) (1.5 mL, 2.51 mmol) was added and the mixture was stirred at r.t. for 2 hours. The mixture was diluted with H₂O (40 mL) and extracted with EtOAc (2×40 mL). The combined organic layers were washed with water (30 mL) and brine (30 mL) then dried over MgSO₄, filtered and concentrated under vacuum to afford 5-[2-(2-bromophenoxy)ethylcarbamoyl]-2-methoxybenzoate (565 mg, 71% yield, 86% purity) as a yellow oil.

¹H NMR (500 MHz, DMSO) δ 8.68 (t, J=5.4 Hz, 1H), 8.19 (d, J=2.4 Hz, 1H), 8.05 (dd, J=8.8, 2.4 Hz, 1H), 7.57 (dd, J=7.9, 1.6 Hz, 1H), 7.34 (ddd, J=8.2, 7.4, 1.6 Hz, 1H), 7.24 (d, J=8.9 Hz, 1H), 7.18 (dd, J=8.3, 1.4 Hz, 1H), 6.90 (td, J=7.6, 1.4 Hz, 1H), 4.20 (t, J=6.1 Hz, 2H), 3.88 (s, 3H), 3.81 (s, 3H), 3.64 (q, J=5.9 Hz, 2H).

LCMS: m/z=407.9/409.9 [M+H]+, (ESI+), RT=0.84, Method B

### Step 2

To a solution of methyl 5-[2-(2-bromophenoxy)ethylcarbamoyl]-2-methoxybenzoate (86%, 550 mg, 1.16 mmol) and 2,4-difluoro-5-(4,4,5,5-tetramethyl-1,3,2-dioxaborolan-2-yl)aniline (95%, 373 mg, 1.39 mmol) in 1,4-dioxane (5 mL) was added potassium carbonate (320 mg, 2.32 mmol) and the mixture was sparged with nitrogen for 5 minutes. Pd(dppf)Cl₂ (85 mg, 0.116 mmol) was added and the reaction mixture was sparged for another 5 minutes. The vessel was sealed and the reaction mixture heated at 100° C. overnight. The mixture was cooled to room temperature, filtered through Celite and washed with EtOAc. The filtrated was concentrated under vacuum. The residue was purified by FCC (25 g SiO₂ column, 0-100% EtOAc in heptane) to afford methyl 5-[2-[2-(5-amino-2,4-difluoro-phenyl)phenoxy]ethylcarbamoyl]-2-methoxybenzoate (425 mg, 76% yield, 95% purity) as a yellow solid.

¹H NMR (400 MHz, DMSO) δ 8.50 (t, J=5.5 Hz, 1H), 8.16 (d, J=2.3 Hz, 1H), 8.00 (dd, J=8.8, 2.4 Hz, 1H), 7.35 (ddd, J=9.0, 7.4, 1.8 Hz, 1H), 7.24 (d, J=8.9 Hz, 1H), 7.16 (td, J=7.2, 1.4 Hz, 2H), 7.01 (td, J=7.4, 1.1 Hz, 1H), 6.88 (dd, J=11.3, 9.7 Hz, 1H), 6.70 (dd, J=10.1, 7.5 Hz, 1H), 4.94 (s, 2H), 4.10 (t, J=6.3 Hz, 2H), 3.89 (s, 3H), 3.81 (s, 3H), 3.51 (q, J=6.1 Hz, 2H).

LCMS: m/z=457.1 [M+H]+, (ESI+), RT=0.83, Method B

### Step 3

To a mixture of methyl 5-[2-[2-(5-amino-2,4-difluoro-phenyl)phenoxy]ethylcarbamoyl]-2-methoxybenzoate (95%, 200 mg, 0.421 mmol), THE (4.5 mL) and MeOH (1.8 mL) was added 4 M aqueous sodium hydroxide (0.53 mL, 2.10 mmol) and the mixture was stirred at r.t. for 3.5 hours. THE was removed under vacuum, the remaining aqueous was diluted with water (20 mL) and acidified with 1 M aqueous HCl. The mixture was extracted with DCM (3×20 mL), the combined organics were passed through a phase separator and then concentrated to afford 5-[2-[2-(5-amino-2,4-difluoro-phenyl)phenoxy]ethylcarbamoyl]-2-methoxybenzoic acid (195 mg, quantitative yield, 100% purity) as a pale-yellow solid.

LCMS: m/z=443.1 [M+Na]+, (ESI+), RT=0.74, Method B

### Step 4

To a solution of T3P (50% in EtOAc) (690 μL, 1.16 mmol) and DIPEA (269 μL, 1.55 mmol) in anhydrous DMF (35 mL) was added dropwise a solution of 5-[2-[2-(5-amino-2,4-difluoro-phenyl)phenoxy]ethylcarbamoyl]-2-methoxybenzoic acid (95%, 180 mg, 0.387 mmol) in anhydrous DMF (5 mL) over 10 minutes. The mixture was stirred at r.t. overnight. The mixture was diluted with EtOAc and water and the layers separated. The aqueous was extracted with further EtOAc (3×). The combined organic was washed with 0.5 M aq. sodium hydroxide followed by brine, and then dried (Na₂SO₄), filtered and concentrated to afford 21,23-difluoro-16-methoxy-8-oxa-11,19-diazatetracyclo[18.3.1.113,17.02,7]pentacosa-1(23),2,4,6,13,15,17(25),20(24),21-nonaene-12,18-dione (15 mg, 6.4% yield, 70% purity) as a pale brown solid.

LCMS: m/z=425.0 [M+H]+, (ESI+), RT=0.73, Method B

### Step 5

A suspension of 21,23-difluoro-16-methoxy-8-oxa-11,19-diazatetracyclo[18.3.1.113,17.02,7]pentacosa-1(23),2,4,6,13,15,17(25),20(24),21-nonaene-12,18-dione (70%, 15 mg, 0.0247 mmol) and lithium iodide (33 mg, 0.247 mmol) in anhydrous pyridine (1.1 mL) was heated at 85° C. for 18 hours and was then cooled to r.t. To the reaction mixture was added lithium iodide (33 mg, 0.247 mmol) and the reaction mixture was heated at 85° C. for 3 hours, then at 100° C. for 18 hours, then at 110° C. for 22.5 hours and was then allowed to cool to r.t. The reaction mixture was acidified with 1 M aq. HCl. The organics were extracted with DCM (3×), combined, passed through a hydrophobic frit and then concentrated in vacuo. Purification was performed by acidic reverse phase FCC (6 g C18 SiO₂, 10-100% MeCN (0.1% formic acid) in water (0.1% formic acid)) followed by lyophilization to afford the title compound as a brown solid (2 mg, 18% yield, 90% purity).

¹H NMR (500 MHz, DMSO) δ 11.33 (broad s, 1H), 10.03 (broad s, 1H), 7.68 (dd, J=8.6, 2.3 Hz, 1H), 7.50-7.42 (m, 2H), 7.43-7.36 (m, 2H), 7.27-7.17 (m, 3H), 7.11-7.07 (m, 1H), 6.95 (d, J=8.6 Hz, 1H), 4.32-3.92 (m, 2H), 3.56-3.40 (m, 2H).

LCMS: m/z=411.0 [M+H]+, (ESI+), RT=2.86, Method A

### Example 13—Synthesis of 21,23-difluoro-16-hydroxy-8-oxa-11,19-diazatetracyclo[18.3.1.1^{13,17}.0^{2,7}]pentacosa-1(23),2,4,6,13,15,17(25),20(24),21-nonaen-12-one (Compound 13)

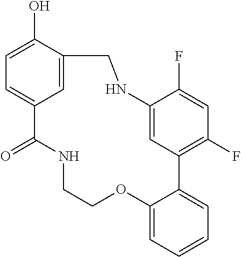 [SMILES: O=C1NCCOc2ccccc2-c2cc(c(F)cc2F)NCc2cc1ccc2O]

### Step 1

To a solution of 5-bromo-2,4-difluoroaniline (500 mg, 2.40 mmol) and methyl 4-benzyloxy-3-formyl-benzoate (780 mg, 2.88 mmol) in DCE (30 mL) was added acetic acid (688 μL, 12.0 mmol) and the mixture was stirred at 85° C. for 5 hours. The mixture was cooled to r.t., STAB (1528 mg, 7.21 mmol) was added and the mixture was stirred at r.t. for 1 hour. The mixture was diluted with DCM (30 mL) and saturated aqueous NaHCO₃ (50 mL). The mixture was stirred for 20 minutes and the layers were separated. The aqueous was extracted with further DCM (2×30 mL), then the combined organics were passed through a phase separator and concentrated. The residue was purified by FCC (50 g SiO₂ column, 0-100% EtOAc in heptane) to afford methyl 4-benzyloxy-3-[(5-bromo-2,4-difluoro-anilino)methyl]benzoate (885 mg, 79% yield, 99% purity) as a white solid.

¹H NMR (500 MHz, DMSO) δ 7.94-7.83 (m, 2H), 7.55-7.49 (m, 2H), 7.44-7.39 (m, 2H), 7.37-7.30 (m, 2H), 7.23 (d, J=8.4 Hz, 1H), 6.72 (dd, J=9.1, 6.9 Hz, 1H), 6.32 (td, J=6.2, 2.1 Hz, 1H), 5.30 (s, 2H), 4.35 (d, J=6.2 Hz, 2H), 3.77 (s, 3H).

LCMS: m/z=461.9/463.9 [M+H]+, (ESI+), RT=1.19, Method B

### Step 2

To a solution of 2-(4,4,5,5-tetramethyl-1,3,2-dioxaborolan-2-yl)phenol (1.50 g, 6.82 mmol), tert-butyl (2-hydroxyethyl)carbamate (1.6 mL, 10.2 mmol) and triphenylphosphine (2.68 g, 10.2 mmol) in anhydrous THE (40 mL) was added DIAD (2.1 mL, 10.9 mmol) and the reaction was stirred at r.t. for 1 hour, then warmed to 35° C. with stirring for 3 hours. Further triphenylphosphine (1.79 g, 6.82 mmol) was added and the mixture was stirred at 35° C. for 30 minutes. Further DIAD (1.3 mL, 6.82 mmol) was added at r.t, and the mixture was stirred at 35° C. for 25 minutes. Further tert-butyl (2-hydroxyethyl)carbamate (1.1 mL, 6.82 mmol) was added and the mixture was stirred at 35° C. for 1 hour. The mixture was then concentrated under reduced pressure and the residue was purified by FCC (100 g SiO₂ column, 0-100% EtOAc in heptane) to afford tert-butyl N-[2-[2-(4,4,5,5-tetramethyl-1,3,2-dioxaborolan-2-yl)phenoxy]ethyl]carbamate (680 mg, 25% yield, 92% purity) as a pale brown oil.

¹H NMR (500 MHz, DMSO) δ 7.52 (d, J=1.8 Hz, 1H), 7.44 (ddd, J=8.3, 7.3, 1.9 Hz, 1H), 6.99-6.85 (m, 2H), 6.50 (t, J=5.6 Hz, 1H), 3.98 (t, J=5.6 Hz, 2H), 3.38-3.34 (m, 2H), 1.40 (s, 9H), 1.31 (s, 12H).

### Step 3

To a solution of tert-butyl N-[2-[2-(4,4,5,5-tetramethyl-1,3,2-dioxaborolan-2-yl)phenoxy]ethyl]carbamate (92%, 282 mg, 0.714 mmol) and methyl 4-benzyloxy-3-[(5-bromo-2,4-difluoro-anilino)methyl]benzoate (300 mg, 0.649 mmol) in 1,4-dioxane (4 mL) and water (0.4 mL) was added potassium carbonate (179 mg, 1.30 mmol) and the mixture was sparged with nitrogen for 5 minutes. Pd(dppf)Cl₂ (48 mg, 0.0649 mmol) was added and the reaction mixture was degassed for another 5 minutes. The vessel was sealed and the reaction mixture heated at 100° C. overnight. The mixture was filtered through Celite, washing with EtOAc and the filtrate was concentrated under reduced pressure. The residue was purified by FCC (25 g SiO₂ column, 0-100% EtOAc in heptane) to afford methyl 4-benzyloxy-3-[[5-[2-[2-(tert-butoxycarbonylamino)ethoxy]phenyl]-2,4-difluoro-anilino]methyl]benzoate (356 mg, 72% yield, 81% purity) as a pale yellow oil.

¹H NMR (500 MHz, DMSO) δ 7.96 (d, J=2.3 Hz, 1H), 7.84 (dd, J=8.6, 2.3 Hz, 1H), 7.40-7.24 (m, 6H), 7.19 (d, J=8.7 Hz, 1H), 7.13-7.00 (m, 3H), 6.95 (td, J=7.3, 1.0 Hz, 1H), 6.65 (t, J=5.7 Hz, 1H), 6.47 (dd, J=9.8, 7.2 Hz, 1H), 6.00 (td, J=6.4, 2.1 Hz, 1H), 5.21 (s, 2H), 4.34 (d, J=6.2 Hz, 2H), 3.85 (t, J=6.5 Hz, 2H), 3.78 (s, 3H), 3.06 (q, J=6.2 Hz, 2H), 1.34 (s, 9H).

LCMS: m/z=619.4 [M+H]+, (ESI+), RT=1.22, Method B

### Step 4

To a mixture of methyl 4-benzyloxy-3-[[5-[2-[2-(tert-butoxycarbonylamino)ethoxy]phenyl]-2,4-difluoro-anilino]methyl]benzoate (81%, 356 mg, 0.466 mmol), THE (4.4367 mL) and MeOH (1.4789 mL) was added 4 M aq. sodium hydroxide (0.58 mL, 2.33 mmol). The mixture was stirred at r.t. for 1 hour, then warmed to 50° C., and stirred for 4.5 hours. THE was removed under vacuum, the remaining aqueous phase was diluted with water (20 mL) and acidified with 1 M aqueous HCl. The mixture was extracted with DCM (4×30 mL), the combined organic phases were passed through a phase separator and then concentrated to afford 5-[2-[2-(5-amino-2,4-difluoro-phenyl)phenoxy]ethylcarbamoyl]-2-methoxybenzoic acid (201 mg, 94% yield, 95% purity) as a pale-yellow oil.

LCMS: m/z=605.3 [M+H]+, (ESI+), RT=1.10, Method B

### Step 5

To 4-benzyloxy-3-[[5-[2-[2-(tert-butoxycarbonylamino)ethoxy]phenyl]-2,4-difluoro-anilino]methyl]benzoic acid (91%, 274 mg, 0.412 mmol) was added 4 M HCl in dioxane (2.1 mL, 8.25 mmol). The mixture was stirred at r.t. for 1 hour, then concentrated under reduced pressure. The residue was sonicated in DCM then concentrated (3×) to afford 3-[[5-[2-(2-aminoethoxy)phenyl]-2,4-difluoro-anilino]methyl]-4-benzyloxybenzoic acid dihydrochloride (294 mg, 99% yield, 80% purity) as a pale brown solid.

LCMS: m/z=505.1 [M+H]+, (ESI+), RT=0.78, Method B

### Step 6

To a solution of T3P (50% in EtOAc) (807 μL, 1.36 mmol) and DIPEA (394 μL, 2.26 mmol) in anhydrous DMF (26 mL) was added dropwise a solution of 3-[[5-[2-(2-aminoethoxy)phenyl]-2,4-difluoro-anilino]methyl]-4-benzyloxybenzoic acid dihydrochloride (80%, 290 mg, 0.452 mmol) in anhydrous DMF (4 mL) over 5 minutes. The mixture was stirred at r.t. for 30 minutes, then diluted with EtOAc (70 mL) and H₂O (70 mL) and the layers were separated. The aqueous layer was extracted with further EtOAc (70 mL). The combined organics were washed with H₂O (70 mL), then brine (70 mL), passed through a phase separator and then concentrated to afford 16-benzyloxy-21,23-difluoro-8-oxa-11,19-diazatetracyclo[18.3.1.113,17.02,7]pentacosa-1(23),2,4,6,13,15,17(25),20(24),21-nonaen-12-one (208 mg, 79% Yield, 84% purity) as a pale brown solid.

LCMS: m/z=487.1 [M+H]+, (ESI+), RT=1.06, Method B

### Step 7

To 16-benzyloxy-21,23-difluoro-8-oxa-11,19-diazatetracyclo[18.3.1.113,17.02,7]pentacosa-1(23),2,4,6,13,15,17(25),20(24),21-nonaen-12-one (84%, 60 mg, 0.104 mmol) at r.t. was added 1 M BBr₃ in DCM (1.0 mL, 1.00 mmol). The reaction mixture was stirred 45 minutes at r.t. The reaction mixture was diluted with DCM (30 mL) and poured onto H₂O (40 mL) and then the layers were separated. The aqueous layer was extracted with DCM (2×30 mL). The organic phases were combined, passed through a hydrophobic frit and concentrated in vacuo. The residue was purified by preparative HPLC (Method P1), followed by further purification by preparative HPLC (Method P2) to afford the title compound as a white solid (20 mg, 49% yield, 100% purity).

¹H NMR (500 MHz, DMSO) δ 10.16 (s, 1H), 7.53 (d, J=2.4 Hz, 1H), 7.44 (dd, J=8.3, 2.2 Hz, 1H), 7.37 (ddd, J=8.3, 7.4, 1.8 Hz, 1H), 7.14 (dd, J=7.5, 1.8 Hz, 1H), 7.13-7.06 (m, 2H), 7.03-6.98 (m, 2H), 6.89 (dd, J=9.8, 7.5 Hz, 1H), 6.82 (d, J=8.3 Hz, 1H), 5.56 (td, J=7.0, 2.5 Hz, 1H), 4.43-4.19 (m, 2H), 4.16-4.01 (m, 2H), 3.57-3.40 (m, 2H).

LCMS: m/z=397.1 [M+H]+, (ESI+), RT=2.97, Method A

### Example 14—Synthesis of 15-chloro-21,23-difluoro-16-hydroxy-8-oxa-18lambda6-thia-11-azatetracyclo[18.3.1.1^{13,17}.0^{2,7}]pentacosa-1(24),2,4,6,13,15,17(25),20,22-nonaene-12,18,18-trione (Compound 14)

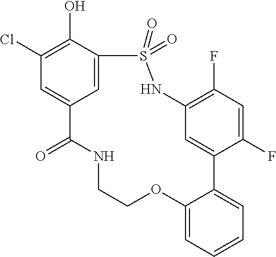 [SMILES: O=C1NCCOc2ccccc2-c2cc(c(F)cc2F)NS(=O)(=O)c2cc1cc(Cl)c2O]

### Step 1

To a solution of Intermediate 2 (98%, 650 mg, 2.01 mmol) and [2,4-difluoro-5-(4,4,5,5-tetramethyl-1,3,2-dioxaborolan-2-yl)phenyl]methanol (58%, 1.00 g, 2.15 mmol) in 1,4-dioxane (12 mL) was added potassium carbonate (550 mg, 3.98 mmol) and water (3 mL) and the mixture was sparged with nitrogen for 10 minutes. Pd(dppf)Cl₂ (150 mg, 0.204 mmol) was added and the reaction mixture was degassed again for another 5 minutes, before the vessel was sealed and the reaction mixture was stirred at reflux for 20 hours. The reaction mixture was cooled to r.t., filtered through celite and washed with EtOAc and the filtrate was added to water (50 mL). The layers were separated and the aqueous phase extracted with EtOAc (2×50 mL). The organic layers were combined, washed with brine (100 mL), dried over Na₂SO₄, filtered and concentrated under reduced pressure. The residue was purified by FCC (50 g SiO₂ column, 0-100% EtOAc in heptane) to afford tert-butyl N-[2-[2-[2,4-difluoro-5-(hydroxymethyl)phenyl]phenoxy]ethyl]carbamate (792 mg, 98% yield, 95% purity) as a viscous yellow oil.

¹H NMR (500 MHz, DMSO) δ 7.45-7.35 (m, 2H), 7.23 (dd, J=7.5, 1.5 Hz, 1H), 7.18 (t, J=10.1 Hz, 1H), 7.12 (d, J=8.2 Hz, 1H), 7.04 (td, J=7.5, 0.9 Hz, 1H), 6.74 (t, J=5.5 Hz, 1H), 5.26 (t, J=5.5 Hz, 1H), 4.54 (d, J=5.1 Hz, 2H), 3.96 (t, J=6.3 Hz, 2H), 3.18 (q, J=6.1 Hz, 2H), 1.35 (s, 9H).

LCMS: m/z=402.0 [M+Na]+, (ESI+), RT=0.94, Method B

### Step 2

To a 0° C. solution of tert-butyl N-[2-[2-[2,4-difluoro-5-(hydroxymethyl)phenyl]phenoxy]ethyl]carbamate (95%, 620 mg, 1.55 mmol) and carbon tetrabromide (800 mg, 2.41 mmol) in anhydrous DCM (50 mL) was added triphenylphosphine (620 mg, 2.36 mmol). The resulting mixture was stirred under nitrogen for 4 hours while it was allowed to warm to r.t. The reaction mixture was concentrated under reduced pressure and was purified by FCC (25 g SiO₂ column, 0-100% EtOAc in heptane) to afford tert-butyl N-[2-[2-[5-(bromomethyl)-2,4-difluoro-phenyl]phenoxy]ethyl]carbamate (630 mg, 90% yield, 98% purity) as a pale yellow oil.

¹H NMR (500 MHz, DMSO) δ 7.60 (t, J=8.4 Hz, 1H), 7.39 (ddd, J=8.4, 7.5, 1.7 Hz, 1H), 7.31 (t, J=10.1 Hz, 1H), 7.27-7.22 (m, 1H), 7.13 (d, J=8.2 Hz, 1H), 7.04 (td, J=7.5, 0.8 Hz, 1H), 6.78 (t, J=5.4 Hz, 1H), 4.72 (s, 2H), 3.98 (t, J=6.1 Hz, 2H), 3.20 (q, J=6.0 Hz, 2H), 1.36 (s, 9H).

LCMS: m/z=342.0/343.9 [M-Boc+H]+, (ESI+), RT=1.13, Method B

### Step 3

To a stirring solution of tert-butyl N-[2-[2-[5-(bromomethyl)-2,4-difluoro-phenyl]phenoxy]ethyl]carbamate (98%, 600 mg, 1.33 mmol) and thioacetic acid (120 mg, 1.58 mmol) in MeOH (60 mL) was added potassium carbonate (220 mg, 1.59 mmol). Following 30 minutes of stirring at r.t. further potassium carbonate (220 mg, 1.59 mmol) was added and the reaction was left to stir at r.t. for another 30 minutes. The reaction mixture was acidified (to pH ˜4/5) with 1 M aq. HCl, before the solution was diluted with water (20 mL) and extracted with DCM (3×30 mL). The organic phases were combined, dried over Na₂SO₄ and concentrated under reduced pressure. The residue was purified by FCC (50 g SiO₂ column, 0-100% ethyl acetate in heptane) to afford tert-butyl N-[2-[2-[2,4-difluoro-5-(sulfanylmethyl)phenyl]phenoxy]ethyl]carbamate (316 mg, 60% yield, 99% purity) as a viscous colorless oil.

¹H NMR (500 MHz, DMSO) δ 7.47 (t, J=8.6 Hz, 1H), 7.38 (ddd, J=8.3, 7.5, 1.8 Hz, 1H), 7.27-7.19 (m, 2H), 7.13 (d, J=8.2 Hz, 1H), 7.04 (td, J=7.5, 0.9 Hz, 1H), 6.76 (t, J=5.4 Hz, 1H), 3.97 (t, J=6.2 Hz, 2H), 3.75 (s, 2H), 3.20 (q, J=6.1 Hz, 2H), 2.98 (s, 1H), 1.36 (s, 9H).

LCMS: m/z=296.0 [M-Boc+H]+, (ESI+), RT=1.11, Method B

### Step 4

To a solution of methyl 3-bromo-5-chloro-4-methoxy-benzoate (95%, 200 mg, 0.680 mmol) in anhydrous 1,4-dioxane (5 mL) was added DIPEA (110 mg, 0.851 mmol) and tert-butyl N-[2-[2-[2,4-difluoro-5-(sulfanylmethyl)phenyl]phenoxy]ethyl]carbamate (99%, 310 mg, 0.776 mmol). The solution was bubbled with nitrogen for 5 minutes, before Pd₂(dba)₃ (20 mg, 0.0218 mmol) and Xantphos (30 mg, 0.0518 mmol) were added and the sealed tube was heated at 100° C. for 3 hours. The reaction mixture was allowed to cool to r.t., before it was added to water (50 mL) and extracted with EtOAc (3×50 mL). The combined organic layers were washed with brine (100 mL), dried over Na₂SO₄, filtered and concentrated. The residue was purified by FCC (100 g SiO₂ cartridge, 0-100% EtOAc in heptane) to afford methyl 3-[[5-[2-[2-(tert-butoxycarbonylamino)ethoxy]phenyl]-2,4-difluoro-phenyl]methylsulfanyl]-5-chloro-4-methoxy-benzoate (357 mg, 88% yield, 99% purity) as a viscous pale yellow oil.

¹H NMR (500 MHz, DMSO) δ 7.86 (d, J=2.0 Hz, 1H), 7.82 (d, J=2.0 Hz, 1H), 7.45 (t, J=8.4 Hz, 1H), 7.38 (ddd, J=8.4, 7.5, 1.7 Hz, 1H), 7.25 (t, J=10.0 Hz, 1H), 7.16-7.10 (m, 2H), 7.06-6.98 (m, 1H), 6.73 (t, J=5.6 Hz, 1H), 4.33 (s, 2H), 3.94 (t, J=6.2 Hz, 2H), 3.82-3.80 (m, 6H), 3.16 (q, J=6.1 Hz, 2H), 1.34 (s, 9H).

LCMS: m/z=494.0 [M-Boc+H]+, (ESI+), RT=1.25, Method B

### Step 5

To a solution of methyl 3-[[5-[2-[2-(tert-butoxycarbonylamino)ethoxy]phenyl]-2,4-difluoro-phenyl]methylsulfanyl]-5-chloro-4-methoxybenzoate (99%, 300 mg, 0.500 mmol) in DCM (10 mL) was added mCPBA (70%, 370 mg, 1.50 mmol) and sodium bicarbonate (50 mg, 0.595 mmol). The resulting solution was stirred at r.t. for 16 hours. The reaction was added to a biphasic mixture of DCM (20 mL) and aqueous sodium sulfite (30 mL) and was mixed thoroughly in a separation funnel. The layers were then separated and the aqueous phase extracted with additional DCM (20 mL). The combined organic layers were then washed with aqueous sodium bicarbonate (40 mL) and the aqueous extracted a second time with additional DCM (20 mL). The organic phases were combined and finally washed with brine (50 mL), then dried over Na₂SO₄, filtered and concentrated under reduced pressure. The residue was purified by FCC (10 g SiO₂ column, 0-100% EtOAc in heptane) to afford methyl 3-[[5-[2-[2-(tert-butoxycarbonylamino)ethoxy]phenyl]-2,4-difluoro-phenyl]methylsulfonyl]-5-chloro-4-methoxy-benzoate (272 mg, 78% yield, 90% purity).

¹H NMR (500 MHz, DMSO) δ 8.36 (d, J=2.1 Hz, 1H), 8.09 (d, J=2.1 Hz, 1H), 7.46-7.35 (m, 2H), 7.27 (t, J=9.9 Hz, 1H), 7.14 (d, J=8.3 Hz, 1H), 7.09 (dd, J=7.4, 1.4 Hz, 1H), 7.04 (t, J=7.3 Hz, 1H), 6.75 (t, J=5.5 Hz, 1H), 4.90 (s, 2H), 4.08 (s, 3H), 3.95 (t, J=6.2 Hz, 2H), 3.87 (s, 3H), 3.19 (q, J=6.1 Hz, 2H), 1.34 (s, 9H).

LCMS: m/z=526.0 [M-Boc+H]+, (ESI+), RT=1.15, Method B

### Step 6

Methyl 3-[[5-[2-[2-(tert-butoxycarbonylamino)ethoxy]phenyl]-2,4-difluoro-phenyl]methylsulfonyl]-5-chloro-4-methoxybenzoate (90%, 265 mg, 0.38 mmol) was dissolved in THE (15 mL) and 2 M aq. sodium hydroxide (3.0 mL, 6.00 mmol) was added. The resulting solution was stirred at r.t. for 3 hours. THE was removed under reduced pressure and the remaining aqueous solution was added to DCM (30 mL) and acidified with 1 M aq. HCl (30 mL). The layers were separated and the aqueous phase was extracted with additional DCM (2×15 mL), before the combined organic phases were washed with brine (30 mL), dried over Na₂SO₄, filtered and concentrated under reduced pressure to afford 3-[[5-[2-[2-(tert-butoxycarbonylamino)ethoxy]phenyl]-2,4-difluoro-phenyl]methylsulfonyl]-5-chloro-4-methoxybenzoic acid (245 mg, 91% yield, 93% purity) as a tan solid.

LCMS: m/z=512.0 [M-Boc+H]+, (ESI+), RT=1.07, Method B

### Step 7

To 3-[[5-[2-[2-(tert-butoxycarbonylamino)ethoxy]phenyl]-2,4-difluoro-phenyl]methylsulfonyl]-5-chloro-4-methoxybenzoic acid (93%, 240 mg, 0.365 mmol) was added 4 M HCl in dioxane (6.0 mL, 24.0 mmol). The solution was stirred at r.t. for 4 hours. The reaction mixture was concentrated under reduced pressure, before the remaining residue was taken up into DCM (with sonication) and concentrated (3×20 mL) to afford 3-[[5-[2-(2-aminoethoxy)phenyl]-2,4-difluoro-phenyl]methylsulfonyl]-5-chloro-4-methoxybenzoic acid-HCl (236 mg, 94% yield, 80% purity) as a golden solid.

LCMS: m/z=512.0 [M+H]+, (ESI+), RT=0.74, Method B

### Step 8

To a solution of DIPEA (0.30 mL, 1.72 mmol) and T3P (50% in EtOAc) (0.70 mL, 1.18 mmol) in DMF (3 mL) was added, via pump-syringe, a solution of 3-[[5-[2-(2-aminoethoxy)phenyl]-2,4-difluoro-phenyl]methylsulfonyl]-5-chloro-4-methoxybenzoic acid; hydrochloride (80%, 230 mg, 0.336 mmol) in DMF (4 mL) over the course of 1 hour. Following this addition, the mixture was stirred at r.t. for 3 hours. A second aliquot of T3P (50% in EtOAc) (0.70 mL, 1.18 mmol) was added dropwise and the mixture was allowed to stir for another 16 hours. Additional DIPEA (0.30 mL, 1.72 mmol) was then added and the reaction was allowed to stir for a final hour. The reaction was then diluted with water (100 mL) and extracted with EtOAc (4×50 mL). The combined organic phases were washed with brine (100 mL), dried over Na₂SO₄, filtered and concentrated in vacuo to afford 15-chloro-21,23-difluoro-16-methoxy-18,18-dioxo-8-oxa-18λ6-thia-11-azatetracyclo[18.3.1.113,17.02,7]pentacosa-1(24),2,4,6,13,15,17(25),20,22-nonaen-12-one (150 mg, 81% yield, 90% purity) as a pale brown solid.

¹H NMR (500 MHz, DMSO) δ 8.14 (t, J=4.3 Hz, 1H), 8.09 (d, J=2.2 Hz, 1H), 7.45 (td, J=8.2, 1.7 Hz, 1H), 7.40 (d, J=2.2 Hz, 1H), 7.36 (t, J=8.2 Hz, 1H), 7.20 (dd, J=7.4, 1.7 Hz, 1H), 7.18-7.14 (m, 1H), 7.07 (td, J=7.4, 0.8 Hz, 1H), 6.88 (t, J=9.9 Hz, 1H), 4.65 (s, 2H), 4.01 (s, 3H).

### Step 9

To a solution of 15-chloro-21,23-difluoro-16-methoxy-18,18-dioxo-8-oxa-18λ6-thia-11-azatetracyclo[18.3.1.113,17.02,7]pentacosa-1(24),2,4,6,13,15,17(25),20,22-nonaen-12-one (90%, 120 mg, 0.22 mmol) in anhydrous DMF (4 mL) at 0° C. was added dropwise 1 M BBr₃ in DCM (2.4 mL, 2.40 mmol). The resulting solution was heated at 80° C. for 20 hours. The reaction mixture was allowed to cool to r.t, and was then placed in ice before additional 1 M BBr₃ in DCM (0.60 mL, 0.6 mmol) was added. The reaction mixture was then re-heated to 80° C., and stirred for a further 4 hours. The reaction mixture was quenched by addition to sat. aq. NaHCO₃ (30 mL) at 0° C., and was diluted into DCM (20 mL). The aqueous phase was extracted again with DCM (20 mL) and the combined organic layers were then washed with brine (50 mL), dried over Na₂SO₄, filtered and concentrated in vacuo. The residue was purified by preparative HPLC (Method P1) to afford the title compound as a white solid (48 mg, 41% yield, 99% purity)

¹H NMR (500 MHz, DMSO) δ 11.20 (broad s, 1H), 7.94 (d, J=2.0 Hz, 1H), 7.91-7.87 (m, 1H), 7.44 (ddd, J=8.2, 7.6, 1.7 Hz, 1H), 7.37-7.32 (m, 1H), 7.27 (d, J=2.1 Hz, 1H), 7.19 (dd, J=7.4, 1.7 Hz, 1H), 7.17-7.12 (m, 1H), 7.09-7.04 (m, 1H), 6.97-6.90 (m, 1H), 4.68 (s, 2H), 4.20 (broad s, 2H). 2H not observed

LCMS: m/z=480.0/481.9 [M+H]+, (ESI+), RT=3.28, Method A

### Example 15—Synthesis of 15-chloro-16-(difluoromethoxy)-21,23-difluoro-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 15)

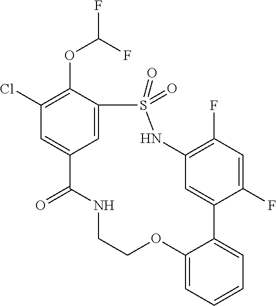 [SMILES: O=C1NCCOc2ccccc2-c2cc(c(F)cc2F)NS(=O)(=O)c2cc1cc(Cl)c2OC(F)F]

To a solution of T3P in EtOAc (50 wt %, 0.52 mL, 0.871 mmol) and DIPEA (0.25 mL, 1.45 mmol) in DMF-anhydrous (12.0 mL) at r.t. was added dropwise a solution of 3-[[5-[2-(2-aminoethoxy)phenyl]-2,4-difluoro-phenyl]sulfamoyl]-5-chloro-4-(difluoromethoxy)benzoic acid·HCl (synthesized according to General Scheme 1, 170 mg, 0.290 mmol) in anhydrous DMF (4.0 mL) over 5 minutes. The reaction mixture was stirred at r.t. for 0.5 hour and was then diluted with H₂O (40 mL). The organics were extracted with EtOAc (2×30 mL), combined, washed with water (2×30 mL) then brine (30 mL), dried over MgSO₄, filtered and concentrated in vacuo. Purification was performed by preparative HPLC (Method P1) to afford the title compound as a white solid (84 mg, 48% yield, 99% purity).

¹H NMR (500 MHz, DMSO) δ 10.58 (s, 1H), 8.35-8.27 (m, 1H), 8.11 (d, J=2.1 Hz, 1H), 7.56 (d, J=2.2 Hz, 1H), 7.48-7.42 (m, 1H), 7.42-7.09 (m, 1H), 7.34-7.28 (m, 1H), 7.26 (dd, J=7.4, 1.7 Hz, 1H), 7.19-7.09 (m, 2H), 7.10-7.03 (m, 1H), 4.29-4.16 (m, 2H), 3.57-3.45 (m, 2H).

LCMS: m/z=531.0/532.9 [M+H]+, (ESI+), RT=3.61, Method A

### Example 16—Synthesis of 15-chloro-21,23-difluoro-16-hydroxy-11-oxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 16)

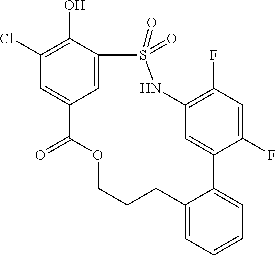 [SMILES: O=C1OCCCc2ccccc2-c2cc(c(F)cc2F)NS(=O)(=O)c2cc1cc(Cl)c2O]

To a solution of 15-chloro-21,23-difluoro-16-methoxy-18,18-dioxo-11-oxa-18λ6-thia-19-azatetracyclo[18.3.1.113,17.02,7]pentacosa-1(24),2,4,6,13,15,17(25),20,22-nonaen-12-one (synthesized according to General Scheme 2, 100%, 24 mg, 0.0486 mmol) in DMF-anhydrous (1.0 mL) at 0° C. was added dropwise 1 M BBr₃ in DCM (0.60 mL, 0.60 mmol). The resulting solution was heated at 80° C. for 16 hours and was then allowed to cool to r.t. The reaction mixture was added to a saturated aqueous solution of NaHCO₃ (30 mL) at 0° C. The organics were extracted with DCM (2×20 mL), combined, washed with brine (50 mL), dried over Na₂SO₄, filtered and concentrated in vacuo. Purification was performed by preparative HPLC (Method P1) to afford the title compound as a white solid (10 mg, 44% yield, 99% purity).

¹H NMR (500 MHz, DMSO) δ 8.00-7.95 (m, 1H), 7.66 (d, J=2.2 Hz, 1H), 7.45-7.41 (m, 2H), 7.40-7.35 (m, 1H), 7.33-7.29 (m, 1H), 7.28-7.24 (m, 1H), 7.24-7.20 (m, 1H), 4.43-4.32 (m, 1H), 4.04-3.95 (m, 1H), 2.68-2.58 (m, 1H), 2.35-2.28 (m, 1H), 2.16-2.03 (m, 1H), 1.81-1.69 (m, 1H).

LCMS: m/z=478.2/480.2 [M−H]−, (ESI−), RT=4.05, Method A

### Example 17—Synthesis of (10S)-15-chloro-21,23-difluoro-16-hydroxy-10-methyl-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 17)

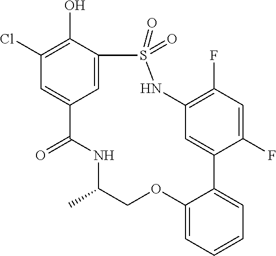 [SMILES: C[C@H]1COc2ccccc2-c2cc(c(F)cc2F)NS(=O)(=O)c2cc(cc(Cl)c2O)C(=O)N1]

To (10S)-15-chloro-21,23-difluoro-16-methoxy-10-methyl-18,18-dioxo-8-oxa-18λ6-thia-11,19-diazatetracyclo[18.3.1.113,17.02,7]pentacosa-1(24),2,4,6,13,15,17(25),20,22-nonaen-12-one (synthesized according to General Scheme 1, 75%, 120 mg, 0.177 mmol) at r.t. was added 1 M BBr₃ in DCM (2.2 mL, 2.2 mmol). The reaction mixture was stirred at r.t. for 18 hours. To the reaction mixture was added H₂O (120 mL). The organics were extracted with DCM (3×120 mL), combined, passed through a hydrophobic frit and concentrated in vacuo. Purification was performed preparative HPLC (Method P1) to afford the title compound as a white solid (45 mg, 49% yield, 97% purity).

¹H NMR (400 MHz, DMSO) δ 10.59-10.12 (m, 2H), 8.03-7.93 (m, 1H), 7.91 (d, J=2.1 Hz, 1H), 7.48-7.39 (m, 1H), 7.33 (d, J=2.2 Hz, 1H), 7.34-7.25 (m, 1H), 7.25 (dd, J=7.5, 1.7 Hz, 1H), 7.17-7.08 (m, 2H), 7.09-7.01 (m, 1H), 4.31 (dd, J=9.4, 4.0 Hz, 1H), 4.15-4.03 (m, 1H), 3.82-3.72 (m, 1H), 1.10 (d, J=6.6 Hz, 3H).

LCMS: m/z=495.0/497.0 [M+H]+, (ESI+), RT=3.62, Method A

### Example 18—Synthesis of 5,15-dichloro-21,23-difluoro-16-hydroxy-8,11-dioxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 18)

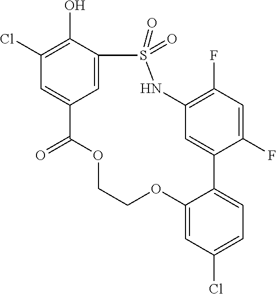 [SMILES: O=C1OCCOc2cc(Cl)ccc2-c2cc(c(F)cc2F)NS(=O)(=O)c2cc1cc(Cl)c2O]

To 5,15-dichloro-21,23-difluoro-16-methoxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (synthesized according to General Scheme 2, 92%, 60 mg, 0.104 mmol) was added dropwise 1 M BBr₃ in DCM (8.0 mL, 8.0 mmol) and the reaction mixture was stirred at r.t. for 2 hours. The reaction was added to a saturated aqueous solution of NaHCO₃ (30 mL). The organics were extracted with DCM (20 mL then 2×30 mL), combined, washed with brine (50 mL), dried over Na₂SO₄, filtered and concentrated in vacuo. Purification was performed by preparative HPLC (Method P1) to afford the title compound as an off-white solid (30 mg, 55% yield, 99% purity).

¹H NMR (400 MHz, DMSO) δ 8.03 (d, J=2.1 Hz, 1H), 7.47 (d, J=2.2 Hz, 1H), 7.34-7.27 (m, 3H), 7.26-7.19 (m, 1H), 7.15 (dd, J=8.1, 1.9 Hz, 1H), 4.34-4.33 (m, 4H). 2H not observed

LCMS: m/z=514.0/516.0 [M−H]−, (ESI−), RT=4.30, Method A

### Example 19—Synthesis of (10R)-15-chloro-21,23-difluoro-16-hydroxy-10-methyl-8-oxa-18lambda6-thia-11,19-diazatetracyclo[18.3.1.1^{13,17}.0^{2,7}]pentacosa-1(24),2,4,6,13,15,17(25),20,22-nonaene-12,18,18-trione (Compound 19)

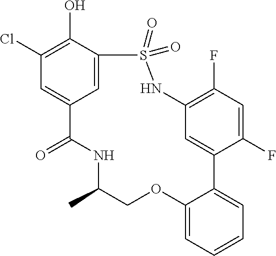 [SMILES: C[C@@H]1COc2ccccc2-c2cc(c(F)cc2F)NS(=O)(=O)c2cc(cc(Cl)c2O)C(=O)N1]

To (10R)-15-chloro-21,23-difluoro-16-methoxy-10-methyl-18,18-dioxo-8-oxa-18λ6-thia-11,19-diazatetracyclo[18.3.1.113,17.02,7]pentacosa-1(24),2,4,6,13,15,17(25),20,22-nonaen-12-one (synthesized according to General Scheme 1, 85%, 123 mg, 0.205 mmol) at r.t. was added 1 M BBr₃ in DCM (2.6 mL, 2.6 mmol). The reaction mixture was stirred at r.t. for 3.5 hours, then 1 M BBr₃ in DCM (1.00 mL, 1.00 mmol) was added and the reaction mixture was stirred for 14 hours at r.t. To the reaction mixture was added H₂O (140 mL). The organics were extracted with DCM (3×140 mL), combined, passed through a hydrophobic frit and concentrated in vacuo. Purification was performed by preparative HPLC (Method P1) to afford the title compound as a white solid (50 mg, 48% yield, 97% purity).

¹H NMR (500 MHz, DMSO) δ 10.77-9.91 (m, 2H), 8.08-7.92 (m, 1H), 7.92-7.89 (m, 1H), 7.46-7.41 (m, 1H), 7.32 (d, J=2.2 Hz, 1H), 7.32-7.27 (m, 1H), 7.25 (dd, J=7.4, 1.7 Hz, 1H), 7.16-7.09 (m, 2H), 7.07-7.03 (m, 1H), 4.31 (dd, J=9.3, 4.0 Hz, 1H), 4.13-4.03 (m, 1H), 3.81-3.73 (m, 1H), 1.10 (d, J=6.6 Hz, 3H).

LCMS: m/z=495.0/497.0 [M+H]+, (ESI+), RT=3.68, Method A

### Example 20—Synthesis of 16-chloro-22,24-difluoro-17-hydroxy-8,12-dioxa-19lambda6-thia-20-azatetracyclo[19.3.1.1^{14,18}.0^{2,7}]hexacosa-1(25),2,4,6,14,16,18(26),21,23-nonaene-13,19,19-trione (Compound 20)

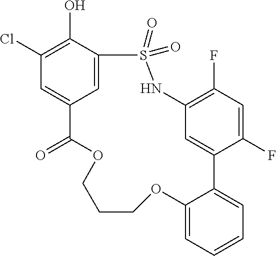 [SMILES: O=C1OCCCOc2ccccc2-c2cc(c(F)cc2F)NS(=O)(=O)c2cc1cc(Cl)c2O]

To 16-chloro-22,24-difluoro-17-methoxy-19,19-dioxo-8,12-dioxa-19λ6-thia-20-azatetracyclo[19.3.1.114,18.02,7]hexacosa-1(25),2,4,6,14,16,18(26),21,23-nonaen-13-one (synthesized according to General Scheme 2, 70 mg, 0.137 mmol) at r.t. was added 1 M BBr₃ in DCM (1.4 mL, 1.40 mmol). The reaction mixture was stirred at r.t. for 1 hour and was then diluted with DCM (30 mL) and poured onto a 5 wt % aqueous solution of NaHCO₃ (40 mL). The layers were separated and the organics were extracted from the aqueous using DCM (2×30 mL). The organics were combined, passed through a hydrophobic frit and concentrated in vacuo. Purification was performed by acidic reverse phase FCC (30 g C18 SiO₂, 10-100% MeCN (0.1% formic acid) in water (0.1% formic acid)) followed by lyophilization to afford the title compound as an off-white solid (27 mg, 39% yield, 98% purity).

¹H NMR (500 MHz, DMSO) δ 11.95-9.45 (m, 1H), 8.00-7.94 (m, 1H), 7.43-7.35 (m, 2H), 7.25-7.18 (m, 1H), 7.15 (dd, J=7.4, 1.7 Hz, 1H), 7.10-7.06 (m, 1H), 7.04-6.98 (m, 1H), 6.98-6.91 (m, 1H), 4.44-4.38 (m, 2H), 4.09 (t, J=5.0 Hz, 2H), 2.01-1.94 (m, 2H).

LCMS: m/z=494.0/496.0 [M−H]−, (ESI−), RT=4.18, Method A

### Example 21—Synthesis of 14-chloro-20,22-difluoro-15-hydroxy-10-oxa-17lambda6-thia-18-azatetracyclo[17.3.1.1^{12,16}.0^{2,7}]tetracosa-1(23),2,4,6,12,14,16(24),19,21-nonaene-11,17,17-trione (Compound 21)

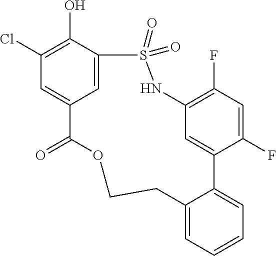 [SMILES: O=C1OCCc2ccccc2-c2cc(c(F)cc2F)NS(=O)(=O)c2cc1cc(Cl)c2O]

To a solution of 15-chloro-21,23-difluoro-16-methoxy-18,18-dioxo-11-oxa-18λ6-thia-19-azatetracyclo[18.3.1.113,17.02,7]pentacosa-1(24),2,4,6,13,15,17(25),20,22-nonaen-12-one (synthesized according to General Scheme 2, 60%, 30 mg, 0.0364 mmol) in anhydrous DCM (0.6 mL) at 0° C. was added 1 M BBr₃ in DCM (0.30 mL, 0.30 mmol) and the reaction mixture was stirred at 0° C. for 1 hour and was then poured onto 5 wt % aqueous solution of NaHCO₃ (30 mL). The organics were extracted with DCM (3×20 mL), combined, passed through a hydrophobic frit and concentrated in vacuo. Purification was performed by basic reverse phase FCC (30 g C18 SiO₂, 10-100% MeCN (0.1% NH₃) in water (0.1% NH₃)) followed by lyophilization to afford the title compound as a white solid (64 mg, 40% yield, 97% purity).

¹H NMR (400 MHz, DMSO) δ 11.03-9.63 (m, 2H), 7.93 (d, J=2.1 Hz, 1H), 7.66-7.56 (m, 1H), 7.53-7.50 (m, 1H), 7.46-7.38 (m, 2H), 7.32-7.23 (m, 1H), 7.01 (dd, J=7.7, 1.4 Hz, 1H), 6.66-6.56 (m, 1H), 4.62-4.43 (m, 1H), 4.30-4.20 (m, 1H), 3.06-2.98 (m, 1H), 2.91-2.75 (m, 1H).

LCMS: m/z=464.0/466.0 [M−H]−, (ESI−), RT=4.06, Method A

### Example 22—Synthesis of 18-chloro-24,26-difluoro-19-hydroxy-14-oxa-3,21lambda6-dithia-22-azapentacyclo[21.3.1.1^{16,20}.0^{2,10}.0^{4,9}]octacosa-1(27),2(10),4,6,8,16,18,20(28),23,25-decaene-15,21,21-trione (Compound 22)

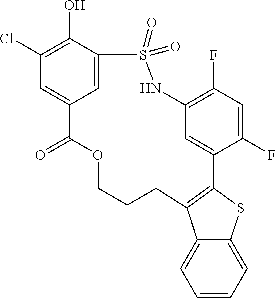 [SMILES: O=C1OCCCc2c(sc3ccccc23)-c2cc(c(F)cc2F)NS(=O)(=O)c2cc1cc(Cl)c2O]

### Step 1

To a solution of methyl 3-(benzothiophen-3-yl)propanoate (98%, 1.55 g, 6.90 mmol) in DCM (21 mL) at 0° C. was added a solution of bromine (0.37 mL, 7.22 mmol) in DCM (4.5 mL) dropwise over 5 minutes. The reaction mixture was stirred at 0° C. for 1.75 hours and then to the reaction mixture was added sat. aq. Na₂SO₃ (20 mL), NaHCO₃ (20 mL of a saturated aqueous solution) and water (5 mL). The organics were separated and the aqueous was extracted with DCM (3×15 mL). The organics were combined, passed through a hydrophobic frit and concentrated in vacuo which gave methyl 3-(2-bromobenzothiophen-3-yl)propanoate (97.0%) (2.09 g, 98% yield, 97% purity) as a pale-yellow oil.

¹H NMR (400 MHz, CDCl₃) δ 7.75-7.67 (m, 2H), 7.40-7.29 (m, 2H), 3.69 (s, 3H), 3.22-3.16 (m, 2H), 2.67-2.60 (m, 2H).

### Step 2

To a solution of methyl 3-(2-bromobenzothiophen-3-yl)propanoate (97%, 1.89 g, 6.13 mmol) in anhydrous THE (44 mL) and anhydrous MeOH (5.8 mL) at 0° C. was added lithium tetrahydroborate (430 mg, 19.7 mmol) portion wise over 1 minute. The resulting solution was stirred at r.t. for 30 minutes, before further lithium tetrahydroborate (50 mg, 2.30 mmol) was added and the mixture was stirred for 10 minutes at r.t. The reaction mixture was cooled to 0° C., and quenched by slow addition of HCl (20 mL of a 1 M aqueous solution). The organics were diluted with EtOAc (150 mL) and then separated. The organics were washed with NaHCO₃ (50 mL of a saturated aqueous solution) and then brine. The combined aqueous was extracted with EtOAc (70 mL). The organics were combined, dried over MgSO₄, filtered and concentrated in vacuo which gave 3-(2-bromobenzothiophen-3-yl)propan-1-ol (1.97 g, 97% yield, 90% purity) as a pale-yellow oil.

¹H NMR (400 MHz, DMSO) δ 7.94-7.90 (m, 1H), 7.83-7.79 (m, 1H), 7.44-7.35 (m, 2H), 4.59 (t, J=5.1 Hz, 1H), 3.49-3.43 (m, 2H), 2.89-2.83 (m, 2H), 1.75-1.66 (m, 2H).

### Step 3

To a solution of 3-(2-bromobenzothiophen-3-yl)propan-1-ol (90%, 1.97 g, 6.54 mmol) in anhydrous DMF (15 mL) was added imidazole (900 mg, 13.2 mmol) and tert-butyl(chloro)dimethylsilane (1.33 g, 8.82 mmol) and the reaction was stirred at r.t. for 1.5 hours. TBSCl (200 mg) was then added and the reaction mixture was stirred at r.t. for 16 hours. The reaction mixture was diluted with EtOAc (120 mL) and was washed with water (150 mL), then brine (2×150 mL), dried over MgSO₄, filtered and concentrated in vacuo. The residue was purified by FCC (50 g SiO₂ column, 0-10% EtOAc in heptane) gave 3-(2-bromobenzothiophen-3-yl)propoxy-tert-butyl-dimethylsilane (2.22 g, 5.47 mmol, 84% yield, 95% purity) as a colorless oil.

¹H NMR (400 MHz, CDCl₃) δ 7.74-7.69 (m, 2H), 7.37-7.28 (m, 2H), 3.69 (t, J=6.1 Hz, 2H), 2.97-2.89 (m, 2H), 1.88-1.79 (m, 2H), 0.94 (s, 9H), 0.08 (s, 6H).

### Step 4

A solution of 2,4-difluoro-5-(4,4,5,5-tetramethyl-1,3,2-dioxaborolan-2-yl)aniline (88%, 1.04 g, 3.57 mmol) and 3-(2-bromobenzothiophen-3-yl)propoxy-tert-butyl-dimethylsilane (95%, 1.65 g, 4.07 mmol) in 1,4-dioxane (30 mL) and water (3 mL) was sparged with nitrogen for 10 minutes. To the reaction mixture was added potassium carbonate (1.08 g, 7.81 mmol) and Pd(dppf)Cl₂ (262 mg, 0.357 mmol) and the reaction mixture was heated at 100° C. for 16 hours. The reaction mixture was allowed to cool to r.t. and then filtered through a pad of Celite topped with silica, washing with EtOAc. The filtrate was concentrated in vacuo and the residue was purified by FCC (50 g SiO₂ column, 0-25% acetone in heptane) gave 5-[3-[3-[tert-butyl(dimethyl)silyl]oxypropyl]benzothiophen-2-yl]-2,4-difluoro-aniline (1.50 g, 73% yield, 75% purity) as a yellow oil.

¹H NMR (400 MHz, DMSO) δ 7.98-7.92 (m, 1H), 7.87-7.82 (m, 1H), 7.47-7.35 (m, 2H), 7.17 (dd, J=11.2, 9.7 Hz, 1H), 6.82 (dd, J=9.8, 7.6 Hz, 1H), 5.19 (s, 2H), 3.55 (t, J=6.0 Hz, 2H), 2.79-2.73 (m, 2H), 1.71-1.63 (m, 2H), 0.81 (s, 9H), −0.03 (s, 6H).

### Step 5

Intermediate 1 (50%, 1.56 g, 2.61 mmol) and 5-[3-[3-[tert-butyl(dimethyl)silyl]oxypropyl]benzothiophen-2-yl]-2,4-difluoro-aniline (75%, 1.50 g, 2.59 mmol) were dissolved in anhydrous pyridine (14 mL) and the mixture was heated at 50° C. for 2 hours. The reaction mixture was allowed to cool to r.t, and was diluted with 1 M aq. HCl (230 mL) and extracted with EtOAc (3×50 mL). The combined organic extracts were washed with brine (70 mL), dried over MgSO₄, filtered and concentrated in vacuo. The residue was purified by FCC (50 g SiO₂ column, 0-80% TBME in heptane) to afford methyl 3-[[5-[3-[3-[tert-butyl(dimethyl)silyl]oxypropyl]benzothiophen-2-yl]-2,4-difluoro-phenyl]sulfamoyl]-5-chloro-4-methoxybenzoate (1.12 g, 46% yield, 75% purity) as a yellow oil.

¹H NMR (400 MHz, DMSO) δ 10.54 (s, 1H), 8.25 (d, J=2.1 Hz, 1H), 8.19 (d, J=2.1 Hz, 1H), 8.00-7.93 (m, 1H), 7.89-7.84 (m, 1H), 7.52-7.39 (m, 3H), 7.35-7.29 (m, 1H), 3.97 (s, 3H), 3.86 (s, 3H), 3.49 (t, J=6.0 Hz, 2H), 2.73-2.66 (m, 2H), 1.64-1.57 (m, 2H), 0.78 (s, 9H), −0.06 (s, 6H).

LCMS: m/z=694.3/696.3 [M−H]−, (ESI−), RT=1.02, Method B

### Step 6

To a solution of methyl 3-[[5-[3-[3-[tert-butyl(dimethyl)silyl]oxypropyl]benzothiophen-2-yl]-2,4-difluoro-phenyl]sulfamoyl]-5-chloro-4-methoxybenzoate (75%, 1.12 g, 1.21 mmol) in MeOH (7 mL) at r.t. was added 4-methylbenzenesulfonic acid hydrate (1:1) (26 mg, 0.137 mmol). The reaction mixture was stirred for 1 hour at r.t, and was then concentrated in vacuo. The residue was purified by FCC (25 g SiO₂ column, 0-100% EtOAc in heptane followed by 0-30% MeOH in EtOAc) to afford methyl 3-chloro-5-[[2,4-difluoro-5-[3-(3-hydroxypropyl)benzothiophen-2-yl]phenyl]sulfamoyl]-4-methoxybenzoate (758 mg, 100% yield, 93% purity) as a white solid.

LCMS: m/z=580.0/582.0 [M−H]−, (ESI−), RT=1.09, Method B

### Step 7

To a solution of methyl 3-chloro-5-[[2,4-difluoro-5-[3-(3-hydroxypropyl)benzothiophen-2-yl]phenyl]sulfamoyl]-4-methoxybenzoate (93%, 421 mg, 0.673 mmol) in THE (6.5 mL) at r.t. was added 2 M aq. sodium hydroxide (2.0 mL, 4.00 mmol). The reaction mixture was stirred at r.t. for 15.5 hours and then the organics were concentrated in vacuo. To the flask was added 1 M aq. HCl (15 mL) and the organics were extracted with DCM (4×15 mL), passed through a hydrophobic frit and concentrated in vacuo to afford 3-chloro-5-[[2,4-difluoro-5-[3-(3-hydroxypropyl)benzothiophen-2-yl]phenyl]sulfamoyl]-4-methoxybenzoic acid (508 mg, 100% yield, 75% purity) as a yellow oil.

LCMS: m/z=566.0/568.0 [M−H]−, (ESI−), RT=0.97, Method B

### Step 8

To a solution of 3-chloro-5-[[2,4-difluoro-5-[3-(3-hydroxypropyl)benzothiophen-2-yl]phenyl]sulfamoyl]-4-methoxybenzoic acid (75%, 508 mg, 0.671 mmol) in anhydrous DCM (22 mL) was added 4-dimethylaminopyridine (17 mg, 0.139 mmol) and N,N-dicyclohexylcarbodiimide (280 mg, 1.36 mmol). The reaction mixture was stirred at r.t. for 2 hours and was then concentrated in vacuo. The residue was purified by FCC (25 g SiO₂ column, 0-60% EtOAc in heptane) followed by FCC (25 g SiO₂ column, 0-100% Et₂O in heptane) to afford 18-chloro-24,26-difluoro-19-methoxy-21,21-dioxo-14-oxa-3,21λ6-dithia-22-azapentacyclo[21.3.1.116,20.02,10.04,9]octacosa-1(27),2(10),4,6,8,16,18,20(28),23,25-decaen-15-one (253 mg, 60% yield, 88% purity) as a white solid.

¹H NMR (400 MHz, DMSO) δ 10.79 (s, 1H), 8.17 (d, J=2.1 Hz, 1H), 8.04-7.97 (m, 2H), 7.82 (d, J=2.1 Hz, 1H), 7.66-7.60 (m, 1H), 7.52-7.43 (m, 2H), 7.41-7.35 (m, 1H), 4.32-4.27 (m, 2H), 4.07 (s, 3H), 2.90-2.83 (m, 2H), 1.97-1.88 (m, 2H).

LCMS: m/z=548.0/550.0 [M−H]−, (ESI−), RT=1.19, Method B

### Step 9

To a solution of 18-chloro-24,26-difluoro-19-methoxy-21,21-dioxo-14-oxa-3,21λ6-dithia-22-azapentacyclo[21.3.1.116,20.02,10.04,9]octacosa-1(27),2(10),4,6,8,16,18,20(28),23,25-decaen-15-one (87%, 80 mg, 0.127 mmol) in anhydrous DCM (4.0 mL) at −16° C. was added 1 M BBr₃ in DCM (0.70 mL, 0.70 mmol). The reaction mixture was allowed to gradually warm to 10° C. over 4 hours. To the reaction mixture was added sat. aq. NaHCO₃ (4 mL). The aqueous phase was extracted with DCM (3×4 mL), and the organic phases were combined, passed through a hydrophobic frit and concentrated in vacuo. The residue was purified by preparative HPLC (Method P1) to afford the title compound as a white solid (7 mg, 10% yield, 99% purity).

¹H NMR (500 MHz, DMSO) δ 8.03-8.00 (m, 1H), 8.00-7.96 (m, 2H), 7.78 (d, J=2.2 Hz, 1H), 7.65-7.58 (m, 1H), 7.52-7.46 (m, 1H), 7.46-7.41 (m, 1H), 7.41-7.33 (m, 1H), 4.30-4.21 (m, 2H), 2.89-2.80 (m, 2H), 1.93-1.82 (m, 2H). 2H not observed.

LCMS: m/z=534.0/536.0 [M−H]−, (ESI−), RT=4.63, Method A

### Example 23—Synthesis of 15-chloro-21-fluoro-16-hydroxy-8,11-dioxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 23)

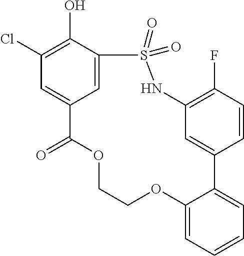 [SMILES: O=C1OCCOc2ccccc2-c2ccc(F)c(c2)NS(=O)(=O)c2cc1cc(Cl)c2O]

To a solution of 15-chloro-21-fluoro-16-methoxy-18,18-dioxo-8,11-dioxa-18a6-thia-19-azatetracyclo[18.3.1.113,17.02,7]pentacosa-1(24),2,4,6,13,15,17(25),20,22-nonaen-12-one (synthesized according to General Scheme 2, 92%, 180 mg, 0.347 mmol) in anhydrous DCM (3.0 mL) at 0° C. was added 1 M BBr₃ in DCM (1.5 mL, 1.5 mmol) and the reaction mixture was stirred at 0° C. for 1.5 hours. The reaction mixture was poured onto a 5 wt % aq. solution of NaHCO₃ (30 mL) at 0° C. The organics were extracted with DCM (3×20 mL), combined, dried over Na₂SO₄, filtered and concentrated in vacuo. The residue was purified by preparative HPLC (Method P1) to afford the title compound as a white solid (26 mg, 16% yield, 99% purity).

¹H NMR (500 MHz, DMSO) δ 11.64-9.76 (m, 2H), 8.04 (d, J=2.1 Hz, 1H), 7.67 (d, J=2.1 Hz, 1H), 7.42-7.33 (m, 2H), 7.25 (dd, J=7.5, 1.7 Hz, 1H), 7.22-7.11 (m, 3H), 7.08-7.03 (m, 1H), 4.40-4.35 (m, 2H), 4.30-4.24 (m, 2H).

LCMS: m/z=462.0/464.0 [M−H]−, (ESI−), RT=4.04, Method A

### Example 24—Synthesis of 4-bromo-15-chloro-21,23-difluoro-16-hydroxy-8,11-dioxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 24)

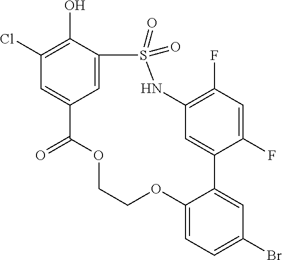 [SMILES: O=C1OCCOc2ccc(Br)cc2-c2cc(c(F)cc2F)NS(=O)(=O)c2cc1cc(Cl)c2O]

To a solution of 4-bromo-15-chloro-21,23-difluoro-16-methoxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (synthesized according to General Scheme 2, 95%, 100 mg, 0.165 mmol) in anhydrous DCM (1.5 mL) at 0° C. was added 1 M BBr₃ in DCM (0.80 mL, 0.80 mmol) and the reaction mixture was stirred at 0° C. for 1.5 hours. The reaction mixture was poured onto a 5 wt % aqueous solution of NaHCO₃ (30 mL) at 0° C. The organics were extracted with DCM (3×20 mL), combined, dried over Na₂SO₄, filtered and concentrated in vacuo. Purification was performed by preparative HPLC (Method P1) to afford the title compound as a white solid (26 mg, 25% yield, 90% purity).

¹H NMR (500 MHz, DMSO) δ 11.67-9.84 (m, 2H), 8.04 (d, J=2.1 Hz, 1H), 7.63 (dd, J=8.8, 2.6 Hz, 1H), 7.51 (d, J=2.5 Hz, 1H), 7.47 (d, J=2.2 Hz, 1H), 7.37-7.31 (m, 1H), 7.28-7.20 (m, 1H), 7.17 (d, J=8.8 Hz, 1H), 4.36-4.28 (m, 4H).

LCMS: m/z=557.9/559.9/561.9 [M−H]−, (ESI−), RT=4.29, Method A

### Example 25—Synthesis of 15-chloro-21-hydroxy-16-methoxy-8,11-dioxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 25)

### Example 26—Synthesis of 15-chloro-16-hydroxy-21-methoxy-8,11-dioxa-18lambda6-thia-19-azatetracyclo[18.3.1.1^{13,17}.0^{2,7}]pentacosa-1(24),2,4,6,13,15,17(25),20,22-nonaene-12,18,18-trione (Compound 26)

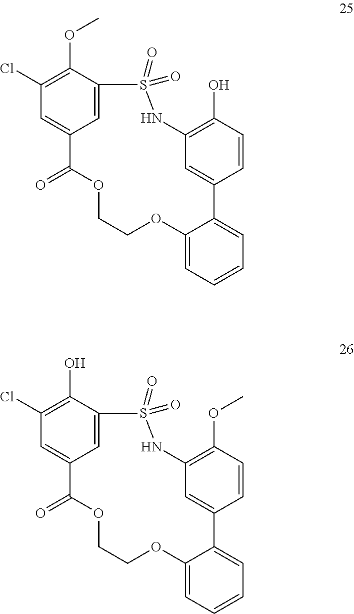 [SMILES: COc1c(Cl)cc2cc1S(=O)(=O)Nc1cc(ccc1O)-c1ccccc1OCCOC2=O ; COc1ccc2cc1NS(=O)(=O)c1cc(cc(Cl)c1O)C(=O)OCCOc1ccccc1-2]

To a solution of 15-chloro-16,21-dimethoxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (synthesized according to General Scheme 2, 95%, 130 mg, 0.252 mmol) in anhydrous DCM (13.0 mL), was added 1 M BBr₃ in DCM (0.26 mL, 0.26 mmol). The reaction mixture was stirred at 0° C. for 40 minutes. To the reaction mixture was added 1 M BBr₃ (0.13 mL, 0.13 mmol) and the reaction mixture was stirred at 0° C. for 1 hour. To the reaction mixture was added H₂O (65 mL) and then the organics were extracted with DCM (3×70 mL). The combined organics were passed through a hydrophobic frit and concentrated in vacuo. Purification was performed by preparative HPLC (Method P1) to afford the following products:

Compound 25: white solid (26 mg, 21% yield, 98% purity)

¹H NMR (500 MHz, DMSO) δ 9.50 (s, 2H), 8.12 (d, J=2.1 Hz, 1H), 7.58 (s, 1H), 7.36-7.31 (m, 1H), 7.27-7.23 (m, 1H), 7.23-7.21 (m, 1H), 7.17-7.14 (m, 1H), 7.07-7.02 (m, 1H), 7.03-6.98 (m, 1H), 6.60 (d, J=8.3 Hz, 1H), 4.41-4.35 (m, 2H), 4.28-4.22 (m, 2H), 4.03 (s, 3H).

LCMS: m/z=474.0/476.1 [M−H]−, (ESI−), RT=3.69, Method A

Compound 26: white solid (31 mg, 25% yield, 98% purity)

¹H NMR (500 MHz, DMSO) δ 10.89 (br s, 1H), 9.45 (s, 1H), 8.00 (d, J=2.1 Hz, 1H), 7.43 (d, J=2.2 Hz, 1H), 7.39-7.34 (m, 1H), 7.28-7.27 (m, 1H), 7.27-7.24 (m, 1H), 7.20-7.17 (m, 1H), 7.17-7.14 (m, 1H), 7.08-7.04 (m, 1H), 6.79 (d, J=8.5 Hz, 1H), 4.35-4.31 (m, 2H), 4.27-4.23 (m, 2H), 3.40 (s, 3H).

LCMS: m/z=474.0/476.0 [M−H]−, (ESI−), RT=4.08, Method A

### Example 27—Synthesis of 18-chloro-24,26-difluoro-19-hydroxy-14-oxa-21lambda6-thia-10,22-diazapentacyclo[21.3.1.1^{16,20}.0^{2,10}.0^{4,9}]octacosa-1(27),2,4,6,8,16,18,20(28),23,25-decaene-15,21,21-trione (Compound 27)

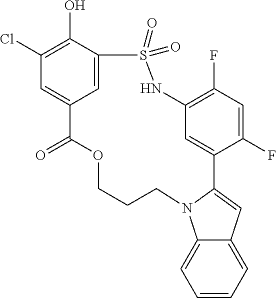 [SMILES: O=C1OCCCn2c(cc3ccccc32)-c2cc(c(F)cc2F)NS(=O)(=O)c2cc1cc(Cl)c2O]

### Step 1

To a solution of 2-iodo-1H-indole (95%, 775 mg, 3.03 mmol) in anhydrous DMF (30.2 mL) was added NaH in mineral oil (60%, 140 mg, 3.50 mmol) followed by 3-bromopropoxy-tert-butyl-dimethylsilane (96%, 822 μL, 3.19 mmol) at 0° C. The reaction mixture was allowed to warm up to r.t, and stirred for 3 hours. The reaction mixture was diluted with sat. aq. NaHCO₃ (80 mL) at 0° C., and extracted with EtOAc (2×80 mL). The combined organic layers were washed with brine (40 mL), dried over Na₂SO₄, filtered and concentrated under reduced pressure. The residue was purified by FCC (100 g SiO₂ column, 0-5% EtOAc in heptane) to afford tert-butyl-[3-(2-iodoindol-1-yl)propoxy]-dimethyl-silane (933 mg, 70% yield, 95% purity).

¹H NMR (500 MHz, DMSO) δ 7.49-7.45 (m, 2H), 7.10-7.06 (m, 1H), 7.02-6.98 (m, 1H), 6.77 (d, J=0.8 Hz, 1H), 4.28-4.22 (m, 2H), 3.62 (t, J=6.0 Hz, 2H), 1.87-1.80 (m, 2H), 0.90 (s, 9H), 0.05 (s, 6H).

### Step 2

A solution of tert-butyl-[3-(2-iodoindol-1-yl)propoxy]-dimethylsilane (95%, 413 mg, 0.945 mmol) and 2,4-difluoro-5-(4,4,5,5-tetramethyl-1,3,2-dioxaborolan-2-yl)aniline (85%, 300 mg, 1.00 mmol) in 1,4-dioxane (4 mL) and water (0.5 mL) was sparged with nitrogen for 10 minutes and then K₂CO₃ (260 mg, 1.88 mmol) and Pd(dppf)Cl₂ (70 mg, 0.0954 mmol) were added. The reaction mixture was heated at 100° C. under nitrogen for 18 hours. The mixture was cooled to room temperature, filtered through Celite and washed with EtOAc. The filtrate was concentrated in vacuo and the residue was purified by FCC (25 g SiO₂ column, 0-20% EtOAc in heptane) to afford 5-[1-[3-[tert-butyl(dimethyl)silyl]oxypropyl]indol-2-yl]-2,4-difluoro-aniline (188 mg, 45% yield, 95% purity) as a brown gum.

¹H NMR (400 MHz, DMSO) δ 7.56 (d, J=7.8 Hz, 1H), 7.51 (d, J=8.2 Hz, 1H), 7.21-7.13 (m, 2H), 7.09-7.03 (m, 1H), 6.82 (dd, J=9.9, 7.5 Hz, 1H), 6.44 (s, 1H), 5.17 (s, 2H), 4.13 (t, J=7.5 Hz, 2H), 3.45 (t, J=5.8 Hz, 2H), 1.72-1.62 (m, 2H), 0.79 (s, 9H), −0.06 (s, 6H).

### Step 3

Intermediate 1 (50%, 263 mg, 0.440 mmol) and 5-[1-[3-[tert-butyl(dimethyl)silyl]oxypropyl]indol-2-yl]-2,4-difluoro-aniline (95%, 188 mg, 0.429 mmol) were dissolved in anhydrous pyridine (2.3 mL) and the mixture was heated at 50° C. for 1 hour. The reaction mixture was diluted with 1 M aq. HCl (5 mL) and extracted with EtOAc (3×10 mL). The combined organic extracts were washed with brine (10 mL), dried over Na₂SO₄, filtered and concentrated under reduced pressure. The residue was purified by FCC (25 g SiO₂ column, 0-20% EtOAc in heptane) to afford methyl 3-[[5-[1-[3-[tert-butyl(dimethyl)silyl]oxypropyl]indol-2-yl]-2,4-difluoro-phenyl]sulfamoyl]-5-chloro-4-methoxybenzoate (161 mg, 50% yield, 90% purity) as a brown gum.

¹H NMR (400 MHz, DMSO) δ 10.55 (s, 1H), 8.24 (d, J=2.1 Hz, 1H), 8.19 (d, J=2.1 Hz, 1H), 7.58 (d, J=7.8 Hz, 1H), 7.52 (d, J=8.3 Hz, 1H), 7.50-7.43 (m, 1H), 7.35 (t, J=8.2 Hz, 1H), 7.22-7.16 (m, 1H), 7.11-7.05 (m, 1H), 6.47 (s, 1H), 4.10-4.04 (m, 2H), 3.98 (s, 3H), 3.85 (s, 3H), 3.39-3.34 (m, 2H), 1.56 (q, J=6.6 Hz, 2H), 0.76 (s, 9H), −0.10 (s, 6H).

LCMS: m/z=679.2/681.2 [M+H]+, (ESI+), RT=0.99, Method B

### Step 4

To a solution of methyl 3-[[5-[1-[3-[tert-butyl(dimethyl)silyl]oxypropyl]indol-2-yl]-2,4-difluoro-phenyl]sulfamoyl]-5-chloro-4-methoxybenzoate (90%, 315 mg, 0.413 mmol) in anhydrous THE (3.3 mL) at r.t. was added 1 M TBAF in THE (1.5 mL, 1.50 mmol) and the reaction mixture was stirred at r.t. for 60 hours. The reaction mixture was diluted with 1 M aq. HCl (5 mL) and extracted with EtOAc (3×10 mL). The combined organic extracts were washed with brine (10 mL), dried over Na₂SO₄, filtered and concentrated under reduced pressure. The residue was purified by FCC (25 g SiO₂ column, 0-100% EtOAc in heptane) to afford methyl 3-chloro-5-[[2,4-difluoro-5-[1-(3-hydroxypropyl)indol-2-yl]phenyl]sulfamoyl]-4-methoxybenzoate (85.0%) (167 mg, 61% yield, 88% purity) as a pale-yellow gum.

LCMS: m/z=563.1/565.0 [M−H]−, (ESI−), RT=1.06, Method B

### Step 5

Methyl 3-chloro-5-[[2,4-difluoro-5-[1-(3-hydroxypropyl)indol-2-yl]phenyl]sulfamoyl]-4-methoxybenzoate (88%, 165 mg, 0.257 mmol) was dissolved in THE (5.4 mL) and MeOH (0.52 mL), and 2 M aq. sodium hydroxide (1.2 mL, 2.40 mmol) was added. The resulting solution was stirred at r.t. for 1 hour. 6.0 M aq. NaOH (0.5 mL) was added and the reaction was stirred for another 1 hour at r.t. THE was removed under reduced pressure and the remaining aqueous solution was acidified with 1 M aq HCl (15 mL) to pH=1, and extracted with DCM (3×15 mL) before the combined organic phases were washed with brine (30 mL), passed through a phase separator and concentrated under reduced pressure to give 3-chloro-5-[[2,4-difluoro-5-[1-(3-hydroxypropyl)indol-2-yl]phenyl]sulfamoyl]-4-methoxybenzoic acid (145 mg, 99% yield, 97% purity) as a pale yellow solid.

LCMS: m/z=549.0/551.0 [M−H]−, (ESI−), RT=0.93, Method B

### Step 6

To a solution of 3-chloro-5-[[2,4-difluoro-5-[1-(3-hydroxypropyl)indol-2-yl]phenyl]sulfamoyl]-4-methoxybenzoic acid (97%, 150 mg, 0.264 mmol) in anhydrous DCM (8.8 mL) was added 4-dimethylaminopyridine (7.0 mg, 0.0573 mmol) and N,N-dicyclohexylcarbodiimide (125 mg, 0.606 mmol). The reaction mixture was stirred at r.t. for 20 hours and was then concentrated in vacuo. The residue was purified by FCC (25 g SiO₂ column, 0-100% EtOAc in heptane) to afford 18-chloro-24,26-difluoro-19-methoxy-21,21-dioxo-14-oxa-21λ6-thia-10,22-diazapentacyclo[21.3.1.116,20.02,10.04,9]octacosa-1(27),2,4,6,8,16,18,20(28),23,25-decaen-15-one (107 mg, 68% yield, 90% purity) as a white solid.

¹H NMR (400 MHz, DMSO) δ 10.79 (s, 1H), 8.17-8.13 (m, 1H), 7.77-7.71 (m, 1H), 7.73-7.64 (m, 1H), 7.66-7.58 (m, 2H), 7.40-7.31 (m, 1H), 7.28-7.20 (m, 1H), 7.15-7.07 (m, 1H), 6.61 (s, 1H), 4.27-4.20 (m, 2H), 4.20-4.11 (m, 2H), 4.07 (s, 3H), 2.05-1.95 (m, 2H).

LCMS: m/z=531.1/533.0 [M−H]−, (ESI−), RT=1.12, Method B

### Step 7

To a solution of 18-chloro-24,26-difluoro-19-methoxy-21,21-dioxo-14-oxa-21λ6-thia-10,22-diazapentacyclo[21.3.1.116,20.02,10.04,9]octacosa-1(27),2,4,6,8,16,18,20(28),23,25-decaen-15-one (90%, 95 mg, 0.160 mmol) in DCM-anhydrous (9.5 mL) was added 1 M BBr₃ in DCM (1.0 mL, 1.00 mmol) and the reaction mixture was stirred at 0° C. for 1.5 hour. To the reaction mixture was added 1 M BBr₃ in DCM (0.50 mL, 0.50 mmol) and the reaction was stirred at 0° C. for 1 hour. To the reaction mixture was added H₂O (50 mL). The aqueous phase was extracted with DCM (3×50 mL), and the organic phases were combined, passed through a hydrophobic frit and concentrated in vacuo. 20 wt % of the crude material was purified by preparative HPLC (Method P1). The remaining 80 wt % of crude material was purified by reverse-phase FCC (12 g of C18 SiO₂, eluted with 0.1% NH₃ in MeCN in 0.1% NH₃ in H₂O, 10-100%). The resultant products were combined and the majority of MeCN was removed in vacuo. The organics were extracted from the remaining aqueous phase using DCM (3×50 mL), combined, passed through a hydrophobic frit and concentrated in vacuo which gave the title compound as an off-white solid (14 mg, 16% yield, 96% purity).

¹H NMR (500 MHz, DMSO) δ 7.99-7.94 (m, 1H), 7.69-7.67 (m, 1H), 7.67-7.64 (m, 1H), 7.64-7.59 (m, 2H), 7.39-7.33 (m, 1H), 7.26-7.21 (m, 1H), 7.14-7.08 (m, 1H), 6.59 (s, 1H), 4.24-4.19 (m, 2H), 4.17-4.12 (m, 2H), 2.01-1.93 (m, 2H).

LCMS: m/z=517.0/519.0 [M−H]−, (ESI−), RT=4.34, Method A

### Example 28—Synthesis of 15-chloro-21,23-difluoro-16-hydroxy-12,18,18-trioxo-8,11-dioxa-18lambda6-thia-19-azatetracyclo[18.3.1.1^{13,17}.0^{2,7}]pentacosa-1(24),2,4,6,13,15,17(25),20,22-nonaene-4-carbonitrile (Compound 28)

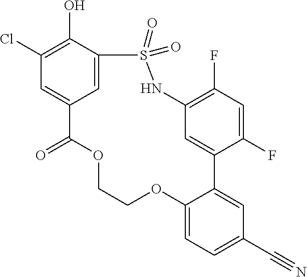 [SMILES: N#Cc1ccc2c(c1)-c1cc(c(F)cc1F)NS(=O)(=O)c1cc(cc(Cl)c1O)C(=O)OCCO2]

To a solution of 15-chloro-21,23-difluoro-16-methoxy-12,18,18-trioxo-8,11-dioxa-18λ6-thia-19-azatetracyclo[18.3.1.113,17.02,7]pentacosa-1(24),2,4,6,13,15,17(25),20,22-nonaene-4-carbonitrile (synthesized according to General Scheme 2, 92%, 100 mg, 0.177 mmol) in anhydrous DCM (1.6 mL) at 0° C. was added 1 M BBr₃ in DCM (0.80 mL, 0.800 mmol) and the reaction mixture was stirred at 0° C. for 3 hours. The reaction mixture was poured onto a saturated aqueous solution of NaHCO₃ (30 mL). The organics were extracted with DCM (3×30 mL), combined, dried over Na₂SO₄, filtered and concentrated in vacuo. Purification was performed by acidic prep HPLC (standard method). The product containing fractions were combined, concentrated in vacuo and dried in the vacuum oven (40° C.) which gave the title compound as a white solid (35 mg, 39% yield, 100% purity).

¹H NMR (500 MHz, DMSO) δ 8.07-7.99 (m, 1H), 7.96 (dd, J=8.6, 2.2 Hz, 1H), 7.83 (d, J=2.1 Hz, 1H), 7.47 (d, J=2.2 Hz, 1H), 7.41-7.33 (m, 2H), 7.32-7.22 (m, 1H), 4.42-4.38 (m, 2H), 4.37-4.34 (m, 2H).

LCMS: m/z=505.0/507.0 [M−H]−, (ESI−), RT=3.81, Method A

### Example 29—Synthesis of 16-chloro-5,22,24-trifluoro-17-hydroxy-8,12-dioxa-19lambda6-thia-20-azatetracyclo[19.3.1^{1,4,18}.0^{2,7}]hexacosa-1(25),2,4,6,14,16,18(26),21,23-nonaene-13,19,19-trione (Compound 29)

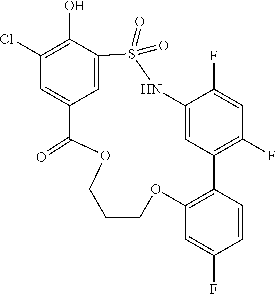 [SMILES: O=C1OCCCOc2cc(F)ccc2-c2cc(c(F)cc2F)NS(=O)(=O)c2cc1cc(Cl)c2O]

To a solution of 16-chloro-5,22,24-trifluoro-17-methoxy-19,19-dioxo-8,12-dioxa-19λ6-thia-20-azatetracyclo[19.3.1.114,18.02,7]hexacosa-1(24),2(7),3,5,14,16,18(26),21(25),22-nonaen-13-one (synthesized according to General Scheme 2, 93%, 230 mg, 0.405 mmol) in anhydrous DCM (4.3 mL) at 0° C. was added 1 M BBr₃ in DCM (2.0 mL, 2.0 mmol) and the reaction mixture was stirred for 1 hour at r.t. The reaction mixture was added dropwise into an ice-cooled sat. aq. NaHCO₃ solution. The organics were extracted with DCM (3×10 mL), combined, passed through a hydrophobic frit and concentrated in vacuo. The crude material was purified by reverse phase column chromatography (12 g C18 silica, 10-100% MeCN in water (0.1% NH₃)), followed by preparative HPLC (Method P3) to afford the title compound (11 mg, 5% yield, 98% purity) as a white solid.

¹H NMR (400 MHz, DMSO) δ 7.90 (br s, 1H), 7.36 (d, J=2.2 Hz, 1H), 7.24-7.12 (m, 2H), 7.02-6.94 (m, 1H), 6.93-6.87 (m, 1H), 6.88-6.78 (m, 1H), 4.42-4.33 (m, 2H), 4.14-4.06 (m, 2H), 2.02-1.91 (m, 2H). 2H not observed

LCMS: m/z=512.1/514.1 [M−H]−, (ESI−), RT=3.93, Method A

### Example 30—Synthesis of 14-chloro-20,22-difluoro-15-hydroxy-17lambda6-thia-10,18-diazatetracyclo[17.3.1.1^{12,16}.0^{2,7}]tetracosa-1(23),2,4,6,12,14,16(24),19,21-nonaene-11,17,17-trione (Compound 30)

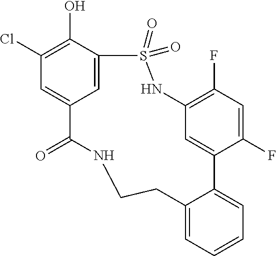 [SMILES: O=C1NCCc2ccccc2-c2cc(c(F)cc2F)NS(=O)(=O)c2cc1cc(Cl)c2O]

To a solution of 14-chloro-20,22-difluoro-15-methoxy-17,17-dioxo-17λ6-thia-10,18-diazatetracyclo[17.3.1.112,16.02,7]tetracosa-1(23),2,4,6,12,14,16(24),19,21-nonaen-11-one (synthesized according to General Scheme 1, 75%, 270 mg, 0.423 mmol) in anhydrous DCM (4 mL) at 0° C. was added 1 M BBr₃ in DCM (2.1 mL, 2.1 mmol) and the reaction mixture was stirred at 0° C. for 5 minutes and then at r.t. for 20 hour. To the reaction mixture at 0° C. was added 1 M BBr₃ in DCM (0.85 mL, 0.85 mmol) and then the reaction mixture was stirred at r.t. for 1 hour. The reaction mixture was poured onto ice-water (60 mL). The aqueous phase was extracted with DCM (3×40 mL), and the organic phases were combined, passed through a hydrophobic frit and concentrated in vacuo. Purification was performed by preparative HPLC (Method P1) followed by FCC (10 g SiO₂ column, 0-20% MeOH in DCM) to afford the title compound (32 mg, 16% yield, 99% purity) as an off-white solid.

¹H NMR (400 MHz, DMSO) δ 8.00-7.89 (m, 1H), 7.78 (s, 1H), 7.51-7.40 (m, 3H), 7.39-7.32 (m, 1H), 7.26-7.16 (m, 1H), 6.91 (d, J=7.7 Hz, 1H), 6.48-6.32 (m, 1H), 3.76-3.57 (m, 1H), 2.89-2.77 (m, 1H), 2.73-2.59 (m, 1H).

LCMS: m/z=463.0/465.0 [M−H]−, (ESI−), RT=3.46, Method A

### Example 31—Synthesis of 15-chloro-4-cyclopropyl-21,23-difluoro-16-hydroxy-8,11-dioxa-18lambda6-thia-19-azatetracyclo[18.3.1.1^{13,17}.0^{2,7}]pentacosa-1(23),2,4,6,13(25),14,16,20(24),21-nonaene-12,18,18-trione (Compound 31)

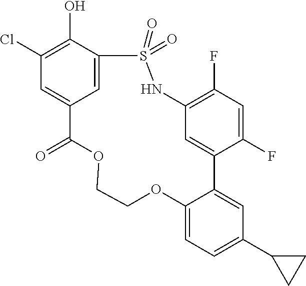 [SMILES: O=C1OCCOc2ccc(C3CC3)cc2-c2cc(c(F)cc2F)NS(=O)(=O)c2cc1cc(Cl)c2O]

A sealed mixture of 15-chloro-4-cyclopropyl-21,23-difluoro-16-methoxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (synthesized according to General Scheme 2, 100%, 100 mg, 0.187 mmol), iodocyclohexane (400 mg, 1.90 mmol) and anhydrous DMF (3 mL) was heated at 150° C. for 3 hours. The reaction mixture was allowed to cool to r.t, and was then added to water (50 mL). The organics were extracted with EtOAc (3×50 mL), combined, washed with brine (100 mL), dried over Na₂SO₄, filtered and concentrated in vacuo.

Purification was performed by preparative HPLC (Method P1) to afford the title compound (27 mg, 27% yield, 96% purity) as a white solid.

¹H NMR (400 MHz, DMSO) δ 8.03 (d, J=2.1 Hz, 1H), 7.45 (d, J=2.2 Hz, 1H), 7.36-7.26 (m, 1H), 7.23-7.11 (m, 2H), 7.06 (d, J=8.5 Hz, 1H), 6.97 (d, J=2.3 Hz, 1H), 4.35-4.29 (m, 2H), 4.27-4.20 (m, 2H), 1.92 (tt, J=8.5, 5.1 Hz, 1H), 0.94-0.86 (m, 2H), 0.74-0.62 (m, 2H).

LCMS: m/z=520.2/522.1 [M−H]−, (ESI−), RT=4.39, Method A

### Example 32—Synthesis of 15-chloro-21,23-difluoro-16-hydroxy-8,11-dioxa-18lambda6-thia-6,19-diazatetracyclo[18.3.1.1^{13,17}.0^{2,7}]pentacosa-1(24),2,4,6,13,15,17(25),20,22-nonaene-12,18,18-trione (Compound 32)

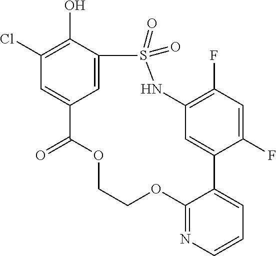 [SMILES: O=C1OCCOc2ncccc2-c2cc(c(F)cc2F)NS(=O)(=O)c2cc1cc(Cl)c2O]

To a solution of 15-chloro-21,23-difluoro-16-methoxy-18,18-dioxo-8,11-dioxa-18λ6-thia-6,19-diazatetracyclo[18.3.1.113,17.02,7]pentacosa-1(24),2,4,6,13,15,17(25),20,22-nonaen-12-one (synthesized according to General Scheme 2, 95%, 110 mg, 0.210 mmol) in anhydrous DCM (2 mL) at 0° C. was added 1 M BBr₃ in DCM (1.1 mL, 1.1 mmol). The reaction mixture was stirred at 0° C. for 10 minutes and then at r.t. for 3 hours. The reaction mixture was poured into sat. aq. solution of NaHCO₃ (30 mL). The aqueous phase was extracted with DCM (3×30 mL), and the organic phases were combined, passed through a hydrophobic frit and concentrated in vacuo. Purification was performed by preparative HPLC (Method P1) to afford the title compound (22 mg, 17% yield, 99% purity) as an off-white solid.

¹H NMR (500 MHz, DMSO) δ 8.27 (dd, J=5.0, 1.9 Hz, 1H), 8.06-7.98 (m, 1H), 7.75 (dd, J=7.3, 1.9 Hz, 1H), 7.68 (d, J=2.2 Hz, 1H), 7.40-7.26 (m, 2H), 7.15 (dd, J=7.3, 5.0 Hz, 1H), 4.51-4.44 (m, 2H), 4.44-4.36 (m, 2H).

LCMS: m/z=481.0/483.0 [M−H]−, (ESI−), RT=3.85, Method A

### Example 33—Synthesis of 16-chloro-22,24-difluoro-17-hydroxy-19,19-dioxo-8-oxa-19λ6-thia-12,20-diazapentacyclo[19.3.1.110,12.114,18.02,7]heptacosa-1(25),2,4,6,14,16,18(26),21,23-nonaen-13-one (Compound 33)

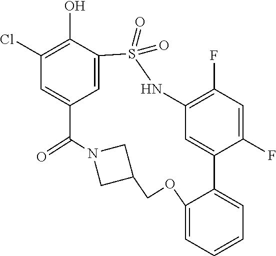 [SMILES: O=C1c2cc(Cl)c(O)c(c2)S(=O)(=O)Nc2cc(c(F)cc2F)-c2ccccc2OCC2CN1C2]

A solution of 16-chloro-22,24-difluoro-17-methoxy-19,19-dioxo-8-oxa-19λ6-thia-12,20-diazapentacyclo[19.3.1.110,12.114,18.02,7]heptacosa-1(25),2,4,6,14,16,18(26),21,23-nonaen-13-one (synthesized using a similar method to Example 1, 95% purity, 50 mg, 0.09 mmol) in anhydrous DCM (2 mL) was cooled to 0° C., before 1 M BBr₃ in DCM (0.40 mL, 0.40 mmol) was added, and the mixture was stirred at 40° C. for 20 h. The mixture was poured into ice cooled sat. aq. NaHCO₃ (30 mL) and DCM (30 mL) was then added. The layers were separated and the aqueous phase was extracted with DCM (4×30 mL). The aqueous was acidified to pH ˜1-2 with 2 M aq. HCl before it was extracted further with DCM (2×30 mL). The combined organic phases were dried over Na₂SO₄, filtered and concentrated. The residue was purified by preparative HPLC (Method P1) to afford the title compound as a white solid (14 mg, 30% Yield, 99% purity).

¹H NMR (400 MHz, DMSO) δ 10.77 (br. s, 1H), 7.67 (s, 1H), 7.54-7.43 (m, 1H), 7.41-7.30 (m, 2H), 7.23 (d, J=7.9 Hz, 1H), 7.20-7.05 (m, 3H), 4.44-4.27 (m, 1H), 4.15-3.85 (m, 2H), 3.70-3.52 (m, 2H), 3.23-3.02 (m, 1H), 2.95-2.72 (m, 1H).

LCMS: m/z=507.0/509.0 [M+H]+, (ESI+), RT=3.40, Method A

### Example 34—Synthesis of 12-chloro-4,6-difluoro-11-hydroxy-9,9-dioxo-2,16-dioxa-9λ6-thia-8-azatetracyclo[16.3.1.13,7.110,14]tetracosa-1(22),3,5,7(24),10(23),11,13,18,20-nonaen-15-one (Compound 34)

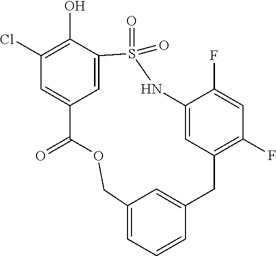 [SMILES: O=C1OCc2cccc(c2)Cc2cc(c(F)cc2F)NS(=O)(=O)c2cc1cc(Cl)c2O]

### Step 1

Methyl 3-iodobenzoate (500 mg, 1.87 mmol), 5-amino-2,4-difluorophenol (326 mg, 2.24 mmol), pyridine-2-carboxylic acid (23 mg, 0.187 mmol), copper(I) iodide (18 mg, 0.09 mmol) and tripotassium phosphate (794 mg, 3.74 mmol) were placed in a pressure vial. The vial was placed under nitrogen using three vacuum/nitrogen cycles. Anhydrous DMSO (4 mL) was added and the vial was heated at 80° C. for 24 hours. The reaction mixture was cooled to r.t., then filtered through a silica pad washing with EtOAc. To the filtrate was added H₂O (20 mL) and the layers were separated. The aqueous layer was extracted with further EtOAc (2×20 mL). The combined organic layers were washed with brine, dried (Na₂SO₄), filtered and concentrated. The residue was purified by FCC (25 g SiO₂ column, 0-100% EtOAc in heptane) to afford methyl 3-(5-amino-2,4-difluoro-phenoxy)benzoate (265 mg, 48% Yield, 94% purity) as a pale-yellow oil.

¹H NMR (400 MHz, DMSO) δ 7.72-7.67 (m, 1H), 7.57-7.49 (m, 1H), 7.38 (dd, J=2.8, 1.5 Hz, 1H), 7.32-7.24 (m, 2H), 6.64-6.56 (m, 1H), 5.22 (s, 2H), 3.84 (s, 3H).

### Step 2

To a solution of methyl 3-(5-amino-2,4-difluoro-phenoxy)benzoate (94% purity, 260 mg, 0.875 mmol) in anhydrous THF (6 mL) was added lithium borohydride (95 mg, 4.38 mmol) and the mixture was stirred at 66° C. for 1 hour. The mixture was cooled to r.t., then DCM (30 mL) and sat. aq. NaHCO₃ (30 mL) were added and the layers were separated. The aqueous was extracted with further DCM (2×20 mL). The combined organics were passed through a phase separator and then concentrated to afford [3-(5-amino-2,4-difluoro-phenoxy)phenyl]methanol (214 mg, 90% Yield, 92% purity) as a pale-yellow oil.

¹H NMR (500 MHz, DMSO) δ 7.32-7.26 (m, 1H), 7.26-7.17 (m, 1H), 7.05-6.98 (m, 1H), 6.89-6.85 (m, 1H), 6.84-6.75 (m, 1H), 6.58-6.50 (m, 1H), 5.22 (t, J=5.8 Hz, 1H), 5.15 (s, 2H), 4.46 (d, J=5.4 Hz, 2H).

### Step 3

Intermediate 1 (50% purity, 552 mg, 0.923 mmol) and [3-(5-amino-2,4-difluoro-phenoxy)phenyl]methanol (92% purity, 210 mg, 0.769 mmol) were dissolved in anhydrous pyridine (4 mL) and the mixture was stirred at 50° C. for 1 hour. The reaction mixture was allowed to cool to r.t., and was diluted with 1 M aq. HCl (30 mL), and extracted with EtOAc (3×30 mL). The combined organic extracts were washed with brine (50 mL), dried over Na₂SO₄, filtered and concentrated under reduced pressure. The residue was purified by FCC (50 g SiO₂ column, 0-100% EtOAc in heptane) to afford methyl 3-chloro-5-[[2,4-difluoro-5-[3-(hydroxymethyl)phenoxy]phenyl]sulfamoyl]-4-methoxy-benzoate (270 mg, 65% Yield, 95% purity) as a pale-yellow oil.

¹H NMR (400 MHz, DMSO) δ 10.44 (s, 1H), 8.23 (d, J=2.1 Hz, 1H), 8.11 (d, J=2.1 Hz, 1H), 7.57-7.47 (m, 1H), 7.32-7.23 (m, 1H), 7.10-7.04 (m, 1H), 6.95-6.85 (m, 2H), 6.73-6.65 (m, 1H), 5.23 (t, J=5.7 Hz, 1H), 4.46 (d, J=5.5 Hz, 2H), 3.93 (s, 3H), 3.87 (s, 3H).

### Step 4

Methyl 3-chloro-5-[[2,4-difluoro-5-[3-(hydroxymethyl)phenoxy]phenyl]sulfamoyl]-4-methoxy-benzoate (95% purity, 270 mg, 0.50 mmol) was dissolved in THE (4 mL) and 2 M aq. sodium hydroxide (1.2 mL, 2.50 mmol) was added. The reaction mixture was stirred at r.t. overnight. THE was removed under vacuum, the remaining aqueous phase was diluted with water (30 mL) and acidified with 1 M aq. HCl. The mixture was extracted with DCM (3×30 mL), the combined organic phases were passed through a phase separator and then concentrated to afford 3-chloro-5-[[2,4-difluoro-5-[3-(hydroxymethyl)phenoxy]phenyl]sulfamoyl]-4-methoxy-benzoic acid (240 mg, 87% Yield, 94% purity) as an off-white solid.

¹H NMR (400 MHz, DMSO) δ 13.66 (br. s, 1H), 10.40 (br. s, 1H), 8.19 (d, J=2.1 Hz, 1H), 8.10 (d, J=2.1 Hz, 1H), 7.57-7.45 (m, 1H), 7.32-7.25 (m, 1H), 7.08 (d, J=7.6 Hz, 1H), 6.95-6.87 (m, 2H), 6.69 (dd, J=8.1, 2.7 Hz, 1H), 5.22 (br. s, 1H), 4.53-4.38 (m, 2H), 3.92 (s, 3H).

LCMS: m/z=498.0/500.0 [M−H]−, (ESI−), RT=0.87, Method B

### Step 5

To a solution of 3-chloro-5-[[2,4-difluoro-5-[3-(hydroxymethyl)phenoxy]phenyl]sulfamoyl]-4-methoxy-benzoic acid (94% purity, 210 mg, 0.395 mmol) in anhydrous DCM (20 mL) was added DMAP (10 mg, 0.079 mmol) and DCC (163 mg, 0.790 mmol). The mixture was stirred at r.t. for 3 hours, then diluted with DCM (20 mL) and water (40 mL), and the layers were separated. The aqueous was extracted with DCM (2×30 mL), and the combined organic phases were passed through a phase separator and concentrated. The residue was purified by FCC (25 g SiO₂ column, 0-100% EtOAc in heptane) to afford 12-chloro-4,6-difluoro-11-methoxy-9,9-dioxo-2,16-dioxa-9λ6-thia-8-azatetracyclo[16.3.1.13,7.110,14]tetracosa-1(22),3,5,7(24),10(23),11,13,18,20-nonaen-15-one (145 mg, 72% Yield, 95% purity) as a white solid.

¹H NMR (400 MHz, DMSO) δ 10.79 (s, 1H), 8.25 (d, J=2.2 Hz, 1H), 8.08 (d, J=2.2 Hz, 1H), 7.54-7.46 (m, 1H), 7.46-7.36 (m, 2H), 7.24-7.17 (m, 1H), 7.16-7.06 (m, 1H), 6.44-6.29 (m, 1H), 5.39 (s, 2H), 4.08 (s, 3H).

LCMS: m/z=480.0/482.0 [M−H]−, (ESI−), RT=1.12, Method B

### Step 6

12-chloro-4,6-difluoro-11-methoxy-9,9-dioxo-2,16-dioxa-9λ6-thia-8-azatetracyclo[16.3.1.13,7.110,14]tetracosa-1(22),3,5,7(24),10(23),11,13,18,20-nonaen-15-one (100 mg, 0.21 mmol), anhydrous DMF (4 mL) and iodocyclohexane (268 μL, 2.08 mmol) were added to pressure vial. The vial was sealed and heated at 100° C. for 3 hours. The reaction mixture was allowed to cool to r.t., then sat. aq. NaHCO₃ (50 mL) was added, and subsequently extracted with EtOAc (3×50 mL). The combined organic extracts were washed with water (2×100 mL) then brine (100 mL), dried over Na₂SO₄, filtered and concentrated under reduced pressure. The residue was purified by FCC (10 g SiO₂ column, 0-20% MeOH in DCM) to afford the title compound (42 mg, 42% Yield, 98% purity) as a white solid.

¹H NMR (500 MHz, DMSO) δ 7.95 (d, J=2.5 Hz, 1H), 7.71-7.60 (m, 1H), 7.51-7.30 (m, 3H), 7.15 (dd, J=8.2, 2.6 Hz, 1H), 7.06 (d, J=7.5 Hz, 1H), 6.35-6.25 (m, 1H), 5.27 (s, 2H).

LCMS: m/z=466.0/468.0 [M−H]−, (ESI−), RT=4.36, Method A

### Example 35—Synthesis of 21,23-difluoro-15-methyl-18,18-dioxo-8,11-dioxa-18λ6-thia-15,19-diazatetracyclo[18.3.1.113,17.02,7]pentacosa-1(23),2(7),3,5,13,17(25),20(24),21-octaene-12,16-dione (Compound 35)

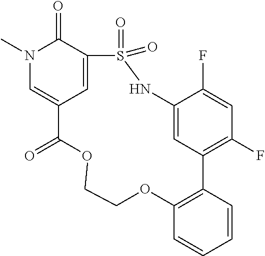 [SMILES: Cn1cc2cc(c1=O)S(=O)(=O)Nc1cc(c(F)cc1F)-c1ccccc1OCCOC2=O]

### Step 1

To a solution of methyl 5-bromo-1-methyl-6-oxo-pyridine-3-carboxylate (2.0 g, 7.97 mmol) in anhydrous 1,4-dioxane (75 mL) was added DIPEA (1.7 mL, 9.97 mmol) and the solution was sparged with nitrogen for 10 minutes. Phenylmethanethiol (1.1 mL, 9.44 mmol), Pd₂(dba)₃ (234 mg, 0.256 mmol), and Xantphos (281 mg, 0.486 mmol) were added, and the reaction mixture was heated at 100° C. for 16 hours. The reaction was cooled to r.t. diluted with water (100 mL), and extracted with EtOAc (3×100 mL). The combined organic layers were washed with brine (100 mL), dried over MgSO₄, filtered and concentrated in vacuo. The residue was purified by FCC (0-100% EtOAc in heptane), to afford methyl 5-benzylsulfanyl-1-methyl-6-oxo-pyridine-3-carboxylate (1.90 g, 82% Yield, 100% purity) as a yellow solid.

¹H NMR (400 MHz, DMSO) δ 8.37 (d, J=2.3 Hz, 1H), 7.59 (d, J=2.3 Hz, 1H), 7.46-7.37 (m, 2H), 7.35-7.30 (m, 2H), 7.28-7.22 (m, 1H), 4.18 (s, 2H), 3.78 (s, 3H), 3.54 (s, 3H).

LCMS: m/z=290.0 [M+H]+, (ESI+), RT=0.78, Method B

### Step 2

Methyl 5-benzylsulfanyl-1-methyl-6-oxo-pyridine-3-carboxylate (500 mg, 1.68 mmol) was dissolved in a mixture of MeCN (10 mL), acetic acid (0.5 mL) and water (0.5 mL). The resulting suspension was cooled to 0° C. in an ice bath. 1,3-Dichloro-5,5-dimethylhydantoin (673 mg, 3.42 mmol) was then added portion wise (over approximately 15 minutes) and the reaction mixture was stirred for 2 hours at 0° C. MeCN was removed under reduced pressure and DCM (20 mL) was added. The solution was cooled to 0° C., and sat. aq. NaHCO₃ solution (20 mL) was slowly added. The organic layer was separated, dried over MgSO₄, filtered, and concentrated under reduced pressure to afford methyl 5-chlorosulfonyl-1-methyl-6-oxo-pyridine-3-carboxylate (800 mg, 90% Yield, 50% purity) as a white solid.

LCMS: Does not ionize, RT=0.63, Method B

### Step 3

Methyl 5-chlorosulfonyl-1-methyl-6-oxo-pyridine-3-carboxylate (50% purity, 600 mg, 1.13 mmol) was added to a stirred solution of Intermediate 8 (95% purity, 450 mg, 1.13 mmol) in anhydrous pyridine (10 mL) at room temperature and the mixture was heated at 50° C. for 1 hour. The reaction mixture was cooled to r.t., quenched with 1 M aq. HCl (30 mL) and extracted with EtOAc (3×30 mL). The combined organic layers were washed with brine (30 mL), dried over MgSO₄, filtered, and concentrated in vacuo. The residue was purified by FCC (0-100% EtOAc in heptane), to afford methyl 5-[[5-[2-[2-[tert-butyl(dimethyl)silyl]oxyethoxy]phenyl]-2,4-difluoro-phenyl]sulfamoyl]-1-methyl-6-oxo-pyridine-3-carboxylate (520 mg, 0.837 mmol, 74% Yield, 98% purity) as a yellow glass.

¹H NMR (400 MHz, CDCl₃) δ 8.50 (d, J=2.5 Hz, 1H), 8.42 (d, J=2.5 Hz, 1H), 7.61 (br s, 1H), 7.54 (dd, J=8.9, 7.4 Hz, 1H), 7.35 (ddd, J=8.3, 7.4, 1.8 Hz, 1H), 7.18 (ddd, J=7.7, 1.8, 0.7 Hz, 1H), 7.02-6.97 (m, 2H), 6.79 (dd, J=10.1, 9.0 Hz, 1H), 4.04 (t, J=5.3 Hz, 2H), 3.89-3.82 (m, 5H), 3.72 (s, 3H), 0.81 (s, 9H), −0.07 (s, 6H).

### Step 4

p-Methylbenzenesulfonic acid hydrate (16 mg, 0.0837 mmol) was added to a stirred solution of methyl 5-[[5-[2-[2-[tert-butyl(dimethyl)silyl]oxyethoxy]phenyl]-2,4-difluoro-phenyl]sulfamoyl]-1-methyl-6-oxo-pyridine-3-carboxylate (98% purity, 520 mg, 0.837 mmol) in MeOH (10 mL) and the mixture was stirred for 1 hour. The reaction mixture was concentrated in vacuo and the residue was diluted with water (25 mL) and extracted with EtOAc (3×25 mL). The combined organic layers were washed with brine (25 mL), dried over MgSO₄, filtered and concentrated in vacuo. The residue was purified by FCC (0-100% EtOAc in heptane), to afford methyl 5-[[2,4-difluoro-5-[2-(2-hydroxyethoxy)phenyl]phenyl]sulfamoyl]-1-methyl-6-oxo-pyridine-3-carboxylate (350 mg, 82% Yield, 97% purity) as a yellow solid.

¹H NMR (400 MHz, CDCl₃) δ 8.52 (d, J=2.5 Hz, 1H), 8.43 (d, J=2.5 Hz, 1H), 7.68 (dd, J=8.9, 7.6 Hz, 1H), 7.62 (br s, 1H), 7.37 (ddd, J=8.3, 7.4, 1.8 Hz, 1H), 7.30-7.22 (m, 1H), 7.09-7.00 (m, 1H), 6.99 (dd, J=8.3, 1.1 Hz, 1H), 6.88-6.77 (m, 1H), 4.11-4.08 (m, 2H), 3.92-3.85 (m, 5H), 3.73 (s, 3H), 2.49 (td, J=6.6, 1.5 Hz, 1H).

LCMS: m/z=495.1 [M+H]+, (ESI+), RT=0.75, Method B

### Step 5

A solution of 2 M aq. sodium hydroxide (1.6 mL, 3.14 mmol) was added to a stirred solution of methyl 5-[[2,4-difluoro-5-[2-(2-hydroxyethoxy)phenyl]phenyl]sulfamoyl]-1-methyl-6-oxo-pyridine-3-carboxylate (97% purity, 320 mg, 0.628 mmol) in THE (10 mL) at r.t., and the mixture was stirred for 2 hours. THE was removed under vacuum, and the remaining aqueous phase was diluted with water (30 mL). The diluted aqueous phase was acidified with 1 M aq. HCl. The mixture was extracted with DCM (3×30 mL). The combined organic layers were washed with brine (30 mL), dried over MgSO₄, filtered and concentrated in vacuo to afford 5-[[2,4-difluoro-5-[2-(2-hydroxyethoxy)phenyl]phenyl]sulfamoyl]-1-methyl-6-oxo-pyridine-3-carboxylic acid (250 mg, 83% Yield, 100% purity) as a white solid.

¹H NMR (400 MHz, DMSO) δ 13.25 (br s, 1H), 9.96 (s, 1H), 8.82 (d, J=2.5 Hz, 1H), 8.24 (d, J=2.5 Hz, 1H), 7.38 (ddd, J=8.3, 7.3, 1.8 Hz, 1H), 7.33-7.19 (m, 2H), 7.17-7.06 (m, 2H), 7.06-6.97 (m, 1H), 4.66 (br s, 1H), 3.98 (t, J=5.4 Hz, 2H), 3.64-3.54 (m, 5H).

LCMS: m/z=481.1 [M+H]+, (ESI+), RT=0.66, Method B

### Step 6

DCC (86 mg, 0.416 mmol) was added to a stirred solution of 5-[[2,4-difluoro-5-[2-(2-hydroxyethoxy)phenyl]phenyl]sulfamoyl]-1-methyl-6-oxo-pyridine-3-carboxylic acid (100 mg, 0.208 mmol) and DMAP (5.1 mg, 0.042 mmol) in anhydrous DCM (10 mL) at room temperature, and the mixture was stirred for 5 hours. The reaction mixture was diluted with DCM (10 mL) and water (10 mL) to obtain a biphasic mixture. The biphasic mixture was separated and the aqueous layer was further extracted with DCM (2×10 mL). The combined organic layers were dried over MgSO₄, filtered and concentrated in vacuo. The residue was purified by preparative HPLC (Method P3), the fractions containing the target compound were combined and concentrated in vacuo, and the resulting solid was dissolved in DCM (10 mL) and washed with 1 M aq. HCl (10 mL). The organic layer was passed through a phase separator and concentrated to afford the title compound (30 mg, 31% Yield, 98% purity) as a white solid.

¹H NMR (400 MHz, DMSO) δ 10.36 (br s, 1H), 8.82 (d, J=2.6 Hz, 1H), 7.68 (d, J=2.6 Hz, 1H), 7.44 (ddd, J=8.3, 7.4, 1.8 Hz, 1H), 7.32-7.15 (m, 4H), 7.13-7.00 (m, 1H), 4.40-4.19 (m, 4H), 3.58 (s, 3H).

LCMS: m/z=463.1 [M+H]+, (ESI+), RT=2.93, Method A

### Example 36—Synthesis of 21,23-difluoro-18,18-dioxo-8,11-dioxa-18λ6-thia-15,19-diazatetracyclo[18.3.1.113,17.02,7]pentacosa-1(23),2(7),3,5,13,17(25),20(24),21-octaene-12,16-dione (Compound 36)

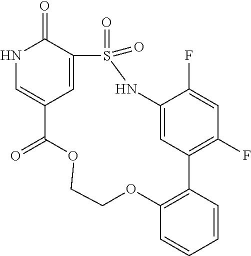 [SMILES: O=C1OCCOc2ccccc2-c2cc(c(F)cc2F)NS(=O)(=O)c2cc1c[nH]c2=O]

### Step 1

To a solution of methyl 5-bromo-6-methoxy-pyridine-3-carboxylate (1.30 g, 5.28 mmol) in anhydrous 1,4-dioxane (50 mL) was added DIPEA (1.2 mL, 6.89 mmol) and the solution was sparged with nitrogen for 10 minutes. Phenylmethanethiol (0.75 mL, 6.40 mmol), Pd₂(dba)₃ (145 mg, 0.158 mmol) and Xantphos (183 mg, 0.317 mmol) were added and the reaction mixture was heated at 100° C. for 16 hours. The reaction was cooled to room temperature and diluted with H₂O (100 mL) and extracted with EtOAc (3×100 mL). The combined organic layers were washed with brine (100 mL), dried over MgSO₄, filtered and concentrated in vacuo. The residue was purified by FCC (0-100% EtOAc in heptane), to afford methyl 5-benzylsulfanyl-6-methoxy-pyridine-3-carboxylate (1.45 g, 85% Yield, 90% purity) as an orange solid.

¹H NMR (500 MHz, CDCl₃) δ 8.62 (d, J=2.1 Hz, 1H), 8.04 (d, J=2.1 Hz, 1H), 7.36-7.33 (m, 2H), 7.34-7.27 (m, 2H), 7.27-7.23 (m, 1H), 4.16 (s, 2H), 4.08 (s, 3H), 3.90 (s, 3H).

LCMS: m/z=290.1 [M+H]+, (ESI+), RT=1.00, Method B

### Step 2

Methyl 5-benzylsulfanyl-6-methoxy-pyridine-3-carboxylate (90% purity, 500 mg, 1.56 mmol) was dissolved in a mixture of MeCN (9 mL), acetic acid (0.45 mL) and water (0.45 mL) and the resulting suspension was cooled to 0° C. in an ice bath. 1,3-Dichloro-5,5-dimethylhydantoin (613 mg, 3.11 mmol) was then added portion wise (over approximately 15 mins) and the reaction mixture was stirred for 2 hours at 0° C. MeCN was removed under reduced pressure and DCM (20 mL) was added. The solution was cooled to 0° C., and sat. aq. NaHCO₃ solution (20 mL) was slowly added. The organic layer was separated, dried over MgSO₄, filtered and concentrated under reduced pressure to afford methyl 5-chlorosulfonyl-6-methoxy-pyridine-3-carboxylate (620 mg, 99% Yield, 66% purity) as a white solid.

LCMS: Does not ionize, RT=0.85, Method B

### Step 3

Methyl 5-chlorosulfonyl-6-methoxy-pyridine-3-carboxylate (66% purity, 600 mg, 1.49 mmol) was added to a stirred solution of Intermediate 8 (95% purity, 400 mg, 1.00 mmol) in anhydrous pyridine (10 mL) at r.t, and the mixture was heated at 50° C. for 1 hour. The reaction mixture was cooled to r.t, and concentrated in vacuo. The residue was purified by FCC (0-100% EtOAc in heptane) to afford methyl 5-[[5-[2-[2-[tert-butyl(dimethyl)silyl]oxyethoxy]phenyl]-2,4-difluoro-phenyl]sulfamoyl]-6-methoxy-pyridine-3-carboxylate (510 mg, 70% Yield, 84% purity) as a yellow oil H NMR (400 MHz, CDCl₃) δ 8.96 (d, J=2.3 Hz, 1H), 8.62 (d, J=2.2 Hz, 1H), 7.58 (dd, J=9.0, 7.4 Hz, 1H), 7.39-7.32 (m, 1H), 7.18-7.11 (m, 2H), 7.05-6.96 (m, 2H), 6.76 (dd, J=10.3, 8.9 Hz, 1H), 4.23 (s, 3H), 4.04 (t, J=5.3 Hz, 2H), 3.91 (s, 3H), 3.83 (t, J=5.3 Hz, 2H), 0.81 (s, 9H), −0.09 (s, 6H).

### Step 4

p-Methylbenzenesulfonic acid hydrate (13 mg, 0.0690 mmol) was added to a stirred solution of methyl 5-[[5-[2-[2-[tert-butyl(dimethyl)silyl]oxyethoxy]phenyl]-2,4-difluoro-phenyl]sulfamoyl]-6-methoxy-pyridine-3-carboxylate (84% purity, 500 mg, 0.690 mmol) in MeOH (8 mL) and the mixture was stirred for 1 hour. The reaction mixture was concentrated in vacuo and the residue was diluted with sat. aq. NaHCO₃ (25 mL) and extracted with EtOAc (3×25 mL). The combined organic layers were washed with brine (25 mL), dried over MgSO₄, filtered and concentrated in vacuo. The residue was purified by FCC (0-1⁰⁰% EtOAc in heptane), to afford methyl 5-[[2,4-difluoro-5-[2-(2-hydroxyethoxy)phenyl]phenyl]sulfamoyl]-6-methoxy-pyridine-3-carboxylate (340 mg, 93% Yield, 93% purity) as a white solid.

¹H NMR (400 MHz, CDCl₃) δ 8.98 (d, J=2.2 Hz, 1H), 8.64 (d, J=2.2 Hz, 1H), 7.71 (dd, J=9.0, 7.6 Hz, 1H), 7.38 (ddd, J=8.3, 7.4, 1.7 Hz, 1H), 7.27-7.22 (m, 1H), 7.16 (s, 1H), 7.09-7.01 (m, 1H), 7.00 (dd, J=8.3, 1.0 Hz, 1H), 6.81 (dd, J=10.2, 9.3 Hz, 1H), 4.24 (s, 3H), 4.13-4.08 (m, 2H), 3.92 (s, 3H), 3.91-3.87 (m, 2H), 2.46 (t, J=6.5 Hz, 1H).

LCMS: m/z=495.1 [M+H]+, (ESI+), RT=0.85, Method B

### Step 5

A solution of 2 M aq. sodium hydroxide (1.7 mL, 3.40 mmol) was added to a stirred solution of methyl 5-[[2,4-difluoro-5-[2-(2-hydroxyethoxy)phenyl]phenyl]sulfamoyl]-6-methoxy-pyridine-3-carboxylate (93% purity, 350 mg, 0.658 mmol) in THE (10 mL) at r.t. and the mixture was stirred for 1 hour. THE was removed under vacuum, the remaining aqueous was diluted with water (30 mL) and carefully acidified with 1 M aq. HCl until pH 3 was obtained. The mixture was extracted with DCM (3×30 mL). The combined organic layers were washed with brine (30 mL), dried over MgSO₄, filtered and concentrated in vacuo to afford 5-[[2,4-difluoro-5-[2-(2-hydroxyethoxy)phenyl]phenyl]sulfamoyl]-6-methoxy-pyridine-3-carboxylic acid (227 mg, 68% Yield, 95% purity).

LCMS: m/z=481.1 [M+H]+, (ESI+), RT=0.73, Method B

### Step 6

DCC (180 mg, 0.870 mmol) was added to a stirred solution of 5-[[2,4-difluoro-5-[2-(2-hydroxyethoxy)phenyl]phenyl]sulfamoyl]-6-methoxy-pyridine-3-carboxylic acid (95% purity, 220 mg, 0.435 mmol) and DMAP (11 mg, 0.0870 mmol) in anhydrous DCM (20 mL) at r.t, and the mixture was stirred for 3 hours. The reaction mixture was diluted with DCM (10 mL) and water (10 mL) and the biphasic mixture was separated. The aqueous layer was further extracted with DCM (2×10 mL). The combined organic layers were dried over MgSO₄, filtered and concentrated in vacuo. The residue was purified by FCC (0-100% EtOAc in heptane) to afford 21,23-difluoro-16-methoxy-18,18-dioxo-8,11-dioxa-18λ6-thia-15,19-diazatetracyclo[18.3.1.113,17.02,7]pentacosa-1(23),2(7),3,5,13,15,17(25),20(24),21-nonaen-12-one (158 mg, 69% Yield, 88% purity) as a white solid.

¹H NMR (400 MHz, DMSO) δ 10.60 (s, 1H), 8.88 (d, J=2.3 Hz, 1H), 7.77 (d, J=2.3 Hz, 1H), 7.46 (ddd, J=8.3, 7.4, 1.8 Hz, 1H), 7.34-7.24 (m, 2H), 7.19 (dd, J=8.3, 1.0 Hz, 1H), 7.17-7.06 (m, 2H), 4.41-4.33 (m, 2H), 4.33-4.26 (m, 2H), 4.11 (s, 3H).

LCMS: m/z=463.1 [M+H]+, (ESI+), RT=0.89, Method B

### Step 7

Iodocyclohexane (320 μL, 2.47 mmol) was added to a stirred solution of 21,23-difluoro-16-methoxy-18,18-dioxo-8,11-dioxa-18λ6-thia-15,19-diazatetracyclo[18.3.1.113,17.02,7]pentacosa-1(23),2(7),3,5,13,15,17(25),20(24),21-nonaen-12-one (88% purity, 130 mg, 0.247 mmol) in anhydrous DMF (5 mL) at r.t, and the mixture was heated at 100° C. for 3 hours. The reaction was cooled to r.t, and diluted with water (10 mL) and DCM (10 mL) and the resulting biphasic mixture separated. The aqueous layer was extracted with DCM (2×10 mL) and the combined organic layers were washed with brine (10 mL), dried over MgSO₄, filtered and concentrated in vacuo. The residue was purified by basic reverse phase FCC (C18 SiO₂ column, 10-100% MeCN in H₂O (0.1% NH₃)), to afford the title compound (84 mg, 0.182 mmol, 73% Yield, 97% purity) as a white solid.

¹H NMR (400 MHz, DMSO) δ 13.03 (br s, 1H), 10.33 (br s, 1H), 8.29 (d, J=2.7 Hz, 1H), 7.64 (d, J=2.7 Hz, 1H), 7.49-7.40 (m, 1H), 7.32-7.15 (m, 4H), 7.13-7.04 (m, 1H), 4.36-4.16 (m, 4H).

LCMS: m/z=449.1 [M+H]+, (ESI+), RT=2.77, Method A

### Example 37—Synthesis of 4,21-difluoro-18,18-dioxo-8,11-dioxa-18λ6-thia-15,19-diazatetracyclo[18.3.1.113,17.02,7]pentacosa-1(23),2(7),3,5,13,17(25),20(24),21-octaene-12,16-dione (Compound 37)

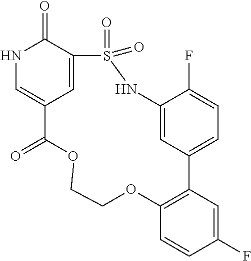 [SMILES: O=C1OCCOc2ccc(F)cc2-c2ccc(F)c(c2)NS(=O)(=O)c2cc1c[nH]c2=O]

The title compound was synthesized using a similar synthetic route as described for Example 36.

¹H NMR (400 MHz, DMSO) δ 12.98 (br. s, 1H), 10.30 (br. s, 1H), 8.26 (d, J=2.7 Hz, 1H), 7.74 (d, J=2.6 Hz, 1H), 7.35 (dd, J=7.6, 2.2 Hz, 1H), 7.27-7.06 (m, 5H), 4.36-4.26 (m, 2H), 4.26-4.15 (m, 2H).

LCMS: m/z=449.0 [M+H]+, (ESI+), RT=2.93, Method A

### Example 38—Synthesis of 21,23-difluoro-18,18-dioxo-8,11-dioxa-18λ6-thia-16,19-diazatetracyclo[18.3.1.113,17.02,7]pentacosa-1(23),2(7),3,5,13,17(25),20(24),21-octaene-12,15-dione (Compound 38)

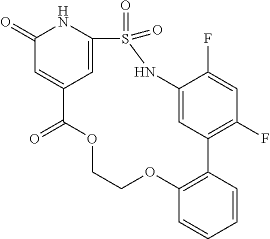 [SMILES: O=C1OCCOc2ccccc2-c2cc(c(F)cc2F)NS(=O)(=O)c2cc1cc(=O)[nH]2]

The title compound was synthesized using a similar synthetic route as described for Example 48.

¹H NMR (500 MHz, DMSO) δ 12.40 (s, 1H), 10.25 (s, 1H), 7.48-7.43 (m, 1H), 7.29 (dd, J=7.5, 1.8 Hz, 1H), 7.28-7.20 (m, 3H), 7.20-7.17 (m, 1H), 7.11-7.07 (m, 1H), 6.94 (s, 1H), 4.44-4.38 (m, 2H), 4.37-4.29 (m, 2H).

LCMS: m/z=447.0 [M−H]−, (ESI−), RT=3.17, Method A

### Example 39—Synthesis of 15-chloro-21,23-difluoro-16-hydroxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one (Compound 39)

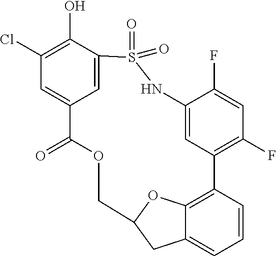 [SMILES: O=C1OCC2Cc3cccc(c3O2)-c2cc(c(F)cc2F)NS(=O)(=O)c2cc1cc(Cl)c2O]

### Step 1

A mixture of 1-allyloxy-2-bromobenzene (3.10 g, 14.5 mmol) and N,N-diethylaniline (5.0 mL, 31.2 mmol) was heated to reflux at 220° C. for 4.5 hours. After cooling to r.t., 1 M aq. HCl (50 mL) and Et₂O (40 mL) were added and then the biphasic mixture was separated. The aqueous layer was extracted with further Et₂O (40 mL). The combined organic layers were extracted with 1 M aq. NaOH (2×60 mL). The basic aqueous extractions were combined and acidified with 6 M aq. HCl. This aqueous phase was extracted with Et₂O (2×80 mL). The resulting organic phases were then combined, washed with brine, dried over MgSO₄, filtered and concentrated to give 2-allyl-6-bromophenol (2.74 g, 84% Yield; 95% purity) as a brown oil.

¹H NMR (500 MHz, DMSO) δ 9.01 (s, 1H), 7.35 (dd, J=8.0, 1.6 Hz, 1H), 7.06 (dd, J=7.5, 1.6 Hz, 1H), 6.78-6.65 (m, 1H), 5.93 (ddt, J=18.0, 9.1, 6.6 Hz, 1H), 5.07-4.99 (m, 2H), 3.38 (d, J=6.6 Hz, 2H).

### Step 2

To a solution of 2-allyl-6-bromophenol (95% purity, 2.74 g, 12.2 mmol) in CHCl₃ (120 mL) was added mCPBA (70% purity, 4.52 g, 18.3 mmol) and trifluoroacetic acid (0.093 mL, 1.22 mmol). The mixture was stirred at 65° C. under reflux for 2.5 hours. Further trifluoroacetic acid (0.093 mL, 1.22 mmol) was added and the mixture was stirred at 65° C. under reflux for 2 hours. The mixture was cooled to r.t., washed with NaHCO₃ (2×70 mL) then brine (70 mL), dried with MgSO₄, filtered and concentrated. The residue was purified by FCC (100 g SiO₂ column, 0-100% EtOAc in heptane) to afford (7-bromo-2,3-dihydrobenzofuran-2-yl)methanol (1.73 g, 57% Yield, 93% purity) as a pale-yellow oil.

¹H NMR (500 MHz, DMSO) δ 7.26 (d, J=7.9 Hz, 1H), 7.20-7.09 (m, 1H), 6.77-6.57 (m, 1H), 5.03 (dd, J=5.5, 5.5 Hz, 1H), 4.93-4.77 (m, 1H), 3.67-3.61 (m, 1H), 3.61-3.53 (m, 1H), 3.32-3.24 (m, 1H), 3.12 (dd, J=15.8, 7.6 Hz, 1H).

### Step 3

A mixture of 2,4-difluoro-5-(4,4,5,5-tetramethyl-1,3,2-dioxaborolan-2-yl)aniline (85% purity, 670 mg, 2.23 mmol), (7-bromo-2,3-dihydrobenzofuran-2-yl)methanol (93% purity, 500 mg, 2.03 mmol), potassium carbonate (561 mg, 4.06 mmol), water (1 mL) and anhydrous 1,4-dioxane (10 mL) was sparged with nitrogen for 5 minutes. Pd(dppf)Cl₂ (149 mg, 0.203 mmol) was added and the mixture was sparged for a further 5 minutes. The vessel was sealed and the reaction mixture was heated at 100° C. for 6 hours. The reaction mixture was cooled to r.t., filtered through Celite, washing with EtOAc and the filtrate was concentrated. The residue was purified by FCC (50 g SiO₂ column, 0-100% EtOAc in heptane) to afford [7-(5-amino-2,4-difluoro-phenyl)-2,3-dihydrobenzofuran-2-yl]methanol (761 mg, 95% Yield, 70% purity) as a yellow oil.

¹H NMR (400 MHz, DMSO) δ 7.22-7.17 (m, 1H), 7.06 (dd, J=11.4, 9.9 Hz, 1H), 7.03-6.95 (m, 1H), 6.90-6.77 (m, 2H), 5.00 (s, 2H), 4.91 (dd, J=5.6, 5.6 Hz, 1H), 4.85-4.70 (m, 1H), 3.59-3.48 (m, 2H), 3.30-3.16 (m, 1H), 3.09-2.98 (m, 1H).

LCMS: m/z=278.0 [M+H]+, (ESI+), RT=0.78, Method B

### Step 4

Intermediate 1 (50% purity, 1.25 g, 2.08 mmol) and [7-(5-amino-2,4-difluoro-phenyl)-2,3-dihydrobenzofuran-2-yl]methanol (70% purity, 750 mg, 1.89 mmol) were dissolved in anhydrous pyridine (8 mL) and the mixture was stirred at 50° C. for 1 hour. The reaction mixture was allowed to cool to r.t., was diluted with 1 M aq. HCl (30 mL) and extracted with EtOAc (3×30 mL). The combined organic extracts were washed with brine (50 mL), dried over Na₂SO₄, filtered and concentrated under reduced pressure. The residue was purified by FCC (50 g SiO₂ column, 0-100% EtOAc in heptane) to afford methyl 3-chloro-5-[[2,4-difluoro-5-[2-(hydroxymethyl)-2,3-dihydrobenzofuran-7-yl]phenyl]sulfamoyl]-4-methoxy-benzoate (790 mg, 66% Yield, 85% purity) as a pale-yellow solid.

¹H NMR (400 MHz, DMSO) δ 10.38 (s, 1H), 8.26 (d, J=2.1 Hz, 1H), 8.16 (d, J=2.2 Hz, 1H), 7.38-7.28 (m, 2H), 7.24 (dd, J=7.3, 1.3 Hz, 1H), 6.99 (d, J=7.7 Hz, 1H), 6.91-6.85 (m, 1H), 4.89 (dd, J=5.5, 5.5 Hz, 1H), 4.83-4.67 (m, 1H), 3.99 (s, 3H), 3.87 (s, 3H), 3.56-3.47 (m, 2H), 3.30-3.20 (m, 1H), 3.08 (dd, J=15.9, 7.2 Hz, 1H).

LCMS: m/z=538.1/540.1 [M−H]−, (ESI−), RT=1.00, Method B

### Step 5

Methyl 3-chloro-5-[[2,4-difluoro-5-[2-(hydroxymethyl)-2,3-dihydrobenzofuran-7-yl]phenyl]sulfamoyl]-4-methoxy-benzoate (85% purity, 785 mg, 1.24 mmol) was dissolved in THE (9 mL) and 2 M aq. sodium hydroxide (3.0 mL, 6.00 mmol) was added. The reaction mixture was stirred at r.t. for 4 hours. THE was removed under vacuum, the remaining aqueous diluted with water (30 mL) and acidified with 1 M aq. HCl. The mixture was extracted with DCM (3×30 mL), and the combined organic passed through a phase separator, then concentrated to afford 3-chloro-5-[[2,4-difluoro-5-[2-(hydroxymethyl)-2,3-dihydrobenzofuran-7-yl]phenyl]sulfamoyl]-4-methoxy-benzoic acid (784 mg, quantitative yield, 86% purity) as an off-white solid.

¹H NMR (500 MHz, DMSO) δ 13.66 (br. s, 1H), 10.34 (br. s, 1H), 8.22 (d, J=2.0 Hz, 1H), 8.15 (d, J=2.0 Hz, 1H), 7.38-7.28 (m, 2H), 7.23 (dd, J=7.3, 1.3 Hz, 1H), 6.97 (d, J=7.7 Hz, 1H), 6.91-6.79 (m, 1H), 4.99-4.83 (m, 1H), 4.81-4.70 (m, 1H), 3.96 (s, 3H), 3.51 (d, J=4.7 Hz, 2H), 3.25 (dd, J=15.9, 9.4 Hz, 1H), 3.08 (dd, J=15.9, 7.2 Hz, 1H).

LCMS: m/z=524.0/526.1 [M−H]−, (ESI−), RT=0.87, Method B

### Step 6

To a mixture of 3-chloro-5-[[2,4-difluoro-5-[2-(hydroxymethyl)-2,3-dihydrobenzofuran-7-yl]phenyl]sulfamoyl]-4-methoxy-benzoic acid (86% purity, 720 mg, 1.18 mmol), DMAP (29 mg, 0.235 mmol) and anhydrous DCM (40 mL) was added DCC (486 mg, 2.35 mmol). The reaction mixture was stirred at r.t. for 2 hours. The mixture was diluted with water (40 mL) and the biphasic mixture was separated. The aqueous was extracted with further DCM (2×30 ml), then the combined organics were passed through a phase separator and concentrated. The residue was purified by FCC (25 g SiO₂ column, 20-100% DCM in heptane) to afford 15-chloro-21,23-difluoro-16-methoxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one (451 mg, 70% Yield, 93% purity) as a white solid.

### Step 7

15-chloro-21,23-difluoro-16-methoxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one (93% purity, 150 mg, 0.275 mmol), anhydrous DMF (4 mL) and iodocyclohexane (178 μL, 1.37 mmol) were added to a pressure vial. The vial was sealed and heated at 120° C. for 1 hour 45 minutes. The reaction mixture was allowed to cool to r.t., and sat aq. NaHCO₃ (30 mL) was added, and the biphasic mixture was separated. The aqueous phase was extracted with EtOAc (2×30 mL). The combined organic extracts were washed with sat. aq. Na₂S₂O₃ (40 mL), 1 M aq. HCl (40 mL), water (40 mL) then brine (40 mL), dried over Na₂SO₄, filtered and concentrated under reduced pressure. The residue was purified by FCC (10 g SiO₂ column, 0-20% MeOH in DCM) to afford the title compound (105 mg, 77% Yield, 99% purity) as a white solid.

¹H NMR (500 MHz, DMSO) δ 8.43-8.33 (m, 1H), 7.85-7.76 (m, 1H), 7.74 (dd, J=8.3, 8.3 Hz, 1H), 7.36 (dd, J=10.3, 10.3 Hz, 1H), 7.27 (d, J=7.3 Hz, 1H), 7.18-7.10 (m, 1H), 6.92 (dd, J=7.5, 7.5 Hz, 1H), 5.21-5.08 (m, 1H), 4.65-4.53 (m, 1H), 4.32-4.21 (m, 1H), 3.36 (dd, J=15.9, 9.1 Hz, 1H), 2.97 (dd, J=15.9, 7.3 Hz, 1H).

LCMS: m/z=492.0/494.0 [M−H]−, (ESI−), RT=4.05, Method A

### Example 40—Synthesis of 15-chloro-21,23-difluoro-16-hydroxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one, Enantiomer 1 (Compound 40)

### Example 41—Synthesis of 15-chloro-21,23-difluoro-16-hydroxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one, Enantiomer 2 (Compound 41)

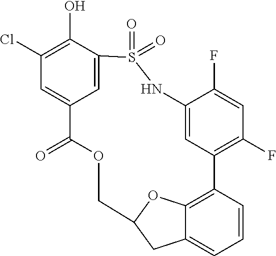 [SMILES: O=C1OCC2Cc3cccc(c3O2)-c2cc(c(F)cc2F)NS(=O)(=O)c2cc1cc(Cl)c2O]

(7-Bromo-2,3-dihydrobenzofuran-2-yl)methanol (as synthesized in Example 39, Step 2; 977 mg, 4.2 mmol) was purified by chiral SFC (Chiralpak IG column, 21×250 mm, 5 μm; flow rate 50 mL/min; 20% MeOH/80% CO₂, with 0.2% v/v NH₃ mobile phase) to afford two components:

7-Bromo-2,3-dihydrobenzofuran-2-yl)methanol, Enantiomer 1 (355 mg, brown oil) H NMR (500 MHz, DMSO) δ 7.28-7.24 (m, 1H), 7.19-7.16 (m, 1H), 6.74 (dd, J=8.0, 7.3 Hz, 1H), 5.03 (t, J=5.4 Hz, 1H), 4.88 (dddd, J=9.3, 7.7, 5.0, 4.1 Hz, 1H), 3.67-3.55 (m, 2H), 3.31-3.27 (m, 1H), 3.15-3.09 (m, 1H).

Chiral analytical SFC: RT=2.02 min (Chiralpak IG column, 4.6×250 mm, 5 μm; flow rate 4 mL/min; 20% MeOH/80% CO₂ with 0.1% v/v NH₃ mobile phase)

7-Bromo-2,3-dihydrobenzofuran-2-yl)methanol, Enantiomer 2 (366 mg, brown oil) H NMR (500 MHz, DMSO) δ 7.28-7.24 (m, 1H), 7.19-7.16 (m, 1H), 6.74 (dd, J=8.0, 7.3 Hz, 1H), 5.03 (t, J=5.5 Hz, 1H), 4.88 (dddd, J=9.3, 7.7, 5.0, 4.1 Hz, 1H), 3.67-3.55 (m, 2H), 3.31-3.26 (m, 1H), 3.15-3.09 (m, 1H).

Chiral analytical SFC: RT=2.75 min (Chiralpak IG column, 4.6×250 mm, 5 μm; flow rate 4 mL/min; 20% MeOH/80% CO₂ with 0.1% v/v NH₃ mobile phase)

7-Bromo-2,3-dihydrobenzofuran-2-yl)methanol, Enantiomer 1 (355 mg) was used in a similar synthetic sequence as described for Example 39, to afford Example 40: 15-chloro-21,23-difluoro-16-hydroxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one, Enantiomer 1 (60 mg, 99% purity) as a pink solid.

¹H NMR (500 MHz, DMSO) δ 8.47 (d, J=2.1 Hz, 1H), 8.04 (d, J=2.1 Hz, 1H), 7.83-7.76 (m, 1H), 7.48-7.40 (m, 1H), 7.30 (dd, J=7.4, 1.2 Hz, 1H), 7.24-7.18 (m, 1H), 6.97-6.91 (m, 1H), 5.19-5.10 (m, 1H), 4.76 (dd, J=11.7, 3.7 Hz, 1H), 4.23 (dd, J=11.7, 10.7 Hz, 1H), 3.40 (dd, J=16.0, 9.3 Hz, 1H), 3.00 (dd, J=16.0, 6.2 Hz, 1H).

LCMS: m/z=492.0/494.0 [M−H]−, (ESI−), RT=3.97, Method A

7-Bromo-2,3-dihydrobenzofuran-2-yl)methanol, Enantiomer 2 (366 mg) was used in a similar synthetic sequence as described for Example 39, to afford Example 41: 15-chloro-21,23-difluoro-16-hydroxy-18,18-dioxo-8,11-dioxa-18λ6-thia-19-azapentacyclo[18.3.1.16,9.113,17.02,7]hexacosa-1(23),2(7),3,5,13,15,17(25),20(24),21-nonaen-12-one, Enantiomer 2 (37 mg, 100% purity) as a pale brown solid.

¹H NMR (500 MHz, DMSO) δ 8.47 (d, J=2.1 Hz, 1H), 8.03 (d, J=2.1 Hz, 1H), 7.82-7.75 (m, 1H), 7.48-7.40 (m, 1H), 7.29 (dd, J=7.3, 1.2 Hz, 1H), 7.24-7.19 (m, 1H), 6.98-6.91 (m, 1H), 5.19-5.10 (m, 1H), 4.75 (dd, J=11.6, 3.7 Hz, 1H), 4.23 (dd, J=11.7, 10.7 Hz, 1H), 3.39 (dd, J=16.2, 9.5 Hz, 1H), 3.00 (dd, J=16.0, 6.2 Hz, 1H).

LCMS: m/z=492.0/494.0 [M−H]−, (ESI−), RT=3.98, Method A

### Example 42—Synthesis of 15-chloro-21-fluoro-16-hydroxy-18,18-dioxo-11-oxa-18λ6-thia-19-azatetracyclo[18.3.1.113,17.02,7]pentacosa-1(23),2(7),3,5,13,15,17(25),20(24),21-nonaen-12-one (Compound 42)

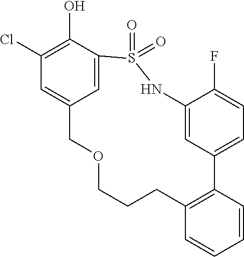 [SMILES: O=S1(=O)Nc2cc(ccc2F)-c2ccccc2CCCOCc2cc(Cl)c(O)c1c2]

15-chloro-21-fluoro-16-methoxy-18,18-dioxo-11-oxa-18λ6-thia-19-azatetracyclo[18.3.1.113,17.02,7]pentacosa-1(23),2(7),3,5,13,15,17(25),20(24),21-nonaen-12-one (synthesized using a similar method to Intermediate 12, 95% purity, 100 mg, 0.20 mmol) and iodocyclohexane (210 mg, 1.00 mmol) were combined and the mixture dissolved into anhydrous DMF (3 mL). The mixture was heated to 120° C., and stirred for 2 hours. The reaction mixture was allowed to cool to r.t., and sat aq. Na₂S₂O₃ (50 mL) was added. The biphasic mixture was separated and the aqueous phase was extracted with DCM (3×50 mL). The combined organic extracts were washed with 1 M aq. HCl (50 mL), then brine (50 mL) and were dried over Na₂SO₄, filtered and concentrated under vacuum. The residue was purified by preparative HPLC (Method P1) to afford the title compound (60 mg, 64% Yield, 99% purity) as an off-white solid.

¹H NMR (500 MHz, DMSO) δ 7.97 (d, J=2.2 Hz, 1H), 7.73 (d, J=2.2 Hz, 1H), 7.42-7.36 (m, 3H), 7.29-7.24 (m, 1H), 7.16 (dd, J=7.5, 1.4 Hz, 1H), 7.11 (dd, J=10.2, 8.4 Hz, 1H), 7.00 (ddd, J=8.4, 4.7, 2.3 Hz, 1H), 4.22-4.16 (m, 2H), 2.58-2.53 (m, 2H), 1.92-1.83 (m, 2H).

LCMS: m/z=460.1/462.1 [M−H]−, (ESI−), RT=4.25, Method A

### Example 43—Synthesis of 15-chloro-21,23-difluoro-16-hydroxy-8-methyl-18,18-dioxo-11-oxa-18λ6-thia-19-azatetracyclo[18.3.1.113,17.02,7]pentacosa-1(23),2(7),3,5,13,15,17(25),20(24),21-nonaen-12-one (Compound 43)

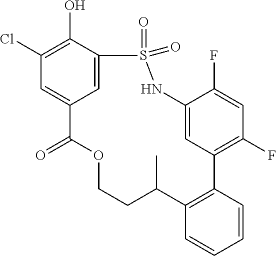 [SMILES: CC1CCOC(=O)c2cc(Cl)c(O)c(c2)S(=O)(=O)Nc2cc(c(F)cc2F)-c2ccccc21]

### Step 1

Thionyl dichloride (1.8 mL, 24.8 mmol) was carefully added dropwise to a stirred solution of 3-(2-bromophenyl)butanoic acid (500 mg, 2.06 mmol) in MeOH (15 mL) at −15° C., and the mixture was stirred for 16 hours at r.t. The mixture was concentrated in vacuo and the resultant material was dissolved in water (10 mL) and EtOAc (10 mL) and separated. The aqueous layer was further extracted by EtOAc (2×10 mL) and the combined organic layers were washed with brine (10 mL), dried over MgSO₄, filtered and concentrated in vacuo. The residue was purified by FCC (SiO₂, 0-100% EtOAc in heptane), to afford methyl 3-(2-bromophenyl)butanoate (470 mg, 84% Yield, 95% purity) as a colorless oil.

¹H NMR (400 MHz, DMSO) δ 7.58 (dd, J=8.0, 1.3 Hz, 1H), 7.43-7.37 (m, 1H), 7.39-7.30 (m, 1H), 7.20-7.09 (m, 1H), 3.67-3.55 (m, 1H), 3.55 (s, 3H), 2.75-2.58 (m, 2H), 1.18 (d, J=6.9 Hz, 3H).

LCMS: m/z=257.0/259.0 [M+H]+, (ESI+), RT=0.96, Method B

### Step 2

A solution of methyl 3-(2-bromophenyl)butanoate (95% purity, 450 mg, 1.66 mmol) in anhydrous THE (9.5 mL) was cooled to 0° C., and DIBAL (1 M in THF) (3.7 mL, 3.70 mmol) was added dropwise the reaction solution, and the mixture was stirred for 2 hours. Saturated potassium sodium tartrate aqueous solution (20 mL) and EtOAc (20 mL) were added to the reaction and the resulting mixture was stirred at r.t. for 1 hour. The mixture was separated and the aqueous layer was further extracted with EtOAc (2×20 mL). The combined organic layers were washed with brine (20 mL), dried over MgSO₄, filtered and concentrated in vacuo to afford 3-(2-bromophenyl)butan-1-ol (360 mg, 92% Yield, 97% purity) as a colorless oil.

¹H NMR (400 MHz, DMSO) δ 7.60-7.52 (m, 1H), 7.38-7.30 (m, 2H), 7.19-7.06 (m, 1H), 4.40 (t, J=5.0 Hz, 1H), 3.42-3.18 (m, 3H), 1.85-1.61 (m, 2H), 1.16 (d, J=6.9 Hz, 3H).

### Step 3

tert-Butyldimethylsilyl chloride (490 mg, 3.25 mmol) was added to a stirred solution of 3-(2-bromophenyl)butan-1-ol (97% purity, 350 mg, 1.48 mmol), N,N-diethylethanamine (0.45 mL, 3.23 mmol), and DMAP (18 mg, 0.147 mmol) in anhydrous DCM (20 mL) at r.t. and the mixture was stirred for 16 hours. The mixture was quenched with water (20 mL) and extracted with DCM (3×20 mL). The combined organic layers were washed with brine (30 mL), dried over MgSO₄, filtered and concentrated in vacuo. The residue was purified by FCC (SiO₂, 0-100% EtOAc in heptane), to afford 3-(2-bromophenyl)butoxy-tert-butyl-dimethylsilane (310 mg, 60% Yield, 98% purity) as a colorless oil.

¹H NMR (400 MHz, DMSO) δ 7.60-7.53 (m, 1H), 7.38-7.33 (m, 2H), 7.17-7.08 (m, 1H), 3.60-3.47 (m, 2H), 3.38-3.26 (m, 1H), 1.89-1.76 (m, 1H), 1.77-1.66 (m, 1H), 1.16 (d, J=6.9 Hz, 3H), 0.84 (s, 9H), −0.01-−0.06 (m, 6H).

### Step 4

A stirred solution of 2,4-difluoro-5-(4,4,5,5-tetramethyl-1,3,2-dioxaborolan-2-yl)aniline (90% purity, 298 mg, 1.05 mmol), 3-(2-bromophenyl)butoxy-tert-butyl-dimethylsilane (97% purity, 310 mg, 0.876 mmol) and potassium carbonate (242 mg, 1.75 mmol) in 1,4-dioxane (5 mL) and water (0.5 mL) was sparged with nitrogen for 10 minutes. Pd(dppf)Cl₂ (64 mg, 0.0875 mmol) was added and the mixture was heated at 100° C. for 4 hours. The mixture was cooled to r.t., quenched with water (50 mL) and extracted with EtOAc (3×50 mL). The combined organic layers were washed with brine (50 mL), dried over MgSO₄, filtered and concentrated in vacuo. The residue was purified by FCC (SiO₂, 0-50% EtOAc in heptane), to afford 5-[2-[3-[tert-butyl(dimethyl)silyl]oxy-1-methyl-propyl]phenyl]-2,4-difluoro-aniline (301 mg, 85% Yield, 97% purity) as a colorless oil. LCMS: m/z=392.3 [M+H]+, (ESI+), RT=1.20, Method C

### Step 5

Intermediate 1 (50% purity, 444 mg, 0.742 mmol) was added to a stirred solution of 5-[2-[3-[tert-butyl(dimethyl)silyl]oxy-1-methyl-propyl]phenyl]-2,4-difluoro-aniline (97% purity, 300 mg, 0.743 mmol) in pyridine (10 mL) at r.t, and the mixture was heated at 50° C. for 1 hour. The mixture was concentrated in vacuo and the residue was purified by FCC (SiO₂, 0-100% EtOAc in heptane) to afford methyl 3-[[5-[2-[3-[tert-butyl(dimethyl)silyl]oxy-1-methyl-propyl]phenyl]-2,4-difluoro-phenyl]sulfamoyl]-5-chloro-4-methoxy-benzoate (420 mg, 0.578 mmol, 78% Yield, 90% purity) as a colorless oil.

LCMS: m/z=654.2/656.2 [M+H]+, (ESI+), RT=1.25, Method C

### Step 6

p-Methylbenzenesulfonic acid hydrate (10 mg, 0.0550 mmol) was added to a stirred solution of methyl 3-[[5-[2-[3-[tert-butyl(dimethyl)silyl]oxy-1-methyl-propyl]phenyl]-2,4-difluoro-phenyl]sulfamoyl]-5-chloro-4-methoxy-benzoate (90% purity, 400 mg, 0.550 mmol) in methanol (10 mL) at r.t, and the mixture was stirred for 1 hour. The mixture was concentrated in vacuo and the residue was diluted with water (25 mL) and extracted with EtOAc (3×25 mL). The combined organic layers were washed with brine (25 mL), dried over MgSO₄, filtered and concentrated in vacuo. The residue was purified by FCC (SiO₂, 0-100% EtOAc in heptane) to afford methyl 3-chloro-5-[[2,4-difluoro-5-[2-(3-hydroxy-1-methyl-propyl)phenyl]phenyl]sulfamoyl]-4-methoxy-benzoate (310 mg, 79% Yield, 76% purity) as a white solid.

¹H NMR (400 MHz, DMSO) δ 10.11 (s, 1H), 8.29-8.11 (m, 2H), 7.47-7.33 (m, 2H), 7.33-7.16 (m, 2H), 7.16-6.92 (m, 2H), 3.99 (s, 3H), 3.88 (s, 3H), 3.18-3.10 (m, 2H), 2.64-2.54 (m, 1H), 1.74-1.55 (m, 2H), 1.10-0.98 (m, 3H). OH not visible.

LCMS: m/z=540.1/541.9 [M+H]+, (ESI+), RT=0.99, Method B

### Step 7

2 M aq. sodium hydroxide solution (0.65 mL, 1.31 mmol) was added to a stirred solution of methyl 3-chloro-5-[[2,4-difluoro-5-[2-(3-hydroxy-1-methyl-propyl)phenyl]phenyl]sulfamoyl]-4-methoxy-benzoate (76% purity, 310 mg, 0.436 mmol) in THE (10 mL) and the mixture was stirred for 2 hours. The mixture was concentrated in vacuo then diluted with water (10 mL), acidified with 1 M HCl (10 mL) and extracted by DCM (3×10 mL). The combined organic layers were washed with brine (10 mL), dried over MgSO₄, filtered and concentrated in vacuo to afford 3-chloro-5-[[2,4-difluoro-5-[2-(3-hydroxy-1-methyl-propyl)phenyl]phenyl]sulfamoyl]-4-methoxy-benzoic acid (210 mg, 71% Yield, 78% purity) as a white solid.

LCMS (MSQ₂, Acidic 2 min): [M+H]+m/z 526.0/528.0, RT 0.86 minutes.

¹H NMR (400 MHz, DMSO) δ 8.23-8.15 (m, 2H), 7.50-7.36 (m, 3H), 7.32-7.17 (m, 2H), 7.12-6.95 (m, 2H), 3.99 (s, 3H), 3.20-3.11 (m, 2H), 2.63-2.53 (m, 1H), 1.73-1.55 (m, 2H), 1.18-0.94 (m, 3H). NH and acidic proton not visible.

LCMS: m/z=526.0/528.0 [M+H]+, (ESI+), RT=0.86, Method B

### Step 8

DCC (122 mg, 0.593 mmol) was added to a stirred solution of 3-chloro-5-[[2,4-difluoro-5-[2-(3-hydroxy-1-methyl-propyl)phenyl]phenyl]sulfamoyl]-4-methoxybenzoic acid (78%, 205 mg, 0.297 mmol) and DMAP (7.2 mg, 0.0593 mmol) in anhydrous DCM (15 mL) and the mixture was stirred for 2 hours at r.t. The mixture was diluted with water (20 mL) and DCM (10 mL) and the biphasic mixture was separated. The aqueous layer was further extracted with DCM (2×10 mL). The combined organic layers were washed with brine (20 mL), dried over MgSO₄, filtered and concentrated in vacuo. The residue was purified by FCC (SiO₂, 0-100% EtOAc in heptane) to afford 15-chloro-21,23-difluoro-16-methoxy-8-methyl-18,18-dioxo-11-oxa-18λ6-thia-19-azatetracyclo[18.3.1.113,17.02,7]pentacosa-1(23),2(7),3,5,13,15,17(25),20(24),21-nonaen-12-one (120 mg, 72% Yield, 90%) as a white solid.

¹H NMR (400 MHz, DMSO) δ 10.71 (s, 1H), 8.16 (d, J=2.2 Hz, 1H), 7.78 (d, J=2.2 Hz, 1H), 7.59-7.36 (m, 3H), 7.36-7.25 (m, 2H), 7.26-7.13 (m, 1H), 4.51-4.34 (m, 1H), 4.15-3.96 (m, 4H), 2.56-2.39 (m, 1H), 2.06-1.93 (m, 1H), 1.86-1.71 (m, 1H), 1.19 (d, J=6.7 Hz, 3H).

LCMS: m/z=506.1/508.2 [M−H]−, (ESI−), RT=1.10, Method B

### Step 9

Iodocyclohexane (275 μL, 2.13 mmol) was added to a solution of 15-chloro-21,23-difluoro-16-methoxy-8-methyl-18,18-dioxo-11-oxa-18λ6-thia-19-azatetracyclo[18.3.1.113,17.02,7]pentacosa-1(23),2(7),3,5,13,15,17(25),20(24),21-nonaen-12-one (90% purity, 120 mg, 0.213 mmol) in anhydrous DMF (5 mL) and the mixture was heated at 120° C. for 3 hours. The mixture was cooled to r.t., diluted with H₂O (20 mL) and DCM (20 mL), and separated. The aqueous layer was washed with DCM (2×20 mL) and the combined organic layers were washed with brine (30 mL), dried over MgSO₄, filtered and concentrated in vacuo. The residue was purified by FCC (C18-SiO₂, 10-100% MeCN in H₂O (0.1% NH₃)), then dissolved in DCM (10 mL) and washed with 1 M HCl solution (3×10 mL), concentrated, and dried in a vacuum oven to afford the title compound (65 mg, 61% Yield, 99% purity) as a white solid.

¹H NMR (500 MHz, DMSO) δ 8.04-7.93 (m, 1H), 7.73 (d, J=2.1 Hz, 1H), 7.51-7.37 (m, 3H), 7.34-7.26 (m, 2H), 7.19 (d, J=7.5 Hz, 1H), 4.48-4.34 (m, 1H), 4.10-3.92 (m, 1H), 2.05-1.93 (m, 1H), 1.84-1.74 (m, 1H), 1.17 (d, J=6.7 Hz, 3H).

LCMS: m/z=492.0/494.0 [M−H]−, (ESI−), RT=4.41, Method A

### Example 44—Synthesis of 15-chloro-21,23-difluoro-16-hydroxy-8-methyl-18,18-dioxo-11-oxa-18λ6-thia-19-azatetracyclo[18.3.1.113,17.02,7]pentacosa-1(23),2(7),3,5,13,15,17(25),20(24),21-nonaen-12-one, Enantiomer 1 (Compound 44)

### Example 45—Synthesis of 15-chloro-21,23-difluoro-16-hydroxy-8-methyl-18,18-dioxo-11-oxa-18λ6-thia-19-azatetracyclo[18.3.1.113,17.02,7]pentacosa-1(23),2(7),3,5,13,15,17(25),20(24),21-nonaen-12-one, Enantiomer 2 (Compound 45)

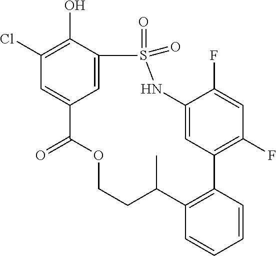 [SMILES: CC1CCOC(=O)c2cc(Cl)c(O)c(c2)S(=O)(=O)Nc2cc(c(F)cc2F)-c2ccccc21]

Example 43 (45 mg) was purified by chiral SFC (Chiralcel OJ-H column, 10×250 mm, 5 μm; flow rate 15 mL/min; 10% MeOH/90% CO₂ mobile phase) to afford two components:

### Example 44: 13.2 mg, 99% purity, >99% e.e

¹H NMR (500 MHz, DMSO) δ 8.00-7.93 (m, 1H), 7.72 (d, J=2.2 Hz, 1H), 7.50-7.37 (m, 3H), 7.33-7.24 (m, 2H), 7.18 (dd, J=7.6, 1.3 Hz, 1H), 4.56-4.27 (m, 1H), 4.09-3.89 (m, 1H), 2.04-1.90 (m, 1H), 1.88-1.74 (m, 1H), 1.16 (d, J=6.7 Hz, 3H).

LCMS: m/z=492.1/494.1 [M−H]−, (ESI−), RT=4.38, Method A

Chiral analytical SFC: RT=3.18 min (Chiralcel OJ-H column, 4.6×250 mm, 5 μm; flow rate 4 mL/min; 20% MeOH/80% CO₂ mobile phase)

### Example 45: 14.8 mg, 98% Purity, 92% e.e

¹H NMR (500 MHz, DMSO) δ 7.90-7.83 (m, 1H), 7.74-7.66 (m, 1H), 7.49-7.38 (m, 3H), 7.34-7.22 (m, 2H), 7.17 (dd, J=7.6, 1.3 Hz, 1H), 4.49-4.30 (m, 1H), 4.03-3.87 (m, 1H), 2.03-1.93 (m, 1H), 1.87-1.71 (m, 1H), 1.15 (d, J=6.8 Hz, 3H).

LCMS: m/z=492.1/494.0 [M−H]−, (ESI−), RT=4.39, Method A

Chiral analytical SFC: RT=3.87 min (Chiralcel OJ-H column, 4.6×250 mm, 5 μm; flow rate 4 mL/min; 20% MeOH/80% CO₂ mobile phase)

### Example 46—Synthesis of 15-chloro-4,21-difluoro-16-hydroxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (Compound 46)

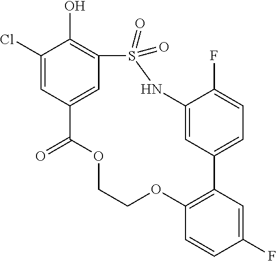 [SMILES: O=C1OCCOc2ccc(F)cc2-c2ccc(F)c(c2)NS(=O)(=O)c2cc1cc(Cl)c2O]

15-chloro-4,21-difluoro-16-methoxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (synthesized using a similar method to Intermediate 12, 98% purity, 1.70 g, 3.36 mmol) and iodocyclohexane (3.50 g, 16.7 mmol) were combined and the mixture was dissolved into anhydrous DMF (50 mL). The mixture was heated to 120° C., and stirred for 3 hours. The reaction mixture was allowed to cool to r.t., and sat aq. Na₂S₂O₃ (100 mL) was added to the reaction mixture. The biphasic mixture was separated and the aqueous phase was extracted with DCM (3×100 mL). The combined organic extracts were washed with 1 M aq. HCl (100 mL), then brine (100 mL) and were dried over Na₂SO₄, filtered and concentrated under vacuum. The residue was purified by FCC (100 g SiO₂ column, eluting with DCM in heptane, 0-100% gradient, then MeOH in DCM, 0-40%) to afford the title compound (820 mg, 1.68 mmol, 50% Yield, 99% purity) as a white solid.

¹H NMR (500 MHz, DMSO) δ 11.63-9.75 (m, 2H), 8.04 (d, J=2.1 Hz, 1H), 7.61 (d, J=2.1 Hz, 1H), 7.41 (dd, J=7.5, 2.2 Hz, 1H), 7.26-7.20 (m, 3H), 7.19-7.15 (m, 1H), 7.13 (dd, J=10.1, 8.5 Hz, 1H), 4.38-4.32 (m, 2H), 4.28-4.23 (m, 2H).

LCMS: m/z=480.0/482.0 [M−H]−, (ESI−), RT=4.04, Method A

### Example 47—Synthesis of 15-chloro-21-fluoro-16-hydroxy-23-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 47)

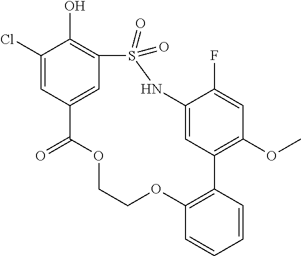 [SMILES: COc1cc(F)c2cc1-c1ccccc1OCCOC(=O)c1cc(Cl)c(O)c(c1)S(=O)(=O)N2]

Iodocyclohexane (130 μL, 1.00 mmol) was added to a suspension of 15-chloro-21-fluoro-16,23-dimethoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 100% purity, 100 mg, 0.197 mmol) in DMF (2.5 mL). The reaction was heated to 80° C. for 40 hours. The reaction was cooled and quenched with sat. aq. NaHCO₃ (20 mL). The biphasic mixture was separated, and the aqueous layer was extracted into EtOAc (3×10 mL). The combined organics washed with brine, dried over MgSO₄ and concentrated in vacuo. The residue was purified by FCC chromatography (10 g SiO₂ column, 50-100% EtOAc in heptane) followed by preparative HPLC (Method P1) to afford the title compound (20 mg, 20% Yield, 96% purity) as a white solid.

¹H NMR (500 MHz, DMSO) δ 10.04 (s, 1H), 8.02 (s, 1H), 7.51 (d, J=2.1 Hz, 1H), 7.39-7.32 (m, 1H), 7.14 (dd, J=7.5, 1.8 Hz, 1H), 7.10-7.05 (m, 2H), 7.03-6.98 (m, 1H), 6.85 (d, J=12.2 Hz, 1H), 4.35-4.30 (m, 2H), 4.25-4.21 (m, 2H), 3.60 (s, 3H).

LCMS: m/z=492.0/494.0 [M+H]+, (ESI+), RT=3.88, Method A

### Example 48—Synthesis of 15-chloro-5,21-difluoro-16-hydroxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 48)

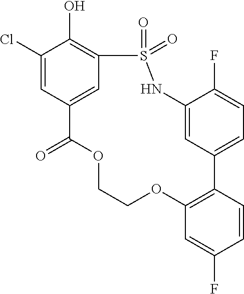 [SMILES: O=C1OCCOc2cc(F)ccc2-c2ccc(F)c(c2)NS(=O)(=O)c2cc1cc(Cl)c2O]

15-chloro-5,21-difluoro-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 99% purity, 115 mg, 0.230 mmol), anhydrous DMF (4 mL) and iodocyclohexane (148 μL, 1.15 mmol) were added to a pressure vial. The vial was sealed and heated at 120° C. for 1.5 hours. The reaction mixture was allowed to cool to r.t., and sat. aq. NaHCO₃ (30 mL) was added, and the biphasic mixture was separated. The aqueous phase was extracted with EtOAc (2×30 mL). The combined organic extracts were washed with sat. aq. Na₂S₂O₃ (40 mL), 1 M aq. HCl (40 mL), water (40 mL) then brine (40 mL), dried over Na₂SO₄, filtered and concentrated under reduced pressure. The residue was purified by FCC (10 g SiO₂ column, 0-20% MeOH in DCM) to afford the title compound (67 mg, 60% Yield, 99% purity) as a white solid.

¹H NMR (500 MHz, DMSO) δ 12.24-9.38 (m, 2H), 8.03 (d, J=2.2 Hz, 1H), 7.71 (d, J=2.1 Hz, 1H), 7.35 (dd, J=7.6, 2.1 Hz, 1H), 7.28 (dd, J=8.4, 6.9 Hz, 1H), 7.21-7.07 (m, 3H), 6.88 (ddd, J=8.4, 8.4, 2.5 Hz, 1H), 4.44-4.36 (m, 2H), 4.33-4.19 (m, 2H)

LCMS: m/z=480.0/482.0 [M−H]−, (ESI−), RT=4.07, Method A

### Example 49—Synthesis of 15-chloro-21,23-difluoro-16-hydroxy-3-methyl-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 49)

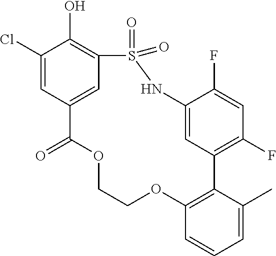 [SMILES: Cc1cccc2c1-c1cc(c(F)cc1F)NS(=O)(=O)c1cc(cc(Cl)c1O)C(=O)OCCO2]

15-chloro-21,23-difluoro-16-methoxy-3-methyl-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 95% purity, 100 mg, 0.186 mmol), anhydrous DMF (2.5 mL) and iodocyclohexane (120 μL, 0.928 mmol) were added to a pressure vial. The vial was sealed and heated at 120° C. for 1.25 hours then allowed to cool to r.t. The mixture was purified by preparative HPLC (Method P1), to afford a yellow solid. This was then triturated with Et₂O (˜5 mL) and filtered to afford the title compound (61 mg, 65% Yield, 99% purity) as a white solid.

¹H NMR (500 MHz, DMSO) δ 8.06 (d, J=2.2 Hz, 1H), 7.59 (d, J=2.1 Hz, 1H), 7.34-7.30 (m, 1H), 7.29-7.24 (m, 1H), 7.24-7.19 (m, 1H), 6.99 (d, J=8.3 Hz, 1H), 6.96 (d, J=7.7 Hz, 1H), 4.44-4.35 (m, 2H), 4.32-4.25 (m, 1H), 4.10-4.02 (m, 1H), 2.02 (s, 3H). OH and NH not observed.

LCMS: m/z=494.0/496.0 [M−H]−, (ESI−), RT=4.12, Method A

### Example 50—Synthesis of 15-chloro-21-fluoro-16-hydroxy-23-methyl-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 50)

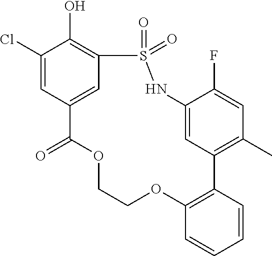 [SMILES: Cc1cc(F)c2cc1-c1ccccc1OCCOC(=O)c1cc(Cl)c(O)c(c1)S(=O)(=O)N2]

15-chloro-21-fluoro-16-methoxy-23-methyl-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 95% purity, 150 mg, 0.290 mmol), anhydrous DMF (3.9 mL) and iodocyclohexane (0.19 mL, 1.47 mmol) were added to a pressure vial. The vial was sealed and heated at 120° C. for 2 hours then allowed to cool to r.t. The organics were diluted with EtOAc (30 mL), washed with Na₂SO₃ (2×20 mL of a saturated aqueous solution), then brine (20 mL), dried over MgSO₄, filtered and concentrated in vacuo. The residue was purified by FCC (10 g SiO₂ column, 0-10% MeOH in DCM) followed by preparative HPLC (Method P1) to afford the title compound (53 mg, 38% Yield, 100% purity) as a white solid.

¹H NMR (400 MHz, DMSO) δ 8.02 (d, J=2.2 Hz, 1H), 7.56 (d, J=2.1 Hz, 1H), 7.40 (ddd, J=8.3, 7.4, 1.8 Hz, 1H), 7.17-7.11 (m, 3H), 7.08-7.03 (m, 1H), 6.99 (d, J=11.2 Hz, 1H), 4.52-4.43 (m, 1H), 4.38-4.28 (m, 2H), 4.11-4.02 (m, 1H), 1.98 (s, 3H).

LCMS: m/z=476.1/478.1 [M−H]−, (ESI−), RT=4.19, Method A

### Example 51—Synthesis of 15-chloro-21,24-difluoro-16-hydroxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 51)

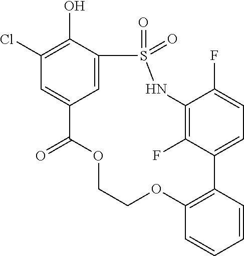 [SMILES: O=C1OCCOc2ccccc2-c2ccc(F)c(c2F)NS(=O)(=O)c2cc1cc(Cl)c2O]

15-chloro-21,24-difluoro-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 95% purity, 95 mg, 0.182 mmol), anhydrous DMF (2.4 mL) and iodocyclohexane (0.12 mL, 0.928 mmol) were added to a pressure vial. The vial was sealed and heated at 120° C. for 2 hours then allowed to cool to r.t. The organics were diluted with EtOAc (30 mL), washed with Na₂SO₃ (2×20 mL of a saturated aqueous solution), then brine (20 mL), dried over MgSO₄, filtered and concentrated in vacuo. The residue was purified by FCC (10 g SiO₂ column, 0-60% MeOH in DCM) followed by preparative HPLC (Method P1) to afford the title compound (53 mg, 60% Yield, 100% purity) as a white solid.

¹H NMR (400 MHz, DMSO) δ 8.08 (d, J=2.1 Hz, 1H), 7.90 (dd, J=2.2, 1.2 Hz, 1H), 7.40 (ddd, J=8.3, 7.3, 1.8 Hz, 1H), 7.35-7.28 (m, 1H), 7.27-7.21 (m, 1H), 7.18-7.14 (m, 1H), 7.10 (dd, J=7.5, 1.8 Hz, 1H), 7.03-6.98 (m, 1H), 4.74-4.62 (m, 1H), 4.42-4.22 (m, 2H), 4.22-4.09 (m, 1H).

LCMS: m/z=480.0/482.0 [M−H]−, (ESI−), RT=3.91, Method A

### Example 52—Synthesis of 4,15-dichloro-21-fluoro-16-hydroxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (Compound 52)

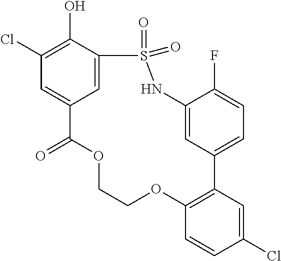 [SMILES: O=C1OCCOc2ccc(Cl)cc2-c2ccc(F)c(c2)NS(=O)(=O)c2cc1cc(Cl)c2O]

4,15-dichloro-21-fluoro-16-methoxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (synthesized using a similar method to Intermediate 12, 95% purity, 55 mg, 0.102 mmol) and iodocyclohexane (68 μL, 0.524 mmol) were added to a pressure vial and the mixture was diluted into anhydrous DMF (2.5 mL). The vial was sealed, heated to 120° C. and stirred for 3 hours. The reaction mixture was allowed to cool to r.t., added to water (50 mL) and extracted with DCM (3×30 mL). The combined organic extracts were washed with brine (100 mL), dried over Na₂SO₄, filtered and concentrated under reduced pressure. The residue was purified by FCC (10 g SiO₂ column, 0-100% MeOH in DCM) followed by preparative HPLC (Method P2) to afford the title compound (23 mg, 45% Yield, 99% purity) as a white solid.

¹H NMR (500 MHz, DMSO) δ 11.96-9.79 (m, 2H), 7.97 (d, J=2.2 Hz, 1H), 7.59 (d, J=2.1 Hz, 1H), 7.37 (dd, J=8.8, 2.7 Hz, 1H), 7.32 (dd, J=7.5, 2.2 Hz, 1H), 7.26 (d, J=2.7 Hz, 1H), 7.17-7.12 (m, 2H), 7.08 (dd, J=10.1, 8.5 Hz, 1H), 4.33-4.27 (m, 2H), 4.24-4.17 (m, 2H).

LCMS: m/z=496.0/498.0/500.1 [M−H]−, (ESI−), RT=4.29, Method A

### Example 53—Synthesis of 18-chloro-24,26-difluoro-19-hydroxy-21,21-dioxo-11,14-dioxa-21λ6-thia-5,6,22-triazapentacyclo[21.3.1.116,20.02,10.04,8]octacosa-1(27),2(10),3,6,8,16(28),17,19,23,25-decaen-15-one (Compound 53)

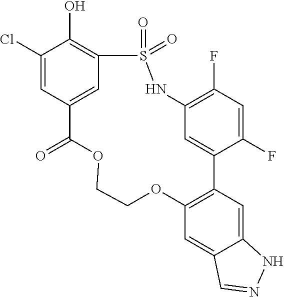 [SMILES: O=C1OCCOc2cc3cn[nH]c3cc2-c2cc(c(F)cc2F)NS(=O)(=O)c2cc1cc(Cl)c2O]

To a solution of 18-chloro-24,26-difluoro-19-methoxy-21,21-dioxo-11,14-dioxa-21λ6-thia-5,6,22-triazapentacyclo[21.3.1.116,20.02,10.04,8]octacosa-1(27),2(10),3,6,8,16(28),17,19,23,25-decaen-15-one (synthesized using a similar method to Intermediate 12, 75 mg, 0.140 mmol) in anhydrous pyridine (5 mL) was added lithium iodide (130 mg, 0.971 mmol). The reaction mixture was heated at 80° C. overnight. The mixture was diluted with 10% aq. citric acid (30 mL) and extracted with EtOAc (2×30 mL). The combined organic was washed with 10% aq. citric acid (30 mL) and brine (40 mL), then dried (Na₂SO₄), filtered and concentrated. The residue was purified by FCC (10 g SiO₂ column, 0-20% MeOH in DCM) followed by preparative HPLC (Method P1) to afford the title compound (32 mg, 43% Yield, 99% purity) as a white solid.

¹H NMR (500 MHz, DMSO) δ 13.09 (s, 1H), 11.75-9.80 (m, 2H), 8.06-8.00 (m, 2H), 7.47 (s, 1H), 7.46 (d, J=2.2 Hz, 1H), 7.43-7.35 (m, 2H), 7.26-7.17 (m, 1H), 4.39-4.22 (m, 4H).

LCMS: m/z=520.2/522.1 [M−H]−, (ESI−), RT=3.44, Method A

### Example 54—Synthesis of 15-chloro-21,22-difluoro-16-hydroxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 54)

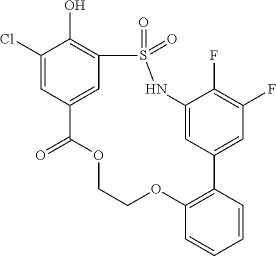 [SMILES: O=C1OCCOc2ccccc2-c2cc(F)c(F)c(c2)NS(=O)(=O)c2cc1cc(Cl)c2O]

To a solution of 15-chloro-21,22-difluoro-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 92% purity, 103 mg, 0.191 mmol) in anhydrous DMF (2.5 mL) was added iodocyclohexane (124 μL, 0.955 mmol) before heating to 120° C. in a pressure vial for 1.5 hours. The volume of the reaction mixture was reduced by approximately 50% and the residue was purified by preparative HPLC (Method P2) to afford the title compound (68 mg, 73% Yield, 99% purity) as a yellow solid.

¹H NMR (400 MHz, DMSO) δ 8.13-8.03 (m, 1H), 7.73 (d, J=2.1 Hz, 1H), 7.46-7.36 (m, 1H), 7.33-7.23 (m, 2H), 7.22-7.14 (m, 2H), 7.11-7.00 (m, 1H), 4.44-4.37 (m, 2H), 4.32-4.23 (m, 2H).

LCMS: m/z=480.2/482.2 [M−H]−, (ESI−), RT=4.20, Method A

### Example 55—Synthesis of 15-chloro-21-(difluoromethoxy)-16-hydroxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 55)

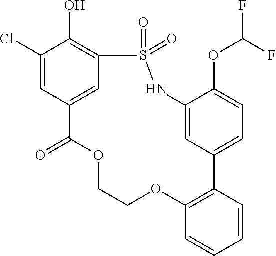 [SMILES: O=C1OCCOc2ccccc2-c2ccc(OC(F)F)c(c2)NS(=O)(=O)c2cc1cc(Cl)c2O]

To a solution of 15-chloro-21-(difluoromethoxy)-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 100% purity, 123 mg, 0.234 mmol) in anhydrous DMF (3 mL) was added iodocyclohexane (151 μL, 1.17 mmol) before heating to 120° C. in a pressure vial for 1 hour. The reaction mixture was concentrated under reduced pressure and the residue was purified by preparative HPLC (Method P1) to afford the title compound (75 mg, 60% Yield, 95% purity) as a beige solid.

¹H NMR (400 MHz, DMSO) δ 10.92 (s, 1H), 10.03 (s, 1H), 8.17-7.94 (m, 1H), 7.67-7.61 (m, 1H), 7.43-7.42 (m, 1H), 7.42-7.36 (m, 1H), 7.28-7.25 (m, 1H), 7.24-7.20 (m, 1H), 7.20-7.16 (m, 1H), 7.12-6.69 (m, 3H), 4.40-4.32 (m, 2H), 4.30-4.22 (m, 2H).

LCMS: m/z=510.3/512.3 [M−H]−, (ESI−), RT=4.20, Method A

### Example 56—Synthesis of 15-chloro-16-hydroxy-18,18-dioxo-21-(trifluoromethoxy)-8,11-dioxa-18λ6-thia-19-azatetracyclo[18.3.1.113,17.02,7]pentacosa-1(23),2(7),3,5,13(25),14,16,20(24),21-nonaen-12-one (Compound 56)

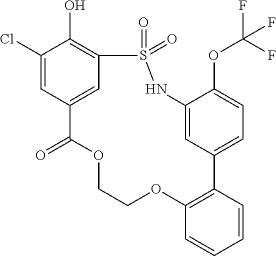 [SMILES: O=C1OCCOc2ccccc2-c2ccc(OC(F)(F)F)c(c2)NS(=O)(=O)c2cc1cc(Cl)c2O]

15-chloro-16-methoxy-18,18-dioxo-21-(trifluoromethoxy)-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 95% purity, 37 mg, 0.0646 mmol), anhydrous DMF (1 mL) and iodocyclohexane (70 μL, 0.541 mmol) were added to pressure vial. The vial was sealed and heated at 120° C. for 2 hours then allowed to cool to r.t. The organics were diluted with EtOAc (20 mL), washed with 1 M aq. HCl (10 mL), Na₂SO₃ (10 mL of a saturated aqueous solution), then brine (10 mL), dried over MgSO₄, filtered and concentrated in vacuo. The residue was purified by preparative HPLC (Method P1) to afford the title compound (21 mg, 61% Yield, 99% purity) as a white solid.

¹H NMR (500 MHz, DMSO) δ 8.03 (d, J=2.1 Hz, 1H), 7.75 (d, J=2.1 Hz, 1H), 7.50-7.48 (m, 1H), 7.40 (ddd, J=8.2, 7.4, 1.7 Hz, 1H), 7.28-7.25 (m, 3H), 7.20-7.17 (m, 1H), 7.09-7.04 (m, 1H), 4.41-4.36 (m, 2H), 4.29-4.25 (m, 2H). OH and NH not observed.

LCMS: m/z=528.1/530.1 [M−H]−, (ESI−), RT=4.35, Method A

### Example 57—Synthesis of 15-chloro-21-cyclopropyl-16-hydroxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 57)

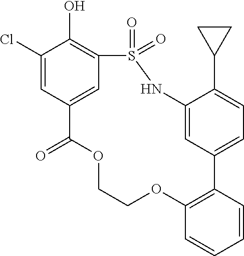 [SMILES: O=C1OCCOc2ccccc2-c2ccc(C3CC3)c(c2)NS(=O)(=O)c2cc1cc(Cl)c2O]

15-chloro-21-cyclopropyl-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (synthesized using a similar method to Intermediate 12, 93% purity, 104 mg, 0.193 mmol), anhydrous DMF (2 mL) and iodocyclohexane (208 μL, 1.61 mmol) were added to pressure vial. The vial was sealed and heated at 120° C. for 2 hours then allowed to cool to r.t. The organics were diluted with EtOAc (30 mL), washed with 1 M aq. HCl (20 mL), then Na₂SO₃ (20 mL of a 1 M aqueous solution) then brine (20 mL), dried over Na₂SO₄, filtered and concentrated in vacuo. The residue was purified by preparative HPLC (Method P1) to afford the title compound as (51 mg, 52% Yield, 97% purity) as an off-white solid.

¹H NMR (500 MHz, DMSO) δ 10.99 (br. S, 1H), 10.04 (br. S, 1H), 7.99 (d, J=2.2 Hz, 1H), 7.53 (d, J=2.2 Hz, 1H), 7.37 (ddd, J=8.2, 7.4, 1.8 Hz, 1H), 7.30 (d, J=1.8 Hz, 1H), 7.23 (dd, J=7.5, 1.8 Hz, 1H), 7.19-7.14 (m, 1H), 7.09 (dd, J=8.0, 1.8 Hz, 1H), 7.07-7.03 (m, 1H), 6.64 (d, J=8.1 Hz, 1H), 4.34-4.28 (m, 2H), 4.28-4.23 (m, 2H), 2.05-1.96 (m, 1H), 0.76-0.66 (m, 2H), 0.26-0.16 (m, 2H).

LCMS: m/z=484.1/486.1 [M−H]−, (ESI−), RT=4.34, Method A

### Example 58—Synthesis of 15-chloro-16-hydroxy-18,18-dioxo-21-(trifluoromethyl)-8,11-dioxa-18λ6-thia-19-azatetracyclo[18.3.1.113,17.02,7]pentacosa-1(24),2(7),3,5,13(25),14,16,20,22-nonaen-12-one (Compound 58)

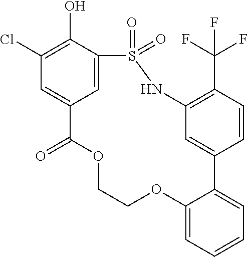 [SMILES: O=C1OCCOc2ccccc2-c2ccc(C(F)(F)F)c(c2)NS(=O)(=O)c2cc1cc(Cl)c2O]

### Step 1

A solution of 5-[2-[2-[tert-butyl(dimethyl)silyl]oxyethoxy]phenyl]-2-(trifluoromethyl)aniline (synthesized using a similar method to Intermediate 8, 96% purity, 344 mg, 0.80 mmol) in anhydrous THE (8.3 mL) was cooled to −78° C., and treated with 1 M LiHMDS in THE (1.2 mL, 1.20 mmol). The reaction was then stirred for 10 mins before the addition of Intermediate 1 (50% purity, 550 mg, 0.919 mmol) in anhydrous THF (8.3 mL). The mixture was stirred for a further 15 mins at −78° C. before warming to r.t, and stirred for 5 hours. The mixture was quenched with water (10 mL) and extracted with EtOAc (3×20 mL). The combined organic layers were washed with brine (20 mL) and dried over MgSO₄, filtered and concentrated in vacuo. The residue was purified by FCC (25 g SiO₂ column, 0 to 100% EtOAc in heptane) to afford 3-[[5-[2-[2-[tert-butyl(dimethyl)silyl]oxyethoxy]phenyl]-2-(trifluoromethyl)phenyl]sulfamoyl]-5-chloro-4-methoxy-benzoate (140 mg, 22% Yield, 84% purity) as a colorless gum.

¹H NMR (400 MHz, CDCl₃) δ 8.41 (d, J=2.1 Hz, 1H), 8.25 (d, J=2.1 Hz, 1H), 7.78 (br. s, J=0.9 Hz, 1H), 7.51 (d, J=0.9 Hz, 1H), 7.36-7.31 (m, 2H), 7.23 (dd, J=7.5, 1.8 Hz, 1H), 7.05-6.98 (m, 2H), 4.14 (s, 3H), 4.06 (t, J=5.2 Hz, 2H), 3.91-3.87 (m, 5H), 0.82 (s, 9H), −0.04 (s, 6H).

### Step 2

A solution of methyl 3-[[5-[2-[2-[tert-butyl(dimethyl)silyl]oxyethoxy]phenyl]-2-(trifluoromethyl)phenyl]sulfamoyl]-5-chloro-4-methoxybenzoate (84% purity, 140 mg, 0.174 mmol) and 4-methylbenzenesulfonic acid hydrate (1:1) (3.3 mg, 0.0174 mmol) in MeOH (2 mL) was stirred at r.t. for 2 hours. The mixture was concentrated in vacuo and the residue was purified by FCC (10 g SiO₂ column, 0 to 100% EtOAc in heptane) to afford methyl 3-chloro-5-[[5-[2-(2-hydroxyethoxy)phenyl]-2-(trifluoromethyl)phenyl]sulfamoyl]-4-methoxy-benzoate (111 mg, 100% Yield, 87% purity) as a white gum.

¹H NMR (400 MHz, DMSO) δ 10.29 (s, 1H), 8.26 (d, J=2.1 Hz, 1H), 8.18 (d, J=2.1 Hz, 1H), 7.77-7.67 (m, 2H), 7.40-7.33 (m, 1H), 7.26 (s, 1H), 7.14 (d, J=8.5 Hz, 1H), 7.08 (dd, J=7.6, 1.8 Hz, 1H), 6.99 (td, J=7.4, 1.0 Hz, 1H), 4.74 (s, 1H), 4.03-3.97 (m, 2H), 3.94 (s, 3H), 3.85 (s, 3H), 3.65 (d, J=6.8 Hz, 2H).

LCMS: m/z=558.1/560.1 [M−H]−, (ESI−), RT=1.05, Method B

### Step 3

A solution of methyl 3-chloro-5-[[5-[2-(2-hydroxyethoxy)phenyl]-2-(trifluoromethyl)phenyl]sulfamoyl]-4-methoxybenzoate (87% purity, 111 mg, 0.173 mmol) in THE (3 mL) and 2 M aq. sodium hydroxide (433 μL, 0.865 mmol) was stirred at r.t. for 4 hours. The mixture was diluted with EtOAc (10 mL) and water (10 mL) and the layers were separated. The aqueous layer was acidified to ˜pH 2 using 1 M aq. HCl, then extracted with EtOAc (3×10 mL). The combined organic layers were filtered through a phase separating filter paper and concentrated in vacuo to afford 3-chloro-5-[[5-[2-(2-hydroxyethoxy)phenyl]-2-(trifluoromethyl)phenyl]sulfamoyl]-4-methoxy-benzoic acid (102 mg, 97% Yield, 90% purity) as a white powder.

¹H NMR (400 MHz, DMSO) δ 10.25 (s, 1H), 8.25 (s, 1H), 8.13 (s, 1H), 7.85-7.42 (m, 2H), 7.33 (t, J=7.6 Hz, 1H), 7.26 (s, 1H), 7.11 (d, J=8.4 Hz, 1H), 7.04 (dd, J=7.6, 1.9 Hz, 1H), 6.96 (t, J=7.4 Hz, 1H), 4.74 (s, 1H), 3.98 (t, J=5.1 Hz, 2H), 3.91 (s, 3H), 3.63 (t, J=5.1 Hz, 2H).

LCMS: m/z=544.0/546.0 [M−H]−, (ESI−), RT=0.93, Method B

### Step 4

A solution of 3-chloro-5-[[5-[2-(2-hydroxyethoxy)phenyl]-2-(trifluoromethyl)phenyl]sulfamoyl]-4-methoxybenzoic acid (90% purity, 102 mg, 0.168 mmol), DMAP (4.1 mg, 0.034 mmol) and DCC (76 mg, 0.370 mmol) in DCM (10 mL) was stirred at r.t. for 1 hour. The mixture was diluted with water (30 mL) and extracted with DCM (3×30 mL), the combined organic layers were filtered through a phase separating cartridge and concentrated in vacuo. The residue was purified by FCC (10 g SiO₂ column, DCM in heptane (0 to 100%) followed by EtOAc in DCM (0 to 100%)) to afford 15-chloro-16-methoxy-18,18-dioxo-21-(trifluoromethyl)-8,11-dioxa-18λ6-thia-19-azatetracyclo[18.3.1.113,17.02,7]pentacosa-1(24),2(7),3,5,13(25),14,16,20,22-nonaen-12-one (40 mg, 42% Yield, 93% purity) as a white powder.

¹H NMR (400 MHz, DMSO) δ 10.50 (s, 1H), 8.33 (d, J=2.1 Hz, 1H), 8.20 (s, 1H), 7.82-7.73 (m, 1H), 7.69 (s, 1H), 7.54-7.46 (m, 1H), 7.41 (ddd, J=8.5, 7.3, 1.8 Hz, 1H), 7.31 (d, J=7.5 Hz, 1H), 7.17 (d, J=8.3 Hz, 1H), 7.07 (td, J=7.5, 1.0 Hz, 1H), 4.60-4.50 (m, 2H), 4.29 (t, J=4.6 Hz, 2H), 3.98 (s, 3H).

LCMS: m/z=526.3/528.3 [M−H]−, (ESI−), RT=1.14, Method B

### Step 5

A solution of 15-chloro-16-methoxy-18,18-dioxo-21-(trifluoromethyl)-8,11-dioxa-18λ6-thia-19-azatetracyclo[18.3.1.113,17.02,7]pentacosa-1(24),2(7),3,5,13(25),14,16,20,22-nonaen-12-one (93% purity, 40 mg, 0.071 mmol) and iodocyclohexane (46 μL, 0.352 mmol) in DMF (1 mL) was heated to 120° C. in a sealed vial for 2 hours. The mixture was cooled to r.t., then purified by preparative HPLC (Method P1) to afford the title compound as a white powder (19 mg, 53% Yield, 100% purity).

¹H NMR (400 MHz, DMSO) δ 8.11 (d, J=2.1 Hz, 1H), 8.05 (d, J=2.1 Hz, 1H), 7.72 (d, J=8.2 Hz, 1H), 7.62 (s, 1H), 7.47-7.38 (m, 2H), 7.28 (dd, J=7.5, 1.8 Hz, 1H), 7.18 (d, J=8.5 Hz, 1H), 7.07 (td, J=7.4, 1.0 Hz, 1H), 4.49-4.44 (m, 2H), 4.30-4.24 (m, 2H).

LCMS: m/z=512.1/514.1 [M−H]−, (ESI−), RT=4.15, Method A

### Example 59—Synthesis of 15-chloro-16-(difluoromethyl)-21,23-difluoro-18,18-dioxo-8,11-dioxa-18λ6-thia-19-azatetracyclo[18.3.1.113,17.02,7]pentacosa-1(24),2,4,6,13,15,17(25),20,22-nonaen-12-one (Compound 59)

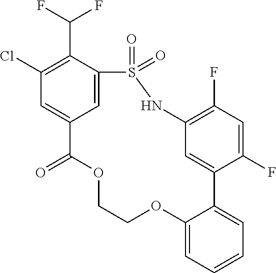 [SMILES: O=C1OCCOc2ccccc2-c2cc(c(F)cc2F)NS(=O)(=O)c2cc1cc(Cl)c2C(F)F]

### Step 1

To a dry flask was added anhydrous THF (3 mL) and anhydrous diisopropylamine (1.0 mL, 7.14 mmol). The solution was cooled to −78° C., and then 2.5 M n-butyllithium in hexanes (3.2 mL, 8.00 mmol) was added dropwise over 2 minutes. The solution was stirred at −78° C. for 1 hour. To a dry 2-neck flask fitted with a thermometer was added methyl 3-chloro-5-fluorobenzoate (0.80 mL, 5.62 mmol) and anhydrous THE (20 mL). The solution was cooled to −78° C., and then the solution prepared in the first step was added via cannula. The reaction mixture was stirred at −78° C. for 40 min before anhydrous DMF (1.3 mL, 16.8 mmol) was added. The reaction mixture was kept at −78° C. for 1 hour before it was allowed to warm to −20° C., and then saturated ammonium chloride solution (12 mL) was added and the biphasic mixture was stirred for 10 min. To the biphasic mixture was added water (20 mL). The organics were extracted with EtOAc (3×20 mL), combined, washed with brine (30 mL), dried over MgSO₄, filtered and concentrated in vacuo. The residue was purified by FCC (25 g SiO₂ cartridge, 0-20% EtOAc in heptane) to afford methyl 3-chloro-5-fluoro-4-formylbenzoate (463 mg, 36% Yield, 95% purity) as a pale-orange solid.

¹H NMR (400 MHz, DMSO) δ 10.35-10.29 (m, 1H), 7.91-7.88 (m, 1H), 7.83 (dd, J=10.6, 1.5 Hz, 1H), 3.91 (s, 3H).

### Step 2

To a suspension of potassium tert-butoxide (240 mg, 2.14 mmol) in anhydrous THE (15 mL) at 0° C. was added phenylmethanethiol (0.22 mL, 1.88 mmol). The reaction mixture was stirred at 0° C. for 5 min before a cooled (0° C.) solution of methyl 3-chloro-5-fluoro-4-formylbenzoate (95% purity, 435 mg, 1.91 mmol) in anhydrous THE (5 mL) was added. The reaction mixture was kept at 0° C. for 1 min before it was allowed to warm to r.t, and stirred for a further 2.5 hours. The mixture was concentrated in vacuo and purified by FCC (25 g SiO₂ cartridge, 0-40% EtOAc in heptane) followed by FCC (25 g SiO₂ cartridge, 0-20% Et₂O in heptane) to afford methyl 3-benzylsulfanyl-5-chloro-4-formylbenzoate (392 mg, 63% Yield, 98% purity) as a yellow solid.

¹H NMR (400 MHz, DMSO) δ 10.47-10.46 (m, 1H), 8.02-7.98 (m, 1H), 7.78 (d, J=1.4 Hz, 1H), 7.47-7.41 (m, 2H), 7.39-7.32 (m, 2H), 7.32-7.25 (m, 1H), 4.36 (s, 2H), 3.91 (s, 3H).

LCMS: m/z=319.0/321.0 [M−H]−, (ESI−), RT=1.12, Method B

### Step 3

To a solution of methyl 3-benzylsulfanyl-5-chloro-4-formylbenzoate (98% purity, 387 mg, 1.18 mmol) in DCM (11 mL) at r.t. was added DAST (0.53 mL, 4.01 mmol). The reaction mixture was stirred at r.t. for 18.5 hours and then DAST (0.2 mL, 1.51 mmol) was added. The reaction mixture was stirred at r.t. for a further 2 hours. To the reaction mixture was cautiously added sat. aq. NaHCO₃ (50 mL) at r.t. The biphasic mixture was stirred rapidly for 5 minutes and was then shaken thoroughly until gas evolution ceased. The layers were separated, and the organics were extracted with DCM (2×20 mL). The organics were combined, passed through a hydrophobic frit and concentrated in vacuo. The residue was purified by FCC (25 g SiO₂ cartridge, 0-10% EtOAc in heptane) to afford methyl 3-benzylsulfanyl-5-chloro-4-(difluoromethyl)benzoate (350 mg, 86% Yield, 100% purity) as a yellow oil.

¹H NMR (400 MHz, DMSO) δ 7.98-7.96 (m, 1H), 7.85-7.83 (m, 1H), 7.43 (t, J=52.6 Hz, 1H), 7.46-7.26 (m, 4H), 7.28-7.22 (m, 1H), 4.36 (s, 2H), 3.89 (s, 3H).

LCMS: m/z=341.1/343.1 [M−H]−, (ESI−), RT=1.17, Method B

### Step 4

Methyl 3-benzylsulfanyl-5-chloro-4-(difluoromethyl)benzoate (350 mg, 1.02 mmol) was dissolved in a mixture of acetonitrile (4.5 mL), acetic acid (0.23 mL) and water (0.23 mL) and the resulting suspension was cooled to 0° C. in an ice bath. 1,3-dichloro-5,5-dimethylhydantoin (250 mg, 1.27 mmol) was then added portion wise and the reaction mixture was stirred for 1 hour at 0° C. The organics were concentrated in vacuo (water bath at room temperature) and were then diluted with DCM (20 mL). The organics were washed with sat. aq. NaHCO₃ (20 mL), separated, passed through a phase separator and then concentrated under reduced pressure (water bath at room temperature). A solution of Intermediate 8 (90% purity, 319 mg, 0.756 mmol) in pyridine (3.6 mL) was added and the mixture was stirred at 50° C. for 50 minutes, then concentrated under reduced pressure. The residue was purified by FCC (25 g SiO₂ cartridge, 0-20% EtOAc in heptanes) to afford methyl 3-[[5-[2-[2-[tert-butyl(dimethyl)silyl]oxyethoxy]phenyl]-2,4-difluoro-phenyl]sulfamoyl]-5-chloro-4-(difluoromethyl)benzoate (453 mg, 68% Yield, 75% purity) as a yellow oil.

¹H NMR (500 MHz, DMSO) δ 11.13 (br. s, 1H), 8.33-8.29 (m, 2H), 7.78 (t, J=51.5 Hz, 1H), 7.41-7.37 (m, 1H), 7.35-7.32 (m, 1H), 7.28-7.23 (m, 1H), 7.14-7.10 (m, 2H), 7.04-7.00 (m, 1H), 4.02-3.99 (m, 2H), 3.89 (s, 3H), 3.78-3.74 (m, 2H), 0.73 (s, 9H), −0.18 (s, 6H).

LCMS: m/z=660.2/662.2 [M−H]−, (ESI−), RT=1.33, Method B

### Step 5

To a solution of methyl 3-[[5-[2-[2-[tert-butyl(dimethyl)silyl]oxyethoxy]phenyl]-2,4-difluoro-phenyl]sulfamoyl]-5-chloro-4-(difluoromethyl)benzoate (75% purity, 447 mg, 0.506 mmol) in methanol (12 mL) at r.t. was added p-methylbenzenesulfonic acid hydrate (1:1) (20 mg, 0.105 mmol). The reaction mixture was stirred at r.t. for 1 hour and was then concentrated in vacuo. The residue was purified by FCC (25 g SiO₂ cartridge, 0-100% EtOAc in heptane) to afford methyl 3-chloro-5-[[2,4-difluoro-5-[2-(2-hydroxyethoxy)phenyl]phenyl]sulfamoyl]-4-(difluoromethyl)benzoate (241 mg, 78% Yield, 90% pure) as a white solid.

¹H NMR (500 MHz, DMSO) δ 11.08 (s, 1H), 8.32 (d, J=1.7 Hz, 1H), 8.29 (d, J=1.6 Hz, 1H), 7.76 (t, J=51.5 Hz, 1H), 7.42-7.33 (m, 2H), 7.32-7.27 (m, 1H), 7.17-7.11 (m, 2H), 7.05-7.01 (m, 1H), 4.67 (br. s, 1H), 3.99 (t, J=5.6 Hz, 2H), 3.90 (s, 3H), 3.59 (t, J=5.5 Hz, 2H).

LCMS: m/z=546.1/548.1 [M−H]−, (ESI−), RT=1.02, Method B

### Step 6

To a solution of methyl 3-chloro-5-[[2,4-difluoro-5-[2-(2-hydroxyethoxy)phenyl]phenyl]sulfamoyl]-4-(difluoromethyl)benzoate (90% purity, 235 mg, 0.386 mmol) in THE (4.5 mL) was added 2 M aq. sodium hydroxide (1.5 mL, 3.00 mmol). The reaction mixture was stirred at r.t. for 15.5 hours and then the organics were removed in vacuo. To the remaining aqueous was added 1 M aq. HCl (10 mL). The mixture was extracted with DCM (3×20 mL), combined, passed through a hydrophobic frit and concentrated in vacuo to afford 3-chloro-5-[[2,4-difluoro-5-[2-(2-hydroxyethoxy)phenyl]phenyl]sulfamoyl]-4-(difluoromethyl)benzoic acid (226 mg, 91% Yield, 83% purity) as a yellow oil/gum.

¹H NMR (400 MHz, DMSO) δ 11.04 (br. s, 1H), 8.31-8.28 (m, 2H), 7.76 (t, J=51.6 Hz, 1H), 7.43-7.34 (m, 2H), 7.31-7.26 (m, 1H), 7.18-7.11 (m, 2H), 7.05-7.00 (m, 1H), 4.00 (t, J=5.5 Hz, 2H), 3.63-3.57 (m, 2H). COOH and OH not observed.

LCMS: m/z=532.0/534.0 [M−H]−, (ESI−), RT=0.90, Method B

### Step 7

To a solution of 3-chloro-5-[[2,4-difluoro-5-[2-(2-hydroxyethoxy)phenyl]phenyl]sulfamoyl]-4-(difluoromethyl)benzoic acid (83% purity, 223 mg, 0.347 mmol) in anhydrous DCM (10.5 mL) was added DMAP (9.0 mg, 0.074 mmol) and DCC (140 mg, 0.679 mmol). The reaction mixture was stirred at r.t. for 1.5 hours and was then concentrated in vacuo. The residue was purified by FCC (25 g SiO₂ cartridge, 20-100% DCM in heptane) to afford the title compound as a white solid (96 mg, 53% Yield, 99% purity).

¹H NMR (500 MHz, DMSO) δ 11.02 (s, 1H), 8.27 (d, J=1.6 Hz, 1H), 8.06 (t, J=51.8 Hz, 1H), 7.63 (s, 1H), 7.47 (ddd, J=8.2, 7.4, 1.7 Hz, 1H), 7.39-7.35 (m, 1H), 7.31 (dd, J=7.5, 1.7 Hz, 1H), 7.25-7.19 (m, 2H), 7.13-7.09 (m, 1H), 4.44-4.39 (m, 2H), 4.35-4.29 (m, 2H).

LCMS: m/z=514.2/516.2 [M−H]−, (ESI−), RT=4.24, Method A

### Example 60—Synthesis of 16-(difluoromethyl)-21,23-difluoro-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one (Compound 60)

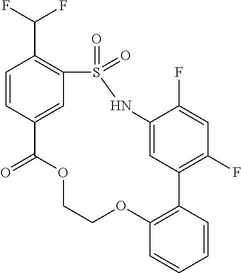 [SMILES: O=C1OCCOc2ccccc2-c2cc(c(F)cc2F)NS(=O)(=O)c2cc1ccc2C(F)F]

A suspension of Example 59 (99% purity, 60 mg, 0.115 mmol), potassium carbonate (48 mg, 0.347 mmol), and t-BuXPhos Pd G3 catalyst (5.0 mg, 6.29 mol) in DMF (0.6 mL) and water (0.06 mL) was sparged with nitrogen for 2 minutes and was then heated in a microwave at 115° C. for 40 minutes. The mixture was filtered through a cotton wool pad and the residue purified by preparative HPLC (Method P1) then lyophilized to afford the title compound (8.7 mg, 15% Yield, 96% purity) as a white solid.

¹H NMR (500 MHz, DMSO) δ 8.21-8.16 (m, 1H), 8.01-7.73 (m, 3H), 7.47-7.41 (m, 1H), 7.40-7.34 (m, 1H), 7.29-7.24 (m, 1H), 7.21-7.15 (m, 1H), 7.11-7.00 (m, 2H), 4.43-4.39 (m, 2H), 4.33-4.28 (m, 2H). NH not observed.

LCMS: m/z=480.1 [M−H]−, (ESI−), RT=3.91, Method A

### Example 61—Synthesis of 15-chloro-21,23-difluoro-18,18-dioxo-8,11-dioxa-18λ6-thia-16,19-diazatetracyclo[18.3.1.113,17.02,7]pentacosa-1(24),2,4,6,13,15,17(25),20,22-nonaen-12-one (Compound 61)

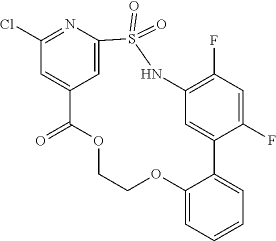 [SMILES: O=C1OCCOc2ccccc2-c2cc(c(F)cc2F)NS(=O)(=O)c2cc1cc(Cl)n2]

### Step 1

To a suspension of potassium tert-butoxide (1.27 g, 11.3 mmol) in anhydrous THE (90 mL) at 0° C. was added phenylmethanethiol (1.1 mL, 9.39 mmol). The reaction mixture was stirred at 0° C. for 10 min before a solution of methyl 2,6-dichloropyridine-4-carboxylate (2.50 g, 12.1 mmol) in anhydrous THE (20 mL) was added. The reaction mixture was kept at 0° C. for 1 min before it was allowed to warm to r.t, and stirred for a further 1 hour. The mixture was concentrated in vacuo and the residue was purified FCC (100 g SiO₂ cartridge, 0-5% EtOAc in heptane) to afford methyl 2-benzylsulfanyl-6-chloropyridine-4-carboxylate (2.55 g, 68% Yield, 74% purity) as a pink oil.

¹H NMR (500 MHz, DMSO) δ 7.69 (d, J=1.1 Hz, 1H), 7.54 (d, J=1.1 Hz, 1H), 7.45-7.42 (m, 2H), 7.34-7.23 (m, 3H), 4.44 (s, 2H), 3.87 (s, 3H).

LCMS: m/z=294.0/296.0 [M+H]+, (ESI+), RT=1.17, Method B

### Step 2

Methyl 2-benzylsulfanyl-6-chloropyridine-4-carboxylate (74% purity, 2.55 g, 6.42 mmol) was dissolved in a mixture of MeCN (24 mL), acetic acid (1.2 mL) and water (1.2 mL) and the resulting suspension was cooled to 0° C. in an ice bath. 1,3-dichloro-5,5-dimethylhydantoin (1.40 g, 7.11 mmol) was then added portion wise and the reaction mixture was stirred for 30 min at 0° C. The organics were concentrated under reduced pressure (water bath at room temperature) and were then diluted with DCM (20 mL). The organics were washed with sat. aq. NaHCO₃ (20 mL), separated, passed through a phase separator and then concentrated under reduce pressure (water bath at room temperature) which gave a yellow oil. A solution of Intermediate 8 (88% purity, 1.70 g, 3.94 mmol) in pyridine (18 mL) was added and the mixture was stirred at 35° C. for 1 hour. The mixture was concentrated in vacuo and the residue was purified by FCC (50 g SiO₂ cartridge, 0-40% EtOAc in heptane) to afford methyl 2-[[5-[2-[2-[tert-butyl(dimethyl)silyl]oxyethoxy]phenyl]-2,4-difluoro-phenyl]sulfamoyl]-6-chloropyridine-4-carboxylate (1.56 g, 55% Yield, 85% purity) as a yellow oil.

¹H NMR (500 MHz, DMSO) δ 10.77 (s, 1H), 8.19 (d, J=1.2 Hz, 1H), 8.15 (d, J=1.2 Hz, 1H), 7.38 (ddd, J=8.3, 7.4, 1.8 Hz, 1H), 7.31-7.25 (m, 2H), 7.15-7.10 (m, 2H), 7.04-7.00 (m, 1H), 4.02-3.99 (m, 2H), 3.92 (s, 3H), 3.79-3.75 (m, 2H), 0.73 (s, 9H), −0.17 (s, 6H).

LCMS: m/z=611.2/613.2 [M−H]−, (ESI−), RT=1.27, Method B

### Step 3

To a solution of methyl 2-[[5-[2-[2-[tert-butyl(dimethyl)silyl]oxyethoxy]phenyl]-2,4-difluoro-phenyl]sulfamoyl]-6-chloropyridine-4-carboxylate (85% purity, 1.56 g, 2.2 mmol) in methanol (25 mL) at r.t. was added p-methylbenzenesulfonic acid hydrate (1:1) (50 mg, 0.263 mmol). The reaction mixture was stirred at r.t. for 17.5 hours and was then concentrated in vacuo. The residue was purified by FCC (25 g SiO₂ cartridge, 0-100% EtOAc in heptane, then 0-20% MeOH in EtOAc) to afford methyl 2-chloro-6-[[2,4-difluoro-5-[2-(2-hydroxyethoxy)phenyl]phenyl]sulfamoyl]pyridine-4-carboxylate (960 mg, 1.85 mmol, 83% Yield, 96% purity) as an off-white solid.

¹H NMR (400 MHz, DMSO) δ 10.74 (s, 1H), 8.18 (d, J=1.2 Hz, 1H), 8.14 (d, J=1.2 Hz, 1H), 7.39 (ddd, J=8.4, 7.4, 1.8 Hz, 1H), 7.36-7.26 (m, 2H), 7.17-7.12 (m, 2H), 7.05-6.99 (m, 1H), 4.66 (t, J=5.5 Hz, 1H), 3.99 (t, J=5.5 Hz, 2H), 3.92 (s, 3H), 3.61-3.56 (m, 2H).

LCMS: m/z=497.1/499.1 [M−H]−, (ESI−), RT=0.93, Method B

### Step 4

To a solution of methyl 2-chloro-6-[[2,4-difluoro-5-[2-(2-hydroxyethoxy)phenyl]phenyl]sulfamoyl]pyridine-4-carboxylate (96% purity, 945 mg, 1.82 mmol) in THE (20 mL) was added 2 M aq. sodium hydroxide (7.0 mL, 14.0 mmol). The reaction mixture was stirred at r.t. for 1.25 hour and then the organics were diluted with EtOAc (40 mL) and washed with 1 M aq. HCl (20 mL). The aqueous layer was extracted with EtOAc (20 mL), the organic layers combined, washed with brine (20 mL), dried over MgSO₄ and concentrated in vacuo to afford 2-chloro-6-[[2,4-difluoro-5-[2-(2-hydroxyethoxy)phenyl]phenyl]sulfamoyl]pyridine-4-carboxylic acid (1.25 g, 99% Yield, 70% purity) as a yellow oil.

¹H NMR (400 MHz, DMSO) δ 10.70 (s, 1H), 8.14 (d, J=1.2 Hz, 1H), 8.13 (d, J=1.1 Hz, 1H), 7.39 (ddd, J=8.4, 7.4, 1.8 Hz, 1H), 7.36-7.26 (m, 2H), 7.16-7.12 (m, 2H), 7.04-7.00 (m, 1H), 4.01-3.96 (m, 2H), 3.62-3.55 (m, 2H), 3.34 (br. s, 1H). COOH not observed LCMS: m/z=483.1/485.1 [M−H]−, (ESI−), RT=0.77, Method B

### Step 5

To a solution of 2-chloro-6-[[2,4-difluoro-5-[2-(2-hydroxyethoxy)phenyl]phenyl]sulfamoyl]pyridine-4-carboxylic acid (70% purity, 1.25 g, 1.80 mmol) in anhydrous DCM (57 mL) was added DMAP (47 mg, 0.385 mmol) and DCC (730 mg, 3.54 mmol). The reaction mixture was stirred at r.t. for 1.5 hour and was then concentrated in vacuo. The residue was purified by FCC (25 g SiO₂ cartridge, 40-100% DCM in EtOAc, then 0-20% EtOAc in DCM) to afford the title compound (63 mg, 7.4% Yield, 99% purity) as a white solid. Further product was isolated at lower purity and used in subsequent reactions (433 mg).

¹H NMR (500 MHz, DMSO) δ 10.62 (s, 1H), 8.17 (d, J=1.2 Hz, 1H), 7.45 (ddd, J=8.2, 7.4, 1.8 Hz, 1H), 7.41 (d, J=1.2 Hz, 1H), 7.29-7.24 (m, 3H), 7.21-7.18 (m, 1H), 7.10-7.06 (m, 1H), 4.49-4.45 (m, 2H), 4.37-4.32 (m, 2H).

LCMS: m/z=465.3/467.3 [M−H]−, (ESI−), RT=3.83, Method A Example 62—Synthesis of 21,23-difluoro-15-methyl-18,18-dioxo-8,11-dioxa-18λ6-thia-16,19-diazatetracyclo[18.3.1.113,17.02,7]pentacosa-1(24),2,4,6,13,15,17(25),20,22-nonaen-12-one (Compound 62)

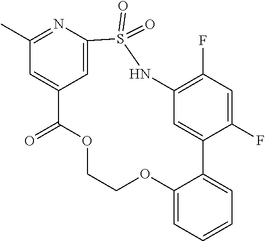 [SMILES: Cc1cc2cc(n1)S(=O)(=O)Nc1cc(c(F)cc1F)-c1ccccc1OCCOC2=O]

2 M Trimethylaluminum solution in toluene (0.11 mL, 0.220 mmol) was added to a sparged solution of Example 61 (87% purity, 20 mg, 0.0373 mmol) and tetrakis(triphenylphosphine)palladium(0) (5.0 mg, 4.33 μmol) in anhydrous 1,4-dioxane (0.6 mL). The vial was sealed and heated at 120° C. under microwave irradiation for 0.75 hour. To the reaction mixture was cautiously added water (0.5 mL). The mixture was extracted with DCM (3×5 mL), combined, passed through a hydrophobic frit and concentrated in vacuo. The residue was purified by preparative HPLC (Method P1) then lyophilized to afford the title compound (7.8 mg, 45% Yield, 96% purity) as a white solid.

¹H NMR (500 MHz, DMSO) δ 10.38 (s, 1H), 8.01-7.80 (m, 1H), 7.48-7.40 (m, 1H), 7.38-7.22 (m, 3H), 7.21-7.11 (m, 2H), 7.11-7.05 (m, 1H), 4.46-4.40 (m, 2H), 4.36-4.28 (m, 2H), 2.64 (s, 3H).

LCMS: m/z=445.1 [M−H]−, (ESI−), RT=3.42, Method A

### Example 63—Synthesis of 15-amino-21,23-difluoro-18,18-dioxo-8,11-dioxa-18λ6-thia-16,19-diazatetracyclo[18.3.1.113,17.02,7]pentacosa-1(23),2(7),3,5,13,15,17(25),20(24),21-nonaen-12-one (Compound 63)

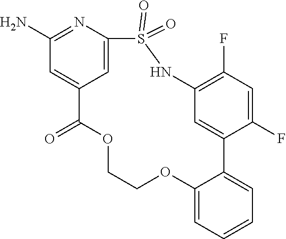 [SMILES: Nc1cc2cc(n1)S(=O)(=O)Nc1cc(c(F)cc1F)-c1ccccc1OCCOC2=O]

A suspension of Example 61 (95% purity, 70 mg, 0.142 mmol), palladium(II) diacetate (4.0 mg, 0.018 mmol) and Xantphos (14 mg, 0.024 mmol) in 1,4-dioxane (1.5 mL) was sparged with nitrogen for 2 minutes and then cesium carbonate (95 mg, 0.292 mmol) and 1,1-diphenylmethanimine (40 μL, 0.238 mmol) were added. The reaction mixture was heated at 100° C. for 3 hours and was then allowed to cool to r.t, and filtered through Celite. The organics were concentrated in vacuo and then re-dissolved in THE (1.5 mL) before 2 M aqueous hydrogen chloride (0.18 mL, 0.360 mmol) was added. The resultant mixture was stirred at r.t. for 15 minutes before sat. aq. NaHCO₃ (5 mL) and water (5 mL) were added. The mixture was extracted with EtOAc (20 mL), the organic layers washed with brine (10 mL) and were then dried over MgSO₄, filtered and concentrated in vacuo. The residue was purified by preparative HPLC (Method P3) followed by lyophilization to afford the title compound (6.7 mg, 8.7% Yield, 83% purity) as a white solid.

¹H NMR (500 MHz, DMSO) δ 10.06 (s, 1H), 7.47-7.42 (m, 1H), 7.31-7.26 (m, 1H), 7.25-7.20 (m, 2H), 7.19-7.16 (m, 1H), 7.10-7.06 (m, 2H), 7.00-6.96 (m, 2H), 6.57 (s, 1H), 4.39-4.36 (m, 2H), 4.33-4.27 (m, 2H).

LCMS: m/z=448.0 [M+H]+, (ESI−), RT=3.16, Method A

### Example 64—Synthesis of 15-chloro-16,21,23-trifluoro-18,18-dioxo-8,11-dioxa-18%6-thia-19-azatetracyclo[18.3.1.113,17.02,7]pentacosa-1(23),2(7),3,5,13(25),14,16,20(24),21-nonaen-12-one (Compound 64)

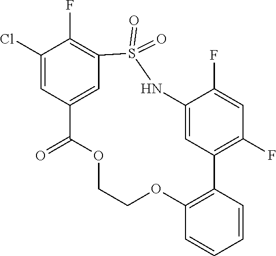 [SMILES: O=C1OCCOc2ccccc2-c2cc(c(F)cc2F)NS(=O)(=O)c2cc1cc(Cl)c2F]

### Step 1

A solution of methyl 3-bromo-5-chloro-4-fluorobenzoate (70% purity, 2.00 g, 5.23 mmol), benzyl mercaptan (725 μL, 6.19 mmol), DIPEA (1.2 mL, 6.89 mmol), Pd₂(dba)₃ (160 mg, 0.175 mmol) and Xantphos (200 mg, 0.346 mmol) in 1,4-dioxane (40 mL) was degassed by sparging with nitrogen. The reaction was heated to 100° C. for 1 hour then stirred for 18 hours at 80° C. The reaction was cooled and the solid material removed by filtration, washing with EtOAc (solid discarded). The filtrate was partitioned between water and EtOAc. The aqueous layer was extracted with EtOAc (3×10 mL), the combined organics washed with brine, dried over MgSO₄ and concentrated in vacuo. The residue was purified by FCC (100 g SiO₂ column, 0-20% EtOAc in heptane) to afford methyl 3-benzylsulfanyl-5-chloro-4-fluorobenzoate (2.20 g, 92% Yield, 68% purity) as a yellow solid.

¹H NMR (500 MHz, CDCl3) δ 7.92 (dd, J=6.6, 2.1 Hz, 1H), 7.88 (dd, J=6.6, 2.1 Hz, 1H), 7.31-7.28 (m, 4H), 7.26-7.23 (m, 1H), 4.17 (s, 2H), 3.90 (s, 3H).

LCMS: m/z=309.1/311.1 [M−H]−, (ESI−), RT=1.19, Method B

### Step 2

1,3-Dichloro-5,5-dimethylhydantoin (450 mg, 2.28 mmol) was added to an ice-cold solution of methyl 3-benzylsulfanyl-5-chloro-4-fluorobenzoate (68% purity, 500 mg, 1.09 mmol) in MeCN (9 mL), water (1 mL) and acetic acid (1 mL). The reaction was stirred for 1 hour then the solvent volume reduced in vacuo and then diluted with DCM (˜15 mL). The mixture was quenched with saturated aq. NaHCO₃ and the aqueous layer was extracted with DCM (3×10 mL). The combined organics were washed with brine, dried over MgSO₄ and concentrated in vacuo. A solution of Intermediate 8 (315 mg, 0.830 mmol) in pyridine (4.5 mL) was added and the mixture was stirred for 2 hours. The mixture was quenched into 1 M aq. HCl and the aqueous layer was extracted into EtOAc (3×10 mL), the combined organics washed with brine, dried over MgSO₄ and concentrated in vacuo. The residue was purified by FCC (25 g SiO₂ column, 0-100% EtOAc in heptane) to afford methyl 3-[[5-[2-[2-[tert-butyl(dimethyl)silyl]oxyethoxy]phenyl]-2,4-difluoro-phenyl]sulfamoyl]-5-chloro-4-fluorobenzoate (243 mg, 39% Yield, 84% purity).

¹H NMR (400 MHz, DMSO) δ 10.92 (s, 1H), 8.42-8.34 (m, 1H), 8.16 (dd, J=6.0, 2.2 Hz, 1H), 7.42-7.33 (m, 1H), 7.34-7.26 (m, 1H), 7.26-7.18 (m, 1H), 7.16-7.09 (m, 2H), 7.05-6.97 (m, 1H), 4.00 (t, J=4.7 Hz, 2H), 3.87 (s, 3H), 3.77 (t, J=4.6 Hz, 2H), 0.73 (s, 9H), −0.18 (s, 6H).

LCMS: m/z=628.2/630.2 [M−H]−, (ESI−), RT=1.29, Method B

### Step 3

p-Toluenesulfonic acid monohydrate (7.0 mg, 0.037 mmol) was added to a solution of methyl 3-[[5-[2-[2-[tert-butyl(dimethyl)silyl]oxyethoxy]phenyl]-2,4-difluoro-phenyl]sulfamoyl]-5-chloro-4-fluorobenzoate (84% purity, 250 mg, 0.333 mmol) in MeOH (10 mL). The mixture was stirred for 1 hour then concentrated in vacuo. The residue was purified by FCC (10 g, SiO₂ column, 20-80% EtOAc in heptane) to afford methyl 3-chloro-5-[[2,4-difluoro-5-[2-(2-hydroxyethoxy)phenyl]phenyl]sulfamoyl]-4-fluorobenzoate (143 mg, 72% Yield, 86% purity) as a white solid.

¹H NMR (500 MHz, DMSO) δ 10.88 (s, 1H), 8.39-8.34 (m, 1H), 8.14 (dd, J=6.0, 2.2 Hz, 1H), 7.41-7.36 (m, 1H), 7.36-7.28 (m, OH), 7.13 (ddd, J=9.7, 7.9, 1.4 Hz, 2H), 7.05-6.98 (m, 1H), 4.67 (t, J=5.5 Hz, 1H), 3.98 (t, J=5.5 Hz, 2H), 3.87 (s, 3H), 3.58 (q, J=5.4 Hz, 2H).

LCMS: m/z=514.1/516.1 [M−H]−, (ESI−), RT=0.97, Method B

### Step 4

2 M aq. sodium hydroxide solution (0.50 mL, 1.00 mmol) was added to a solution of methyl 3-chloro-5-[[2,4-difluoro-5-[2-(2-hydroxyethoxy)phenyl]phenyl]sulfamoyl]-4-fluorobenzoate (86% purity, 143 mg, 0.238 mmol) in THE (2 mL). The mixture was stirred for 1 hour then quenched into water/EtOAc. The layers were separated and the organics discarded. The aqueous layer was acidified to pH 4 with aq. HCl and extracted into EtOAc (3×5 mL). The combined organics were washed with brine/HCl, dried over MgSO₄ and concentrated in vacuo to afford 3-chloro-5-[[2,4-difluoro-5-[2-(2-hydroxyethoxy)phenyl]phenyl]sulfamoyl]-4-fluorobenzoic acid (115 mg, 85% Yield, 88% purity) as a colorless oil.

LCMS: m/z=500.1/502.0 [M−H]−, (ESI−), RT=0.86, Method B

### Step 5

DMAP (2.0 mg, 0.0164 mmol) was added to a solution of 3-chloro-5-[[2,4-difluoro-5-[2-(2-hydroxyethoxy)phenyl]phenyl]sulfamoyl]-4-fluorobenzoic acid (88% purity, 105 mg, 0.184 mmol) and DCC (85 mg, 0.412 mmol) in DCM (20 mL). The mixture was stirred for 1 hour then quenched into water. The aqueous layer was extracted with DCM (3×5 mL), the combined organics washed with brine, dried over MgSO₄ and concentrated in vacuo. The residue was purified by preparative HPLC (Method P1) to afford the title compound (30 mg, 33% Yield, 98% purity) as a white solid.

¹H NMR (500 MHz, DMSO) δ 10.93 (s, 1H), 8.34-8.29 (m, 1H), 7.52-7.42 (m, 2H), 7.34-7.26 (m, 2H), 7.26-7.16 (m, 2H), 7.14-7.06 (m, 1H), 4.43-4.38 (m, 2H), 4.34-4.28 (m, 2H).

LCMS: m/z=482.0,484.0 [M−H]−, (ESI−), RT=3.97, Method A

### Example 65—Synthesis of 21,23-difluoro-15-hydroxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (Compound 65)

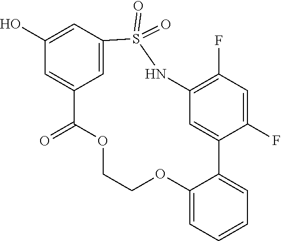 [SMILES: O=C1OCCOc2ccccc2-c2cc(c(F)cc2F)NS(=O)(=O)c2cc(O)cc1c2]

To 21,23-difluoro-15-methoxy-18,18-dioxo-8,11-dioxa-18λ6-thia-19-azatetracyclo[18.3.1.113,17.02,7]pentacosa-1(24),2,4,6,13,15,17(25),20,22-nonaen-12-one (synthesized using a similar method to Intermediate 12, 90% purity, 90 mg, 0.176 mmol) was added dropwise 1 M BBr₃ in DCM (9.0 mL, 9.00 mmol). The resulting solution was stirred at r.t. for 2 hours. The reaction mixture was slowly added to a stirring, ice-cooled sat. aq. solution of NaHCO₃ (50 mL) and extracted with DCM (3×50 mL). The combined organic layers were washed with brine (100 mL), dried over Na₂SO₄ and concentrated under reduced pressure. The residue was purified by preparative HPLC (Method P1) to afford the title compound as a white solid (20 mg, 25% Yield, 99% purity).

¹H NMR (500 MHz, DMSO) δ 10.68 (s, 1H), 10.09 (s, 1H), 7.48-7.41 (m, 3H), 7.31-7.21 (m, 3H), 7.20-7.16 (m, 1H), 7.11-7.06 (m, 1H), 6.99 (s, 1H), 4.38-4.34 (m, 2H), 4.33-4.29 (m, 2H).

LCMS: m/z=446.1 [M−H]−, (ESI−), RT=3.32, Method A

### Example 66—Synthesis of 21-fluoro-16-hydroxy-18,18-dioxo-8,11-dioxa-18λ6-thia-14,19-diazatetracyclo[18.3.1.113,17.02,7]pentacosa-1(24),2,4,6,13,15,17(25),20,22-nonaen-12-one (Compound 66)

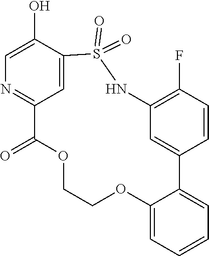 [SMILES: O=C1OCCOc2ccccc2-c2ccc(F)c(c2)NS(=O)(=O)c2cc1ncc2O]

### Step 1

To a solution of methyl 4-chloro-5-methoxy-pyridine-2-carboxylate (860 mg, 4.27 mmol) in anhydrous 1,4-dioxane (22 mL) was added DIPEA (0.90 mL, 5.17 mmol) and the solution was sparged with nitrogen for 10 minutes. Phenylmethanethiol (0.45 mL, 3.84 mmol), Pd₂(dba)₃ (115 mg, 0.126 mmol) and Xantphos (150 mg, 0.259 mmol) were added and the reaction mixture was sealed and heated at 100° C. for 16 hours. The reaction mixture was cooled to r.t, and concentrated in vacuo. The residue was purified by FCC (50 g SiO₂ cartridge, 0-50% acetone in heptane) to afford gave methyl 4-benzylsulfanyl-5-methoxy-pyridine-2-carboxylate (465 mg, 40% Yield, 95% purity) as a pale-yellow solid.

¹H NMR (500 MHz, DMSO) δ 8.29 (s, 1H), 7.93 (s, 1H), 7.47-7.43 (m, 2H), 7.37-7.32 (m, 2H), 7.30-7.25 (m, 1H), 4.36 (s, 2H), 4.01 (s, 3H), 3.84 (s, 3H).

### Step 2

Methyl 4-benzylsulfanyl-5-methoxy-pyridine-2-carboxylate (95% purity, 230 mg, 0.755 mmol) was dissolved in a mixture of MeCN (3.6 mL), acetic acid (0.28 mL) and water (0.28 mL) and the resulting suspension was cooled to 0° C. in an ice bath. 1,3-dichloro-5,5-dimethylhydantoin (295 mg, 1.50 mmol) was then added portion wise and the reaction mixture was stirred for 1 minute at 0° C. before a solution of 5-[2-[2-[tert-butyl(dimethyl)silyl]oxyethoxy]phenyl]-2-fluoroaniline (synthesized using a similar method to Intermediate 8, 95% purity, 200 mg, 0.526 mmol) in pyridine (4 mL) was added. The reaction mixture was heated at 50° C. for 20 minutes and was then allowed to cool to r.t. The mixture was diluted with EtOAc (40 mL) and washed with 1 M aq. HCl (40 mL), then sat. aq. NaHCO₃ (30 mL), then brine (20 mL), dried over MgSO₄, filtered and concentrated in vacuo. The residue was purified by FCC (25 g SiO₂ cartridge, 0-70% EtOAc in heptane) to afford methyl 4-[[5-[2-[2-[tert-butyl(dimethyl)silyl]oxyethoxy]phenyl]-2-fluoro-phenyl]sulfamoyl]-5-methoxy-pyridine-2-carboxylate (175 mg) as an off-white solid. LCMS: m/z=589.3 [M−H]−, (ESI−), RT=1.21, Method B

### Step 3

To a solution of methyl 4-[[5-[2-[2-[tert-butyl(dimethyl)silyl]oxyethoxy]phenyl]-2-fluoro-phenyl]sulfamoyl]-5-methoxy-pyridine-2-carboxylate (30% purity, 170 mg, 0.086 mmol) in MeOH (1 mL) at r.t. was added p-methylbenzenesulfonic acid hydrate (1:1) (3.0 mg, 0.0158 mmol). The reaction mixture was stirred at r.t. for 1.25 hours and was then concentrated in vacuo. The residue was purified by FCC (10 g SiO₂ cartridge, 20-100% EtOAc in heptane) to afford methyl 4-[[2-fluoro-5-[2-(2-hydroxyethoxy)phenyl]phenyl]sulfamoyl]-5-methoxy-pyridine-2-carboxylate (46 mg, 73% Yield, 65% purity) as a yellow oil.

LCMS: m/z=475.2 [M−H]−, (ESI−), RT=0.79, Method B

### Step 4

To a solution of methyl 4-[[2-fluoro-5-[2-(2-hydroxyethoxy)phenyl]phenyl]sulfamoyl]-5-methoxy-pyridine-2-carboxylate (65% purity, 43 mg, 0.059 mmol) in THE (1 mL) was added 2 M aq. sodium hydroxide (0.25 mL, 0.50 mmol). The reaction mixture was stirred at r.t. for 2.25 hours and then to the reaction mixture was added 1 M aq. HCl (8 mL). The mixture was extracted with EtOAc (2×30 mL), the organic layers were passed through a hydrophobic frit and concentrated in vacuo to afford 4-[[2-fluoro-5-[2-(2-hydroxyethoxy)phenyl]phenyl]sulfamoyl]-5-methoxy-pyridine-2-carboxylic acid (27 mg, quantitative yield) as a yellow oil.

LCMS: m/z=461.1 [M−H]−, (ESI−), RT=0.70, Method B

### Step 5

To a solution of 4-[[2-fluoro-5-[2-(2-hydroxyethoxy)phenyl]phenyl]sulfamoyl]-5-methoxy-pyridine-2-carboxylic acid (27 mg, 0.058 mmol) in DCM (10 mL) was added DMAP (3.0 mg, 0.0246 mmol) and DCC (30 mg, 0.145 mmol). The reaction mixture was stirred at r.t. for 1.5 hours and was then concentrated in vacuo. The residue was purified by preparative HPLC (Method P3), followed by FCC (10 g SiO₂ cartridge, 0-100% acetone in heptane) to afford 21-fluoro-16-methoxy-18,18-dioxo-8,11-dioxa-18λ6-thia-14,19-diazatetracyclo[18.3.1.113,17.02,7]pentacosa-1(24),2,4,6,13,15,17(25),20,22-nonaen-12-one (10 mg, 35% Yield, 90% purity) as a white solid.

¹H NMR (500 MHz, CDCl₃) δ 8.61 (s, 1H), 7.83 (s, 1H), 7.61 (dd, J=7.6, 2.2 Hz, 1H), 7.37 (ddd, J=8.2, 7.4, 1.8 Hz, 1H), 7.21 (dd, J=7.5, 1.8 Hz, 1H), 7.12 (ddd, J=8.4, 5.1, 2.2 Hz, 1H), 7.08-7.04 (m, 1H), 7.03-6.98 (m, 1H), 6.98-6.83 (m, 1H), 6.77 (dd, J=10.1, 8.5 Hz, 1H), 4.50-4.47 (m, 2H), 4.32-4.28 (m, 2H), 4.21 (s, 3H).

LCMS: m/z=445.3 [M+H]+, (ESI+), RT=0.87, Method B

### Step 6

To a solution of 21-fluoro-16-methoxy-18,18-dioxo-8,11-dioxa-18λ6-thia-14,19-diazatetracyclo[18.3.1.113,17.02,7]pentacosa-1(24),2,4,6,13,15,17(25),20,22-nonaen-12-one (90% purity, 10 mg, 0.0203 mmol) in anhydrous pyridine (1.2 mL) was added lithium iodide (30 mg, 0.224 mmol). The reaction mixture was heated at 80° C. for 50 hours and then cooled to r.t. The mixture was diluted with EtOAc (30 mL), washed with 1 M aq. HCl (2×30 mL), then sat. aq. Na₂SO₃ (10 mL), then brine (2×20 mL), dried over MgSO4, filtered and concentrated in vacuo to afford the title compound (5.5 mg, 56% Yield, 89% purity) as an off-white solid.

¹H NMR (500 MHz, DMSO) δ 8.07-7.92 (m, 1H), 7.75 (s, 1H), 7.39 (dd, J=7.8, 2.1 Hz, 1H), 7.38-7.31 (m, 1H), 7.20 (dd, J=7.4, 1.8 Hz, 1H), 7.17-7.11 (m, 1H), 7.08-6.97 (m, 3H), 4.38-4.26 (m, 2H), 4.26-4.14 (m, 2H).

LCMS: m/z=431.0 [M+H]+, (ESI+), RT=3.04, Method A

### Example 67—Synthesis of 15-chloro-21,23-difluoro-18,18-dioxo-8,11-dioxa-18λ6-thia-19-azatetracyclo[18.3.1.113,17.02,7]pentacosa-1(23),2(7),3,5,13,15,17(25),20(24),21-nonaen-16-ol (Compound 67)

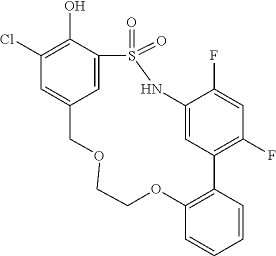 [SMILES: O=S1(=O)Nc2cc(c(F)cc2F)-c2ccccc2OCCOCc2cc(Cl)c(O)c1c2]

### Step 1

To a solution of Intermediate 9 (90% purity, 1.90 g, 2.66 mmol) in MeOH (15.5 mL) at r.t. was added p-methylbenzenesulfonic acid hydrate (1:1) (50 mg, 0.263 mmol). The reaction mixture was stirred for 1 hour at r.t, and was then concentrated in vacuo. The residue was purified by FCC (25 g SiO₂ cartridge, 0-40% EtOAc in heptane) to afford methyl 3-chloro-5-[[2,4-difluoro-5-[2-(2-hydroxyethoxy)phenyl]phenyl]sulfamoyl]-4-methoxybenzoate (1.20 g, 81% Yield, 95% purity) as a white solid.

¹H NMR (400 MHz, DMSO) δ 10.36 (s, 1H), 8.25 (d, J=2.1 Hz, 1H), 8.17 (d, J=2.1 Hz, 1H), 7.38 (ddd, J=8.3, 7.3, 1.8 Hz, 1H), 7.34-7.27 (m, 1H), 7.24-7.18 (m, 1H), 7.15-7.12 (m, 1H), 7.09 (dd, J=7.5, 1.8 Hz, 1H), 7.01 (s, 1H), 4.66 (t, J=5.5 Hz, 1H), 3.98 (t, J=5.5 Hz, 2H), 3.96 (s, 3H), 3.86 (s, 3H), 3.60-3.55 (m, 2H).

LCMS: m/z=526.1/528.1 [M−H]−, (ESI−), RT=0.99, Method B

### Step 2

To a solution of methyl 3-chloro-5-[[2,4-difluoro-5-[2-(2-hydroxyethoxy)phenyl]phenyl]sulfamoyl]-4-methoxy-benzoate (95% purity, 400 mg, 0.720 mmol) and tetrabromomethane (264 mg, 0.796 mmol) in anhydrous DCM (4 mL) at 0° C. was added triphenylphosphine (216 mg, 0.824 mmol). The reaction mixture was allowed to warm to r.t, and was stirred for 3 hours, then concentrated in vacuo. The residue was purified by FCC (25 g SiO₂ cartridge, 0-22% EtOAc in heptane) to afford methyl 3-[[5-[2-(2-bromoethoxy)phenyl]-2,4-difluoro-phenyl]sulfamoyl]-5-chloro-4-methoxybenzoate (405 mg, 90% Yield, 95% purity) as a colorless solid.

¹H NMR (400 MHz, DMSO) δ 10.36 (s, 1H), 8.24 (d, J=2.1 Hz, 1H), 8.15 (d, J=2.1 Hz, 1H), 7.40 (ddd, J=8.3, 7.3, 1.8 Hz, 1H), 7.34-7.28 (m, 1H), 7.28-7.23 (m, 1H), 7.17-7.11 (m, 2H), 7.08-7.02 (m, 1H), 4.32-4.26 (m, 2H), 3.96 (s, 3H), 3.86 (s, 3H), 3.65-3.61 (m, 2H).

LCMS: m/z=588.0/590.0/591.9 [M−H]−, (ESI−), RT=1.12, Method B

### Step 3

To a solution of methyl 3-[[5-[2-(2-bromoethoxy)phenyl]-2,4-difluoro-phenyl]sulfamoyl]-5-chloro-4-methoxy-benzoate (95% purity, 400 mg, 0.643 mmol) in dry Et₂O (7 mL) at −78° C. was added 1 M DIBAL in DCM (1.9 mL, 1.90 mmol). The reaction mixture was stirred for 3 hours 45 minutes at −78° C., and then at 0° C. for 1 hour before 1 M DIBAL in DCM (3.0 mL, 3.00 mmol) was added. The reaction mixture was stirred at 0° C. for 40 minutes. To the reaction mixture was added water (20 mL), sat. aq. Rochelle's salt (50 mL) and sat. aq. NH₄Cl (20 mL). The mixture was extracted with DCM (3×30 mL), the organics were dried over MgSO₄, filtered and concentrated in vacuo to afford N-[5-[2-(2-bromoethoxy)phenyl]-2,4-difluoro-phenyl]-3-chloro-5-(hydroxymethyl)-2-methoxy-benzenesulfonamide (350 mg, 82% Yield, 85% purity) as a white solid.

¹H NMR (500 MHz, DMSO) δ 10.08 (s, 1H), 7.69 (d, J=2.0 Hz, 1H), 7.64 (d, J=2.0 Hz, 1H), 7.42-7.37 (m, 1H), 7.32-7.26 (m, 1H), 7.23-7.18 (m, 1H), 7.15-7.12 (m, 1H), 7.12-7.10 (m, 1H), 7.08-7.02 (m, 1H), 5.45 (t, J=5.7 Hz, 1H), 4.47 (d, J=5.3 Hz, 2H), 4.29 (t, J=5.5 Hz, 2H), 3.86 (s, 3H), 3.63 (t, J=5.5 Hz, 2H).

LCMS: m/z=560.0/562.0/564.0 [M−H]−, (ESI−), RT=1.01, Method B

### Step 4

To a solution of N-[5-[2-(2-bromoethoxy)phenyl]-2,4-difluoro-phenyl]-3-chloro-5-(hydroxymethyl)-2-methoxy-benzenesulfonamide (85% purity, 340 mg, 0.513 mmol) in anhydrous THE (40 mL) at 0° C. was added sodium hydride in mineral oil (60%, 70 mg, 1.75 mmol). The reaction mixture was heated at 50° C. for 45 minutes and then to the reaction mixture was added sodium hydride in mineral oil (60%, 45 mg, 1.13 mmol) and the reaction mixture was heated at 50° C. for 4 hours. The reaction mixture was allowed to cool to r.t, and then to the reaction mixture was cautiously added water (10 mL) and 1 M aq. HCl (30 mL). The mixture was diluted with EtOAc, the organic layer was separated, washed with sat. aq. NaHCO₃ (50 mL of a saturated aqueous solution), then brine (50 mL), dried over MgSO₄, filtered and concentrated in vacuo. The residue was purified by FCC (25 g SiO₂ cartridge, 0-100% acetone in heptane, then 30% MeOH in acetone) to afford 15-chloro-21,23-difluoro-16-methoxy-8,11-dioxa-18λ6-thia-19-azatetracyclo[18.3.1.113,17.02,7]pentacosa-1(24),2(7),3,5,13(25),14,16,20,22-nonaene 18,18-dioxide (44 mg, 80% purity) as a white solid.

¹H NMR (500 MHz, DMSO) δ 10.39 (s, 1H), 7.69 (d, J=2.1 Hz, 1H), 7.60 (d, J=2.0 Hz, 1H), 7.40-7.36 (m, 1H), 7.32-7.25 (m, 2H), 7.22-7.17 (m, 1H), 7.14 (d, J=8.3 Hz, 1H), 7.03-6.99 (m, 1H), 4.45 (s, 2H), 4.10-4.06 (m, 2H), 3.97 (s, 3H), 3.50-3.47 (m, 2H).

LCMS: m/z=480.0/482.0 [M−H]−, (ESI−), RT=1.06, Method B

### Step 5

Iodocyclohexane (77 μL, 0.595 mmol) was added to a stirred solution of 15-chloro-21,23-difluoro-16-methoxy-8,11-dioxa-18λ6-thia-19-azatetracyclo[18.3.1.113,17.02,7]pentacosa-1(23),2(7),3,5,13,15,17(25),20(24),21-nonaene 18,18-dioxide (80% purity, 35 mg, 0.0581 mmol) in anhydrous DMF (1.2 mL) at r.t, and the mixture was heated at 100° C. for 18 hours. The reaction was cooled to r.t, and the purified by FCC (12 g C18-SiO₂ cartridge, 10-100% MeCN (0.1% formic acid) in water (0.1% formic acid)) followed by preparative HPLC (Method P1) then lyophilized to afford the title compound (6.3 mg, 22% Yield, 93% purity) as a white solid.

¹H NMR (500 MHz, DMSO) δ 7.53-7.51 (m, 1H), 7.51-7.50 (m, 1H), 7.39-7.35 (m, 1H), 7.35-7.30 (m, 1H), 7.27-7.21 (m, 1H), 7.16 (d, J=7.5 Hz, 1H), 7.14 (d, J=8.3 Hz, 1H), 7.03-6.97 (m, 1H), 4.36 (s, 2H), 4.08-4.03 (m, 2H), 3.45-3.40 (m, 2H). OH and NH not observed.

LCMS: m/z=466.0/468.0 [M−H]−, (ESI−), RT=3.73, Method A

### Example 68—Synthesis of 14-chloro-20,22-difluoro-15-hydroxy-17,17-dioxo-9-oxa-17λ6-thia-18-azatetracyclo[17.3.1.112,16.02,7]tetracosa-1(22),2(7),3,5,12(24),13,15,19(23),20-nonaen-10-one (Compound 68)

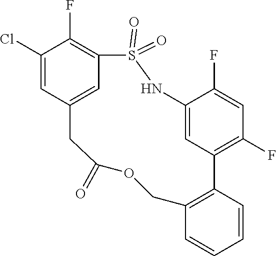 [SMILES: O=C1Cc2cc(Cl)c(F)c(c2)S(=O)(=O)Nc2cc(c(F)cc2F)-c2ccccc2CO1]

### Step 1

A solution of bromine (641 μL, 12.5 mmol) in DCE (25 mL) was added slowly over 15 minutes to an ice-cold solution of methyl 2-(3-chloro-4-hydroxy-phenyl)acetate (2.50 g, 12.5 mmol) in DCE (50 mL). The mixture was stirred for 20 hours then quenched into water. The mixture was extracted into DCM (3×20 mL), the combined organics were washed with sat. aq. sodium thiosulfate (30 mL), dried over MgSO₄ and concentrated in vacuo. The residue was purified by FCC (50 g SiO₂ cartridge, 0-70% EtOAc in heptane) to afford methyl 2-(3-bromo-5-chloro-4-hydroxy-phenyl)acetate (2.48 g, 71% Yield) as a white solid.

¹H NMR (400 MHz, DMSO) δ 7.42 (d, J=2.1 Hz, 1H), 7.31 (d, J=2.1 Hz, 1H), 3.65-3.59 (m, 5H).

LCMS: m/z=276.7/278.9/280.7 [M−H]−, (ESI−), RT=0.79, Method B

### Step 2

Iodomethane (600 μL, 9.64 mmol) was added to a suspension of methyl 2-(3-bromo-5-chloro-4-hydroxy-phenyl)acetate (97% purity, 2.48 g, 8.61 mmol) and potassium carbonate (2.30 g, 16.6 mmol) in DMF (38 mL). The mixture was heated to 50° C. for 1 hour then cooled and quenched with water. The mixture was extracted EtOAc (3×20 mL), the combined organics were washed with brine, dried over MgSO₄ and concentrated in vacuo. The residue was purified by FCC (50 g SiO₂ column, 0-50% EtOAc in heptane) to afford methyl 2-(3-bromo-5-chloro-4-methoxy-phenyl)acetate (2.41 g, 89% Yield, 90% purity) as a colorless oil.

¹H NMR (400 MHz, DMSO) δ 7.56 (d, J=1.9 Hz, 1H), 7.46 (d, J=2.0 Hz, 1H), 3.80 (s, 3H), 3.71 (s, 2H), 3.63 (s, 3H).

### Step 3

A mixture of methyl 2-(3-bromo-5-chloro-4-methoxy-phenyl)acetate (90% purity, 1.20 g, 3.68 mmol), benzyl mercaptan (500 μL, 4.27 mmol), Pd₂(dba)₃ (100 mg, 0.109 mmol), Xantphos (125 mg, 0.216 mmol) and DIPEA (775 μL, 4.45 mmol) in 1,4-dioxane (20 mL) was sparged with nitrogen. The mixture was heated to 100° C. for 18 hours then allowed to cool to r.t. The mixture was filtered and the filtrate was partitioned between EtOAc (20 mL) and water (20 mL). The biphasic mixture was separated and the aqueous layer extracted with EtOAc (3×10 mL). The combined organics were washed with brine, dried over MgSO₄ and concentrated in vacuo. The residue was purified by FCC (50 g SiO₂ column, 0-20% EtOAc in heptane) to afford methyl 2-(3-benzylsulfanyl-5-chloro-4-methoxy-phenyl)acetate (940 mg, 68% Yield, 90% purity) as a yellow oil.

¹H NMR (400 MHz, DMSO) δ 7.42-7.37 (m, 2H), 7.35-7.28 (m, 2H), 7.27-7.22 (m, 2H), 7.20 (d, J=2.0 Hz, 1H), 4.22 (s, 2H), 3.71 (s, 3H), 3.66 (s, 2H), 3.61 (s, 3H).

### Step 4

1,3-Dichloro-5,5-dimethylhydantoin (615 mg, 3.12 mmol) was added portion wise to an ice-cold solution of methyl 2-(3-benzylsulfanyl-5-chloro-4-methoxy-phenyl)acetate (500 mg, 1.48 mmol) in MeCN (12 mL), water (1.5 mL) and acetic acid (1.5 mL). The mixture was stirred for 1 hour then the solvent volume was reduced in vacuo. The residue was taken up in DCM (˜20 mL) and cooled in ice-water. Sat. aq. NaHCO₃ was added and the phases separated. The aqueous layer was extracted with DCM (3×10 mL), the combined organics were washed with brine, dried over MgSO₄ and concentrated in vacuo to yield methyl 2-(3-chloro-5-chlorosulfonyl-4-methoxy-phenyl)acetate (850 mg, 91% Yield, 50% purity) as a white solid.

### Step 5

A solution of Intermediate 8 (429 mg, 1.23 mmol) in pyridine (6 mL) was added to methyl 2-(3-chloro-5-chlorosulfonyl-4-methoxy-phenyl)acetate (50% purity, 850 mg, 1.36 mmol) and the mixture was stirred for 1 hour then quenched with 1 M aq. HCl. The mixture was extracted with EtOAc (3×10 mL), the combined organics were washed with brine, dried over MgSO₄ and concentrated in vacuo. The residue was purified by FCC (50 g SiO₂ column, 50-100% DCM in heptane) to afford methyl 2-[3-[[5-[2-[[tert-butyl(dimethyl)silyl]oxymethyl]phenyl]-2,4-difluoro-phenyl]sulfamoyl]-5-chloro-4-methoxy-phenyl]acetate (651 mg, 80% Yield, 95% purity) as a colorless oil.

¹H NMR (500 MHz, DMSO) δ 10.22 (s, 1H), 7.73 (d, J=2.1 Hz, 1H), 7.60 (d, J=2.2 Hz, 1H), 7.53 (dd, J=7.8, 1.3 Hz, 1H), 7.48-7.42 (m, 1H), 7.40-7.33 (m, 2H), 7.16-7.10 (m, 1H), 7.09 (dd, J=7.6, 1.3 Hz, 1H), 4.44 (s, 2H), 3.86 (s, 3H), 3.75 (s, 2H), 3.58 (s, 3H), 0.80 (s, 9H), −0.09 (s, 6H).

LCMS: m/z=624.2/626.1 [M−H]−, (ESI−), RT=1.31, Method B

### Step 6

p-Toluenesulfonic acid monohydrate (20 mg, 0.105 mmol) was added to a solution of methyl 2-[3-[[5-[2-[[tert-butyl(dimethyl)silyl]oxymethyl]phenyl]-2,4-difluoro-phenyl]sulfamoyl]-5-chloro-4-methoxy-phenyl]acetate (95% purity, 651 mg, 0.988 mmol) in MeOH (25 mL). The mixture was stirred for 1 hour then concentrated in vacuo. The residue was purified by FCC (25 g SiO₂ column, 0-80% EtOAc in heptane) to afford methyl 2-[3-chloro-5-[[2,4-difluoro-5-[2-(hydroxymethyl)phenyl]phenyl]sulfamoyl]-4-methoxy-phenyl]acetate (363 mg, 69% Yield, 96% purity) as a white solid.

¹H NMR (400 MHz, DMSO) δ 10.17 (s, 1H), 7.72 (d, J=2.1 Hz, 1H), 7.60 (d, J=2.2 Hz, 1H), 7.58 (dd, J=7.9, 1.2 Hz, 1H), 7.46-7.40 (m, 1H), 7.39-7.27 (m, 2H), 7.12-7.02 (m, 2H), 5.09 (t, J=5.6 Hz, 1H), 4.21 (d, J=5.6 Hz, 2H), 3.87 (s, 3H), 3.76 (s, 2H), 3.59 (s, 3H).

LCMS: m/z=510.1/512.0 [M−H]−, (ESI−), RT=0.95, Method B

### Step 7

2 M aqueous sodium hydroxide (1.5 mL, 3.00 mmol) was added to a solution of methyl 2-[3-chloro-5-[[2,4-difluoro-5-[2-(hydroxymethyl)phenyl]phenyl]sulfamoyl]-4-methoxy-phenyl]acetate (96% purity, 363 mg, 0.681 mmol) in THE (6.5 mL). The mixture was stirred for 2 hours then quenched with 1 M aq. HCl. The mixture was extracted with EtOAc (3×5 mL), the combined organics were washed with brine, dried over MgSO₄ and concentrated in vacuo to afford 2-[3-chloro-5-[[2,4-difluoro-5-[2-(hydroxymethyl)phenyl]phenyl]sulfamoyl]-4-methoxy-phenyl]acetic acid (350 mg, 99% Yield, 96% purity) as an off white solid.

¹H NMR (500 MHz, DMSO) δ 10.15 (br s, 1H), 7.71 (d, J=2.1 Hz, 1H), 7.61-7.55 (m, 2H), 7.46-7.39 (m, 1H), 7.38-7.28 (m, 2H), 7.12-7.02 (m, 2H), 5.09 (br s, 1H), 4.21 (s, 2H), 3.86 (s, 3H), 3.66 (s, 2H).

LCMS: m/z=496.1/498.1 [M−H]−, (ESI−), RT=0.83, Method B

### Step 8

DMAP (10 mg, 0.0819 mmol) was added to a solution of 2-[3-chloro-5-[[2,4-difluoro-5-[2-(hydroxymethyl)phenyl]phenyl]sulfamoyl]-4-methoxy-phenyl]acetic acid (96%, 330 mg, 0.636 mmol) and DCC (280 mg, 1.36 mmol) in DCM (50 mL). The mixture was stirred for 3 hours then quenched with water. The mixture was extracted into DCM (3×5 mL), the combined organics were washed with brine, dried over MgSO₄ and concentrated in vacuo. The residue was purified by FCC (25 g SiO₂ column, 0-100% EtOAc in heptane), then further purified by FCC (5 g SiO₂ column, 50-100% DCM in heptane) to afford 14-chloro-20,22-difluoro-15-methoxy-17,17-dioxo-9-oxa-17λ6-thia-18-azatetracyclo[17.3.1.112,16.02,7]tetracosa-1(22),2(7),3,5,12(24),13,15,19(23),20-nonaen-10-one (60 mg, 19% Yield, 98% purity) as a white solid.

¹H NMR (500 MHz, DMSO) δ 10.26 (s, 1H), 7.67-7.63 (m, 1H), 7.56-7.49 (m, 1H), 7.48-7.37 (m, 3H), 7.29 (d, J=2.2 Hz, 1H), 7.23-7.16 (m, 1H), 6.72-6.57 (m, 1H), 5.23-4.40 (m, 2H), 3.95 (s, 3H), 3.67 (br s, 2H).

LCMS: m/z=478.1/480.1 [M−H]−, (ESI−), RT=1.02, Method B

### Step 9

Iodocyclohexane (150 μL, 1.16 mmol) was added to a solution of 14-chloro-20,22-difluoro-15-methoxy-17,17-dioxo-9-oxa-17λ6-thia-18-azatetracyclo[17.3.1.112,16.02,7]tetracosa-1(22),2(7),3,5,12(24),13,15,19(23),20-nonaen-10-one (98% purity, 60 mg, 0.123 mmol) in DMF (2 mL) and the mixture was heated to 100° C. for 18 hours. The mixture was cooled and quenched into sat. aq. NaHCO₃. The aqueous layer was extracted into EtOAc (3×5 mL), the combined organics washed with sat. aq. sodium thiosulfate then brine, dried over MgSO₄ and concentrated in vacuo. The residue was purified by preparative HPLC (Method P1) to afford the title compound (19 mg, 33% Yield, 98% purity) as a white solid.

¹H NMR (400 MHz, DMSO) δ 7.56-7.49 (m, 1H), 7.50-7.42 (m, 3H), 7.36-7.26 (m, 1H), 7.24-7.16 (m, 2H), 6.76 (dd, J=8.7, 7.7 Hz, 1H), 4.84 (s, 2H), 3.56 (s, 2H).

LCMS: m/z=464.0,466.0 [M−H]−, (ESI−), RT=3.50, Method A

### Example 69—Synthesis of 14-chloro-20-fluoro-17,17-dioxo-10-oxa-17λ6-thia-18-azatetracyclo[17.3.1.112,16.02,7]tetracosa-1(23),2,4,6,12,14,16(24),19,21-nonaen-15-ol (Compound 69)

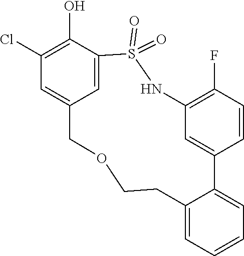 [SMILES: O=S1(=O)Nc2cc(ccc2F)-c2ccccc2CCOCc2cc(Cl)c(O)c1c2]

### Step 1

Lithium borohydride (100 mg, 4.59 mmol) was added to an ice-cold solution of methyl 3-[[5-[2-[2-[tert-butyl(dimethyl)silyl]oxyethyl]phenyl]-2-fluoro-phenyl]sulfamoyl]-5-chloro-4-methoxybenzoate (synthesized using a similar method to Intermediate 9, 600 mg, 0.987 mmol) in THE (6 mL) and MeOH (1.2 mL). The mixture was stirred for 1 hour then additional lithium borohydride (100 mg, 4.59 mmol) was added. The reaction was stirred for 1 hour then additional lithium borohydride (100 mg, 4.59 mmol) was added. The reaction was stirred for 1 hour then additional lithium borohydride (100 mg, 4.59 mmol) was added. Additional THE (6 mL) was added. The reaction was stirred for 1 hour then additional lithium borohydride (100 mg, 4.59 mmol) was added. The reaction was stirred for 1 hour then additional lithium borohydride (100 mg, 4.59 mmol) was added. The reaction was stirred for 1 hour then cautiously quenched by addition of water. The mixture was extracted with EtOAc (3×5 mL), the combined organics were washed with brine, dried over MgSO₄ and concentrated in vacuo. The residue was purified by FCC (25 g SiO₂ column, 0-50% EtOAc in heptane) to afford N-[5-[2-[2-[tert-butyl(dimethyl)silyl]oxyethyl]phenyl]-2-fluorophenyl]-3-chloro-5-(hydroxymethyl)-2-methoxybenzenesulfonamide (500 mg, 76% Yield, 87% purity) as a colorless oil.

¹H NMR (400 MHz, DMSO) δ 10.21 (s, 1H), 7.70-7.64 (m, 2H), 7.35-7.27 (m, 2H), 7.26-7.19 (m, 2H), 7.15-7.08 (m, 2H), 7.02 (dd, J=7.2, 1.4 Hz, 1H), 5.46 (t, J=5.7 Hz, 1H), 4.46 (d, J=5.7 Hz, 2H), 3.51 (t, J=6.9 Hz, 2H), 2.62 (t, J=6.9 Hz, 2H), 0.74 (s, 9H), −0.18 (s, 6H).

LCMS: m/z=578.2/580.3 [M−H]−, (ESI−), RT=1.28, Method B

### Step 2

Triphenylphosphine (145 mg, 0.553 mmol) was added to a solution of N-[5-[2-[2-[tert-butyl(dimethyl)silyl]oxyethyl]phenyl]-2-fluorophenyl]-3-chloro-5-(hydroxymethyl)-2-methoxybenzenesulfonamide (87% purity, 300 mg, 0.450 mmol), imidazole (75 mg, 1.10 mmol) and carbon tetrabromide (175 mg, 0.528 mmol) in DCM (10 mL). The reaction was stirred for 20 minutes at r.t, and then to the reaction mixture was added sequentially 1H-imidazole (40 mg), carbon tetrabromide (80 mg) and triphenylphosphine (75 mg). The reaction mixture was stirred at r.t. for 45 minutes before it was concentrated in vacuo. The residue was purified by FCC (25 g SiO₂ cartridge, 0-40% EtOAc in heptane) to afford 5-(bromomethyl)-N-[5-[2-[2-[tert-butyl(dimethyl)silyl]oxyethyl]phenyl]-2-fluorophenyl]-3-chloro-2-methoxybenzenesulfonamide (267 mg, 74% Yield, 80% purity) as a colorless oil, H NMR (500 MHz, DMSO) δ 10.33 (s, 1H), 7.90 (d, J=2.3 Hz, 1H), 7.79 (d, J=2.2 Hz, 1H), 7.33-7.27 (m, 2H), 7.26-7.21 (m, 2H), 7.17-7.12 (m, 1H), 7.10 (dd, J=7.5, 2.2 Hz, 1H), 7.04-7.01 (m, 1H), 4.70 (s, 2H), 3.89 (s, 3H), 3.53 (t, J=7.0 Hz, 2H), 2.64 (t, J=6.9 Hz, 2H), 0.74 (s, 9H), −0.17 (s, 6H).

LCMS: m/z=640.2/642.1/644.1 [M−H]−, (ESI−), RT=1.29, Method B

### Step 3

To a solution of 5-(bromomethyl)-N-[5-[2-[2-[tert-butyl(dimethyl)silyl]oxyethyl]phenyl]-2-fluorophenyl]-3-chloro-2-methoxybenzenesulfonamide (80% purity, 262 mg, 0.326 mmol) in MeOH (5 mL) at r.t. was added p-methylbenzenesulfonic acid hydrate (1:1) (15 mg, 0.0789 mmol). The reaction mixture was stirred at r.t. for 30 minutes and was then concentrated in vacuo. The residue was purified FCC (10 g SiO₂ cartridge, 0-40% EtOAc in heptane) to afford 5-(bromomethyl)-3-chloro-N-[2-fluoro-5-[2-(2-hydroxyethyl)phenyl]phenyl]-2-methoxybenzenesulfonamide (189 mg, 96% Yield, 88% purity) as a white gum/solid.

¹H NMR (400 MHz, DMSO) δ 10.28 (s, 1H), 7.90 (d, J=2.2 Hz, 1H), 7.78 (d, J=2.2 Hz, 1H), 7.33-7.26 (m, 2H), 7.25-7.19 (m, 2H), 7.17-7.12 (m, 1H), 7.08 (dd, J=7.5, 2.2 Hz, 1H), 7.03-7.00 (m, 1H), 4.71 (s, 2H), 4.63-4.43 (m, 1H), 3.91 (s, 3H), 3.38 (t, J=7.3 Hz, 2H), 2.59 (t, J=7.3 Hz, 2H).

LCMS: m/z=526.0/528.0/530.0 [M−H]−, (ESI−), RT=1.03, Method B

### Step 4

To a solution of 5-(bromomethyl)-3-chloro-N-[2-fluoro-5-[2-(2-hydroxyethyl)phenyl]phenyl]-2-methoxybenzenesulfonamide (88% purity, 175 mg, 0.291 mmol) in anhydrous THE (23 mL) at 0° C. was added sodium hydride in mineral oil (60%, 35 mg, 0.875 mmol). The reaction mixture was heated at 50° C. for 2.5 hours and was then allowed to cool to r.t. To the reaction mixture was added 1 M aq. HCl (10 mL of a 1 M aqueous solution) and water (20 mL). The mixture was diluted with EtOAc (20 mL), and the biphasic mixture was separated. The organic phase was washed with brine (20 mL), dried over MgSO₄, filtered and concentrated in vacuo. The residue was purified by FCC (10 g SiO₂ cartridge, 20-100% DCM in heptane) to afford 14-chloro-20-fluoro-15-methoxy-10-oxa-17λ6-thia-18-azatetracyclo[17.3.1.112,16.02,7]tetracosa-1(23),2(7),3,5,12(24),13,15,19,21-nonaene 17,17-dioxide (107 mg, 78% Yield, 95% purity) as a white solid.

¹H NMR (400 MHz, DMSO) δ 10.24 (s, 1H), 7.69 (d, J=2.1 Hz, 1H), 7.49 (d, J=2.1 Hz, 1H), 7.31-7.20 (m, 4H), 7.10-7.04 (m, 2H), 6.76 (dd, J=7.5, 2.3 Hz, 1H), 4.42 (s, 2H), 3.96 (s, 3H), 3.10-3.02 (m, 2H), 2.69-2.61 (m, 2H).

LCMS: m/z=446.1/448.1 [M−H]−, (ESI−), RT=1.09, Method B

### Step 5

To a solution of 14-chloro-20-fluoro-15-methoxy-10-oxa-17λ6-thia-18-azatetracyclo[17.3.1.112,16.02,7]tetracosa-1(23),2,4,6,12,14,16(24),19,21-nonaene 17,17-dioxide (95% purity, 107 mg, 0.227 mmol) in anhydrous pyridine (5 mL) was added lithium iodide (200 mg, 1.49 mmol). The reaction mixture was heated at 80° C. for 18.5 hours and was then allowed to cool to r.t. The mixture was diluted with EtOAc (30 mL) and the organic phase was washed sequentially with 1 M aq. HCl (2×20 mL), Na₂SO₃ (20 mL), then brine (10 mL), dried over MgSO₄, filtered and concentrated in vacuo. The residue was purified by preparative HPLC (Method P1) to afford the title compound (46 mg, 46% Yield, 99% purity) as a white solid.

¹H NMR (500 MHz, DMSO) δ 10.59-9.91 (m, 2H), 7.56 (d, J=2.1 Hz, 1H), 7.34 (d, J=2.1 Hz, 1H), 7.31-7.21 (m, 4H), 7.08-7.04 (m, 2H), 6.81 (dd, J=7.5, 2.3 Hz, 1H), 4.32 (s, 2H), 3.11-3.05 (m, 2H), 2.66-2.60 (m, 2H).

LCMS: m/z=432.2/434.3 [M−H]−, (ESI−), RT=3.88, Method A

### Example 70—Synthesis of (6R)-13-chloro-19-fluoro-14-hydroxy-16,16-dioxo-9-oxa-16%6-thia-2,17-diazatetracyclo[16.3.1.111,15.02,6]tricosa-1(21),11(23),12,14,18(22),19-hexaen-10-one (Compound 70)

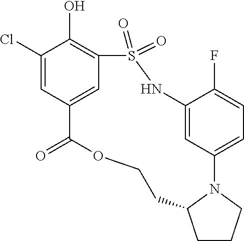 [SMILES: O=C1OCC[C@H]2CCCN2c2ccc(F)c(c2)NS(=O)(=O)c2cc1cc(Cl)c2O]

### Step 1

To a solution of 1-bromo-2,5-difluoro-4-nitrobenzene (900 mg, 3.78 mmol) in MeCN (12 mL) was added methyl 2-[(2R)-pyrrolidin-2-yl]acetate hydrochloride (600 mg, 3.34 mmol) and potassium carbonate (1.20 g, 8.68 mmol). The mixture was heated at 40° C. for 22 hours and was then filtered. The filtrate was concentrated in vacuo. The residue was purified by FCC (25 g SiO₂ cartridge, 0-20% Et₂O in heptane) to afford methyl 2-[(2R)-1-(5-bromo-4-fluoro-2-nitrophenyl)218pyrrolidine-2-yl]acetate (1.15 g, 3.02 mmol, 91% Yield, 95% purity) as a yellow solid.

¹H NMR (400 MHz, DMSO) δ 7.92 (d, J=8.6 Hz, 1H), 7.48 (d, J=6.0 Hz, 1H), 4.26-4.15 (m, 1H), 3.57 (s, 3H), 3.40-3.27 (m, 1H), 2.70-2.61 (m, 2H), 2.38 (dd, J=15.4, 8.4 Hz, 1H), 2.31-2.22 (m, 1H), 1.95-1.86 (m, 1H), 1.84-1.66 (m, 2H).

LCMS: m/z=360.9/362.9 [M+H]+, (ESI+), RT=1.05, Method B

### Step 2

To a solution of methyl 2-[(2R)-1-(5-bromo-4-fluoro-2-nitrophenyl)218pyrrolidine-2-yl]acetate (95% purity, 1.15 g, 3.02 mmol) in EtOH (30 mL) was added iron powder (1.70 g, 30.4 mmol) and sat. aq. NH₄Cl solution (6.7 mL, 3.02 mmol). The mixture was heated at 55° C. for 1.3 hours and was then allowed to cool to r.t. The mixture was filtered through Celite, washing with EtOH. The filtrate was concentrated in vacuo and the residue was treated with sat. aq. NaHCO₃ (30 mL) and water (10 mL). The mixture was extracted with EtOAc (80 mL), the organic layer washed with brine (30 mL), dried over MgSO₄, filtered and concentrated in vacuo. The residue was purified by FCC (25 g SiO₂ cartridge, 0-30% Et₂O in heptane) to afford methyl 2-[(2R)-1-(2-amino-5-bromo-4-fluorophenyl)218yrrolidine-2-yl]acetate (703 mg, 67% Yield, 95% purity) as an orange-brown oil.

¹H NMR (400 MHz, DMSO) δ 7.15 (d, J=7.4 Hz, 1H), 6.57 (d, J=11.1 Hz, 1H), 5.27 (s, 2H), 3.69-3.61 (m, 1H), 3.49 (s, 3H), 3.31-3.26 (m, 1H), 2.61-2.53 (m, 1H), 2.31-2.16 (m, 2H), 2.16-2.08 (m, 1H), 1.91-1.73 (m, 2H), 1.64-1.52 (m, 1H).

LCMS: m/z=330.9/332.9 [M+H]+, (ESI+), RT=1.00, Method B

### Step 3

To a solution of methyl 2-[(2R)-1-(2-amino-5-bromo-4-fluorophenyl)219pyrrolidine-2-yl]acetate (95% purity, 700 mg, 2.01 mmol) in 1,4-dioxane (15 mL) and water (7.2 mL) at 0° C. was added pre-cooled (0° C.) 50% phosphinic acid in water (9.0 mL, 68.2 mmol) before sodium nitrite (360 mg, 5.22 mmol) was added portion wise over 1 minute. The mixture was stirred at 0° C. for 45 minutes and was carefully poured onto a pre-cooled (0° C.) biphasic stirring mixture of sat. aq. NaHCO₃ (200 mL), water (30 mL) and TBME (150 mL). The organics were separated. The aqueous was extracted with TBME (100 mL). The organics were combined, washed with brine (50 mL), dried over MgSO₄, filtered and concentrated in vacuo. The residue was purified by FCC (25 g SiO₂ cartridge, 0-40% Et₂O in heptane) to afford methyl 2-[(2R)-1-(3-bromo-4-fluorophenyl)219yrrolidine-2-yl]acetate (315 mg, 50% Yield, 100% purity) as a yellow oil.

¹H NMR (500 MHz, CDCl₃) δ 7.01-6.95 (m, 1H), 6.70 (dd, J=5.6, 3.0 Hz, 1H), 6.48-6.44 (m, 1H), 4.12-4.06 (m, 1H), 3.71 (s, 3H), 3.39-3.34 (m, 1H), 3.16-3.09 (m, 1H), 2.71 (dd, J=15.1, 3.1 Hz, 1H), 2.24 (dd, J=15.1, 10.3 Hz, 1H), 2.13-2.00 (m, 3H), 1.91-1.86 (m, 1H).

LCMS: m/z=315.9/317.9 [M+H]+, (ESI+), RT=1.10, Method B

### Step 4

A suspension of methyl 2-[(2R)-1-(3-bromo-4-fluorophenyl)219yrrolidine-2-yl]acetate (305 mg, 0.965 mmol), palladium(II) diacetate (22 mg, 0.0980 mmol) and Xantphos (84 mg, 0.145 mmol) in 1,4-dioxane (4.8 mL) was sparged with nitrogen with 2 minutes, and cesium carbonate (630 mg, 1.93 mmol) and 1,1-diphenylmethanimine (0.24 mL, 1.43 mmol) were added. The mixture was heated at 100° C. for 10 hours and was then allowed to cool to r.t, and filtered through Celite. The filtrate was concentrated in vacuo and then re-dissolved in THE (2.4 mL) before 2 M aqueous HCl (1.2 mL, 2.40 mmol) was added. The resultant mixture was stirred at r.t. for 15 minutes before sat. aq. NaHCO₃ (10 mL) and water (5 mL) were added. The organics were extracted with EtOAc (40 mL), separated, washed with brine (10 mL) and were then dried over MgSO₄, filtered and concentrated in vacuo. The residue was purified by FCC (25 g SiO₂ cartridge, 0-70% Et₂O in heptane) to afford methyl 2-[(2R)-1-(3-amino-4-fluorophenyl)220yrrolidine-2-yl]acetate (225 mg, 65% Yield, 70% purity) as a yellow oil.

¹H NMR (500 MHz, DMSO) δ 6.79 (dd, J=11.3, 8.8 Hz, 1H), 5.99 (dd, J=7.8, 2.9 Hz, 1H), 5.71-5.66 (m, 1H), 4.89 (s, 2H), 3.96-3.88 (m, 1H), 3.63 (s, 3H), 3.28-3.20 (m, 1H), 3.04-2.96 (m, 1H), 2.63 (dd, J=15.3, 3.0 Hz, 1H), 2.26 (dd, J=15.3, 10.2 Hz, 1H), 2.03-1.89 (m, 3H), 1.80-1.72 (m, 1H).

LCMS: m/z=253.0 [M+H]+, (ESI+), RT=0.70, Method B

### Step 5

To a solution of methyl 2-[(2R)-1-(3-amino-4-fluorophenyl)220yrrolidine-2-yl]acetate (70% purity, 225 mg, 0.624 mmol) in dry Et₂O (5.7 mL) at 0° C. was added 2.4 M LiAlH₄ in THE (0.40 mL, 0.960 mmol). The mixture was stirred for 15 minutes at 0° C. then Et₂O (20 ml) was added, followed by water (40 mL) then 15 wt % aq. NaOH (40 mL), then water (0.1 mL) and the mixture was allowed to warm to r.t, and was stirred for 20 minutes. MgSO₄ was added and the mixture was stirred for 30 minutes at r.t. The mixture was then filtered, the filter cake washed with Et₂O (2×10 mL), and the combined organic filtrates were concentrated in vacuo. The residue was purified by FCC (10 g SiO₂ cartridge, 0-30% acetone in heptane) to afford 2-[(2R)-1-(3-amino-4-fluorophenyl)220yrrolidine-2-yl]ethanol (140 mg, 90% Yield, 90% purity) as a yellow oil.

¹H NMR (400 MHz, DMSO) δ 6.74 (dd, J=11.3, 8.8 Hz, 1H), 5.97 (dd, J=7.9, 2.9 Hz, 1H), 5.78-5.71 (m, 1H), 4.79 (s, 2H), 4.48 (t, J=4.9 Hz, 1H), 3.67-3.59 (m, 1H), 3.54-3.41 (m, 2H), 3.26-3.19 (m, 1H), 3.00-2.91 (m, 1H), 1.96-1.84 (m, 3H), 1.83-1.73 (m, 2H), 1.38-1.28 (m, 1H).

LCMS: m/z=225.0 [M+H]+, (ESI+), RT=0.35, Method B

### Step 6

To a solution of 2-[(2R)-1-(3-amino-4-fluorophenyl)220yrrolidine-2-yl]ethanol (90% purity, 180 mg, 0.72 mmol) in pyridine (3.8 mL) was added Intermediate 1 (50% purity, 500 mg, 0.836 mmol). The mixture was heated at 50° C. for 1 hour 20 minutes and was then left standing at r.t. for 16 hours. The mixture was diluted with EtOAc (50 mL), washed with 1 M aq. HCl (80 mL), then brine (50 mL), dried over MgSO₄, filtered and concentrated in vacuo. To the aqueous was added sat. aq. NaHCO₃ (80 mL) and the organics were extracted with EtOAc (50 mL), then dried over MgSO₄, filtered and concentrated in vacuo. Combined residues were purified by FCC (SiO₂ cartridge, 0-30% acetone in heptane) followed by further FCC purification (25 g SiO₂ cartridge, 0-100% Et₂O in heptane) to afford methyl 3-chloro-5-[[2-fluoro-5-[(2R)-2-(2-hydroxyethyl)221pyrrolidine-1-yl]phenyl]sulfamoyl]-4-methoxybenzoate (237 mg, 55% Yield, 81% purity) as a yellow oil.

¹H NMR (400 MHz, DMSO) δ 10.13 (s, 1H), 8.23 (d, J=2.1 Hz, 1H), 8.16 (d, J=2.1 Hz, 1H), 6.94 (dd, J=10.2, 9.0 Hz, 1H), 6.39-6.32 (m, 1H), 6.27 (dd, J=6.6, 3.0 Hz, 1H), 4.49 (t, J=4.9 Hz, 1H), 4.02 (s, 3H), 3.85 (s, 3H), 3.63-3.56 (m, 1H), 3.47-3.39 (m, 2H), 3.21-3.14 (m, 1H), 2.92-2.84 (m, 1H), 1.96-1.76 (m, 4H), 1.69-1.59 (m, 1H), 1.34-1.25 (m, 1H).

LCMS: m/z=485.1/487.1 [M−H]−, (ESI−), RT=0.96, Method B

### Step 7

To a solution of methyl 3-chloro-5-[[2-fluoro-5-[(2R)-2-(2-hydroxyethyl)221pyrrolidine-1-yl]phenyl]sulfamoyl]-4-methoxybenzoate (81% purity, 230 mg, 0.383 mmol) in THE (4 mL) was added 2 M aq. Sodium hydroxide (1.3 mL, 2.60 mmol). The reaction mixture was stirred at r.t. for 90 hours and then the organics were removed in vacuo. To the aqueous was added 1 M aq. HCl (2.1 mL) (resulting Ph was 6-7). The mixture was extracted with DCM (3×20 mL). To the aqueous was added 1 M aq. HCl (0.5 mL) (the aqueous phase Ph was 2-3). The aqueous phase was further extracted with DCM (4×20 mL). All organic phases were combined, dried over MgSO₄, filtered and concentrated in vacuo to afford 3-chloro-5-[[2-fluoro-5-[(2R)-2-(2-hydroxyethyl)221pyrrolidine-1-yl]phenyl]sulfamoyl]-4-methoxybenzoic acid (208 mg, 92% Yield, 80% purity) as a grey solid.

### Step 8

To a solution of 3-chloro-5-[[2-fluoro-5-[(2R)-2-(2-hydroxyethyl)221pyrrolidine-1-yl]phenyl]sulfamoyl]-4-methoxybenzoic acid (80% purity, 200 mg, 0.338 mmol) in anhydrous DCM (13 mL) was added DMAP (8.0 mg, 0.0655 mmol) and DCC (150 mg, 0.727 mmol). The mixture was stirred at r.t. for 2 hours and was then concentrated in vacuo. The residue was purified by FCC (25 g SiO₂ cartridge, 30-100% DCM in heptane, then 0-20% EtOAc in DCM) to afford (6R)-13-chloro-19-fluoro-14-methoxy-16,16-dioxo-9-oxa-16λ6-thia-2,17-diazatetracyclo[16.3.1.111,15.02,6]tricosa-1(22),11,13,15(23),18,20-hexaen-10-one (125 mg, 77% Yield, 95% purity) as a white solid.

¹H NMR (400 MHz, DMSO) δ 10.22 (s, 1H), 8.98-8.87 (m, 1H), 8.19 (d, J=2.0 Hz, 1H), 7.14-7.07 (m, 1H), 7.02-6.95 (m, 1H), 6.55-6.46 (m, 1H), 4.52-4.41 (m, 1H), 4.39-4.30 (m, 1H), 3.96 (s, 3H), 3.46-3.39 (m, 1H), 3.32-3.26 (m, 1H), 2.95-2.86 (m, 1H), 2.10-1.73 (m, 6H).

LCMS: m/z=453.1/455.1 [M+H]+, (ESI+), RT=1.02, Method B

### Step 9

(6R)-13-Chloro-19-fluoro-14-methoxy-16,16-dioxo-9-oxa-16λ6-thia-2,17-diazatetracyclo[16.3.1.111,15.02,6]tricosa-1(21),11(23),12,14,18(22),19-hexaen-10-one (95% purity, 120 mg, 0.251 mmol), anhydrous DMF (3.6 mL) and iodocyclohexane (162 mL, 1.25 mmol) were added to a pressure vial. The vial was sealed and heated at 120° C. for 1.25 hours. The mixture was purified by preparative HPLC (Method P1), followed by trituration with MeCN (2 mL) to afford the title compound (61 mg, 53% Yield, 96% purity) as a white solid.

¹H NMR (500 MHz, DMSO) δ 11.41 (br. S, 1H), 10.04 (br. S, 1H), 8.85-8.68 (m, 1H), 8.06 (d, J=2.1 Hz, 1H), 7.15-7.06 (m, 1H), 6.98-6.86 (m, 1H), 6.55-6.46 (m, 1H), 4.46-4.36 (m, 1H), 4.35-4.25 (m, 1H), 3.51-3.42 (m, 1H), 3.29-3.21 (m, 1H), 2.95-2.85 (m, 1H), 2.02-1.72 (m, 6H).

LCMS: m/z=439.0/441.0 [M−H]−, (ESI−), RT=3.65, Method A

### Example 71—Synthesis of 14-chloro-20,22-difluoro-15-hydroxy-10-methyl-17,17-dioxo-17λ6-thia-10,18-diazatetracyclo[17.3.1.112,16.02,7]tetracosa-1(22),2(7),3,5,12,14,16(24),19(23),20-nonaen-11-one (Compound 71)

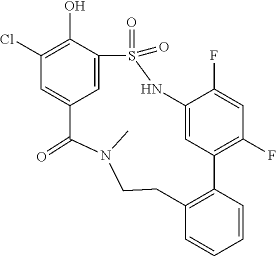 [SMILES: CN1CCc2ccccc2-c2cc(c(F)cc2F)NS(=O)(=O)c2cc(cc(Cl)c2O)C1=O]

14-Chloro-20,22-difluoro-15-methoxy-10-methyl-17,17-dioxo-17λ6-thia-10,18-diazatetracyclo[17.3.1.112,16.02,7]tetracosa-1(22),2(7),3,5,12,14,16(24),19(23),20-nonaen-11-one (synthesized using a similar method to Example 8, 98% purity, 100 mg, 0.199 mmol), iodocyclohexane (139 μL, 1.07 mmol) and anhydrous DMF (5 mL) were added to a pressure vial, the vial was sealed and heated to 120° C. with stirring for 3 hours. The mixture was allowed to cool to r.t., added to water (50 mL) and extracted with DCM (3×50 mL). The combined organic extracts were washed with brine (100 mL), dried over Na₂SO₄, filtered and concentrated under reduced pressure. The residue was purified by preparative HPLC (Method P1) to afford the title compound (42 mg, 44% Yield, 100% purity) as a white solid.

¹H NMR (500 MHz, DMSO) δ 10.93 (br. s, 1H), 10.11 (br. s, 1H), 7.64 (d, J=1.8 Hz, 1H), 7.54-7.42 (m, 2H), 7.41-7.33 (m, 1H), 7.31-7.22 (m, 1H), 7.19-7.05 (m, 2H), 6.76-6.53 (m, 1H), 3.08-2.95 (m, 5H), 2.27-2.13 (m, 1H).

LCMS: m/z=479.0/480.9 [M+H]+, (ESI+), RT=3.19, Method A

### Example 72—Synthesis of 14-chloro-20-fluoro-15-hydroxy-17,17-dioxo-10-oxa-17λ6-thia-18-azatetracyclo[17.3.1.112,16.02,7]tetracosa-1(22),2(7),3,5,12,14,16(24),19(23),20-nonaen-11-one (Compound 72)

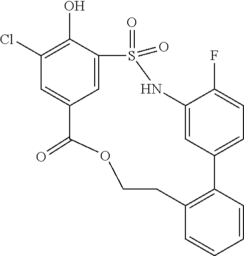 [SMILES: O=C1OCCc2ccccc2-c2ccc(F)c(c2)NS(=O)(=O)c2cc1cc(Cl)c2O]

14-Chloro-20-fluoro-15-methoxy-17,17-dioxo-10-oxa-17λ6-thia-18-azatetracyclo[17.3.1.112,16.02,7]tetracosa-1(22),2(7),3,5,12,14,16(24),19(23),20-nonaen-11-one (synthesized using a similar method to Intermediate 12, 97% purity, 85 mg, 0.179 mmol), iodocyclohexane (123 μL, 0.952 mmol) and anhydrous DMF (5 mL) were added to a pressure vial, the vial was sealed and heated to 120° C. with stirring for 3 hours. The mixture was allowed to cool to r.t., added to water (50 mL) and extracted with DCM (3×50 mL). The combined organic extracts were washed with brine (100 mL), dried over Na₂SO₄, filtered and concentrated under reduced pressure. The residue was purified by preparative HPLC (Method P1) to afford the title compound (65 mg, 79% Yield, 97% purity) an off-white solid.

¹H NMR (500 MHz, DMSO) δ 10.31 (br. s, 1H), 7.91 (d, J=2.0 Hz, 1H), 7.55-7.46 (m, 1H), 7.46-7.39 (m, 1H), 7.39-7.33 (m, 3H), 7.28-7.18 (m, 1H), 7.09-6.87 (m, 1H), 6.74-6.51 (m, 1H), 4.52-4.20 (m, 2H), 3.06-2.86 (m, 2H).

LCMS: m/z=446.0/448.0 [M−H]−, (ESI−), RT=3.99, Method A

### Example 73—Synthesis of 14-chloro-5,20-difluoro-15-hydroxy-17,17-dioxo-10-oxa-17λ6-thia-18-azatetracyclo[17.3.1.112,16.02,7]tetracosa-1(22),2(7),3,5,12,14,16(24),19(23),20-nonaen-11-one (Compound 73)

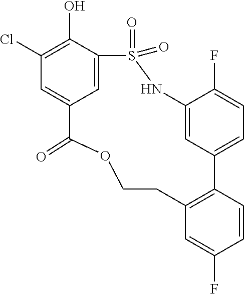 [SMILES: O=C1OCCc2cc(F)ccc2-c2ccc(F)c(c2)NS(=O)(=O)c2cc1cc(Cl)c2O]

14-Chloro-5,20-difluoro-15-methoxy-17,17-dioxo-10-oxa-17λ6-thia-18-azatetracyclo[17.3.1.112,16.02,7]tetracosa-1(22),2(7),3,5,12,14,16(24),19(23),20-nonaen-11-one (synthesized using a similar method to Intermediate 12, 99% purity, 100 mg, 0.206 mmol), iodocyclohexane (135 μL, 1.05 mmol) and anhydrous DMF (5 mL) were added to a pressure vial, the vial was sealed and heated to 120° C. with stirred for 3 hours. The mixture was allowed to cool to r.t., added to water (50 mL) and extracted with DCM (3×50 mL). The combined organic extracts were washed with brine (100 mL), dried over Na₂SO₄, filtered and concentrated under reduced pressure. The residue was purified by preparative HPLC (Method P1) to afford the title compound (71 mg, 73% Yield, 99% purity) as a white solid.

¹H NMR (500 MHz, DMSO) δ 10.86-9.71 (m, 2H), 7.93 (d, J=2.1 Hz, 1H), 7.47-7.33 (m, 4H), 7.11-7.03 (m, 2H), 6.60 (dd, J=7.3, 2.3 Hz, 1H), 4.63-4.05 (m, 2H), 3.20-2.83 (m, 2H).

LCMS: m/z=464.0/466.0 [M−H]−, (ESI−), RT=4.04, Method A

### Example 74—Synthesis of 14-chloro-4,20-difluoro-15-hydroxy-17,17-dioxo-10-oxa-17λ6-thia-18-azatetracyclo[17.3.1.112,16.02,7]tetracosa-1(22),2(7),3,5,12(24),13,15,19(23),20-nonaen-11-one (Compound 74)

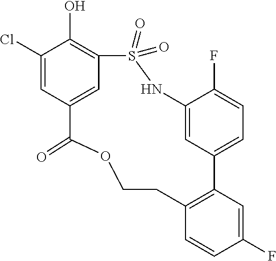 [SMILES: O=C1OCCc2ccc(F)cc2-c2ccc(F)c(c2)NS(=O)(=O)c2cc1cc(Cl)c2O]

1 M BBr₃ in DCM (1.3 mL, 1.25 mmol) was added to an ice cold solution of 14-chloro-4,20-difluoro-15-methoxy-17,17-dioxo-10-oxa-17λ6-thia-18-azatetracyclo[17.3.1.112,16.02,7]tetracosa-1(22),2(7),3,5,12(24),13,15,19(23),20-nonaen-11-one (synthesized using a similar method to Intermediate 12, 89% purity, 140 mg, 0.260 mmol) in DCM (3.5 mL). The mixture was stirred for 3 hours then quenched into sat. aq. NaHCO₃. The aqueous layer was extracted with DCM (4×5 mL), the combined organics washed with brine, dried over MgSO₄ and concentrated in vacuo. The residue was purified by preparative HPLC (Method P1) to afford the title compound (23 mg, 18% Yield, 95% purity) as a white solid.

¹H NMR (500 MHz, DMSO) δ 7.93-7.89 (m, 1H), 7.54 (dd, J=8.7, 6.0 Hz, 1H), 7.44 (dd, J=9.9, 8.4 Hz, 1H), 7.40-7.34 (m, 2H), 7.25-7.17 (m, 1H), 6.86 (dd, J=9.6, 2.8 Hz, 1H), 6.67 (d, J=7.1 Hz, 1H), 4.48-4.22 (m, 2H), 3.08-2.86 (m, 2H).

LCMS: m/z=464.0,466.0 [M−H]−, (ESI−), RT=4.00, Method A

### Example 75—Synthesis of 20,22-difluoro-15-hydroxy-14-methyl-17,17-dioxo-10-oxa-17λ6-thia-18-azatetracyclo[17.3.1.112,16.02,7]tetracosa-1(23),2,4,6,12,14,16(24),19,21-nonaen-11-one (Compound 75)

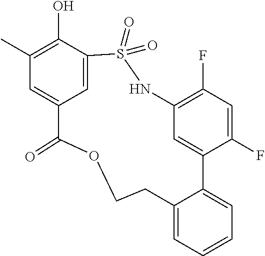 [SMILES: Cc1cc2cc(c1O)S(=O)(=O)Nc1cc(c(F)cc1F)-c1ccccc1CCOC2=O]

### Step 1

A solution of 14-chloro-20,22-difluoro-15-methoxy-17,17-dioxo-10-oxa-17λ6-thia-18-azatetracyclo[17.3.1.112,16.02,7]tetracosa-1(23),2,4,6,12,14,16(24),19,21-nonaen-11-one (synthesized using a similar method to Intermediate 12, 95% purity, 110 mg, 0.218 mmol) and potassium methyltrifluoroborate (80 mg, 0.656 mmol) in toluene (1.5 mL) and water (0.15 mL) was sparged with nitrogen for 2 mins before potassium carbonate (170 mg, 1.23 mmol) and RuPhos Pd G3 (6.0 mg, 7.17 mol) were added. The mixture was sealed and heated at 80° C. for 19.5 hours. Further RuPhos Pd G3 (6.0 mg, 7.17 mol) was added and the mixture was heated at 80° C. for 2 hours. The mixture was purified by FCC (25 g SiO₂ column, 0-100% DCM in heptane, then 0-30% MeOH in DCM) followed by preparative HPLC (Method P1) to afford 20,22-difluoro-15-methoxy-14-methyl-17,17-dioxo-10-oxa-17λ6-thia-18-azatetracyclo[17.3.1.112,16.02,7]tetracosa-1(23),2,4,6,12,14,16(24),19,21-nonaen-11-one (35 mg, 33% Yield, 94% purity) as a white solid.

¹H NMR (500 MHz, DMSO) δ 9.92 (s, 1H), 7.84 (s, 1H), 7.59-7.47 (m, 2H), 7.47-7.43 (m, 1H), 7.42-7.38 (m, 1H), 7.30-7.23 (m, 1H), 7.00 (dd, J=7.6, 1.4 Hz, 1H), 6.60-6.40 (m, 1H), 4.58-4.48 (m, 1H), 4.35-4.28 (m, 1H), 3.87 (s, 3H), 3.07-2.94 (m, 1H), 2.90-2.75 (m, 1H), 2.31 (s, 3H).

LCMS: m/z=458.1 [M−H]−, (ESI−), RT=1.01, Method B

### Step 2

Iodocyclohexane (93 μL, 0.716 mmol) was added to a solution of 20,22-difluoro-15-methoxy-14-methyl-17,17-dioxo-10-oxa-17λ6-thia-18-azatetracyclo[17.3.1.112,16.02,7]tetracosa-1(23),2,4,6,12,14,16(24),19,21-nonaen-11-one (94% purity, 35 mg, 0.072 mmol) in anhydrous DMF (1.9 mL) and the mixture was heated at 125° C. for 2 hours, then heated to 130° C. for 5 hours. The mixture was cooled to r.t., diluted with EtOAc (20 mL) and washed with sat. aq. Na₂S₂O₃ (20 mL). The aqueous layer was extracted with further EtOAc (20 mL), the combined organics washed with 1 M aq. HCl (30 mL) and brine (30 mL), then passed through a phase separator and concentrated. The residue was purified by FCC (10 g SiO₂ column, 0-30% MeOH in DCM) to afford the title compound (23 mg, 65% Yield, 90% purity) as a white solid.

¹H NMR (500 MHz, DMSO) δ 10.16 (br s, 1H), 9.75 (br s, 1H), 7.76-7.72 (m, 1H), 7.65-7.55 (m, 1H), 7.51 (dd, J=8.0, 1.4 Hz, 1H), 7.43-7.36 (m, 1H), 7.34-7.29 (m, 1H), 7.29-7.23 (m, 1H), 7.00 (dd, J=7.6, 1.4 Hz, 1H), 6.61-6.50 (m, 1H), 4.62-4.50 (m, 1H), 4.25-4.14 (m, 1H), 3.05-2.96 (m, 1H), 2.91-2.79 (m, 1H), 2.21 (s, 3H).

LCMS: m/z=444.1 [M−H]−, (ESI−), RT=3.98, Method A

### Example 76—Synthesis of 16-chloro-22,24-difluoro-17-hydroxy-19,19-dioxo-12-oxa-19λ6-thia-8,20-diazapentacyclo[19.3.1.18,10.114,18.02,7]heptacosa-1(25),2,4,6,14,16,18(26),21,23-nonaen-13-one (Compound 76)

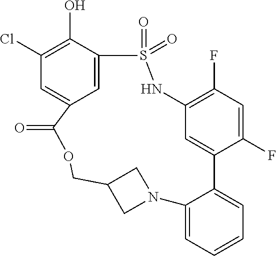 [SMILES: O=C1OCC2CN(C2)c2ccccc2-c2cc(c(F)cc2F)NS(=O)(=O)c2cc1cc(Cl)c2O]

### Step 1

A sealed vial was charged with 227zetidine-3-ylmethanol hydrochloride (1:1) (481 mg, 3.89 mmol), 1,2-dimethoxyethane-dibromonickel (1:1) (46 mg, 0.149 mmol) and (4,4′-di-t-butyl-2,2′-bipyridine)bis[3,5-difluoro-2-[5-trifluoromethyl-2-pyridinyl-Kn)phenyl-Kc]iridium(III) hexafluorophosphate (4.0 mg, 3.57 mol) under nitrogen. To the vial was added dry DMA (12 mL), 1-bromo-2-chlorobenzene (0.35 mL, 2.99 mmol) and 1-methyl-1,3,4,6,7,8-hexahydro-2H-pyrimido[1,2-a]pyrimidine (MTBD) (1.1 mL, 7.66 mmol). The reaction mixture was sparged with nitrogen for 15 minutes and was then placed in the Penn photoreactor (450 nm, stir speed=1000 rpm, fan speed=2800 rpm, LED intensity=100%) for 16 hours. The mixture was diluted with EtOAc (120 mL), then washed with brine (2×120 mL), dried over MgSO₄, filtered and concentrated in vacuo. The residue was purified by FCC (25 g SiO₂ cartridge, 0-30% acetone in heptane) to afford [1-(2-chlorophenyl)228azetidine-3-yl]methanol (82 mg, 14% Yield, 99% purity) as a colorless oil.

¹H NMR (400 MHz, DMSO) δ 7.20 (dd, J=7.9, 1.5 Hz, 1H), 7.15-7.13 (m, 1H), 6.74-6.69 (m, 1H), 6.56 (dd, J=8.1, 1.5 Hz, 1H), 4.74 (t, J=5.3 Hz, 1H), 4.03-3.96 (m, 2H), 3.71 (dd, J=7.5, 5.6 Hz, 2H), 3.57 (t, J=5.7 Hz, 2H), 2.73-2.66 (m, 1H).

### Step 2

A solution of [1-(2-chlorophenyl)228azetidine-3-yl]methanol (99% purity, 82 mg, 0.411 mmol) and 2,4-difluoro-5-(4,4,5,5-tetramethyl-1,3,2-dioxaborolan-2-yl)aniline (95% purity, 150 mg, 0.559 mmol) in 1,4-dioxane (2.7 mL) and water (1 mL) was sparged with nitrogen for 5 minutes. To the mixture was added potassium carbonate (170 mg, 1.23 mmol) and Xphos Pd G3 (20 mg, 0.0236 mmol) and the reaction mixture was heated at 90° C. for 1 hours 20 minutes. The mixture was allowed to cool to r.t. whereby two layers formed. The organic phase was separated and the aqueous layer was extracted with EtOAc (3×4 mL). The organics were combined and were concentrated in vacuo. The residue was purified by FCC (10 g SiO₂ cartridge, 0-30% acetone in heptane) to afford [1-[2-(5-amino-2,4-difluorophenyl)phenyl]228azetidine-3-yl]methanol (114 mg, 96% Yield, 100% purity) as an orange oil.

LCMS: m/z=291.1 [M+H]+, (ESI+), RT=0.74, Method B

### Step 3

Intermediate 1 (50%, 440 mg, 0.735 mmol) and [1-[2-(5-amino-2,4-difluoro-phenyl)phenyl]228azetidine-3-yl]methanol (202 mg, 0.68 mmol) were dissolved in anhydrous pyridine (3.6 mL) and the mixture was heated at 50° C. for 45 minutes. The mixture was concentrated in vacuo and the residue was purified by FCC (25 g SiO₂ cartridge, 0-30% acetone in heptane), then further purified by FCC (10 g SiO₂ cartridge, 30-100% Et₂O in heptane) which gave methyl 3-chloro-5-[[2,4-difluoro-5-[2-[3-(hydroxymethyl)228azetidine-1-yl]phenyl]phenyl]sulfamoyl]-4-methoxybenzoate (240 mg, 48% Yield, 75% purity) as an off-white solid.

¹H NMR (500 MHz, CDCl₃) δ 8.27 (d, J=2.1 Hz, 1H), 8.26 (d, J=2.1 Hz, 1H), 7.54 (dd, J=8.9, 7.5 Hz, 1H), 7.28-7.24 (m, 1H), 7.14 (s, 1H), 7.01-6.98 (m, 1H), 6.83-6.79 (m, 1H), 6.76 (dd, J=9.9, 8.7 Hz, 1H), 6.55 (dd, J=8.2, 1.1 Hz, 1H), 4.20 (s, 3H), 3.90 (s, 3H), 3.78-3.73 (m, 2H), 3.56-3.52 (m, 2H), 3.26 (dd, J=7.3, 5.0 Hz, 2H), 2.70-2.61 (m, 1H), 1.94 (t, J=5.5 Hz, 1H).

LCMS: m/z=551.1/553.1 [M−H]−, (ESI−), RT=1.01, Method B

### Step 4

To a solution of methyl 3-chloro-5-[[2,4-difluoro-5-[2-[3-(hydroxymethyl)229azetidine-1-yl]phenyl]phenyl]sulfamoyl]-4-methoxybenzoate (75% purity, 234 mg, 0.317 mmol) in THE (3.3 mL) was added 2 M aq. sodium hydroxide (1.1 mL, 2.20 mmol). The mixture was stirred at r.t. for 17 hours and then the organics were removed in vacuo. To the remaining aqueous was added 1 M aq. HCl (6 mL). The mixture was extracted with DCM (3×25 mL), the organic layers combined, passed through a hydrophobic frit and concentrated in vacuo to afford 3-chloro-5-[[2,4-difluoro-5-[2-[3-(hydroxymethyl)229azetidine-1-yl]phenyl]phenyl]sulfamoyl]-4-methoxybenzoic acid (229 mg, 80% Yield, 60% yield) as a colorless gum.

LCMS: m/z=537.2/539.2 [M−H]−, (ESI−), RT=0.88, Method B

### Step 5

To a solution of 3-chloro-5-[[2,4-difluoro-5-[2-[3-(hydroxymethyl)229azetidine-1-yl]phenyl]phenyl]sulfamoyl]-4-methoxybenzoic acid (60% purity, 225 mg, 0.250 mmol) in anhydrous DCM (10 mL) was added DMAP (8.0 mg, 0.0655 mmol) and DCC (135 mg, 0.654 mmol). The mixture was stirred at r.t. for 1.5 hours and was then concentrated in vacuo. The residue was purified by FCC (10 g SiO₂ cartridge, 20-100% DCM in heptane) to afford 16-chloro-22,24-difluoro-17-methoxy-19,19-dioxo-12-oxa-19λ6-thia-8,20-diazapentacyclo[19.3.1.18,10.114,18.02,7]heptacosa-1(25),2,4,6,14,16,18(26),21,23-nonaen-13-one (95.0%) (EV-ICD001-653-002) (83 mg, 60% Yield, 95% purity) as a white solid.

¹H NMR (500 MHz, CDCl₃) δ 8.38 (d, J=2.1 Hz, 1H), 8.31 (d, J=2.0 Hz, 1H), 7.23 (ddd, J=8.2, 7.3, 1.6 Hz, 1H), 7.09 (br. S, 1H), 7.04 (d, J=7.3 Hz, 1H), 6.99 (dd, J=10.3, 8.4 Hz, 1H), 6.89-6.80 (m, 2H), 6.53-6.50 (m, 1H), 5.07 (d, J=11.7 Hz, 1H), 4.16 (s, 3H), 4.08-4.00 (m, 2H), 3.52-3.48 (m, 1H), 3.41-3.35 (m, 1H), 3.19-3.13 (m, 1H), 2.78-2.70 (m, 1H).

LCMS: m/z=519.1/521.1 [M−H]−, (ESI−), RT=4.24, Method A

### Step 6

To a solution of 16-chloro-22,24-difluoro-17-methoxy-19,19-dioxo-12-oxa-19a6-thia-8,20-diazapentacyclo[19.3.1.18,10.114,18.02,7]heptacosa-1(25),2,4,6,14,16,18(26),21,23-nonaen-13-one (95% purity, 80 mg, 0.146 mmol) in anhydrous pyridine (5 mL) was added lithium iodide (120 mg, 0.897 mmol). The mixture was heated at 80° C. for 6 hours and was then allowed to cool to r.t. The mixture was diluted with EtOAc (30 mL), then washed with 10% aq. citric acid (3×20 mL), then brine (20 mL), dried over MgSO₄, filtered and concentrated in vacuo. The residue was purified by FCC (10 g SiO₂ cartridge, 0-15% MeOH in DCM). The resulting solid was dissolved in EtOAc (30 mL), washed with 10% aq. citric acid (2×20 mL), then brine (20 mL), dried over MgSO₄, filtered and concentrated in vacuo. The residue was then triturated with MeCN (0.3 mL) to afford the title compound (26 mg, 33% Yield, 92% purity) as an off-white solid.

¹H NMR (500 MHz, DMSO) δ 8.20 (d, J=2.1 Hz, 1H), 8.13 (d, J=2.1 Hz, 1H), 7.56-7.50 (m, 1H), 7.20-7.16 (m, 1H), 6.98 (d, J=7.5 Hz, 1H), 6.79-6.72 (m, 2H), 6.53-6.50 (m, 1H), 5.08-5.00 (m, 1H), 4.00-3.88 (m, 2H), 3.11-3.06 (m, 1H), 2.75-2.69 (m, 1H). OH and NH not observed. 2Xch under water peak, observed by HSQC.

LCMS: m/z=505.1/507.1 [M−H]−, (ESI−), RT=4.15, Method A

### Example 77—Synthesis of 25-chloro-17,19-difluoro-22,22-dioxo-9-oxa-2λ6-thia-3,4,6,21-tetrazapentacyclo[21.3.1.116,20.02,6.010,15]octacosa-1(26),2,4,10,12,14,16(28),17,19,23(27),24-undecaen-24-ol (Compound 77)

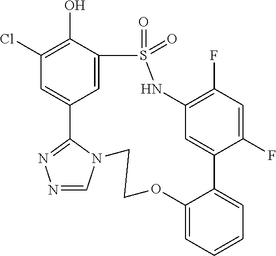 [SMILES: O=S1(=O)Nc2cc(c(F)cc2F)-c2ccccc2OCCn2cnnc2-c2cc(Cl)c(O)c1c2]

### Step 1

Hydrazine monohydrate (0.50 mL, 9.99 mmol) was added to a solution of Intermediate 4 (97% purity, 315 mg, 0.487 mmol) in MeOH (5 mL) and the mixture was heated at 60° C. for 6 hours. The mixture was cooled to r.t, and concentrated in vacuo before acetone (30 mL) was added and the mixture was concentrated in vacuo. The residue was purified by FCC (25 g SiO₂ cartridge, 0-50% acetone in heptane). The resultant white solid was dissolved in THE (10 mL) before 1 M aq. HCl (10 mL) was added. The mixture was stirred rapidly for 10 minutes before sat. aq. NaHCO₃ (30 mL). The organics were extracted with EtOAc (2×40 mL), combined, dried over MgSO₄, filtered and concentrated in vacuo. The resultant solid was dissolved in MeCN (30 mL) and to the solution was added water (10 mL) and 1 M aq. HCl (1 mL of a 1 M aqueous solution). The solution was stirred for 30 minutes at r.t, and was then concentrated in vacuo to afford N-[5-[2-(2-aminoethoxy)phenyl]-2,4-difluorophenyl]-3-chloro-5-(hydrazinecarbonyl)-2-methoxybenzenesulfonamide hydrochloride as a yellow solid (273 mg, 80% purity).

LCMS: m/z=527.1 [M+H]+, (ESI+), RT=0.64, Method B

### Step 2

To a suspension of N-[5-[2-(2-aminoethoxy)phenyl]-2,4-difluorophenyl]-3-chloro-5-(hydrazinecarbonyl)-2-methoxybenzenesulfonamide hydrochloride (80% purity, 273 mg, 0.388 mmol) and di-tert-butyl dicarbonate (85 mg, 0.389 mmol) in DCM (4 mL) at r.t. was added DIPEA (0.14 mL, 0.804 mmol). The mixture was stirred at r.t. for 15 minutes before 1 M aq. HCl (4 mL). The biphasic mixture was separated and the aqueous phase was extracted with DCM (2×5 mL). The combined organic phases were passed through a hydrophobic frit, washed with a mixture of sat. aq. NaHCO₃ (5 mL) and brine (2 mL), passed through a hydrophobic frit and concentrated in vacuo to afford tert-butyl N-[2-[2-[5-[[3-chloro-5-(hydrazinecarbonyl)-2-methoxyphenyl]sulfonylamino]-2,4-difluorophenyl]phenoxy]ethyl]carbamate (326 mg, 74% Yield, 55% purity) as an orange oil.

¹H NMR (500 MHz, DMSO) δ 10.03 (s, 1H), 8.22 (d, J=2.1 Hz, 1H), 8.15 (d, J=2.1 Hz, 1H), 7.38 (ddd, J=8.3, 7.2, 1.9 Hz, 1H), 7.27-7.22 (m, 1H), 7.17-7.13 (m, 1H), 7.13-7.10 (m, 1H), 7.08-7.05 (m, 1H), 7.04-7.00 (m, 1H), 6.74 (t, J=5.8 Hz, 1H), 4.57 (s, 2H), 3.95-3.92 (m, 2H), 3.92 (s, 3H), 3.18-3.13 (m, 2H), 1.34 (d, J=2.9 Hz, 9H). NH of hydrazone not observed.

LCMS: m/z=625.2/627.2 [M−H]−, (ESI−), RT=0.95, Method B

### Step 3

To a solution of tert-butyl N-[2-[2-[5-[[3-chloro-5-(hydrazinecarbonyl)-2-methoxyphenyl]sulfonylamino]-2,4-difluorophenyl]phenoxy]ethyl]carbamate (55% purity, 260 mg, 0.228 mmol) in anhydrous MeCN (5 mL) was added 1,1-dimethoxy-N,N-dimethylmethanamine (60 μL, 0.452 mmol). The mixture was heated at 60° C. for 40 minutes and was then concentrated in vacuo. The residue was purified by FCC (10 g SiO₂ cartridge, 0-70% acetone in heptane) to afford tert-butyl N-[2-[2-[5-[[3-chloro-5-[[(E)-dimethylaminomethyleneamino]carbamoyl]-2-methoxyphenyl]sulfonylamino]-2,4-difluorophenyl]phenoxy]ethyl]carbamate (126 mg, 63% Yield, 78% purity) as a yellow oil. LCMS: m/z=680.3/682.3 [M−H]−, (ESI−), RT=0.85, Method B

### Step 4

A solution of tert-butyl N-[2-[2-[5-[[3-chloro-5-[[(E)-dimethylaminomethyleneamino]carbamoyl]-2-methoxyphenyl]sulfonylamino]-2,4-difluorophenyl]phenoxy]ethyl]carbamate (78% purity, 126 mg, 0.144 mmol) and trifluoroacetic acid (15 μL, 0.196 mmol) in acetic acid (8 mL) was heated at 130° C. in a sealed vial for 1.25 hours. The mixture was allowed to cool to r.t. before it was added portion wise to a rapidly stirring biphasic mixture of EtOAc (60 mL) and sat. aq. NaHCO₃ (150 mL). The biphasic mixture was stirred for 10 minutes at r.t, and was then separated. The organic phase was washed with sat. aq. NaHCO₃ (40 mL). The aqueous layers were combined and were extracted with EtOAc (50 mL). The organic phases were combined, washed with brine (40 mL), dried over MgSO₄, filtered and concentrated in vacuo. The residue was purified by FCC (10 g SiO₂ cartridge, 0-100% acetone in DCM), followed by preparative HPLC (Method P3) to afford 25-chloro-17,19-difluoro-24-methoxy-9-oxa-22λ6-thia-3,4,6,21-tetrazapentacyclo[21.3.1.116,20.02,6.010,15]octacosa-1(27),2,4,10(15),11,13,16,18,20(28),23,25-undecaene 22,22-dioxide (19 mg, 23% Yield, 90% purity) as a colorless oil.

¹H NMR (500 MHz, CDCl3) δ 8.40-8.32 (m, 1H), 8.07 (d, J=2.2 Hz, 1H), 7.84 (d, J=2.1 Hz, 1H), 7.53-7.47 (m, 1H), 7.43-7.36 (m, 1H), 7.24-7.19 (m, 1H), 7.16-7.09 (m, 1H), 7.06-7.01 (m, 1H), 6.86-6.80 (m, 1H), 4.28-4.24 (m, 2H), 4.24-4.20 (m, 2H), 4.19 (s, 3H). 1H not observed.

### Step 5

To a solution of 25-chloro-17,19-difluoro-24-methoxy-9-oxa-22λ6-thia-3,4,6,21-tetrazapentacyclo[21.3.1.116,20.02,6.010,15]octacosa-1(26),2,4,10,12,14,16(28),17,19,23(27),24-undecaene 22,22-dioxide (90% purity, 19 mg, 0.0330 mmol) in anhydrous pyridine (0.8 mL) was added lithium iodide (35 mg, 0.261 mmol). The mixture was heated at 80° C. for 4.5 hours and was then allowed to cool to r.t. The mixture was diluted with EtOAc (25 mL), washed with a mixture of 1 M aq. HCl (20 mL) and brine (5 mL), then sat. aq. Na₂SO₃ (20 mL), then brine (10 mL). The organic phase was passed through a hydrophobic frit and concentrated in vacuo. The residue was purified by preparative HPLC (Method P1) to afford the title compound (12 mg, 76% Yield, 97% purity) as a white solid.

¹H NMR (400 MHz, DMSO) δ 8.71 (s, 1H), 7.92 (d, J=2.1 Hz, 1H), 7.61 (d, J=2.2 Hz, 1H), 7.44-7.38 (m, 1H), 7.34-7.27 (m, 1H), 7.24-7.13 (m, 3H), 7.10-7.03 (m, 1H), 4.29-4.20 (m, 4H). NH and OH not observed.

LCMS: m/z=503.1/505.1 [M−H]−, (ESI−), RT=2.77, Method A

### Example 78—Synthesis of 13-Chloro-19,21-difluoro-14-hydroxy-16,16-dioxo-9-oxa-16λ6-thia-17-azatetracyclo[16.3.1.111,15.02,7]tricosa-1(21),2(7),3,5,11(23),12,14,18(22),19-nonaen-10-one (Compound 78)

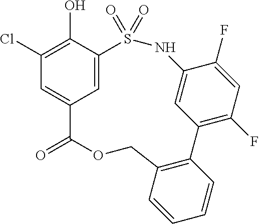 [SMILES: O=C1OCc2ccccc2-c2cc(c(F)cc2F)NS(=O)(=O)c2cc1cc(Cl)c2O]

### Step 1

A solution of Intermediate 15 (88 mg, 0.189 mmol) and iodocyclohexane (0.12 mL, 0.947 mmol) in anhydrous DMF (2 mL) was heated to 120° C. in a sealed vial for 3 hours. The mixture was cooled to r.t, and concentrated in vacuo, the residue was then purified by preparative HPLC (Method P2) then lyophilized to afford the title compound (27 mg, 30% Yield, 95% purity) as a yellow powder.

¹H NMR (500 MHz, DMSO) δ 10.11 (s, 1H), 7.95 (d, J=2.1 Hz, 1H), 7.60-7.54 (m, 1H), 7.52-7.43 (m, 3H), 7.28-7.25 (m, 1H), 7.23 (d, J=2.2 Hz, 1H), 6.79 (t, J=8.1 Hz, 1H), 5.44 (d, J=12.7 Hz, 1H), 5.13 (d, J=12.7 Hz, 1H).

LCMS: m/z=449.9/451.8 [M−H]−, (ESI−), RT=4.00, Method A

### Example 79—Synthesis of 15-chloro-4,21,22-trifluoro-16-hydroxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (Compound 79)

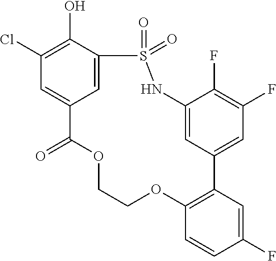 [SMILES: O=C1OCCOc2ccc(F)cc2-c2cc(F)c(F)c(c2)NS(=O)(=O)c2cc1cc(Cl)c2O]

15-chloro-4,21,22-trifluoro-16-methoxy-18,18-dioxo-8,11-dioxa-18λ6-thia-19-azatetracyclo[18.3.1.113,17.02,7]pentacosa-1(23),2(7),3,5,13,15,17(25),20(24),21-nonaen-12-one (synthesized using a similar method to Intermediate 12, 98% purity, 110 mg, 0.210 mmol) and iodocyclohexane (0.14 mL, 1.08 mmol) were combined and the mixture was dissolved into anhydrous DMF (3.3 mL). The mixture was heated to 120° C., and stirred for 1 hour. The reaction mixture was allowed to cool to r.t., and sat. aq. Na₂S₂O₃ (50 mL) was added, and the biphasic mixture was separated. The aqueous phase was extracted with DCM (3×50 mL). The combined organic extracts were washed with 1 M aq. HCl (50 mL), then brine (50 mL) and were dried over Na₂SO₄, filtered and concentrated under vacuum. The residue was purified by preparative HPLC (Method P1) to afford the title compound (61 mg, 58% Yield, 99% purity) as a brown solid.

¹H NMR (400 MHz, DMSO) δ 8.08-8.01 (m, 1H), 7.70 (d, J=2.1 Hz, 1H), 7.34-7.15 (m, 5H), 4.41-4.35 (m, 2H), 4.30-4.22 (m, 2H). NH and OH proton not observed.

LCMS: m/z=498.0/500.0 [M−H]−, (ESI−), RT=4.07, Method A

### Example 80—Synthesis of 14-chloro-20,21-difluoro-15-hydroxy-17,17-dioxo-10-oxa-17λ6-thia-18-azatetracyclo[17.3.1.112,16.02,7]tetracosa-1(23),2,4,6,12,14,16(24),19,21-nonaen-11-one (Compound 80)

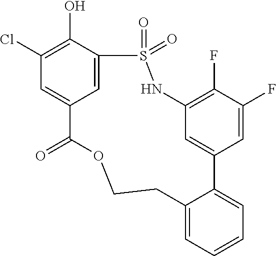 [SMILES: O=C1OCCc2ccccc2-c2cc(F)c(F)c(c2)NS(=O)(=O)c2cc1cc(Cl)c2O]

14-chloro-20,21-difluoro-15-methoxy-17,17-dioxo-10-oxa-17λ6-thia-18-azatetracyclo[17.3.1.112,16.02,7]tetracosa-1(23),2,4,6,12,14,16(24),19,21-nonaen-11-one (synthesized using a similar method to Intermediate 12, 214.0 mg, 0.36 mmol) and iodocyclohexane (0.23 mL, 1.74 mmol) were added to a pressure vial and anhydrous DMF (5 mL) was added. The mixture was heated to 120° C., and stirred for 1 hour, then cooled to r.t. The mixture was diluted with EtOAc (40 mL), washed with 1 M aq. HCl (100 mL), sat. aq. Na₂SO₃ (40 mL), then brine (40 mL). The EtOAc layer was passed through phase separator paper and concentrated in vacuo. The residue was purified by FCC (10 g SiO₂ column, 0-80% EtOAc in heptane) followed by lyophilization to afford the title compound (144 mg, 83% Yield, 96% purity) as a white solid.

¹H NMR (400 MHz, DMSO) δ 7.92 (d, J=2.1 Hz, 1H), 7.58-7.47 (m, 2H), 7.41-7.33 (m, 2H), 7.24 (td, J=7.5, 1.4 Hz, 1H), 7.02 (dd, J=7.7, 1.4 Hz, 1H), 6.46 (d, J=6.1 Hz, 1H), 4.37 (s, 2H).

LCMS: m/z=464.0 & 466.0 [M+H]+, (ESI+), RT=4.07, Method A

### Example 81—Synthesis of 13-chloro-4,19,21-trifluoro-14-hydroxy-16,16-dioxo-9-oxa-16λ6-thia-17-azatetracyclo[16.3.1.111,15.02,7]tricosa-1(21),2,4,6,11(23),12,14,18(22),19-nonaen-10-one (Compound 81)

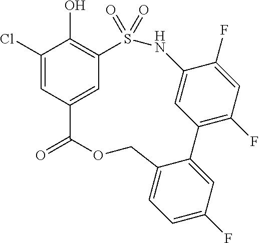 [SMILES: O=C1OCc2ccc(F)cc2-c2cc(c(F)cc2F)NS(=O)(=O)c2cc1cc(Cl)c2O]

A solution of 13-chloro-4,19,21-trifluoro-14-methoxy-16,16-dioxo-9-oxa-16λ6-thia-17-azatetracyclo[16.3.1.111,15.02,7]tricosa-1(21),2,4,6,11(23),12,14,18(22),19-nonaen-10-one (synthesized using a similar method to Intermediate 15, 99% purity, 45 mg, 0.0930 mmol) and iodocyclohexane (60 μL, 0.465 mmol) in anhydrous DMF (1.13 mL) was heated to 100° C. for 1 hour, then further heated to 120° C. for 1 hour. The mixture was cooled to r.t., diluted with EtOAc (20 mL) and washed with sat. aq. Na₂SO₃ (30 mL), 1 M aq. HCl 1M (30 mL) and brine (50 mL). The organic phases were dried over MgSO₄, filtered, and evaporated under vacuum. The residue was purified by preparative HPLC (Method P1) to afford the title compound (25 mg, 55% Yield, 97% purity) as an off-white solid.

¹H NMR (500 MHz, DMSO) δ 8.02-7.73 (m, 1H), 7.62 (dd, J=8.5, 5.8 Hz, 1H), 7.51-7.41 (m, 1H), 7.30 (ddd, J=8.6, 8.5, 2.8 Hz, 1H), 7.24-7.16 (m, 2H), 6.94-6.79 (m, 1H), 5.57-5.31 (m, 1H), 5.05 (d, J=12.7 Hz, 1H). OH and NH not integrated. LCMS: m/z=468.1, 470.1 [M−H]−, (ESI−), RT=4.01, Method A

### Example 82—Synthesis of 13-chloro-19,20-difluoro-14-hydroxy-16,16-dioxo-9-oxa-16λ6-thia-17-azatetracyclo[16.3.1.111,15.02,7]tricosa-1(21),2,4,6,11(23),12,14,18(22),19-nonaen-10-one (Compound 82)

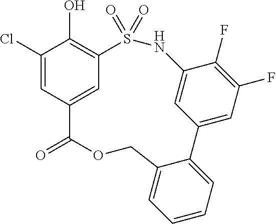 [SMILES: O=C1OCc2ccccc2-c2cc(F)c(F)c(c2)NS(=O)(=O)c2cc1cc(Cl)c2O]

A solution of 13-chloro-19,20-difluoro-14-methoxy-16,16-dioxo-9-oxa-16λ6-thia-17-azatetracyclo[16.3.1.111,15.02,7]tricosa-1(21),2,4,6,11(23),12,14,18(22),19-nonaen-10-one (synthesized using a similar method to Intermediate 15, 95 mg, 0.204 mmol) and iodocyclohexane (0.13 mL, 1.02 mmol) in anhydrous DMF (2 mL) was heated in a sealed vial at 100° C. for 3.25 hours. The mixture was cooled to r.t., diluted with EtOAc (30 mL) and sat. aq. NaHCO₃ (30 mL) then the layers separated. The aqueous phase was extracted with EtOAc (30 mL). The combined organic was washed with sat. aq. Na₂S₂O₃ (30 mL), water (30 mL), 1 M aq. HCl (30 mL) and then brine (30 ml). The organic phase was passed through a phase separator and then concentrated. The residue was purified by preparative HPLC (Method P2) then lyophilized to afford the title compound (46 mg, 49% Yield, 99% purity) as an off-white solid.

¹H NMR (400 MHz, DMSO) δ 10.36 (br. s, 1H), 7.93 (d, J=2.2 Hz, 1H), 7.62 (ddd, J=10.8, 6.8, 2.1 Hz, 1H), 7.58-7.49 (m, 1H), 7.48-7.36 (m, 2H), 7.30-7.17 (m, 2H), 6.63 (d, J=6.0 Hz, 1H), 5.63-5.19 (m, 2H).

LCMS: m/z=450.0/452.0 [M−H]−, (ESI−), RT=4.02, Method A

### Example 83—Synthesis of 13-chloro-4,19,20-trifluoro-14-hydroxy-16,16-dioxo-9-oxa-16λ6-thia-17-azatetracyclo[16.3.1.111,15.02,7]tricosa-1(21),2,4,6,11,13,15(23),18(22),19-nonaen-10-one (Compound 83)

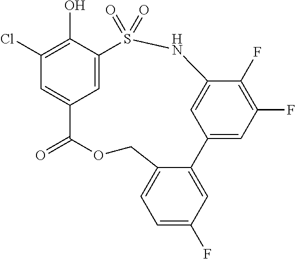 [SMILES: O=C1OCc2ccc(F)cc2-c2cc(F)c(F)c(c2)NS(=O)(=O)c2cc1cc(Cl)c2O]

13-chloro-4,19,20-trifluoro-14-methoxy-16,16-dioxo-9-oxa-16λ6-thia-17-azatetracyclo[16.3.1.111,15.02,7]tricosa-1(21),2(7),3,5,11,13,15(23),18(22),19-nonaen-10-one (synthesized using a similar method to Intermediate 15, 99% purity, 70 mg, 0.143 mmol) and iodocyclohexane (150 mg, 0.716 mmol) were added to a pressure vial and anhydrous DMF (2 mL) was added. The mixture was placed at 120° C., and stirred for 1 hour, then cooled to r.t. Sat aq. Na₂S₂O₃ (25 mL) was added and the biphasic mixture was separated. The aqueous phase was extracted with DCM (3×20 mL). The combined organic extracts were washed with 1 M aq. HCl (25 mL), then brine (25 mL) and were dried over MgSO₄, filtered, and concentrated under reduced pressure. The residue was purified by preparative HPLC (Method P1) then lyophilized to afford the title compound (16 mg, 22% Yield, 94% purity) as white solid.

¹H NMR (400 MHz, DMSO) δ 7.94-7.89 (m, 1H), 7.68-7.62 (m, 1H), 7.60 (dd, J=8.6, 5.9 Hz, 1H), 7.27 (ddd, J=8.6, 8.6, 2.8 Hz, 1H), 7.21 (d, J=2.2 Hz, 1H), 7.15 (dd, J=9.3, 2.8 Hz, 1H), 6.69 (d, J=5.8 Hz, 1H), 5.49-5.24 (m, 2H). NH and OH not observed.

LCMS: m/z=468.0, 470.0 [M−H]−, (ESI−), RT=4.04, Method A

### Example 84—Synthesis of 13-chloro-19-fluoro-14-hydroxy-16,16-dioxo-9-oxa-16λ6-thia-17-azatetracyclo[16.3.1.111,15.02,7]tricosa-1(21),2(7),3,5,11(23),12,14,18(22),19-nonaen-10-one (Compound 84)

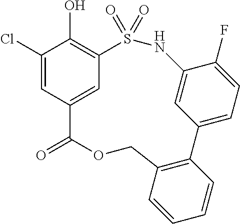 [SMILES: O=C1OCc2ccccc2-c2ccc(F)c(c2)NS(=O)(=O)c2cc1cc(Cl)c2O]

13-chloro-19-fluoro-14-methoxy-16,16-dioxo-9-oxa-16λ6-thia-17-azatetracyclo[16.3.1.111,15.02,7]tricosa-1(21),2(7),3,5,11(23),12,14,18(22),19-nonaen-10-one (synthesized using a similar method to Intermediate 15, 95% purity, 230 mg, 0.488 mmol), anhydrous DMF (5 mL) and iodocyclohexane (0.50 mL, 3.87 mmol) were added to pressure vial. The vial was sealed and heated at 120° C. for 1 hour and 20 minutes, then allowed to cool to r.t, and stirred for a further 16 hours. The reaction mixture was diluted with EtOAc (40 mL), washed with 1 M aq. HCl (100 mL), sat. aq. Na₂SO₃ (40 mL), then brine (2×40 mL), dried over MgSO₄, filtered, and concentrated in vacuo. The mixture was twice purified by preparative HPLC (Method P1), followed by FCC (10 g SiO₂ column, 0-50% MeOH in DCM) then lyophilized to afford the title compound (34 mg, 16% Yield, 99% purity) as a white solid.

¹H NMR (500 MHz, DMSO) δ 10.07 (br. s, 1H), 7.88 (d, J=2.2 Hz, 1H), 7.53 (dd, J=7.2, 1.8 Hz, 1H), 7.45-7.36 (m, 3H), 7.26-7.22 (m, 2H), 7.20 (dd, J=10.2, 8.4 Hz, 1H), 6.88 (dd, J=7.4, 2.2 Hz, 1H), 5.48-5.36 (m, 1H), 5.31-5.20 (m, 1H). 1H not observed.

LCMS: m/z=432.0/434.1 [M−H]−, (ESI−), RT=3.91, Method A

### Example 85—Synthesis of 13-chloro-19,21-difluoro-14-hydroxy-10,16,16-trioxo-9-oxa-16λ6-thia-17-azatetracyclo[16.3.1.111,15.02,7]tricosa-1(21),2(7),3,5,11,13,15(23),18(22),19-nonaene-5-carbonitrile (Compound 85)

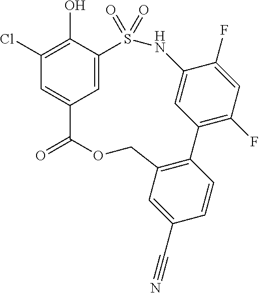 [SMILES: N#Cc1ccc2c(c1)COC(=O)c1cc(Cl)c(O)c(c1)S(=O)(=O)Nc1cc-2c(F)cc1F]

To a solution of 13-chloro-19,21-difluoro-14-methoxy-10,16,16-trioxo-9-oxa-16λ6-thia-17-azatetracyclo[16.3.1.111,15.02,7]tricosa-1(21),2(7),3,5,11,13,15(23),18(22),19-nonaene-5-carbonitrile (synthesized using a similar method to Intermediate 15, 87% purity, 32 mg, 0.0567 mmol) in anhydrous pyridine (4 mL) was added lithium iodide (73 mg, 0.545 mmol). The mixture was heated at 80° C. for 6 hours and then cooled to r.t, and stirred at r.t. for 8 hours. The reaction mixture was concentrated in vacuo and the residue was purified by preparative HPLC (Method P1) then lyophilized to afford the title compound (21 mg, 75% Yield, 98% purity) as an off-white solid.

¹H NMR (500 MHz, DMSO) δ 10.14 (br. s, 1H), 8.11 (d, J=1.7 Hz, 1H), 7.98-7.91 (m, 2H), 7.56-7.48 (m, 2H), 7.21 (d, J=2.2 Hz, 1H), 6.92-6.84 (m, 1H), 5.50 (d, J=13.0 Hz, 1H), 5.13 (d, J=13.0 Hz, 1H). 1H not observed.

LCMS: m/z=475.0/477.0 [M−H]−, (ESI−), RT=3.81, Method

### Example 86—Synthesis of 13-chloro-19,21-difluoro-14-hydroxy-10,16,16-trioxo-9-oxa-16λ6-thia-17-azatetracyclo[16.3.1.111,15.02,7]tricosa-1(21),2,4,6,11(23),12,14,18(22),19-nonaene-4-carbonitrile (Compound 86)

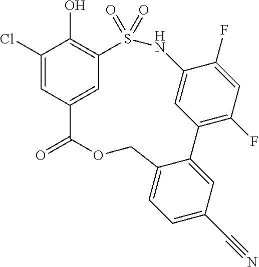 [SMILES: N#Cc1ccc2c(c1)-c1cc(c(F)cc1F)NS(=O)(=O)c1cc(cc(Cl)c1O)C(=O)OC2]

13-chloro-19,21-difluoro-14-methoxy-10,16,16-trioxo-9-oxa-16λ6-thia-17-azatetracyclo[16.3.1.111,15.02,7]tricosa-1(21),2,4,6,11(23),12,14,18(22),19-nonaene-4-carbonitrile (synthesized using a similar method to Intermediate 15, 96% purity, 110 mg, 0.215 mmol) and iodocyclohexane (230 mg, 1.09 mmol) were dissolved in anhydrous DMF (3 mL) and the mixture was heated to 120° C. with stirring for 2 hours. The reaction mixture was allowed to cool to r.t., and sat aq. Na₂S₂O₃ (50 mL) was added. The biphasic mixture was separated and the aqueous phase was extracted with DCM (3×50 mL). The combined organic extracts were washed with 1 M aq. HCl (50 mL), then brine (50 mL) and were dried over Na₂SO₄, filtered, and concentrated under vacuum. The residue was purified by preparative HPLC (Method P1) to afford the title compound (67 mg, 63% Yield, 97% purity) as an off-white solid.

¹H NMR (500 MHz, DMSO) δ 10.15 (br. s, 1H), 7.97 (dd, J=7.9, 1.8 Hz, 1H), 7.92 (d, J=2.2 Hz, 1H), 7.83-7.77 (m, 2H), 7.53-7.47 (m, 1H), 7.22 (d, J=2.2 Hz, 1H), 6.95-6.90 (m, 1H), 5.51 (d, J=13.2 Hz, 1H), 5.16 (d, J=13.1 Hz, 1H).

### Example 87—Synthesis of 13-chloro-19,21-difluoro-14-hydroxy-16,16-dioxo-9-oxa-16λ6-thia-3,17-diazatetracyclo[16.3.1.111,15.02,7]tricosa-1(21),2(7),3,5,11,13,15(23),18(22),19-nonaen-10-one (Compound 87)

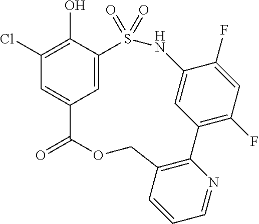 [SMILES: O=C1OCc2cccnc2-c2cc(c(F)cc2F)NS(=O)(=O)c2cc1cc(Cl)c2O]

To a solution of 13-chloro-19,21-difluoro-14-methoxy-16,16-dioxo-9-oxa-16λ6-thia-3,17-diazatetracyclo[16.3.1.111,15.02,7]tricosa-1(21),2(7),3,5,11,13,15(23),18(22),19-nonaen-10-one (synthesized using a similar method to Intermediate 15, 80% purity, 105 mg, 0.180 mmol) in anhydrous pyridine (6 mL) was added lithium iodide (190 mg, 1.42 mmol). The reaction mixture was heated at 80° C. for 6 hours and then cooled to r.t, and stirred at r.t. for 8 hours. The reaction mixture was concentrated in vacuo. The residue was purified by preparative HPLC (Method P1) then lyophilized to afford the title compound (67 mg, 81% Yield, 99% yield) as an off-white solid.

¹H NMR (500 MHz, DMSO) δ 10.11 (s, 1H), 8.63 (dd, J=4.9, 1.7 Hz, 1H), 8.01 (dd, J=7.8, 1.7 Hz, 1H), 7.94 (d, J=2.1 Hz, 1H), 7.56-7.48 (m, 2H), 7.20 (d, J=2.2 Hz, 1H), 6.88-6.74 (m, 1H), 5.48 (d, J=13.1 Hz, 1H), 5.17 (d, J=13.0 Hz, 1H). 1H not observed.

LCMS: m/z=451.1/453.0 [M−H]−, (ESI−), RT=3.33, Method A

### Example 88—Synthesis of 13-chloro-19,21-difluoro-14-hydroxy-16,16-dioxo-9-oxa-16λ6-thia-6,17-diazatetracyclo[16.3.1.111,15.02,7]tricosa-1(21),2(7),3,5,11,13,15(23),18(22),19-nonaen-10-one (Compound 88)

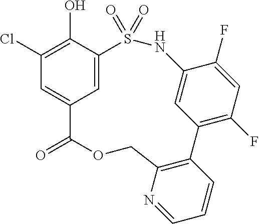 [SMILES: O=C1OCc2ncccc2-c2cc(c(F)cc2F)NS(=O)(=O)c2cc1cc(Cl)c2O]

To a solution of 13-chloro-19,21-difluoro-14-methoxy-16,16-dioxo-9-oxa-16λ6-thia-6,17-diazatetracyclo[16.3.1.111,15.02,7]tricosa-1(21),2(7),3,5,11,13,15(23),18(22),19-nonaen-10-one (synthesized using a similar method to Intermediate 15, 65% purity, 33 mg, 0.0459 mmol) in anhydrous pyridine (4 mL) was added lithium iodide (65 mg, 0.486 mmol). The reaction mixture was heated at 80° C. for 18 hours. The reaction mixture was concentrated in vacuo and the residue was purified by preparative HPLC (Method P1), then lyophilized to afford the title compound (15 mg, 69% Yield, 99% purity) as an off-white solid.

¹H NMR (500 MHz, DMSO) δ 8.62 (dd, J=4.9, 1.7 Hz, 1H), 7.94 (d, J=2.2 Hz, 1H), 7.78 (dd, J=7.7, 1.7 Hz, 1H), 7.52 (dd, J=7.7, 4.8 Hz, 1H), 7.47-7.43 (m, 1H), 7.27 (d, J=2.2 Hz, 1H), 7.07-7.02 (m, 1H), 5.42 (d, J=13.2 Hz, 1H), 5.37 (d, J=13.2 Hz, 1H). Exchangeable protons not observed.

LCMS: m/z=451.0/453.0 [M−H]−, (ESI−), RT=3.37, Method A

### Example 89—Synthesis of 13-chloro-19,21-difluoro-14-hydroxy-16,16-dioxo-16λ6-thia-9,17-diazapentacyclo[16.3.1.16,9.111,15.02,7]tetracosa-1(21),2(7),3,5,11,13,15(23),18(22),19-nonaen-10-one (Compound 89)

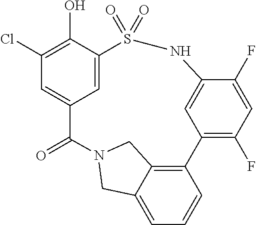 [SMILES: O=C1c2cc(Cl)c(O)c(c2)S(=O)(=O)Nc2cc(c(F)cc2F)-c2cccc3c2CN1C3]

To a solution of Intermediate 19 (83% purity, 60 mg, 0.104 mmol) in anhydrous pyridine (6.6 mL) was added lithium iodide (156 mg, 1.17 mmol). The mixture was heated at 80° C. for 24 hours and then cooled to r.t. The reaction mixture was diluted with EtOAc (30 mL), washed with 1 M aq. HCl (2×30 mL), then sat. aq. Na₂SO₃ (10 mL), then brine (2×20 mL), dried over MgSO₄, filtered and concentrated in vacuo. The residue was purified by preparative HPLC (Method P1) to afford the title compound (17 mg, 35% Yield, 99% purity) as an off-white solid.

¹H NMR (500 MHz, DMSO) δ 11.05 (s, 1H), 7.82-7.63 (m, 2H), 7.48 (d, J=7.4 Hz, 1H), 7.44-7.34 (m, 2H), 7.33 (d, J=7.5 Hz, 1H), 7.19-7.12 (m, 1H), 5.17-4.37 (m, 2H), 4.01-3.78 (m, 2H). 1H not observed.

LCMS: m/z=461.1/463.0 [M−H]−, (ESI−), RT=3.15, Method A

### Example 90—Synthesis of 13-chloro-19,21-difluoro-14-hydroxy-16,16-dioxo-16λ6-thia-6,9,17-triazatetracyclo[16.3.1.111,15.02,7]tricosa-1(22),2,4,6,11,13,15(23),18,20-nonaen-10-one (Compound 90)

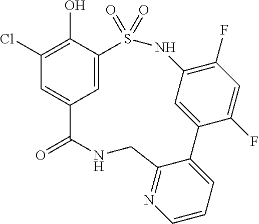 [SMILES: O=C1NCc2ncccc2-c2cc(c(F)cc2F)NS(=O)(=O)c2cc1cc(Cl)c2O]

To a solution of 13-chloro-19,21-difluoro-14-methoxy-16,16-dioxo-16λ6-thia-6,9,17-triazatetracyclo[16.3.1.111,15.02,7]tricosa-1(22),2,4,6,11,13,15(23),18,20-nonaen-10-one (synthesized using a similar method to Intermediate 19, 80% purity, 85 mg, 0.146 mmol) in anhydrous pyridine (6.4 mL) was added lithium iodide (216 mg, 1.61 mmol). The mixture was heated at 80° C. for 2 hours then cooled to r.t and stirred overnight. The reaction mixture was diluted with EtOAc (30 mL), washed with 1 M aq. HCl (2×20 mL), then sat. aq. Na₂SO₃ (10 mL), then brine (10 mL), dried over MgSO₄, filtered, and concentrated in vacuo. The residue was purified by preparative HPLC (Method P2) to afford the title compound (16 mg, 24% Yield, 99% purity) as a white solid.

¹H NMR (353 K, 400 MHz, DMSO) δ 8.61 (dd, J=4.9, 1.6 Hz, 1H), 8.17-7.99 (m, 1H), 7.70 (s, 1H), 7.61 (dd, J=7.6, 1.7 Hz, 1H), 7.42-7.33 (m, 2H), 7.28 (dd, J=9.9, 9.9 Hz, 1H), 6.81 (dd, J=8.2, 8.2 Hz, 1H), 4.43-4.22 (m, 2H). 2H not observed.

LCMS: m/z=452.0, 453.7 [M+H]+, (ESI+), RT=2.38, Method A

### Example 91—Synthesis of 13-chloro-19,21-difluoro-14-hydroxy-9-methyl-16,16-dioxo-16λ6-thia-9,17-diazatetracyclo[16.3.1.111,15.02,7]tricosa-1(22),2,4,6,11(23),12,14,18,20-nonaen-10-one (Compound 91)

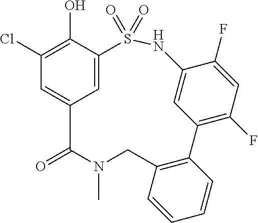 [SMILES: CN1Cc2ccccc2-c2cc(c(F)cc2F)NS(=O)(=O)c2cc(cc(Cl)c2O)C1=O]

Iodocyclohexane (191 μL, 1.48 mmol) was added to a solution of 4,21-difluoro-16-methoxy-18,18-dioxo-8,11-dioxa-18λ6-thia-15,19-diazatetracyclo[18.3.1.113,17.02,7]pentacosa-1(23),2(7),3,5,13,15,17(25),20(24),21-nonaen-12-one (synthesized using a similar method to Intermediate 19, 76% purity, 90 mg, 0.148 mmol) in anhydrous DMF (3 mL) and the mixture was heated at 120° C. for 2 hours. The mixture was cooled to r.t., diluted with DCM (5 mL) and sat. aq. Na₂S₂O₃ (3 mL), then the layers separated. The aqueous layer was extracted with further DCM (3 mL) and the combined organic layers were passed through a phase separator and then concentrated. The residue was purified by preparative HPLC (Method P2) to afford the title compound (4.4 mg, 19% Yield, 98% purity) as an off-white solid.

¹H NMR (400 MHz, DMSO) δ 7.54-7.41 (m, 2H), 7.33-7.10 (m, 5H), 5.54 (s, 1H), 4.33 (d, J=17.6 Hz, 1H), 4.04 (d, J=18.1 Hz, 1H), 3.22 (s, 3H).

LCMS: m/z=465.3 [M+H]+, (ESI+), RT=3.11, Method A

### Example 92—Synthesis of 15-chloro-21-fluoro-18,18-dioxo-11-oxa-18λ6-thia-19-azatetracyclo[18.3.1.113,17.02,7]pentacosa-1(24),2,4,6,13,15,17(25),20,22-nonaen-16-ol (Compound 92)

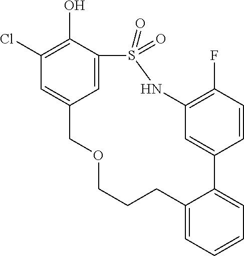 [SMILES: O=S1(=O)Nc2cc(ccc2F)-c2ccccc2CCCOCc2cc(Cl)c(O)c1c2]

### Step 1

Intermediate 20 (50% purity, 550 mg, 1.01 mmol) and 5-[2-[3-[tert-butyl(dimethyl)silyl]oxypropyl]phenyl]-2-fluoro-aniline (synthesized using a similar method to Intermediate 8, 90% purity, 405 mg, 1.01 mmol) were dissolved in anhydrous pyridine (6 mL) and the mixture was heated to 50° C., and stirred for 1 hour. The mixture was allowed to cool to r.t, and was diluted with 1 M aq. HCl (50 mL) and extracted with EtOAc (3×50 mL). The combined organic extracts were washed with brine (50 mL), dried over Na₂SO₄, filtered and concentrated under reduced pressure. The residue was purified by FCC (25 g SiO₂ cartridge, 0-100% EtOAc in heptane) to afford N-[5-[2-[3-[tert-butyl(dimethyl)silyl]oxypropyl]phenyl]-2-fluorophenyl]-3-chloro-5-(hydroxymethyl)-2-methoxybenzenesulfonamide (444 mg, 70% Yield, 95% purity) as a viscous colorless oil.

¹H NMR (500 MHz, DMSO) δ 10.21 (s, 1H), 7.68-7.65 (m, 2H), 7.32-7.25 (m, 2H), 7.24-7.18 (m, 2H), 7.09 (dd, J=7.6, 2.0 Hz, 2H), 7.01 (dd, J=7.6, 1.4 Hz, 1H), 5.44 (t, J=5.7 Hz, 1H), 4.45 (d, J=5.7 Hz, 2H), 3.87 (s, 3H), 3.37 (t, J=6.3 Hz, 2H), 2.48-2.44 (m, 2H), 1.50-1.41 (m, 2H), 0.79 (s, 9H), −0.07 (s, 6H).

LCMS: m/z=594.2/596.2 [M+H]+, (ESI+), RT=1.21, Method C Step 2

Triphenylphosphine (400 mg, 1.5 mmol) and carbon tetrabromide (480 mg, 1.5 mmol) were added to DCM (4 mL) and stirred for 10 minutes until white suspension formed. A separate solution of N-[5-[2-[3-[tert-butyl(dimethyl)silyl]oxypropyl]phenyl]-2-fluorophenyl]-3-chloro-5-(hydroxymethyl)-2-methoxybenzenesulfonamide (95% purity, 400 mg, 0.64 mmol) in DCM (15 mL) was prepared and the two components were combined. The resulting mixture was stirred for 1 hour at r.t. The mixture was concentrated in vacuo and the residue was purified by FCC (25 g SiO₂ cartridge, 0-30% EtOAc in heptane) to afford 5-(bromomethyl)-N-[5-[2-[3-[tert-butyl(dimethyl)silyl]oxypropyl]phenyl]-2-fluorophenyl]-3-chloro-2-methoxybenzenesulfonamide (105 mg, 30% Yield, 90% purity) as a colorless oil.

¹H NMR (500 MHz, DMSO) δ 10.32 (s, 1H), 7.88 (s, 1H), 7.79 (d, J=2.2 Hz, 1H), 7.32-7.16 (m, 4H), 7.09-6.97 (m, 3H), 4.69 (s, 2H), 3.88 (s, 3H), 3.39 (t, J=6.3 Hz, 2H), 2.56-2.53 (m, 2H), 1.47 (p, J=6.0 Hz, 2H), 0.79 (s, 9H), −0.06 (s, 6H).

LCMS: m/z=654.0/656.3/658.2 [M−H]−, (ESI−), RT=1.31, Method C

### Step 3

To a solution of 5-(bromomethyl)-N-[5-[2-[3-[tert-butyl(dimethyl)silyl]oxypropyl]phenyl]-2-fluorophenyl]-3-chloro-2-methoxy-benzenesulfonamide (90% purity, 100 mg, 0.137 mmol) in methanol (2 mL) was added p-toluenesulfonic acid monohydrate (3.0 mg, 0.0158 mmol). The reaction mixture was stirred for 1 hour at r.t, and was then concentrated in vacuo. The residue was purified by FCC (25 g SiO2 cartridge, 0-100% ethyl acetate in heptane) to afford 5-(bromomethyl)-3-chloro-N-[2-fluoro-5-[2-(3-hydroxypropyl)phenyl]phenyl]-2-methoxybenzenesulfonamide (77 mg, 93% Yield, 90% purity) as a sticky white solid.

¹H NMR (500 MHz, DMSO) δ 10.29 (s, 1H), 7.89 (s, 1H), 7.78 (d, J=2.2 Hz, 1H), 7.31-7.25 (m, 2H), 7.23-7.18 (m, 2H), 7.12-7.03 (m, 2H), 7.01 (dd, J=7.3, 1.3 Hz, 1H), 4.70 (s, 2H), 4.34 (t, J=5.1 Hz, 1H), 3.90 (s, 3H), 3.24-3.19 (m, 2H), 2.47-2.43 (m, 2H), 1.48-1.41 (m, 2H).

LCMS: m/z=540.0/542.1/544.0 [M−H]−, (ESI−), RT=1.02, Method B

### Step 4

To a solution of 5-(bromomethyl)-3-chloro-N-[2-fluoro-5-[2-(3-hydroxypropyl)phenyl]phenyl]-2-methoxybenzenesulfonamide (90% purity, 75 mg, 0.13 mmol) in anhydrous THE (10 mL) at 0° C. was added sodium hydride in mineral oil (60%, 16 mg, 0.400 mmol). The reaction mixture was heated at 50° C. for 2 hours. After cooling the reaction mixture to r.t., 1 M aq. HCl (10 mL) and water (20 mL) were added. The biphasic mixture was diluted with EtOAc (20 mL), and the resulting biphasic mixture was separated. The organic phase was washed with brine (20 mL). The organic phase was then dried over MgSO₄, filtered, and concentrated in vacuo. The residue was purified by FCC (10 g SiO₂ cartridge, 20-100% DCM in heptane) to afford 15-chloro-21-fluoro-16-methoxy-11-oxa-18λ6-thia-19-azatetracyclo[18.3.1.113,17.02,7]pentacosa-1(24),2(7),3,5,13,15,17(25),20,22-nonaene 18,18-dioxide (29 mg, 46% Yield, 97% purity) as a white solid.

¹H NMR (400 MHz, DMSO) δ 7.63-7.61 (m, 2H), 7.29-7.26 (m, 2H), 7.22-7.18 (m, 1H), 7.17-7.13 (m, 2H), 7.10-7.06 (m, 2H), 4.35 (s, 2H), 3.96 (s, 3H), 3.05 (t, J=6.4 Hz, 2H), 2.44-2.38 (m, 2H), 1.51 (p, J=6.5 Hz, 2H).

LCMS: m/z=460.1/462.1 [M−H]−, (ESI−), RT=1.12, Method B

### Step 5

To a solution of 15-chloro-21-fluoro-16-methoxy-11-oxa-18λ6-thia-19-azatetracyclo[18.3.1.113,17.02,7]pentacosa-1(24),2,4,6,13,15,17(25),20,22-nonaene 18,18-dioxide (97% purity, 27 mg, 0.055 mmol) in anhydrous pyridine (1.5 mL) was added lithium iodide (50 mg, 0.374 mmol). The mixture was heated at 80° C. for 72 hours and was then allowed to cool to r.t. The reaction mixture was added to EtOAc (50 mL) and the solution was washed sequentially with 1 M aq. HCl (30 mL), Na₂SO₃ (30 mL), and brine (20 mL). The organic phase was dried over MgSO₄, filtered and concentrated in vacuo. The residue was purified by preparative HPLC (Method P1) to afford the title compound (19 mg, 74% Yield, 99% purity) as a white solid.

¹H NMR (400 MHz, DMSO) δ 7.56-7.52 (m, 2H), 7.32-7.29 (m, 2H), 7.25-7.18 (m, 3H), 7.10 (d, J=7.5 Hz, 1H), 7.00-6.95 (m, 1H), 4.30 (s, 2H), 3.04 (t, J=6.4 Hz, 2H), 2.42-2.33 (m, 2H), 1.55-1.44 (m, 2H).

LCMS: m/z=446.1/448.1 [M−H]−, (ESI−), RT=3.94, Method A

### Example 93—Synthesis of 15-chloro-21-fluoro-16-hydroxy-11-methyl-18,18-dioxo-8-oxa-18λ6-thia-11,19-diazatetracyclo[18.3.1.113,17.02,7]pentacosa-1(24),2,4,6,13(25),14,16,20,22-nonaen-10-one (Compound 93)

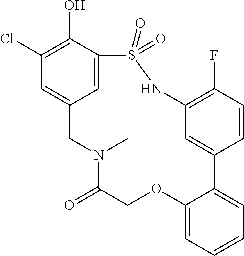 [SMILES: CN1Cc2cc(Cl)c(O)c(c2)S(=O)(=O)Nc2cc(ccc2F)-c2ccccc2OCC1=O]

### Step 1

A solution of Intermediate 20 (4 mmol, 50% purity) and methyl 2-[2-(3-amino-4-fluoro-phenyl)phenoxy]acetate (synthesized using a similar method to Intermediate 8, 85% purity, 1.07 g, 3.29 mmol) in pyridine (8 mL) was stirred at 50° C. for 1 hour. The mixture was cooled to r.t., diluted with EtOAc (40 mL) and 1 M aq. HCl (40 mL) and the biphasic layers were separated. The aqueous phase was extracted with further EtOAc (30 mL). The combined organic was washed with water (40 mL) and brine (40 mL), then dried over MgSO₄, filtered, and concentrated. The residue was purified by FCC (100 g SiO₂ column, 0-100% EtOAc in heptane) to afford methyl 2-[2-[3-[[3-chloro-5-(hydroxymethyl)-2-methoxy-phenyl]sulfonylamino]-4-fluorophenyl]phenoxy]acetate (898 mg, 53% Yield, 95% purity) as an off-white solid.

¹H NMR (500 MHz, DMSO) δ 10.12 (s, 1H), 7.71-7.63 (m, 2H), 7.43-7.35 (m, 2H), 7.30 (ddd, J=8.3, 7.4, 1.8 Hz, 1H), 7.21 (dd, J=10.3, 8.4 Hz, 1H), 7.16 (dd, J=7.6, 1.8 Hz, 1H), 7.06-7.01 (m, 1H), 7.01-6.96 (m, 1H), 5.45 (t, J=5.8 Hz, 1H), 4.78 (s, 2H), 4.46 (d, J=5.6 Hz, 2H), 3.89 (s, 3H), 3.68 (s, 3H).

LCMS: m/z=508.1/510.1 [M−H]−, (ESI−), RT=0.93, Method B

### Step 2

Methyl 2-[2-[3-[[3-chloro-5-(hydroxymethyl)-2-methoxy-phenyl]sulfonylamino]-4-fluorophenyl]phenoxy]acetate (95% purity, 180 mg, 0.335 mmol) was dissolved in THE (2.7 mL) and 2 M aq. sodium hydroxide (0.90 mL, 1.80 mmol) was added. The mixture was stirred at r.t. for 1 hour. THE was removed under vacuum and the remaining aqueous mixture was diluted with 1 M aq. HCl (20 mL). The mixture was extracted with DCM (3×30 mL), and the combined organic phases were passed through a phase separator and then concentrated to give 3-chloro-5-[[2-fluoro-5-[4-fluoro-2-(2-hydroxyethoxy)phenyl]phenyl]sulfamoyl]-4-methoxybenzoic acid (164 mg, 93% Yield, 95% purity) as an off-white solid.

¹H NMR (500 MHz, DMSO) δ 13.00 (br. s, 1H), 10.11 (br. s, 1H), 7.69-7.65 (m, 2H), 7.42 (ddd, J=8.5, 4.7, 2.3 Hz, 1H), 7.37 (dd, J=7.7, 2.2 Hz, 1H), 7.29 (ddd, J=8.5, 7.4, 1.7 Hz, 1H), 7.20 (dd, J=10.3, 8.5 Hz, 1H), 7.14 (dd, J=7.5, 1.8 Hz, 1H), 7.04-6.99 (m, 1H), 6.95 (dd, J=8.4, 1.0 Hz, 1H), 5.72-5.06 (m, 1H), 4.65 (s, 2H), 4.46 (s, 2H), 3.89 (s, 3H).

LCMS: m/z=494.1/496.1 [M−H]−, (ESI−), RT=0.83, Method B

### Step 3

A mixture of 2-[2-[3-[[3-chloro-5-(hydroxymethyl)-2-methoxyphenyl]sulfonylamino]-4-fluorophenyl]phenoxy]acetic acid (95% purity, 50 mg, 0.095 mmol), MnO₂ (82 mg, 0.95 mmol) and CHCl₃ (3 mL) was stirred at r.t. for 6.5 hours, then at 35° C. overnight. Further MnO₂ (82 mg, 0.945 mmol) was added and the mixture was stirred at 50° C. for 4 hours. The mixture was filtered through Celite, washing with DCM. The filtrate was concentrated in vacuo to give 2-[2-[3-[(3-chloro-5-formyl-2-methoxyphenyl)sulfonylamino]-4-fluorophenyl]phenoxy]acetic acid (41 mg, 85% Yield, 97% purity) as a pale-yellow solid.

¹H NMR (400 MHz, DMSO) δ 13.00 (br. s, 1H), 10.39 (br. s, 1H), 9.95 (s, 1H), 8.29 (d, J=2.0 Hz, 1H), 8.19 (d, J=2.0 Hz, 1H), 7.49-7.38 (m, 2H), 7.33-7.26 (m, 1H), 7.25-7.14 (m, 2H), 7.05-6.98 (m, 1H), 6.96 (d, J=8.3 Hz, 1H), 4.65 (s, 2H), 4.01 (s, 3H).

LCMS: m/z=492.1/494.0 [M−H]−, (ESI−), RT=0.88, Method B

### Step 4

To a mixture of 2-[2-[3-[(3-chloro-5-formyl-2-methoxyphenyl)sulfonylamino]-4-fluorophenyl]phenoxy]acetic acid (97% purity, 41 mg, 0.0805 mmol) and DCM (4 mL) was added methylamine in EtOH (33%, 15 μL, 0.121 mmol) and then sodium triacetoxyborohydride (34 mg, 0.161 mmol). The mixture was stirred at r.t. overnight. The mixture was then diluted with water (30 mL) and extracted with 1:3 IPA:CHCl₃ (3×20 mL). The combined organic phases were passed through a phase separator and then concentrated to give 2-[2-[3-[[3-chloro-2-methoxy-5-(methylaminomethyl)phenyl]sulfonylamino]-4-fluorophenyl]phenoxy]acetic acid (37 mg, 81% Yield, 90% purity) as an off-white solid.

LCMS: m/z=509.1/511.1 [M+H]+, (ESI+), RT=0.68, Method B

### Step 5

To a solution of 2-[2-[3-[[3-chloro-2-methoxy-5-(methylaminomethyl)phenyl]sulfonylamino]-4-fluorophenyl]phenoxy]acetic acid (90% purity, 49 mg, 0.09 mmol) and DIPEA (134 μL, 0.77 mmol) in anhydrous DMF (8 mL) was added T3P (50% in EtOAc) (137 μL, 0.23 mmol) and the mixture was stirred at r.t. for 30 minutes. The mixture was diluted with water (30 mL) and EtOAc (30 mL) and the layers were separated. The aqueous phase was extracted with further EtOAc (30 mL). The combined organic phases were washed with water (40 mL) and brine (40 mL), then passed through a phase separator and concentrated to give 15-chloro-21-fluoro-16-methoxy-11-methyl-18,18-dioxo-8-oxa-18λ6-thia-11,19-diazatetracyclo[18.3.1.113,17.02,7]pentacosa-1(24),2,4,6,13,15,17(25),20,22-nonaen-10-one (35 mg, 75% Yield, 92% purity) as a pale yellow solid.

¹H NMR (400 MHz, DMSO) δ 10.32 (s, 1H), 7.72-7.62 (m, 1H), 7.62-7.51 (m, 1H), 7.45-7.34 (m, 1H), 7.33-7.26 (m, 1H), 7.22-7.12 (m, 2H), 7.13-7.00 (m, 2H), 6.97-6.83 (m, 1H), 4.70 (s, 2H), 4.62-4.26 (m, 2H), 3.98 (s, 3H), 2.37-2.26 (m, 3H).

LCMS: m/z=489.1/491.1 [M−H]−, (ESI−), RT=0.93, Method B

### Step 6

15-chloro-21-fluoro-16-methoxy-11-methyl-18,18-dioxo-8-oxa-18λ6-thia-11,19-diazatetracyclo[18.3.1.113,17.02,7]pentacosa-1(24),2,4,6,13(25),14,16,20,22-nonaen-10-one (92%, 35 mg, 0.066 mmol), anhydrous DMF (1 mL) and iodocyclohexane (43 μL, 0.332 mmol) were added to pressure vial, the vial was sealed and heated at 120° C. for 4 hours. The mixture was allowed to cool, diluted with EtOAc (40 mL) and washed with sat. aq. Na₂S₂O₃ (30 mL), water (30 mL), 1 M aq. HCl (30 mL) and brine (30 mL). The organic layer was passed through a phase separator and then concentrated. The residue was purified by preparative HPLC (Method P1) to afford the title compound (8.0 mg, 25% Yield, 97% purity) as an off-white solid.

¹H NMR (400 MHz, DMSO) δ 7.70-7.60 (m, 1H), 7.56-7.50 (m, 1H), 7.42-7.34 (m, 1H), 7.26-7.21 (m, 1H), 7.21-7.00 (m, 4H), 6.96-6.83 (m, 1H), 4.70 (s, 2H), 4.65-4.01 (m, 2H), 2.36 (s, 3H).

LCMS: m/z=475.1/477.1 [M−H]−, (ESI−), RT=3.21, Method A

### Example 94—Synthesis of Intermediate 1: Methyl 3-chloro-5-chlorosulfonyl-4-methoxy-benzoate

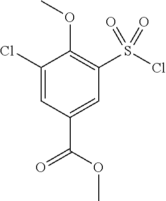 [SMILES: COC(=O)c1cc(Cl)c(OC)c(S(=O)(=O)Cl)c1]

### Step 1

To a solution of methyl 3-bromo-5-chloro-4-methoxy-benzoate (95%, 10.0 g, 34.0 mmol) in anhydrous 1,4-dioxane (200 mL) was added DIPEA (5.50 g, 42.6 mmol). The solution was bubbled with nitrogen for 5 mins, before phenylmethanethiol (5.00 g, 40.3 mmol), Pd₂(dba)₃ (1.00 g, 1.09 mmol) and Xantphos (1.20 g, 2.07 mmol) were added. The reaction mixture was heated at 100° C. for 18 hours. The reaction mixture was allowed to cool to r.t., before it was added to water (100 mL) and extracted with EtOAc (3×100 mL). The combined organic layers were washed with brine (100 mL) and dried over Na₂SO₄, filtered and concentrated. The residue was purified by FCC (350 g SiO₂ column, ethyl acetate in heptane, 0-60% EtOAc in heptane) to afford methyl 3-benzylsulfanyl-5-chloro-4-methoxy-benzoate (11.90 g, 98% yield, 90% purity) as a yellow solid.

¹H NMR (500 MHz, DMSO) δ 7.81 (d, J=2.0 Hz, 1H), 7.76 (d, J=2.0 Hz, 1H), 7.44-7.39 (m, 2H), 7.36-7.31 (m, 2H), 7.28-7.23 (m, 1H), 4.31 (s, 2H), 3.84 (s, 3H), 3.82 (s, 3H).

### Step 2

Methyl 3-benzylsulfanyl-5-chloro-4-methoxy-benzoate (95%, 11.00 g, 32.4 mmol) was suspended in a mixture of acetonitrile (200 mL), acetic acid (10 mL) and water (7 mL) and the resulting mixture was cooled to 0° C. in an ice bath. 1,3-Dichloro-5,5-dimethylhydantoin (13.00 g, 66.0 mmol) was then added portion wise (over approx. 15 mins) and the reaction mixture was stirred for 2 hours at 0° C. MeCN was removed under reduced pressure and DCM (20 mL) was added. The solution was cooled to 0° C., and aq. 5% NaHCO₃ (20 mL) solution was slowly added. The organic layer was separated, dried over Na₂SO₄, filtered and concentrated under reduced pressure to give methyl 3-chloro-5-chlorosulfonyl-4-methoxy-benzoate (50.0%) (17.70 g, 29.6 mmol, 91% Yield) as a clear oil.

LCMS: m/z=278.9 [M−H]−, (ESI−), RT=1.02 min, Method B (ionises as the sulfonic acid)

### Example 95—Synthesis of Intermediate 2: tert-Butyl N-[2-(2-bromophenoxy)ethyl]carbamate

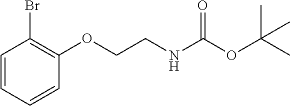 [SMILES: CC(C)(C)OC(=O)NCCOc1ccccc1Br]

2-bromophenol (0.41 mL, 3.57 mmol) and tert-butyl (2-hydroxyethyl)carbamate (0.48 mL, 3.10 mmol) were placed in anhydrous THE (15 mL) with triphenylphosphane (1399 mg, 5.33 mmol), and the mixture was cooled to 0° C. DIAD (1.1 mL, 5.33 mmol) was added portion-wise over 30 minutes. The mixture was warmed to r.t, and stirred for 56 h. Further tert-butyl (2-hydroxyethyl)carbamate (0.14 mL, 0.931 mmol), DIAD (0.61 mL, 3.10 mmol) and triphenylphosphane (814 mg, 3.10 mmol) were added and the mixture was stirred for 4 h. The mixture was diluted with water (100 mL), extracted with Et₂O (2×30 mL), washed with brine, dried over MgSO₄ and concentrated under vacuum. The residue was dissolved in EtOH (27.5 mL) and zinc dichloride (682 mg, 5.00 mmol) was added and stirred for 1 h. The solution was filtered and washed with EtOH, the filtrate was concentrated under vacuum and dissolved in EtOH (11 mL) and filtered again. The residue was purified by FCC (350 g SiO₂ column, 5-100% EtOAc in heptane) to afford the title compound (772 mg, 61% yield, 80% purity) as a yellow oil.

¹H NMR (400 MHz, DMSO) δ 7.56 (dd, J=7.9, 1.6 Hz, 1H), 7.35-7.30 (m, 1H), 7.13-7.10 (m, 1H), 6.95-6.91 (m, 1H), 6.91-6.86 (m, 1H), 4.03 (t, J=6.0 Hz, 2H), 3.34-3.32 (m, 2H), 1.38 (s, 9H).

LCMS: m/z=217.8/219.8 [M-Boc+H]+, (ESI+), RT=1.01 min, Method B

### Example 96—Synthesis of Intermediate 3: tert-Butyl N-[2-[2-(5-amino-2,4-difluoro-phenyl)phenoxy]ethyl]carbamate

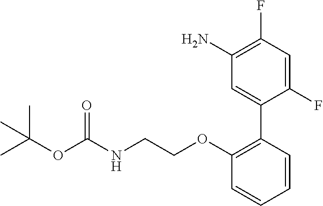 [SMILES: CC(C)(C)OC(=O)NCCOc1ccccc1-c1cc(N)c(F)cc1F]

Intermediate 2 (80%, 840 mg, 2.13 mmol) and 2,4-difluoro-5-(4,4,5,5-tetramethyl-1,3,2-dioxaborolan-2-yl)aniline (80%, 813 mg, 2.55 mmol) were dissolved in 1,4-dioxane (10.2 mL) and stirred at r.t. To this was added potassium carbonate (0.68 g, 4.90 mmol) and the mixture was degassed with nitrogen for 5 mins. Pd(dppf)Cl₂ (90 mg, 0.123 mmol) was added to the reaction mixture and degassed again for another 5 mins. The reaction mixture was heated to 100° C., and stirred under reflux for 3 h. Further 2,4-difluoro-5-(4,4,5,5-tetramethyl-1,3,2-dioxaborolan-2-yl)aniline (80%, 60 mg, 0.18 mmol), Pd(dppf)Cl₂ (77 mg, 0.106 mmol) and potassium carbonate (0.35 g, 2.27 mmol) were then added and the mixture degassed for 5 mins. The reaction mixture was then stirred for 18 h at 100° C. The reaction mixture was cooled to r.t., filtered through celite and washed with EtOAc and water. Water (15 mL) was added to the filtrate and extracted with EtOAc (3×15 mL). The organic layers were combined, washed with brine, dried over MgSO₄, filtered and concentrated under vacuum. The residue was purified by FCC (100 g SiO₂ column, 5-100% EtOAc in heptane) to afford the title compound (0.65 g, 76% yield, 90% purity) as a clear oil.

¹H NMR (400 MHz, DMSO) δ 7.37-7.32 (m, 1H), 7.20-7.15 (m, 1H), 7.12-7.07 (m, 1H), 7.04-6.96 (m, 2H), 6.75-6.68 (m, 2H), 4.95 (s, 2H), 3.94 (t, J=6.4 Hz, 2H), 3.18 (q, J=6.3 Hz, 2H), 1.36 (s, 9H).

LCMS: m/z=264.9 [M-Boc+H]+, (ESI+), RT=0.99 min, Method B

### Example 97—Synthesis of Intermediate 4: Methyl 3-[[5-[2-[2-(tert-butoxycarbonylamino)ethoxy]phenyl]-2,4-difluoro-phenyl]sulfamoyl]-5-chloro-4-methoxy-benzoate

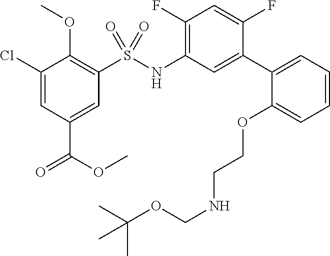 [SMILES: COC(=O)c1cc(Cl)c(OC)c(S(=O)(=O)Nc2cc(-c3ccccc3OCCNCOC(C)(C)C)c(F)cc2F)c1]

Intermediate 1 (70%, 686 mg, 1.61 mmol) and Intermediate 3 (90%, 650 mg, 1.61 mmol) were dissolved in Pyridine-Anhydrous (9 mL) and heated to 50° C. for 2.5 h. Further Intermediate 1 (70%, 69 mg, 0.161 mmol) was added and the mixture was stirred at 50° C. for 2 hours. The reaction mixture was allowed to cool to r.t. The mixture was then diluted with 1M aq. HCl (14 mL) and extracted with EtOAc (3×15 mL) and the combined organic extracts were washed with brine (20 mL), dried over MgSO₄, filtered and concentrated under reduced pressure. The residue was purified by FCC (100 g SiO₂ column, 0-30% EtOAc in heptane) to afford the title compound as an off white solid (595 mg, 53% yield, 90% purity).

¹H NMR (500 MHz, DMSO) δ 10.35 (s, 1H), 8.25 (d, J=1.6 Hz, 1H), 8.17 (d, J=2.1 Hz, 1H), 7.42-7.36 (m, 1H), 7.27 (t, J=10.2 Hz, 1H), 7.19 (t, J=8.2 Hz, 1H), 7.14-7.08 (m, 2H), 7.02 (t, J=7.4 Hz, 1H), 6.72 (t, J=5.6 Hz, 1H), 3.99-3.91 (m, 5H), 3.86 (s, 3H), 3.16 (q, J=6.2 Hz, 2H), 1.34 (s, 9H).

LCMS: m/z=625.1 [M−H]−, (ESI−), RT=1.13, Method B

### Example 98—Synthesis of Intermediate 5: 3-[[5-[2-[2-(tert-butoxycarbonylamino)ethoxy]phenyl]-2,4-difluoro-phenyl]sulfamoyl]-5-chloro-4-methoxy-benzoic acid

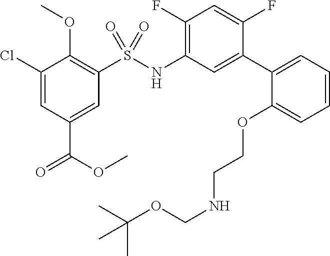 [SMILES: COC(=O)c1cc(Cl)c(OC)c(S(=O)(=O)Nc2cc(-c3ccccc3OCCNCOC(C)(C)C)c(F)cc2F)c1]

Intermediate 4 (595 mg, 0.949 mmol) was dissolved in THE (10 mL) and 1M aqueous sodium hydroxide (0.47 mL, 0.474 mmol) was added. The reaction mixture was stirred for 4 hours at r.t. Further 1M aqueous sodium hydroxide (0.47 mL, 0.474 mmol) was added and the mixture was stirred for 2 h at r.t. Further 1M aqueous sodium hydroxide (0.47 mL, 0.474 mmol) was added and the mixture was stirred at r.t. overnight (approximately 16 hours). THF was removed under a steady stream of nitrogen and the aqueous layer was acidified to pH 1 with 2M aq. HCl resulting in formation of a precipitate. This was filtered, washed with 2M aq. HCl (10 mL) and dried in the vacuum oven to afford the title compound as a white solid (495 mg, 79% yield, 93% purity).

¹H NMR (400 MHz, DMSO) δ 10.32 (s, 1H), 8.21 (d, J=2.1 Hz, 1H), 8.17 (d, J=2.1 Hz, 1H), 7.42-7.36 (m, 1H), 7.27 (t, J=9.9 Hz, 1H), 7.21-7.15 (m, 1H), 7.12 (d, J=8.3 Hz, 1H), 7.12-7.05 (m, 1H), 7.04-6.99 (m, 1H), 6.70 (t, J=6.0 Hz, 1H), 3.97-3.93 (m, 5H), 3.16 (q, J=6.5 Hz, 2H), 1.34 (s, 9H).

LCMS: m/z=611.0 [M−H]−, (ESI−), RT=1.01, Method B

### Example 99—Synthesis of Intermediate 6: 3-[[5-[2-(2-aminoethoxy)phenyl]-2,4-difluoro-phenyl]sulfamoyl]-5-chloro-4-methoxy-benzoic acid

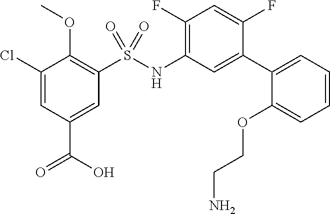 [SMILES: COc1c(Cl)cc(C(=O)O)cc1S(=O)(=O)Nc1cc(-c2ccccc2OCCN)c(F)cc1F]

Intermediate 5 (93%, 495 mg, 0.751 mmol) was suspended in anhydrous 1,4-dioxane (4 mL) and 4 M hydrogen chloride in dioxane (5.1 mL, 20.4 mmol) was added and the mixture was stirred at r.t. for 2.5 h. The reaction mixture was concentrated under reduced pressure, the residue was dissolved in MeOH and purified using an SCX cartridge (5 g); eluting first with MeOH (2 CV), followed by 7M ammonia in MeOH (2 CV). The ammonia fractions were combined and concentrated to afford the title compound (333 mg, 78% yield, 90% purity) as an off white solid.

¹H NMR (500 MHz, DMSO) δ 8.01 (d, J=2.0 Hz, 1H), 7.93 (d, J=1.9 Hz, 1H), 7.42-7.35 (m, 1H), 7.23-7.18 (m, 3H), 7.14 (d, J=7.8 Hz, 1H), 7.05 (td, J=7.5, 0.9 Hz, 1H), 4.12 (t, J=5.5 Hz, 2H), 3.97 (s, 3H), 3.11 (t, J=5.5 Hz, 2H). 4H not observed.

LCMS: m/z=513.0 [M+H]+, (ESI+), RT=0.71, Method B

### Example 100—Synthesis of Intermediate 7: 2-(2-bromophenoxy)ethoxy-tert-butyl-dimethyl-silane

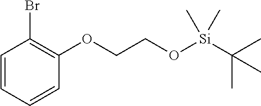 [SMILES: CC(C)(C)[Si](C)(C)OCCOc1ccccc1Br]

To a solution of 2-bromophenol (1.00 g, 5.78 mmol), 2-{[tert-butyl(dimethyl)silyl]oxy}ethanol (1.20 g, 6.81 mmol) and triphenylphosphine (1.80 g, 6.86 mmol) in anhydrous THE (40 mL) was added DIAD (1.40 g, 6.92 mmol) and the reaction was stirred at 40° C. for 3 h. The reaction was cooled to RT and then copper(I) chloride (600 mg, 6.00 mmol) was added and the reaction was stirred for a further 5 mins at r.t. The mixture was passed through a pad of celite which was washed with EtOAc and the filtrate was concentrated under reduced pressure. The residue was purified by FCC (100 g SiO₂ column, 0-50% ethyl acetate in heptane) to afford the title compound (1.91 g, 100% yield) as a colorless oil which solidified upon standing to provide a white solid.

¹H NMR (500 MHz, CDCl3) δ 7.53 (dd, J=7.9, 1.6 Hz, 1H), 7.26-7.22 (m, 1H), 6.93 (dd, J=8.2, 1.2 Hz, 1H), 6.82 (td, J=7.7, 1.3 Hz, 1H), 4.11 (t, J=5.2 Hz, 2H), 4.02 (t, J=5.2 Hz, 2H), 0.91 (s, 9H), 0.12 (s, 6H).

LCMS: m/z=no clear mass ion (ESI+), RT=1.34, Method B

### Example 101—Synthesis of Intermediate 8: 5-[2-[2-[tert-butyl(dimethyl)silyl]oxyethoxy]phenyl]-2,4-difluoro-aniline

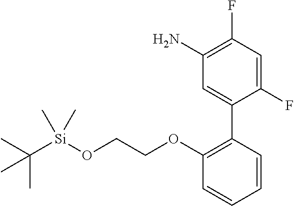 [SMILES: CC(C)(C)[Si](C)(C)OCCOc1ccccc1-c1cc(N)c(F)cc1F]

To a solution of Intermediate 7 (700 mg, 2.11 mmol) and 2,4-difluoro-5-(4,4,5,5-tetramethyl-1,3,2-dioxaborolan-2-yl)aniline (99%, 650 mg, 2.52 mmol) in 1,4-dioxane (10 mL) was added potassium carbonate (600 mg, 4.34 mmol) and the mixture was degassed with N₂ for 10 mins. Pd(dppf)Cl₂ (160 mg, 0.218 mmol) was added the reaction mixture was degassed again for another 5 mins, before the vessel was sealed and the reaction mixture was heated to 100° C., and stirred under reflux for 18 h. The reaction mixture was cooled to room temperature, filtered through celite and washed with EtOAc (30 mL) and water (50 mL). The biphasic filtrate was separated and the aqueous phase was extracted with EtOAc (2×30 mL). The organic layers were combined, washed with brine (50 mL), dried over Na₂SO₄, filtered and concentrated under reduced pressure. The residue was purified by FCC (100 g SiO₂ column, 0-50% ethyl acetate in heptane) to afford the title compound (635 mg, 71% yield, 90% purity) as a pale yellow oil.

¹H NMR (500 MHz, CDCl3) δ 7.32 (td, J=8.0, 1.7 Hz, 1H), 7.21 (dd, J=7.7, 1.3 Hz, 1H), 7.02-6.97 (m, 2H), 6.81 (dd, J=10.8, 9.3 Hz, 1H), 6.75 (dd, J=9.9, 7.1 Hz, 1H), 4.04 (t, J=5.3 Hz, 2H), 3.87 (t, J=5.3 Hz, 2H), 0.85 (s, 9H), −0.02 (s, 6H). 2H not observed.

LCMS: m/z=380.1 [M+H]+, (ESI−), RT=1.20, Method B

### Example 102—Synthesis of Intermediate 9: Methyl 3-[[5-[2-[2-[tert-butyl(dimethyl)silyl]oxyethoxy]phenyl]-2,4-difluoro-phenyl]sulfamoyl]-5-chloro-4-methoxy-benzoate

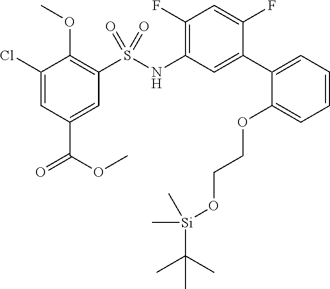 [SMILES: COC(=O)c1cc(Cl)c(OC)c(S(=O)(=O)Nc2cc(-c3ccccc3OCCO[Si](C)(C)C(C)(C)C)c(F)cc2F)c1]

Intermediate 1 (50%, 600 mg, 1.00 mmol) and Intermediate 8 (90%, 350 mg, 0.830 mmol) were dissolved in anhydrous pyridine (6 mL) and the mixture was heated to 50° C., and stirred for 1.5 h. Further Intermediate 1 (50%, 300 mg, 0.501 mmol) was added and the reaction was stirred at 50° C. for 1 h. The reaction mixture was allowed to cool to r.t, and was diluted with 1M aqueous HCl (30 mL) and extracted with EtOAc (3×30 mL). The combined organic extracts were washed with brine (50 mL), dried over Na₂SO₄, filtered and concentrated under reduced pressure. The residue was purified by FCC (50 g SiO₂ column, 0-100% ethyl acetate in heptane) to afford the title compound (560 mg, 83% yield, 80% purity) as a viscous yellow oil.

¹H NMR (400 MHz, CDCl3) δ 8.32 (d, J=2.1 Hz, 1H), 8.27 (d, J=2.1 Hz, 1H), 7.52 (dd, J=8.8, 7.4 Hz, 1H), 7.38-7.31 (m, 1H), 7.17-7.10 (m, 2H), 7.04-6.97 (m, 2H), 6.75 (dd, J=10.0, 9.1 Hz, 1H), 4.19 (s, 3H), 4.03 (t, J=5.3 Hz, 2H), 3.90 (s, 3H), 3.84 (t, J=5.3 Hz, 2H), 0.81 (s, 9H), −0.08 (s, 6H).

LCMS: m/z=640.2 [M−H]−, (ESI−), RT=1.28, Method B

### Example 103—Synthesis of Intermediate 10: 3-[[5-[2-[2-[tert-butyl(dimethyl)silyl]oxyethoxy]phenyl]-2,4-difluoro-phenyl]sulfamoyl]-5-chloro-4-methoxy-benzoic acid

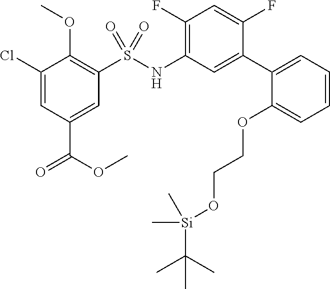 [SMILES: COC(=O)c1cc(Cl)c(OC)c(S(=O)(=O)Nc2cc(-c3ccccc3OCCO[Si](C)(C)C(C)(C)C)c(F)cc2F)c1]

Intermediate 9 (80%, 560 mg, 0.698 mmol) was dissolved in THE (7 mL) and 2M aqueous sodium hydroxide (1.2 mL, 2.40 mmol) was added. The reaction mixture was stirred at r.t. for 4 hours. The THF was removed under vacuum and the remaining aqueous solution was acidified with 1M aqueous HCl. DCM (30 mL) was added and the layers were separated. The aqueous phase was extracted with additional DCM (2×30 mL) and the combined organic phases were washed with brine (50 mL), dried over Na₂SO₄ and concentrated under reduced pressure. The residue was purified by FCC (25 g SiO₂ column, 0-60% EtOAc in heptane) to afford the title compound (428 mg, 97% yield, 99% purity) as a viscous orange oil.

¹H NMR (400 MHz, DMSO) δ 13.64 (broad s, 1H), 10.35 (broad s, 1H), 8.19 (d, J=2.0 Hz, 1H), 8.17 (d, J=2.1 Hz, 1H), 7.37 (td, J=7.9, 7.4, 1.8 Hz, 1H), 7.26 (t, J=10.0 Hz, 1H), 7.21-7.15 (m, 1H), 7.13-7.06 (m, 2H), 7.04-6.97 (m, 1H), 4.01-3.97 (m, 2H), 3.96 (s, 3H), 3.75 (t, J=4.7 Hz, 2H), 0.73 (s, 9H), −0.18 (s, 6H).

LCMS: m/z=626.1 [M−H]−, (ESI−), RT=1.17, Method B

### Example 104—Synthesis of Intermediate 11: 3-chloro-5-[[2,4-difluoro-5-[2-(2-hydroxyethoxy)phenyl]phenyl]sulfamoyl]-4-methoxy-benzoic acid

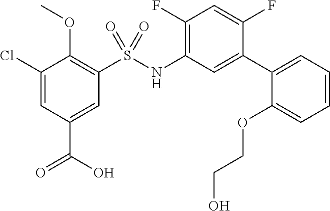 [SMILES: COc1c(Cl)cc(C(=O)O)cc1S(=O)(=O)Nc1cc(-c2ccccc2OCCO)c(F)cc1F]

Intermediate 10 (425 mg, 0.670 mmol) was dissolved in anhydrous THF (9 mL) and to the stirring solution was added 1 M TBAF (1M solution in THF) (0.80 mL, 0.800 mmol) dropwise. The mixture was stirred at r.t. for 3 h, before additional 1 M TBAF (1M solution in THF) (0.40 mL, 0.400 mmol) was added and the stirring solution left for an additional 15 h. The THF was removed under reduced pressure and the residue taken into a mixture of DCM (30 mL) and water (30 mL). The mixture was separated and the aqueous phase was extracted again with DCM (20 mL). The combined organics were washed with brine (30 mL), dried over Na₂SO₄ and concentrated under vacuum to afford the title compound (429 mg, 100% yield, 80% purity) as a viscous colorless oil.

¹H NMR (500 MHz, DMSO) δ 8.16 (d, J=1.6 Hz, 1H), 8.10 (s, 1H), 7.40-7.32 (m, 1H), 7.21 (s, 1H), 7.18-7.10 (m, 2H), 7.05 (d, J=6.7 Hz, 1H), 6.98 (t, J=7.4 Hz, 1H), 3.95 (t, J=5.5 Hz, 2H), 3.90 (s, 3H), 3.56 (t, J=5.5 Hz, 2H). 3H not observed

LCMS: m/z=512.0 [M−H]−, (ESI−), RT=0.82, Method B

### Example 105—Synthesis of Intermediate 12: 15-chloro-21,23-difluoro-16-methoxy-18,18-dioxo-8,11-dioxa-18λ6-thia-19-azatetracyclo[18.3.1.113,17.02,7]pentacosa-1(23),2(7),3,5,13(25),14,16,20(24),21-nonaen-12-one

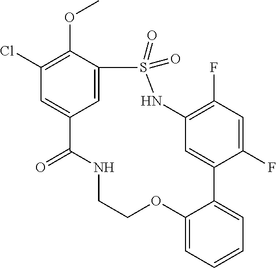 [SMILES: COc1c(Cl)cc2cc1S(=O)(=O)Nc1cc(c(F)cc1F)-c1ccccc1OCCNC2=O]

To a solution of Intermediate 11 (80%, 420 mg, 0.654 mmol) in anhydrous THE (50 mL) was added 2,4,6-trichlorobenzoyl chloride (200 mg, 0.820 mmol) and DIPEA (0.30 mL, 1.72 mmol) and the mixture was stirred at r.t. for 1 h. 4-Dimethylaminopyridine (10 mg, 0.0819 mmol) was then added and the mixture stirred for an additional 3 h at 65° C. The solvent volume was reduced under vacuum and the residue was taken into a mixture of 1M aqueous HCl (50 mL) and EtOAc (50 mL). The layers were separated and the aqueous phase extracted with additional EtOAc (2×50 mL). The combined organic fractions were washed with brine (100 mL), dried over Na₂SO₄ and concentrated under reduced pressure. The residue was purified by FCC (10 g SiO₂ column, 0-20% MeOH in DCM) followed by preparative HPLC (Method P1) to afford the title compound (61 mg, 18% yield, 98% purity) as a white solid.

¹H NMR (500 MHz, DMSO) δ 10.59 (s, 1H), 8.18 (s, 1H), 7.56 (broad s, 1H), 7.49-7.39 (m, 1H), 7.35-7.27 (m, 2H), 7.21-7.13 (m, 2H), 7.09 (t, J=7.4 Hz, 1H), 4.42-4.36 (m, 2H), 4.32-4.26 (m, 2H), 4.03 (s, 3H).

LCMS: m/z=494.0 [M−H]−, (ESI−), RT=1.01, Method B

### Example 106—Synthesis of Intermediate 13: Methyl 3-chloro-5-[[2,4-difluoro-5-[2-(hydroxymethyl)phenyl]phenyl]sulfamoyl]-4-methoxy-benzoate

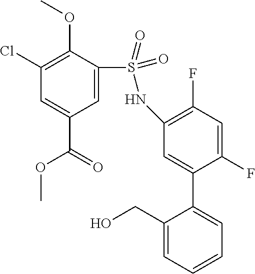 [SMILES: COC(=O)c1cc(Cl)c(OC)c(S(=O)(=O)Nc2cc(-c3ccccc3CO)c(F)cc2F)c1]

To a solution of [2-(5-amino-2,4-difluoro-phenyl)phenyl]methanol (synthesized using a similar method to Intermediate 8, 88% purity, 268 mg, 1.00 mmol) in anhydrous pyridine (5 mL) was added Intermediate 1 (50% purity, 720 mg, 1.20 mmol). The mixture was stirred at 50° C. in a pressure tube for 1 hour, then 1 M aq. HCl (10 mL) was added and the mixture was extracted with EtOAc (3×15 mL). The combined organic layers were washed with brine (10 mL), dried over MgSO₄ and concentrated under reduced pressure. The residue was purified by FCC (10 g SiO₂ cartridge, 0-100% EtOAc in heptanes) to afford the title compound (356 mg, 44% Yield, 61% purity) as a yellow gum.

¹H NMR (500 MHz, DMSO) δ 10.40 (s, 1H), 8.27-8.22 (m, 1H), 8.20-8.10 (m, 1H), 7.61-7.52 (m, 1H), 7.48-7.26 (m, 5H), 5.06 (s, 1H), 4.20 (d, J=5.4 Hz, 2H), 3.96 (s, 3H), 3.87 (s, 3H).

LCMS: m/z=496.2/498.2 [M−H]−, (ESI−), RT=0.99, Method B

### Example 107—Synthesis of Intermediate 14: 3-Chloro-5-[[2,4-difluoro-5-[2-(hydroxymethyl)phenyl]phenyl]sulfamoyl]-4-methoxy-benzoic acid

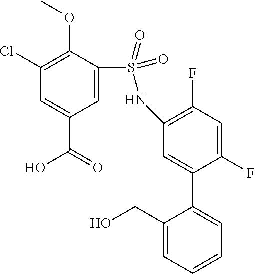 [SMILES: COc1c(Cl)cc(C(=O)O)cc1S(=O)(=O)Nc1cc(-c2ccccc2CO)c(F)cc1F]

2 M aq. sodium hydroxide (1.1 mL, 2.18 mmol) was added to a solution of Intermediate 13 (61% purity, 356 mg, 0.436 mmol) in THE (5 mL) and the mixture was stirred at r.t. for 3 hours. The mixture was diluted with water (20 mL), washed with EtOAc (25 mL) and the organic layer was discarded. The aqueous layer was acidified to approximately pH 6 by the addition of 1 M aq. HCl. The mixture was extracted with EtOAc (3×25 mL) and the combined organic layers were dried over MgSO₄ and concentrated under reduced pressure to afford the title compound (248 mg, quantitative yield, 85% purity) as a yellow foam.

¹H NMR (500 MHz, DMSO) δ 8.20-8.14 (m, 2H), 7.63-7.51 (m, 1H), 7.48-7.39 (m, 1H), 7.38-7.24 (m, 2H), 7.14-7.08 (m, 1H), 7.08-7.02 (m, 1H), 4.21 (s, 2H), 3.93 (s, 3H).

LCMS: m/z=482.2/484.2 [M−H]−, (ESI−), RT=0.86, Method B

### Example 108—Synthesis of Intermediate 15: 13-Chloro-19,21-difluoro-14-methoxy-16,16-dioxo-9-oxa-16λ6-thia-17-azatetracyclo[16.3.1.111,15.02,7]tricosa-1(21),2(7),3,5,11(23),12,14,18(22),19-nonaen-10-one

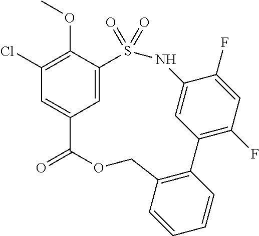 [SMILES: COc1c(Cl)cc2cc1S(=O)(=O)Nc1cc(c(F)cc1F)-c1ccccc1COC2=O]

DMAP (11 mg, 0.0871 mmol) was added to a solution of Intermediate 14 (85% purity, 248 mg, 0.436 mmol) and DCC (198 mg, 0.958 mmol) in DCM (20 mL). The mixture was stirred at r.t. for 4.5 hours. Further DCC (45 mg, 0.218 mmol) and DMAP (2.7 mg, 0.0218 mmol) were added and the mixture was stirred at r.t. for 22 hours. The mixture was washed with water (15 mL) and separated using a phase separator. The aqueous layer was further extracted with DCM (2×mL), each time separating the layers with a phase separator. The combined organic layers were then concentrated under reduced pressure. The residue was purified by FCC (25 g SiO₂ cartridge, 0-100% DCM in heptanes) to afford the title compound (88 mg, 43% Yield) as a white solid.

¹H NMR (400 MHz, DMSO) δ 10.30 (s, 1H), 8.11-8.02 (m, 1H), 7.63-7.53 (m, 1H), 7.51-7.45 (m, 2H), 7.46-7.33 (m, 2H), 7.33-7.26 (m, 1H), 7.03-6.92 (m, 1H), 5.42 (d, J=12.7 Hz, 1H), 5.18 (d, J=12.6 Hz, 1H), 3.98 (s, 3H).

LCMS: m/z=464.2/466.3 [M−H]−, (ESI−), RT=1.07, Method B

### Example 109—Synthesis of Intermediate 16: tert-Butyl 4-[5-[(3-chloro-2-methoxy-5-methoxycarbonyl-phenyl)sulfonylamino]-2,4-difluoro-phenyl]isoindoline-2-carboxylate

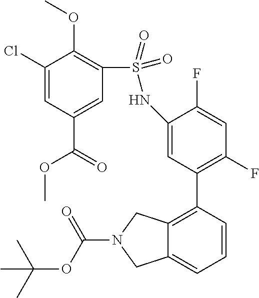 [SMILES: COC(=O)c1cc(Cl)c(OC)c(S(=O)(=O)Nc2cc(-c3cccc4c3CN(C(=O)OC(C)(C)C)C4)c(F)cc2F)c1]

Intermediate 1 (50%, 700 mg, 1.17 mmol) and tert-butyl 4-(5-amino-2,4-difluoro-phenyl)isoindoline-2-carboxylate (synthesized using a similar method to Intermediate 3, 99% purity, 300 mg, 0.857 mmol) were dissolved in pyridine (7.875 mL) and the mixture was heated to 50° C., and stirred for 1 hour and 20 minutes. The mixture was allowed to cool to r.t, and was diluted with 1 M aq. HCl (80 mL) and extracted with EtOAc (3×30 mL). The combined organic extracts were washed with brine (50 mL), dried over Na₂SO₄, filtered and concentrated in vacuo. The residue was purified by FCC (25 g SiO₂ cartridge, 0-30% EtOAc in heptane) to afford the title compound as an off-white solid (168 mg, 26% Yield, 81% purity).

¹H NMR (500 MHz, DMSO) δ 10.48 (s, 1H), 8.26-8.20 (m, 1H), 8.20-8.14 (m, 1H), 7.48-7.10 (m, 5H), 4.66 (d, J=5.6 Hz, 2H), 4.45 (d, 1H), 4.32 (s, 1H), 3.94 (d, J=4.5 Hz, 3H), 3.85 (d, J=5.5 Hz, 3H), 1.43 (d, J=19.7 Hz, 9H).

LCMS: m/z=607.2/609.2 [M−H]−, (ESI−), RT=1.17, Method B

### Example 110—Synthesis of Intermediate 17-3-[[5-(2-tert-butoxycarbonylisoindolin-4-yl)-2,4-difluoro-phenyl]sulfamoyl]-5-chloro-4-methoxy-benzoic acid

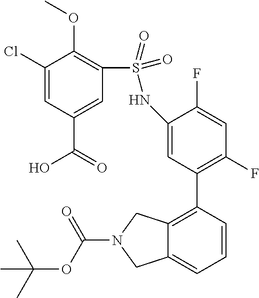 [SMILES: COc1c(Cl)cc(C(=O)O)cc1S(=O)(=O)Nc1cc(-c2cccc3c2CN(C(=O)OC(C)(C)C)C3)c(F)cc1F]

Two batches of Intermediate 16 (166 mg, 81% purity, 0.22 mmol and 135 mg, 93% purity, 0.201 mmol) were dissolved in THE (4 mL) and 2 M aq. sodium hydroxide (3.33 mL, 6.66 mmol) was added. The mixture was stirred at r.t. for 26 hours. THF was removed under vacuum and the residue was taken up in 1 M aq. HCl (20 mL) and DCM (20 mL) and layers were separated. The aqueous layer was extracted with further DCM (2×10 mL). The combined organic phases were passed through a phase separator and concentrated to give the title compound (250 mg, 91% Yield, 92% purity) as pale-orange solid.

¹H NMR (500 MHz, DMSO) δ 11.96 (s, 1H), 8.21-8.17 (m, 1H), 8.15 (s, 1H), 7.43-7.06 (m, 5H), 4.65 (d, J=8.6 Hz, 2H), 4.44 (s, 1H), 4.35 (s, 1H), 3.91 (s, 3H), 1.47-1.37 (m, 9H). NH not observed

LCMS: m/z=593.2/595.2 [M−H]−, (ESI−), RT=1.04, Method B

### Example 111—Synthesis of Intermediate 18: 3-Chloro-5-[(2,4-difluoro-5-isoindolin-4-yl-phenyl)sulfamoyl]-4-methoxy-benzoic acid hydrochloride

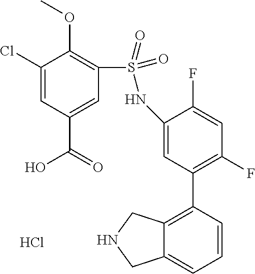 [SMILES: COc1c(Cl)cc(C(=O)O)cc1S(=O)(=O)Nc1cc(-c2cccc3c2CNC3)c(F)cc1F ; Cl]

Intermediate 17 (91% purity, 248 mg, 0.379 mmol) was dissolved in 4 M HCl in dioxane (4.0 mL, 16.0 mmol) and the mixture was stirred at r.t. for 1 hour. The reaction mixture was concentrated under reduced pressure. The residue was taken up in DCM (20 mL), with sonication, and concentrated. This was repeated three times to afford the title compound (232 mg, quantitative yield, 90% purity) as a purple solid.

¹H NMR (500 MHz, DMSO) δ 10.52 (s, 1H), 9.59 (br. s, 2H), 8.23 (d, J=2.1 Hz, 1H), 8.17 (d, J=2.1 Hz, 1H), 7.54-7.43 (m, 3H), 7.36-7.25 (m, 2H), 4.59 (s, 2H), 4.38 (s, 2H), 3.93 (s, 3H).

LCMS: m/z=493.1/495.1 [M−H]−, (ESI−), RT=0.64, Method B

### Example 112—Synthesis of Intermediate 19: 13-Chloro-19,21-difluoro-14-methoxy-16,16-dioxo-16λ6-thia-9,17-diazapentacyclo[16.3.1.16,9.111,15.02,7]tetracosa-1(21),2(7),3,5,11,13,15(23),18(22),19-nonaen-10-one

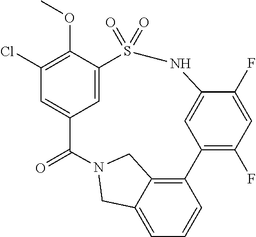 [SMILES: COc1c(Cl)cc2cc1S(=O)(=O)Nc1cc(c(F)cc1F)-c1cccc3c1CN(C3)C2=O]

To a solution of T3P (50% in EtOAc) (0.60 mL, 1.01 mmol) and DIPEA (0.32 mL, 1.84 mmol) in anhydrous DMF (10 mL) was added dropwise a solution of Intermediate 18 (90% purity, 208 mg, 0.352 mmol) in anhydrous DMF (10 mL). The mixture was stirred at r.t. for 10 minutes. The mixture was immediately diluted with water (40 mL) and then extracted with EtOAc (3×30 mL). The combined organic phases were washed with brine (50 mL) then dried over MgSO₄, filtered, and concentrated. The residue was purified by FCC (25 g SiO₂ cartridge, 0-100% DCM in heptane, then EtOAc, 0-100% in DCM) to afford the title compound (112 mg, 55% Yield, 83% purity) as a white solid.

¹H NMR (500 MHz, DMSO) δ 11.28 (s, 1H), 7.98-7.93 (m, 1H), 7.89 (s, 1H), 7.51-7.28 (m, 4H), 7.17-7.09 (m, 1H), 4.02 (s, 3H), 3.80 (s, 2H).

LCMS: m/z=475.0/477.1 [M−H]−, (ESI−), RT=0.92, Method B

### Example 113—Synthesis of Intermediate 20: 3-Chloro-5-(hydroxymethyl)-2-methoxy-benzenesulfonyl chloride

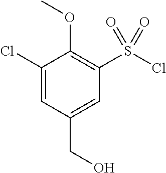

### Step 1

To a solution of methyl 3-benzylsulfanyl-5-chloro-4-methoxy-benzoate (2.0 g, 6.2 mmol) in THE (30 mL) and MeOH (5 mL) was added lithium borohydride (675 mg, 31.0 mmol) and the mixture was stirred at r.t. for 4 hours. The mixture was cautiously quenched by slow addition of water (20 mL) and then concentrated to remove THE and MeOH. The aqueous residue was extracted with EtOAc (3×50 mL), and the combined organic phases were washed with brine, dried over Na₂SO₄, filtered, and concentrated in vacuo to afford (3-benzylsulfanyl-5-chloro-4-methoxy-phenyl)methanol (1.90 g, 99% Yield, 95% purity) as a green oil.

¹H NMR (500 MHz, DMSO) δ 7.42-7.38 (m, 2H), 7.34-7.30 (m, 2H), 7.28-7.23 (m, 2H), 7.21-7.19 (m, 1H), 5.30 (t, J=5.8 Hz, 1H), 4.42 (d, J=5.5 Hz, 2H), 4.22 (s, 2H), 3.72 (s, 3H).

LCMS: m/z=293.2 [M−H]−, (ESI−), RT=0.98, Method B

### Step 2

(3-Benzylsulfanyl-5-chloro-4-methoxy-phenyl)methanol (95% purity, 1.90 g, 6.12 mmol) was added to a mixture of MeCN (35 mL), acetic acid (1.8 mL) and water (1.2 mL). The resulting suspension was cooled to 0° C. in an ice bath. 1,3-Dichloro-5,5-dimethylhydantoin (2.45 g, 12.4 mmol) was then added portion wise over approximately 15 mins) and the reaction mixture was stirred for 2 hours at 0° C. MeCN was removed under reduced pressure and DCM (20 mL) was added. The solution was cooled to 0° C., and aq. 5% aq. NaHCO₃ (20 mL) solution was slowly added. The organic layer was separated, dried over Na₂SO₄, filtered, and concentrated under reduced pressure to give the title compound (2.70 g, 81% Yield, 50% purity) as a colorless oil, which was used without purification in subsequent reactions.

LCMS: m/z=251.2/253.1 [M−H]−, (ESI−), RT=0.83, Method B

### Example 114—Human ACLY (hACLY) RF/MS Activity Assay

Compounds of the disclosure were evaluated for their efficacy in inhibiting hACLY using rapid fire mass spectrometry (RF/MS).

The final reaction volume for each compound was 20 μl and consisted of buffer (50 mM Hepes pH 8.0, 10 mM MgCl₂, 0.003% BSA, 0.01% Brij35, 50 mM NaCl, 4 mM DTT) and 1 nM hACLY, EV12992, PP6692) using Greiner, 384 well small volume, deep well plates (Cat #784201). A two-fold dilution series with a top concentration of 10 μM was used to record a concentration response curve. Both the substrate (CoA) and product (Acetyl-CoA) were quantified, and given a ratio. The ratio was normalized using both a negative (0% inhibition) and positive (100% inhibition) control to determine the % inhibition. The final DMSO-concentration was 1% (v/v). Compounds were pre-incubated for 30 min with the buffered enzyme solution at RT (20° C.), and substrate solution was added (final concentrations: 15 μM Coenzyme A, 50 μM ATP and 50 μM citrate) to initiate the enzyme reaction. The enzyme reaction was incubated for additional 30 min at RT. The reaction was quenched upon addition of 40 μl of 5% Formic acid in H₂O and centrifuged (4350 rpm at 20° C. for 10 min).

Product formation was quantified using a RF/MS-based method based on mass change of CoA (substrate) to Acetyl-CoA (product). The results are provided in Table 2.

Table 2. A is less than 20 nM; B is at least 20 nM but less than 100 nM; C is at least 100 nM but less than 10000 nM; and D is 10000 nM or greater.

TABLE 2
hACLY RM/MS activity assay data

| Compound No. | RFMS IC50 GMean |
|---|---|
| 2 | B |
| 3 | C |
| 4 | B |
| 5 | C |
| 6 | C |
| 7 | A |
| 8 | B |
| 9 | D |
| 10 | B |
| 11 | D |
| 12 | D |
| 13 | D |
| 14 | D |
| 15 | D |
| 16 | B |
| 17 | C |
| 18 | B |
| 19 | C |
| 20 | A |
| 21 | A |
| 22 | B |
| 23 | A |
| 24 | B |
| 25 | D |
| 26 | A |
| 27 | B |
| 28 | B |
| 29 | B |
| 30 | B |
| 31 | C |
| 32 | A |
| 33 | B |
| 34 | C |
| 35 | D |
| 36 | B |
| 37 | C |
| 38 | B |
| 39 | A |
| 40 | C |
| 41 | A |
| 42 | B |
| 43 | B |
| 44 | C |
| 45 | B |
| 46 | A |
| 47 | B |
| 48 | A |
| 49 | D |
| 50 | A |
| 51 | C |
| 52 | B |
| 53 | C |
| 54 | A |
| 55 | A |
| 56 | A |
| 57 | B |
| 58 | B |
| 59 | D |
| 60 | D |
| 61 | D |
| 62 | D |
| 63 | D |
| 64 | D |
| 65 | D |
| 66 | B |
| 67 | A |
| 68 | A |
| 69 | C |
| 70 | B |
| 71 | A |
| 72 | A |
| 73 | B |
| 74 | B |
| 75 | C |
| 76 | B |
| 77 | A |
| 78 | A |
| 79 | A |
| 80 | A |
| 81 | A |
| 82 | A |
| 83 | A |
| 84 | C |
| 85 | A |
| 86 | C |
| 87 | B |
| 88 | C |
| 89 | C |
| 90 | C |
| 91 | C |
| 92 | C |
| 93 | B |

### Example 115—hACLY ADP-Glo™ Activity Assay

Compounds of the disclosure were evaluated for their efficacy in inhibiting hACLY using an ADP-Glo™ assays that measure ADP formed from the enzymatic activity of hACLY.

Test compounds were 3-fold serially diluted in DMSO over 11-point concentration range and dispensed onto a 384-well plate. Recombinant human ACLY full length protein was purified. Concentrations of ACLY protein, sodium citrate, coenzyme A, and ATP in the reaction were optimized for standardized homogenous enzyme assay using ADP-Glo™ Kinase (Promega Inc.). The assay measured ADP formed from the enzymatic reaction.

The reaction buffer consisted of the assay buffer (50 mM HEPES pH 8.0, 10 mM MgCl₂, 4 mM 1,4-Dithiothreitol, 0.01% Brij® 35).

ACLY protein (0.5 nM) was added to the prepared reaction buffer, and the mixture was dispensed into the assay plate and incubated for 30 minutes at room temperature. Next, 15 μM sodium citrate, 1 μM coenzyme A, and 80 μM ATP were added into the assay plate and incubated for 60 minutes at room temperature. The final reaction volume for each well was 5 μL.

5 μL of ADP-Glo™ reagent was added, and the mixture was incubated for 40 minutes at room temperature. 10 μl ADP-Glo™ detection reagent was added, and the mixture was incubated for 30 minutes at room temperature. Luminescence was determined using EnVision microplate reader with ultra-luminescence module (Perkin Elmer Inc). Concentration-response curve-fitting and IC50 determination was performed using Xlfit Software (version 5.5.0) with a four-parameter logistic regression fit model. The results are provided in Table 3.

Table 3. A is less than 1 nM; B is at least 1 nM but less than 10 nM; C is at least 10 nM but less than 1000 nM; and D is 1000 nM or greater.

TABLE 3
hACLY ADP-Glo ™ activity assay data

| Compound No. | ADP-Glo IC50 GMean |
|---|---|
| 1 | D |
| 2 | B |
| 3 | C |
| 4 | B |
| 5 | C |
| 6 | C |
| 7 | A |
| 8 | B |
| 9 | D |
| 10 | B |
| 15 | D |
| 16 | A |
| 17 | C |
| 18 | B |
| 19 | C |
| 20 | A |

### Example 116—InCell Pulse Assay

This assay demonstrates the ability of the exemplified compounds described herein to permeate HEK293 cells and bind to and stabilize ACLY. HEK293 cells transfected for 24 hours with pICP-ACLY(FL)-ePL (3 μg DNA per T25 flask) using FuGene HD (Promega Corp.) were harvested and cryopreserved. On the day of the assay, the frozen transfected cells were thawed at 37° C., and the storage medium (DMEM with 2 mM L-Glutamine, 10% FBS and a final concentration of DMSO 10%) was exchanged with assay medium (OptiMEM; Gibco™, ThermoFischer Scientific). Compounds were 3-fold serially diluted in DMSO over 11-point concentration range. 100 nL of the corresponding dilutions were spotted into the assay plate (Greiner AG; 384 Well, PP, Small Volume, Deep Well, Natural, cat. no. 784201) followed by the addition of 20 μL of cell suspension adjusted with assay medium to 7.5E4 cells/mL. Subsequently, the plates were sealed and incubated for 1 hour at 37° C. Afterwards, the plates were fixed upside down in a rack and incubated in a 55° C. water bath for 10 minutes. Following this heat pulse step, plates were allowed to reach room temperature for 10 minutes and centrifuged for a few seconds at 1000 rpm and room temperature. 25 μL EA detection solution (working solution: 0.0167× InCell EA Reagent, 0.15× InCell Dilution Buffer, 0.167× InCell Lysis Buffer; 0.667× InCell Substrate Reagent; InCell Hunter Detection kit; Eurofins DiscoverX; cat. no. 96-0079) were added to each well. Afterwards, 42 μL were transferred from the assay plate to the detection plate (Corning Inc., 384-well Low Flange Black Flat Bottom Polystyrene NBS Microplate, cat. no. 3575), followed by an incubation for 1 hour at room temperature, a centrifugation step for 1 minute at 1000 rpm and the detection of the luminescence signal using an EnVision microplate reader (Perkin Elmer Inc.). Concentration-response curve-fitting and IC50 determination was performed using Evotec's in-house evaluation software APlus with a four-parameter non-linear regression fit model. Data was normalized to 0.5% (final concentration) DMSO as low/negative control and 1 μM (final concentration) NDI-094311 as high/positive control. The results are provided in Table 4.

Table 4. A is less than 0.3 μM; B is at least 0.3 μM but less than 1 μM; C is at least 1 μM but less than 50 μM; and D is 50 μM or greater.

TABLE 4
InCell Pulse assay data

| Compound No. | InCell Pulse IC50 GMean |
|---|---|
| 2 | C |
| 3 | D |
| 4 | C |
| 5 | D |
| 6 | D |
| 7 | A |
| 8 | B |
| 9 | D |
| 10 | A |
| 12 | D |
| 13 | D |
| 14 | D |
| 15 | D |
| 16 | A |
| 17 | D |
| 18 | B |
| 19 | D |
| 20 | B |
| 21 | A |
| 22 | D |
| 23 | A |
| 24 | A |
| 26 | B |
| 27 | D |
| 28 | B |
| 29 | B |
| 30 | C |
| 31 | C |
| 32 | A |
| 33 | B |
| 34 | D |
| 35 | D |
| 36 | D |
| 37 | D |
| 38 | D |
| 39 | A |
| 40 | B |
| 41 | A |
| 42 | A |
| 43 | B |
| 44 | C |
| 45 | A |
| 46 | A |
| 47 | A |
| 48 | A |
| 49 | D |
| 50 | A |
| 51 | D |
| 52 | B |
| 53 | D |
| 54 | A |
| 55 | B |
| 56 | C |
| 57 | C |
| 58 | A |
| 59 | D |
| 60 | D |
| 61 | D |
| 62 | D |
| 63 | D |
| 64 | D |
| 65 | D |
| 66 | |
| 67 | B |
| 68 | A |
| 69 | A |
| 70 | D |
| 71 | D |
| 72 | A |
| 73 | A |
| 74 | A |
| 75 | B |
| 76 | C |
| 77 | C |
| 78 | A |
| 79 | A |
| 80 | A |
| 81 | A |
| 82 | A |
| 83 | A |
| 84 | A |
| 85 | D |
| 86 | B |
| 87 | D |
| 88 | D |
| 89 | D |
| 90 | D |
| 91 | D |
| 92 | C |
| 93 | D |

### Example 117—Fatty Acid Synthesis (FAS) Assay in HepG2 Cells

This assay demonstrates the ability of the exemplified compounds described herein to inhibit fatty acid synthesis. HepG2 cells were seeded into white clear-bottom 96-well plates (50,000/well) coated with collagen and incubated at 37° C. for 16-24 hours. Thereafter the plates were washed once with 100 μL PBS (+CaCl₂+MgCl₂) and 50 μL assay medium (RPMI 1860 containing 11 mM glucose, 10 mM HEPES and 1 nM Insulin) per well was added. Test substances in assay medium were added (10 μL, 0.5% DMSO) and incubated for 20 min at 37° C. After that 10 μL assay medium containing ¹⁴C-citrate (final concentration 50 μM) and PrestoBlue reagent (lx final concentration; ThermoFisher) were added and incubated for 90 min (37° C., 5% CO₂). Cell viability was measured (Multiskan FC, microplate reader, absorbance 570 nm). Medium was removed by inverting the plate and cells were washed once with 100 μL ice-cold PBS. Cells were lysed with 50 μL lysis buffer (0.1 N NaOH, 0.1% Triton X-100) and plate was sealed with Tape Pads and vortexed. For the saponification reaction plates were incubated for 16-24 hours at 80° C. Seals were removed and 200 μL 0.1 N HCl was added for neutralisation of the pH. 150 μL from each well was transferred into the corresponding wells of a 96-well Flash plate (Perkin Elmer) and sealed with TopSeal A (Perkin Elmer). Plates were incubated for 4 h at 70° C., and for 1 h at room temperature in the dark. Signal of ¹⁴C radioactivity was measured (TopCount). As a positive control 5 mM unlabelled citrate or C75 (Sigma, C5490) was applied. The results are provided in Table 5.

Table 5. A is less than 2 μM; B is at least 2 μM but less than 10 μM; C is at least 10 μM but less than 50 μM; and D is 50 μM or greater.

TABLE 5
Fatty acid synthesis (FAS) assay data

| Compound No. | FAS IC50 GMean |
|---|---|
| 2 | D |
| 4 | C |
| 6 | C |
| 7 | A |
| 8 | C |
| 9 | D |
| 10 | B |
| 15 | B |
| 16 | A |
| 17 | D |
| 18 | B |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein.

Scope of the disclosure is thus indicated by the appended claims rather than by the foregoing description, and all changes that come within the meaning and range of equivalency of the claims are intended to be embraced therein.

We claim:

1. A compound of formula (I) 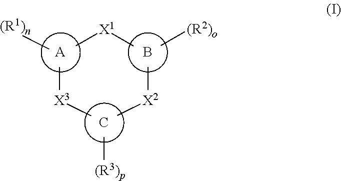
or a stereoisomer and/or a pharmaceutically acceptable salt thereof, wherein:
Ring A is phenyl, pyridinyl, or pyridonyl, wherein the nitrogen atom of the pyridonyl may optionally be substituted by C₁₋₆alkyl;
Ring B is phenyl or 5-10 membered heterocyclyl;
Ring C is phenyl, 5-10 membered heterocyclyl, or 5-10 membered heteroaryl; or
Ring C is absent;
R¹ is independently, for each occurrence, selected from the group consisting of halogen, hydroxyl, C₁₋₆alkyl, C₁₋₆haloalkyl, C₁₋₆alkoxy, C₁₋₆haloalkoxy, —O—C(O)C₁₋₆alkyl, —O—C(O)C₃₋₆cycloalkyl, and N(R^{E})₂;
R² is independently, for each occurrence, selected from the group consisting of halogen, hydroxyl, C₁₋₆alkyl, C₁₋₆haloalkyl, C₁₋₆alkoxy, C₁₋₆haloalkoxy, C₃₋₆cycloalkyl, and COOH;
R³ is selected from the group consisting of cyano, halogen, C₁₋₆alkyl, and C₃₋₆cycloalkyl;
X¹ is selected from the group consisting of *—S(O)₂N(R^{A})—**, —C(O)—, *—C(O)N(R^{A})—**, *—CH₂N(R^{A})—**, and *—S(O)₂CH₂—**, wherein * denotes the point of attachment to Ring A and ** denotes the point of attachment to Ring B;
X² is a bond or —O—;
X³ is #-L¹-L²-L³-##, wherein # denotes the point of attachment to Ring A and ##denotes the point of attachment to Ring C or to Ring B when Ring C is absent;
L¹ is selected from the group consisting of —CH₂—, —O—, —C(O)—, —C(O)N(R^{B})—, —C(O)O—C₁₋₆alkyl-O—, —CH₂—C(O)O—, —CH₂—N(R^{B})C(O)—, and 5-6 membered heteroaryl;
L² is C₁₋₆alkyl or 4-6 membered heterocyclyl, wherein the 4-6 membered heterocyclyl may be optionally substituted with oxo; or
L² is absent;
L³ is selected from the group consisting of a bond, —O—, —O—C₁₋₆alkyl, C₁₋₆alkyl-O—, and 4-6 membered heterocyclyl;
R^{A} is hydrogen or C₁₋₆alkyl;
R^{B} is hydrogen or C₁₋₆alkyl;
R^{E} is independently, for each occurrence, hydrogen or C₁₋₆alkyl;
n is 0, 1, or 2;
o is 1 or 2; and
p is 0 or 1.

2. The compound of claim 1, wherein Ring A is phenyl, pyridinyl, or 2-pyridonyl, wherein the nitrogen atom of the 2-pyridonyl may optionally be substituted by C₁₋₆alkyl.

3. The compound of claim 1 or 2, wherein Ring A is phenyl, pyridinyl, or 2-pyridonyl, wherein the nitrogen atom of the 2-pyridonyl may optionally be substituted by CH₃.

4. The compound of any one of claims 1-3, wherein Ring A is selected from the group consisting of 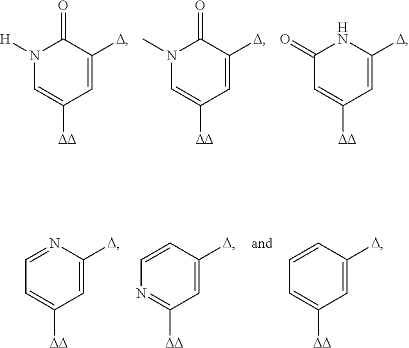
wherein Δ denotes the point of attachment to X¹ and AA denotes the point of attachment to X³.

5. The compound of any one of claims 1-4, wherein n is 0.

6. The compound of any one of claims 1-4, wherein n is 1.

7. The compound of claim 6, wherein R¹ is selected from the group consisting of chloro, hydroxyl, CH₃, CHF₂, and NH₂.

8. The compound of any one of claims 1-4, wherein n is 2.

9. The compound of claim 8, wherein R¹ is independently, for each occurrence, selected from the group consisting of chloro, fluoro, hydroxyl, CH₃, CHF₂, —O—CH₃, —O—CHF₂, —O—C(O)CH₃, and

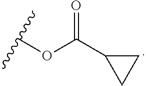 [SMILES: *OC(=O)C1CC1]

10. The compound of any one of claims 1-9, wherein Ring B is phenyl or 9-membered heterocyclyl.

11. The compound of any one of claims 1-10, wherein Ring B is phenyl or

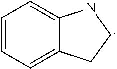

12. The compound of any one of claims 1-10, wherein Ring B is 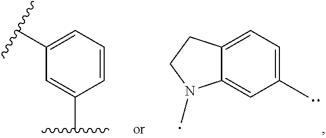
wherein ● denotes the point of attachment to X¹ and ●● denotes the point of attachment to X².

13. The compound of any one of claims 1-12, wherein o is 1.

14. The compound of claim 13, wherein R² is selected from the group consisting of fluoro, hydroxyl, cyclopropyl, CF₃, —O—CH₃, —O—CHF₂, —O—CF₃, and C(O)OH.

15. The compound of any one of claims 1-12, wherein o is 2.

16. The compound of claim 15, wherein R² is independently, for each occurrence, selected from the group consisting of chloro, fluoro, CH₃, CF₃, and —O—CH₃.

17. The compound of any one of claims 1-16, wherein Ring C is absent.

18. The compound of any one of claims 1-16, wherein Ring C is selected from the group consisting of phenyl, pyrrolidinyl, piperidinyl, pyridinyl,

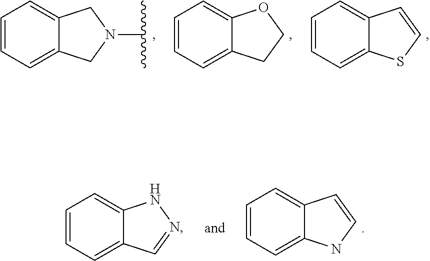

19. The compound of any one of claims 1-16 or 18, wherein Ring C is selected from the group consisting of 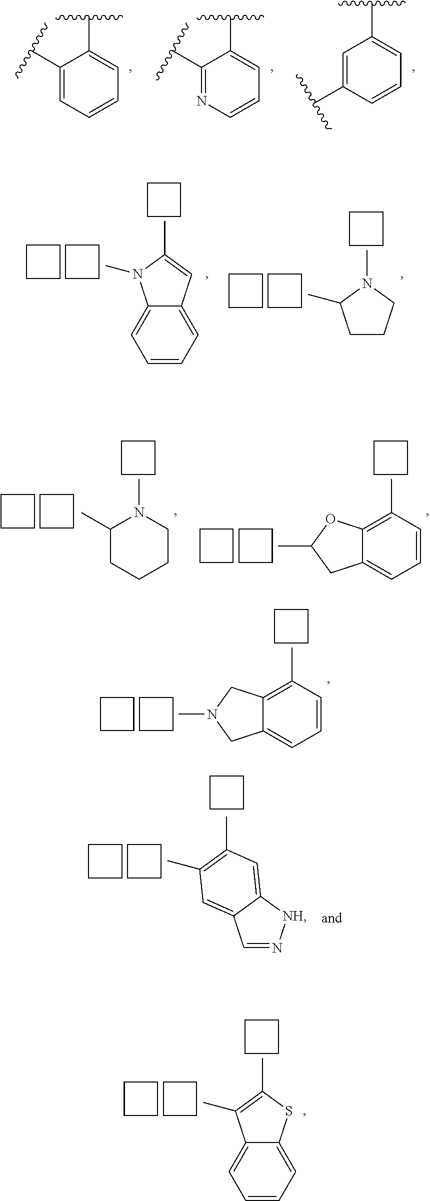
wherein □ denotes the point of attachment to X² and □□ denotes the point of attachment to X³.

20. The compound of any one of claims 1-16, 18, and 19, wherein p is 1.

21. The compound of claim 20, wherein R³ is selected from the group consisting of bromo, chloro, fluoro, cyano, CH₃, and cyclopropyl.

22. The compound of any one of claims 1-16, 18, and 19, wherein p is 0.

23. The compound of any one of claims 1-22, wherein X¹ is selected from the group consisting of *—S(O)₂N(H)—**, *—S(O)₂N(CH₃)—**, —C(O)—, *—C(O)N(H)—**, *—CH₂N(H)—**, and *—S(O)₂CH₂—**, wherein * denotes the point of attachment to Ring A and ** denotes the point of attachment to Ring B.

24. The compound of any one of claims 1-23, wherein X² is a bond.

25. The compound of any one of claims 1-23, wherein X² is —O—.

26. The compound of any one of claims 1-25, wherein L¹ is selected from the group consisting of —C(O)N(H)—, —C(O)N(CH₃)—, —C(O)O—, —CH₂—, —CH₂—O—, —C(O)—, —CH₂—C(O)O—, —CH₂—N(CH₃)C(O)—, —O—, and

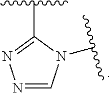 [SMILES: *c1nncn1*]

27. The compound of any one of claims 1-26, wherein L² is selected from the group consisting of —CH₂CH₂—, —(CH₂)₃—, —CH₂—, —(CH₂)₄—, —CH(CH₃)CH₂—, —CH₂CH₂C(H)(CH₃)—,

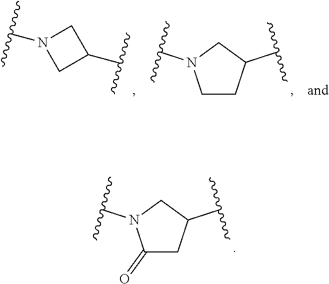

28. The compound of any one of claims 1-26, wherein L² is absent.

29. The compound of any one of claims 1-28, wherein L³ is a bond.

30. The compound of any one of claims 1-28, wherein L³ is —O—, —CH₂—O—, or

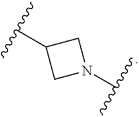 [SMILES: *C1CN(*)C1]

31. The compound of any one of claims 1-25, wherein X³ is selected from the group consisting of 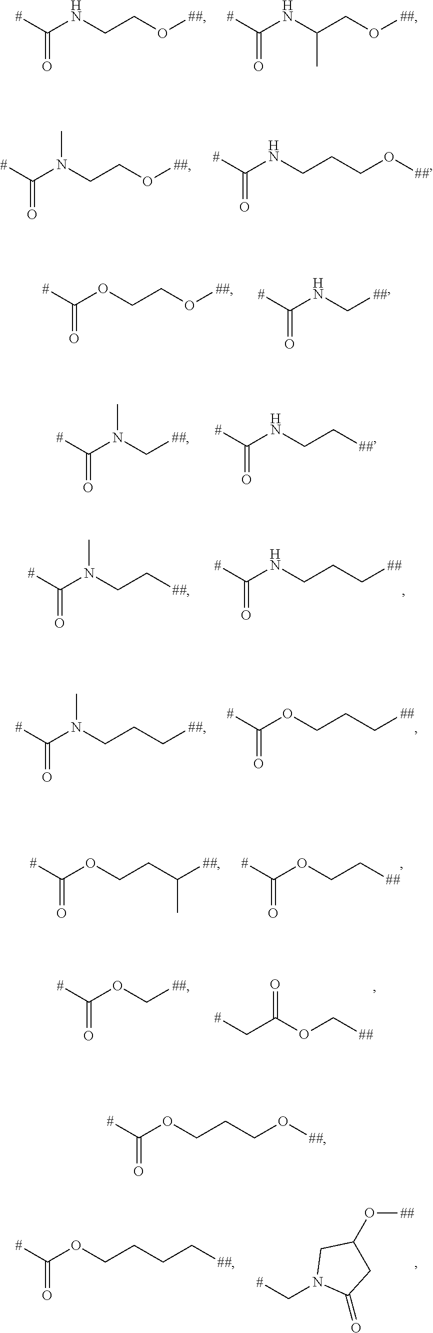 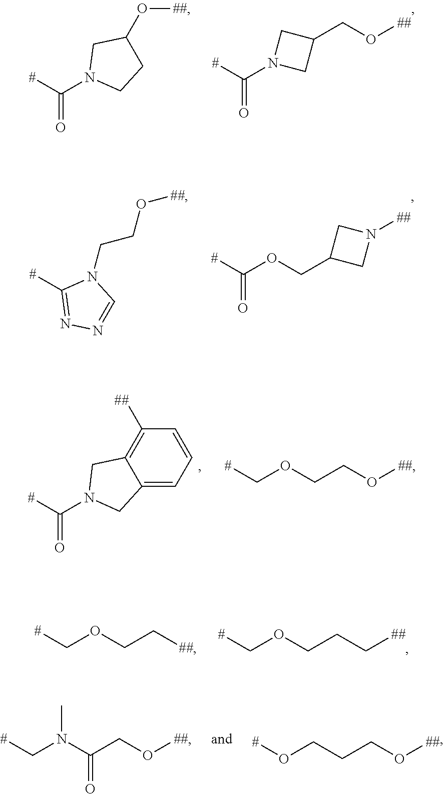
wherein # denotes the point of attachment to Ring A and ## denotes the point of attachment to Ring C.

32. A compound of formula (Ia) 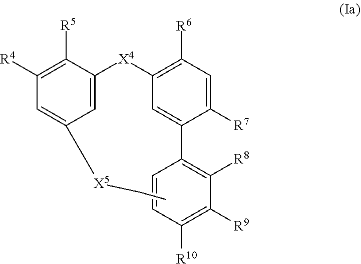
or a stereoisomer and/or a pharmaceutically acceptable salt thereof, wherein:
R⁴ is selected from the group consisting of hydrogen, hydroxyl, halogen, and C₁₋₆alkyl;
R⁵ is selected from the group consisting of hydrogen, halogen, hydroxyl, C₁₋₆alkoxy, C₁₋₆haloalkyl, C₁₋₆haloalkoxy, —C(O)OC₁₋₆alkyl, and —C(O)OC₁₋₆cycloalkyl;
R⁶ is selected from the group consisting of hydrogen, halogen, hydroxyl, C₁₋₆alkoxy, C₁₋₆haloalkoxy, C₁₋₆alkyl, C₁₋₆haloalkyl, C₃₋₆cycloalkyl, and C(O)OH;
R⁷ is selected from the group consisting of hydrogen, halogen, C₁₋₆alkyl, C₁₋₆haloalkyl, and C₁₋₆alkoxy;
R⁸ is hydrogen, halogen, or C₁₋₆alkyl;
R⁹ is selected from the group consisting of hydrogen, cyano, halogen, and C₃₋₆cycloalkyl;
R¹⁰ is hydrogen, cyano, or halogen;
X⁴ is selected from the group consisting of *—S(O)₂N(R^{C})—**, *—C(O)N(R^{C})—**, *—CH₂N(R^{C})—**, and *—S(O)₂CH₂—**, wherein * denotes the point of attachment to 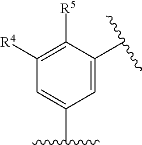
and ** denotes the point of attachment to 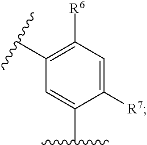
X⁵ is #-L⁴-L⁵-L⁶-##, wherein # denotes the point of attachment to 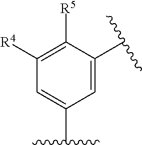
and ## denotes the point of attachment to 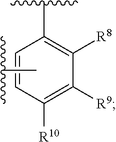
L⁴ is selected from the group consisting of CH₂, C₁₋₆alkyl-O—, —O—, —C(O)—, —C(O)N(R^{D})—, —C(O)O—, —CH₂—C(O)O—, —CH₂—N(R^{D})C(O)—, and 5-6 membered heteroaryl;
L⁵ is C₁₋₆alkyl or 4-6 membered heterocyclyl, wherein the 4-6 membered heterocyclyl may be optionally substituted with oxo;
L⁶ is selected from the group consisting of a bond, —O—, C₁₋₆alkyl-O—, and 4-6 membered heterocyclyl;
R^{C} is hydrogen or C₁₋₆alkyl; and
R^{D} is hydrogen or C₁₋₆alkyl.

33. The compound of claim 32, wherein R⁴ is selected from the group consisting of hydrogen, hydroxyl, chloro, and CH₃.

34. The compound of claim 32 or 33, wherein R⁵ is selected from the group consisting of hydrogen, fluoro, hydroxyl, CHF₂, —O—CH₃, —O—CHF₂, —O—C(O)CH₃, and

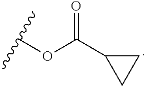 [SMILES: *OC(=O)C1CC1]

35. The compound of any one of claims 32-34, wherein R⁶ is selected from the group consisting of hydrogen, hydroxyl, fluoro, chloro, cyclopropyl, CF₃, —O—CH₃, —O—CHF₂, —O—CF₃, and C(O)OH.

36. The compound of any one of claims 32-35, wherein R⁷ is selected from the group consisting of hydrogen, chloro, fluoro, —O—CH₃, CH₃, and CF₃.

37. The compound of any one of claims 32-36, wherein R⁸ is hydrogen, CH₃, or chloro.

38. The compound of any one of claims 32-37, wherein R⁹ is selected from the group consisting of hydrogen, cyano, chloro, bromo, fluoro, and cyclopropyl.

39. The compound of any one of claims 32-38, wherein R¹⁰ is selected from the group consisting of hydrogen, cyano, chloro, and fluoro.

40. The compound of any one of claims 32-39, wherein X⁴ is selected from the group consisting of *—S(O)₂N(H)—**, *—S(O)₂N(CH₃)—**, *—C(O)N(H)—**, *—CH₂N(H)—**, and *—S(O)₂CH₂—**, wherein * denotes the point of attachment to 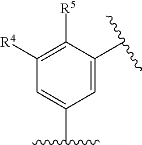
and ** denotes the point of attachment

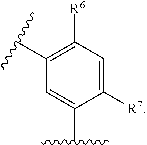

41. The compound of any one of claims 32-40, wherein L⁴ is selected from the group consisting of —CH₂—, —O—, —C(O)—, —C(O)N(H)—, —C(O)N(CH₃)—, —C(O)O—, —CH₂—C(O)O—, —CH₂—N(CH₃)C(O)—, —CH₂—O—, and

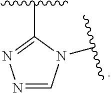 [SMILES: *c1nncn1*]

42. The compound of any one of claims 32-41, wherein L⁵ is selected from the group consisting of —CH₂—, —CH₂CH₂—, —(CH₂)₃—, —C(H)(CH₃)CH₂—, —CH₂CH₂C(H)(CH₃)—, —(CH₂)₄—,

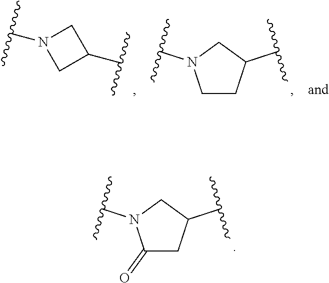

43. The compound of any one of claims 32-42, wherein L⁶ is a bond.

44. The compound of any one of claims 32-42, wherein L⁶ is —O—, —CH₂—O—, or

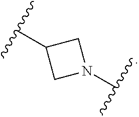 [SMILES: *C1CN(*)C1]

45. The compound of any one of claims 32-40, wherein X⁵ is selected from the group consisting of 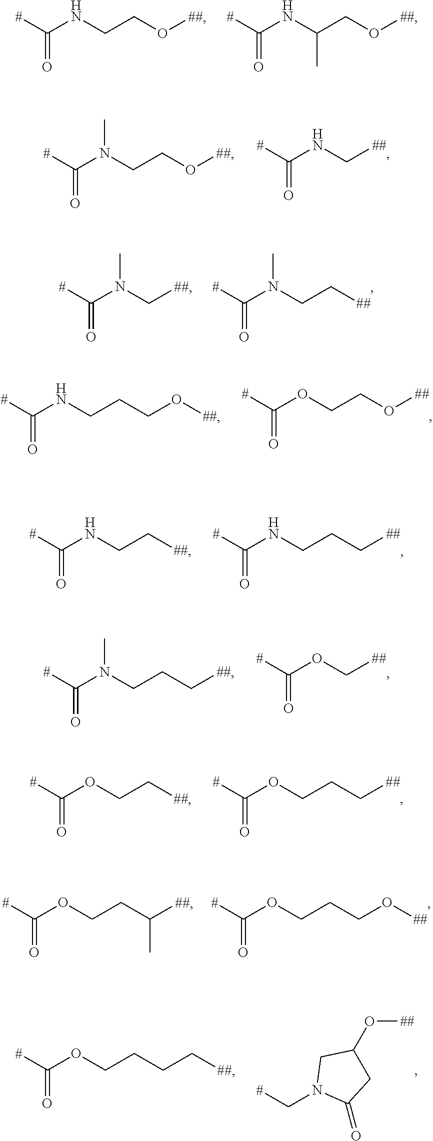 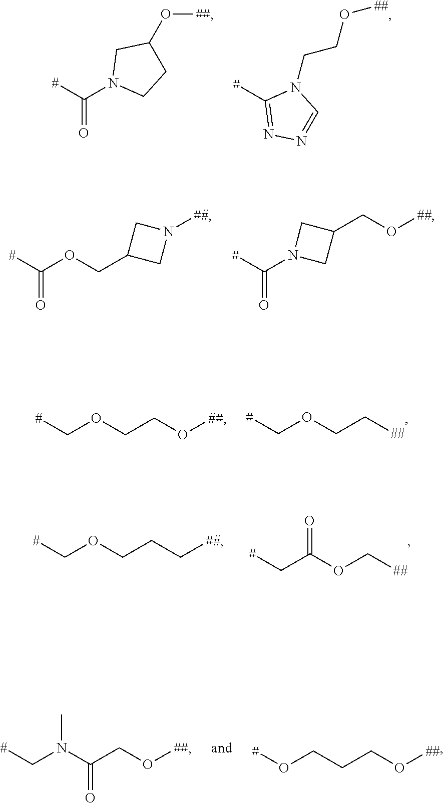
wherein # denotes the point of attachment to 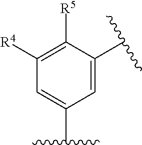
and ## denotes the point of attachment to

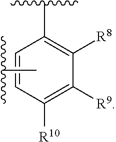

46. A compound selected from any compound set forth in Table 1, or a pharmaceutically acceptable salt thereof.

47. A pharmaceutical composition comprising a compound of any one of claims 1-46; and a pharmaceutically acceptable carrier.

48. A method of inhibiting ACLY in a subject in need thereof, the method comprising administering to the subject a therapeutically effective amount of a compound of any one of claims 1-46 or a pharmaceutical composition of claim 47.

49. The method of claim 48, wherein the subject has a liver condition, disease, or disorder.

50. The method of claim 49, wherein the liver condition, disease, or disorder is NAFLD or NASH.

51. The method of claim 48, wherein the subject has type-2 diabetes.

52. The method of claim 48, wherein the subject has inflammation.

53. The method of claim 48, wherein the subject has chronic kidney disease.

54. The method of claim 48, wherein the subject has autoimmunity.

55. The method of claim 48, wherein the subject has cancer.

56. A method of treating NAFLD in a subject in need thereof, the method comprising administering to the subject a therapeutically effective amount of a compound of any one of claims 1-46 or a pharmaceutical composition of claim 47.

57. A method of treating NASH in a subject in need thereof, the method comprising administering to the subject a therapeutically effective amount of a compound of any one of claims 1-46 or a pharmaceutical composition of claim 47.

58. A method of treating type-2 diabetes in a subject in need thereof, the method comprising administering to the subject a therapeutically effective amount of a compound of any one of claims 1-46 or a pharmaceutical composition of claim 47.

59. A method of treating inflammation in a subject in need thereof, the method comprising administering to the subject a therapeutically effective amount of a compound of any one of claims 1-46 or a pharmaceutical composition of claim 47.

60. A method of treating chronic kidney disease in a subject in need thereof, the method comprising administering to the subject a therapeutically effective amount of a compound of any one of claims 1-46 or a pharmaceutical composition of claim 47.

61. A method of treating autoimmunity in a subject in need thereof, the method comprising administering to the subject a therapeutically effective amount of a compound of any one of claims 1-46 or a pharmaceutical composition of claim 47.

62. A method of treating cancer in a subject in need thereof, the method comprising administering to the subject a therapeutically effective amount of a compound of any one of claims 1-46 or a pharmaceutical composition of claim 47.

63. A method of treating a condition, disease, or disorder as described herein in a subject in need thereof, the method comprising administering to the subject a therapeutically effective amount of a compound of any one of claims 1-46 or of the pharmaceutical composition of claim 47.
